(19) United States
(12) Patent Application Publication
(10) Pub. No.: US 2008/0176870 A1
(43) Pub. Date: Jul. 24, 2008

(54) Heterobicyclic metalloprotease inhibitors

(76) Inventors: Bert Nolte, Aachen (DE); Irving Sucholeiki, Winchester, MA (US); Tim Feuerstein, Neckargemuend (DE); Brian M. Gallagher, Merrimac, MA (US); Xinyuan Wu, Newton, MA (US); Christoph Steeneck, Dossenheim (DE); Christian Gege, Ehingen (DE); Hongbo Deng, Southborough, MA (US); Joshua Van Veldhuizen, Seattle, WA (US); Arthur Taveras, Southborough, MA (US)

Correspondence Address: AMGEN INC., MAIL STOP 28-2-C, ONE AMGEN CENTER DRIVE, THOUSAND OAKS, CA, 91320-1799, US

(21) Appl. No.: 11/986,622

(22) Filed: Nov. 20, 2007

Related U.S. Application Data
(60) Provisional application No. 60/860,194, filed on Nov. 20, 2006.

(51) Int. Cl. A61K 31/495; C07D 487/04; A61P 19/02; A61P 29/00; A61P 9/10

(52) U.S. Cl. 514/259.31; 544/263; 544/281; 514/259.1

(57) ABSTRACT

The present invention relates generally to heterobicyclic containing pharmaceutical agents, and in particular, to heterobicyclic metalloprotease inhibitor compounds. More particularly, the present invention provides a new class of heterobicyclic metalloprotease inhibiting compounds that exhibit an increased potency in relation to currently known metalloprotease inhibitors.

This application claims the benefit of U.S. Provisional Application No. 60/860,194, filed Nov. 20, 2006, which is hereby incorporated by reference.

### FIELD OF THE INVENTION

The present invention relates generally to heterobicyclic metalloprotease inhibiting compounds, and more particularly to heterobicyclic MMP inhibiting compounds.

### BACKGROUND OF THE INVENTION

Matrix metalloproteinases (MMPs) and aggrecanases (ADAMTS=a disintegrin and metalloproteinase with thrombospondin motif) are a family of structurally related zinc-containing enzymes that have been reported to mediate the breakdown of connective tissue in normal physiological processes such as embryonic development, reproduction, and tissue remodelling. Over-expression of MMPs and aggrecanases or an imbalance between extracellular matrix synthesis and degradation has been suggested as factors in inflammatory, malignant and degenerative disease processes. MMPs and aggrecanases are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The ADAMTSs are a group of proteases that are encoded in 19 ADAMTS genes in humans. The ADAMTSs are extracellular, multidomain enzymes whose functions include collagen processing, cleavage of the matrix proteoglycans, inhibition of angiogenesis and blood coagulation homeostasis (Biochem. J. 2005, 386, 15-27; Arthritis Res. Ther. 2005, 7, 160-169; Curr. Med. Chem. Anti-Inflammatory Anti-Allergy Agents 2005, 4, 251-264).

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, II, III) and gelatins, proteoglycans, cytokines and other components of ECM (extracellular matrix).

The activation of the MMPs involves the removal of a propeptide, which features an unpaired cysteine residue complexes the catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective MMP inhibiting compounds comprises several factors, including choice of selective versus broad-spectrum MMP inhibitors and rendering such compounds bioavailable via an oral route of administration.

### SUMMARY OF THE INVENTION

The present invention relates to a new class of heterobicyclic amide containing pharmaceutical agents which inhibits metalloproteases. In particular, the present invention provides a new class of metalloprotease inhibiting compounds that exhibit potent MMP-13 inhibiting activity and/or activity towards MMP-3, MMP-8, MMP-12, ADAMTS-4, and ADAMTS-5.

The present invention provides several new classes of amide containing heterobicyclic metalloprotease compounds of the following general formula:

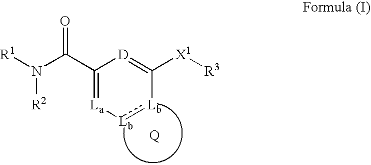

wherein all variables in the preceeding Formula (I) are as defined hereinbelow.

The heterobicyclic metalloprotease inhibiting compounds of the present invention may be used in the treatment of metalloprotease mediated diseases, such as rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, chronic wound healing, wound healing, hemorrhoid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, liver fibrosis, lupus, malaria, meningitis, multi-organ dysfunction, necrotizing enterocolitis, osteoporosis, periodontitis, chronic periodontitis, peritonitis associated with continuous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

In particular, the heterobicyclic metalloprotease inhibiting compounds of the present invention may be used in the treatment of MMP-13 mediated osteoarthritis and may be used for other MMP-13 mediated symptoms, inflammatory, malignant and degenerative diseases characterized by excessive extracellular matrix degradation and/or remodelling, such as cancer, and chronic inflammatory diseases such as arthritis, rheumatoid arthritis, osteoarthritis atherosclerosis, abdominal aortic aneurysm, inflammation, multiple sclerosis, and chronic obstructive pulmonary disease, and pain, such as inflammatory pain, bone pain and joint pain.

The present invention also provides heterobicyclic metalloprotease inhibiting compounds that are useful as active ingredients in pharmaceutical compositions for treatment or prevention of metalloprotease—especially MMP-13, MMP-3, MMP-8, MMP-12, ADAMTS-4, and ADAMTS-5—mediated diseases. The present invention also contemplates use of such compounds in pharmaceutical compositions for oral or parenteral administration, comprising one or more of the heterobicyclic metalloprotease inhibiting compounds disclosed herein.

The present invention further provides methods of inhibiting metalloproteases, by administering formulations, including, but not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular formulations, comprising the heterobicyclic metalloprotease inhibiting compounds by standard methods known in medical practice, for the treatment of diseases or symptoms arising from or associated with metalloprotease, especially MMP-13, MMP-3, MMP-8, MMP-12, ADAMTS-4, and ADAMTS-5, including prophylactic and therapeutic treatment. Although the most suitable route in any given case will depend on the nature and severity of the conditions being treated and on the nature of the active ingredient. The compounds from this invention are conveniently presented in unit dosage form and prepared by any of the methods well-known in the art of pharmacy.

The heterobicyclic metalloprotease inhibiting compounds of the present invention may be used in combination with a disease modifying antirheumatic drug, a nonsteroidal anti-inflammatory drug, a COX-2 selective inhibitor, a COX-1 inhibitor, an immunosuppressive, a steroid, a biological response modifier or other anti-inflammatory agents or therapeutics useful for the treatment of chemokines mediated diseases.

### DETAILED DESCRIPTION OF THE INVENTION

One aspect of the invention relates to a compound having the structure:

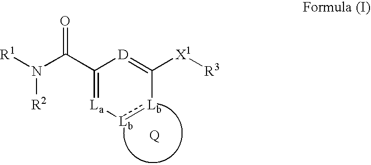

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is hydrogen, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰SR²¹, CH₂R²⁰, CHR²⁰R²¹, OR¹⁰, OR²¹, NR¹⁰NR⁹, R⁵²,

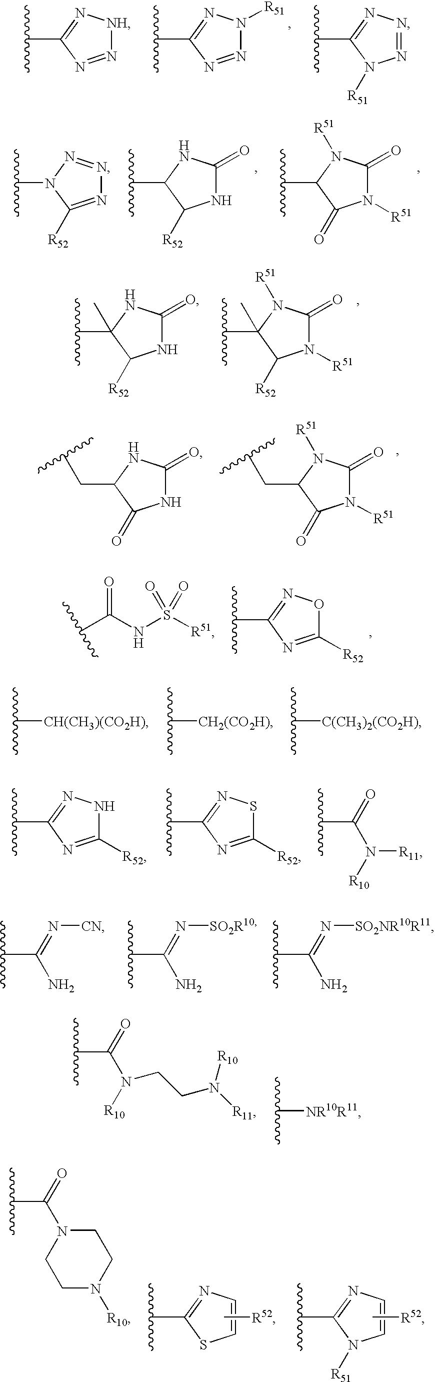

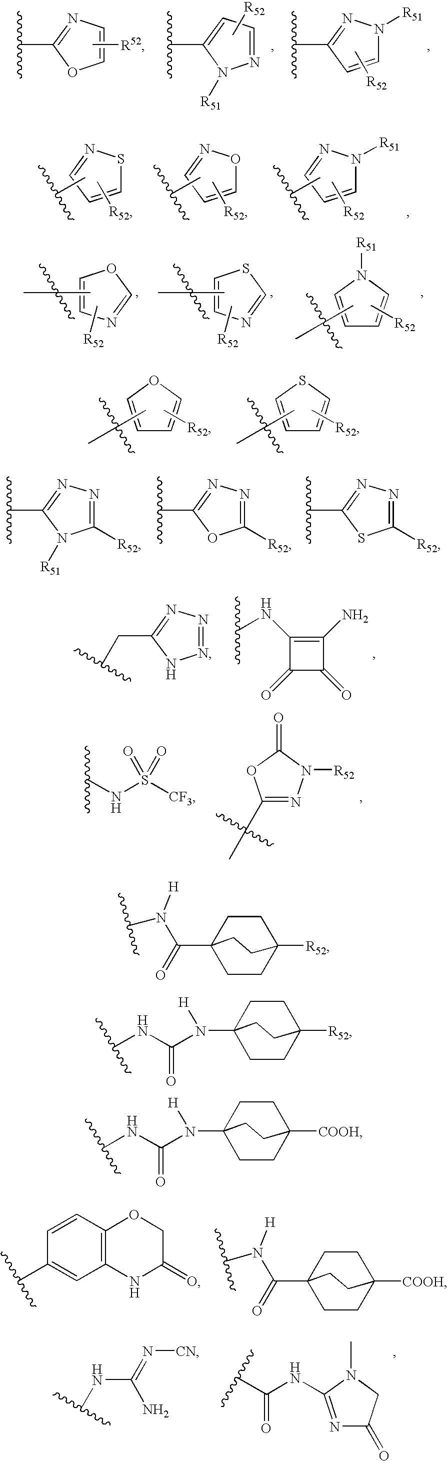

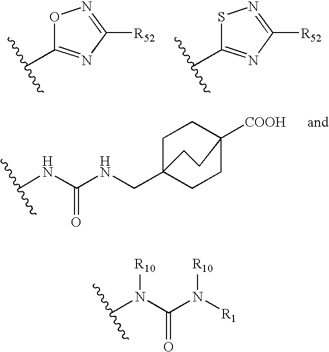

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹″, S(O), —(C₀-C₆)-alkyl-C(O)OR¹⁰, S(O), —(C₀-C₆)-alkyl-C(O)NR¹⁰R″, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R″, (C₀-C₆)-alkyl-NR¹⁰—S(O)_{y}R¹⁰, O—(C₀-C₆)-alkyl-aryl and 0-(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R″, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R^{3e}, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R″, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R″, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

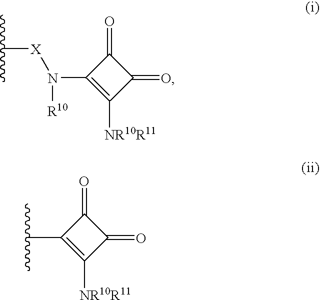

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R″, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R¹¹, SO₂R⁸⁰ and SO₂NR⁸⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

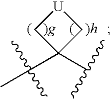

D is a member selected from CR²² and N;

Lₐ is selected from CR⁹ and N;

L_{b} is independently selected from C and N with the provisos that both L_{b} are not N, and that the bond between L_{b} and L_{b} is optionally a double bond only if both are L_{b} are carbon;

Q is a 5- or 6-membered ring selected from aryl and heteroaryl, wherein aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In one embodiment, in conjunction with any above or below embodiments, the compound is selected from:

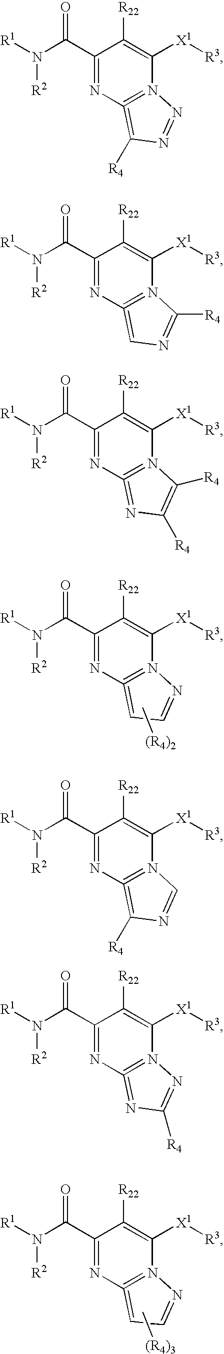

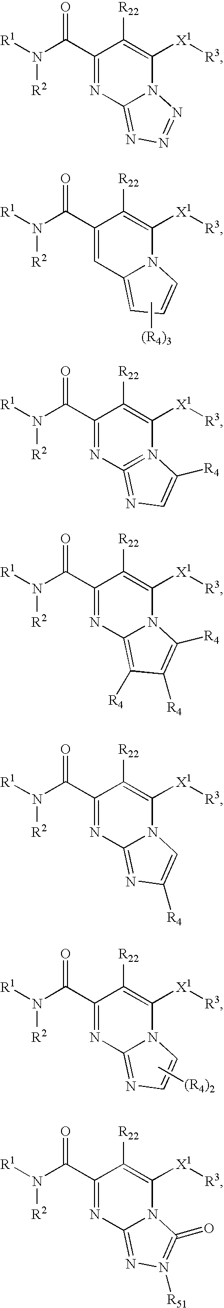

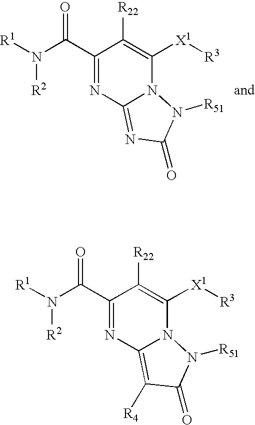

In another embodiment, in conjunction with any above or below embodiments, the compound is selected from:

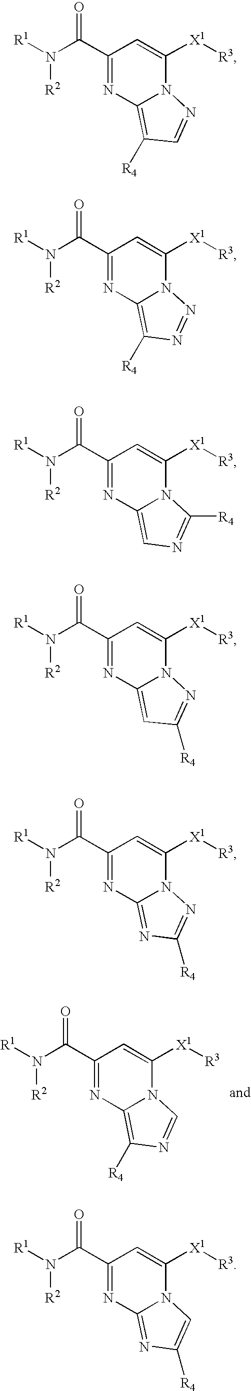

In another embodiment, in conjunction with any above or below embodiments, the compound is selected from:

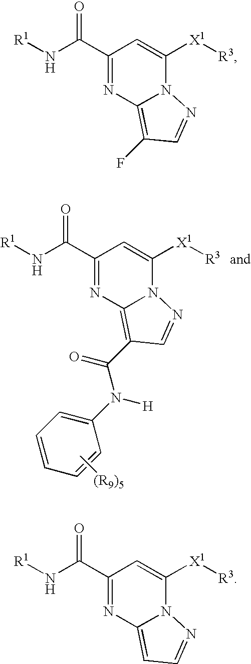

In another embodiment, in conjunction with any above or below embodiments, R³ is selected from:

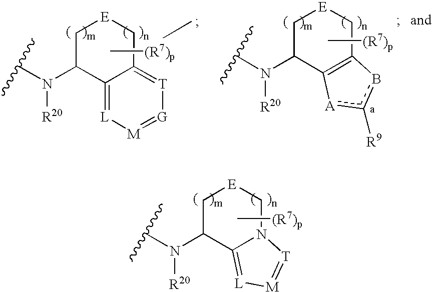

wherein:

R⁷ is independently selected from hydrogen, alkyl, cycloalkyl, halo, R⁴ and NR¹⁰R¹¹, or optionally two R⁷ groups together at the same carbon atom form ═O, ═S or ═NR¹⁰;

A and B are independently selected from CR⁹, CR⁹R¹⁰, NR¹⁰, N, O and S(O)ₓ;

G, L, M and T are independently selected from CR⁹ and N;

m and n are independently selected from 0-3, provided that:
(1) when E is present, m and n are not both 3;
(2) when E is —CH₂—W¹—, m and n are not 3; and
(3) when E is a bond, m and n are not 0; and

p is selected from 0-6;

wherein the dotted line represents a double bond between one of: carbon “a” and A, or carbon “a” and B.

In another embodiment, in conjunction with any above or below embodiments, R³ is selected from:

hydrogen, NR²⁰R²¹, NR¹⁰R¹¹, COR¹⁰, COR²¹, COOR¹⁰, COOR²¹, CR²⁰R²¹R¹, SO₂R¹⁰, SO₂R²¹, SO₂NR¹⁰R¹¹, SO₂NR²⁰R²¹, SOR¹⁰, SOR²¹, PO₂R¹⁰, PO₂R²¹, SR¹⁰, SR²¹, CH₂R²⁰, CHR²⁰R²¹, OR¹⁰, OR²¹, NR¹⁰NR⁹, R⁵²,

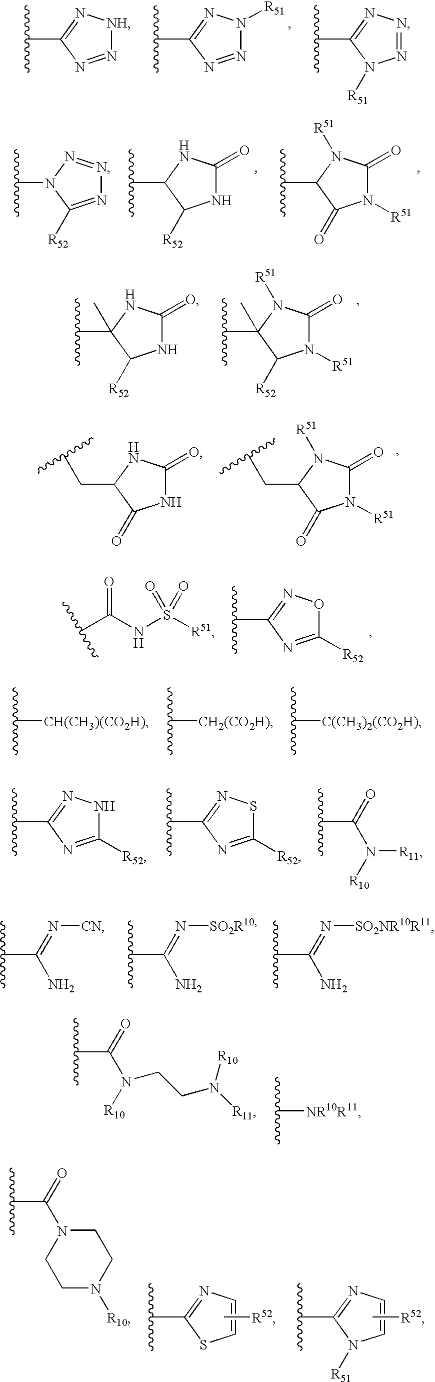

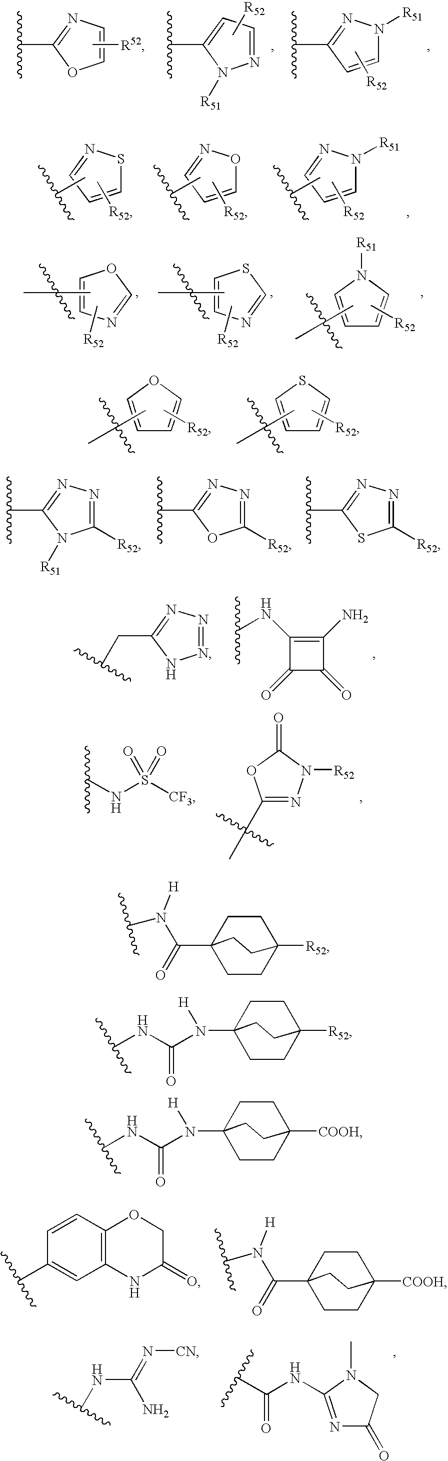

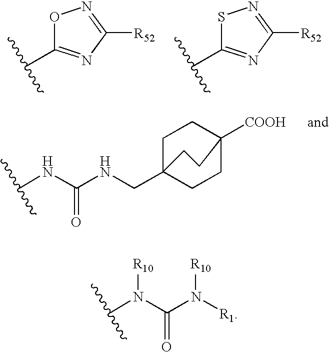

In another embodiment, in conjunction with any above or below embodiments, R³ is selected from:

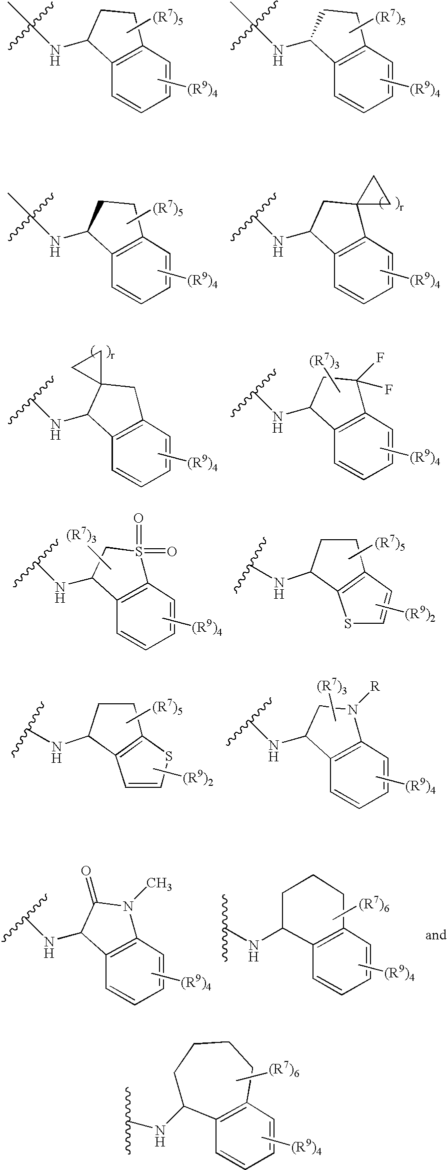

wherein:

R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and

r is selected from 1-4.

In another embodiment, in conjunction with any above or below embodiments, R³ selected from the group consisting of:

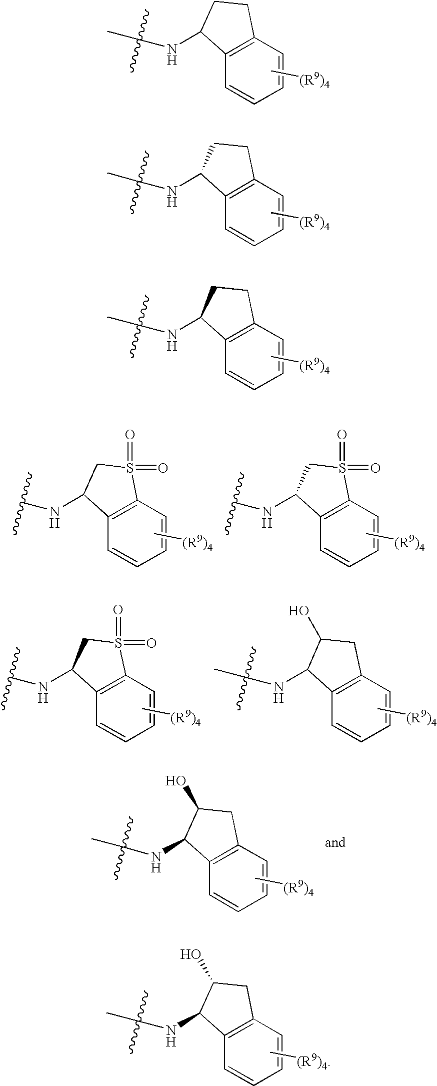

In another embodiment, in conjunction with any above or below embodiments, R⁹ is selected from:

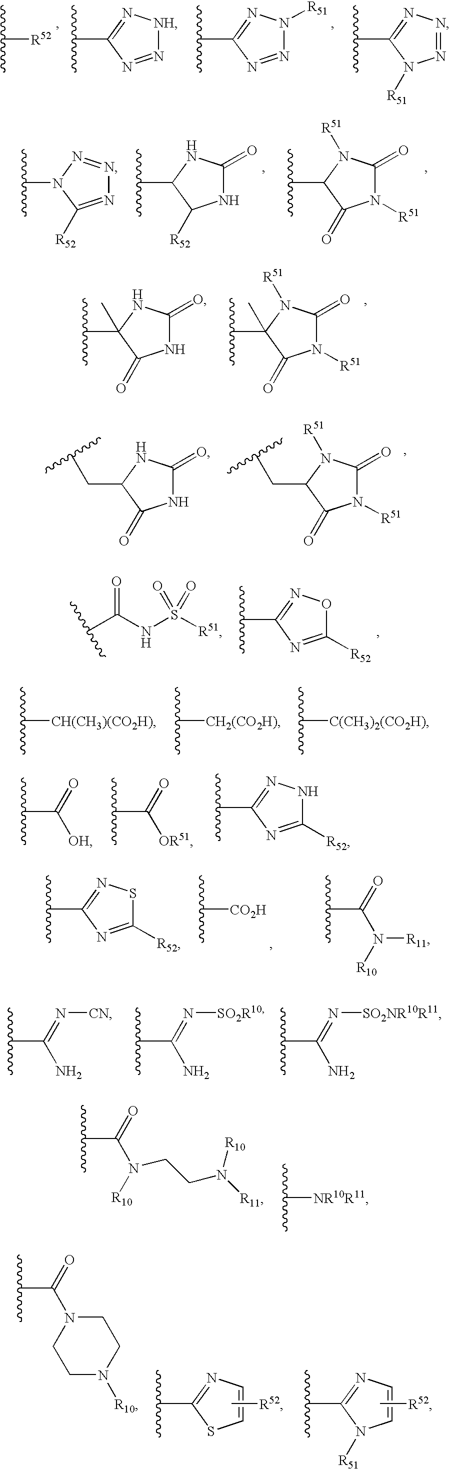

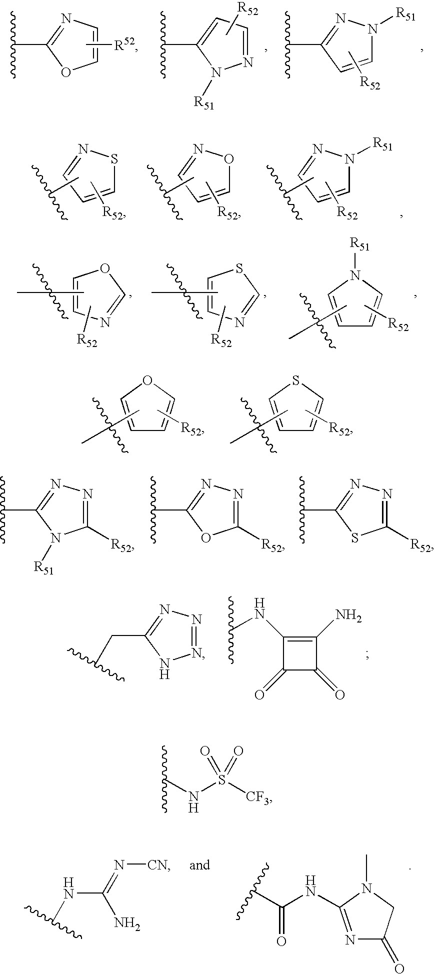

In another embodiment, in conjunction with any above or below embodiments, R³ is

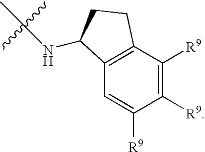

In another embodiment, in conjunction with any above or below embodiments, R³ is selected from:

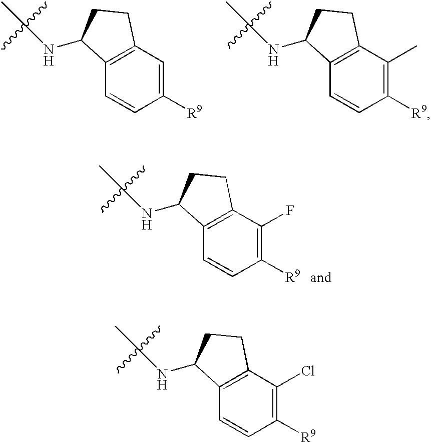

wherein:

R⁹ is selected from hydrogen, fluoro, halo, CN, alkyl, CO₂H,

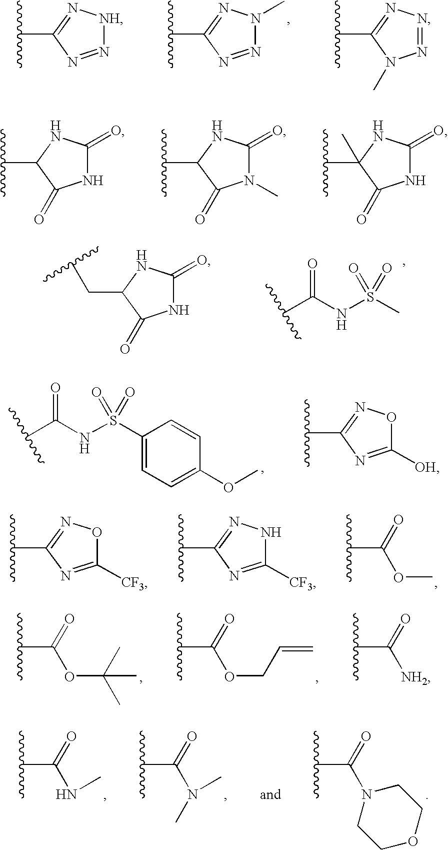

In another embodiment, in conjunction with any above or below embodiments, R¹ is selected from:

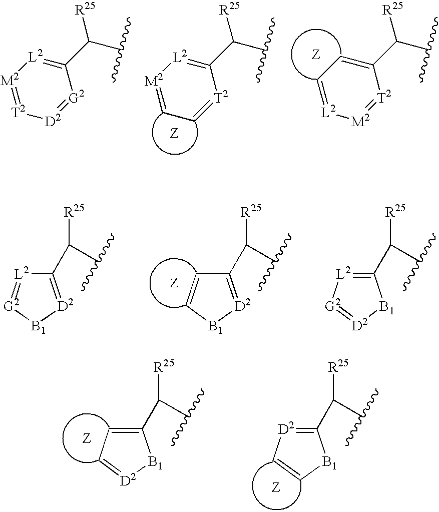

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S(O)ₓ; D², G², L², M² and T² are independently selected from CR¹⁸ and N; and Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

In another embodiment, in conjunction with any above or below embodiments, R¹ is selected from:

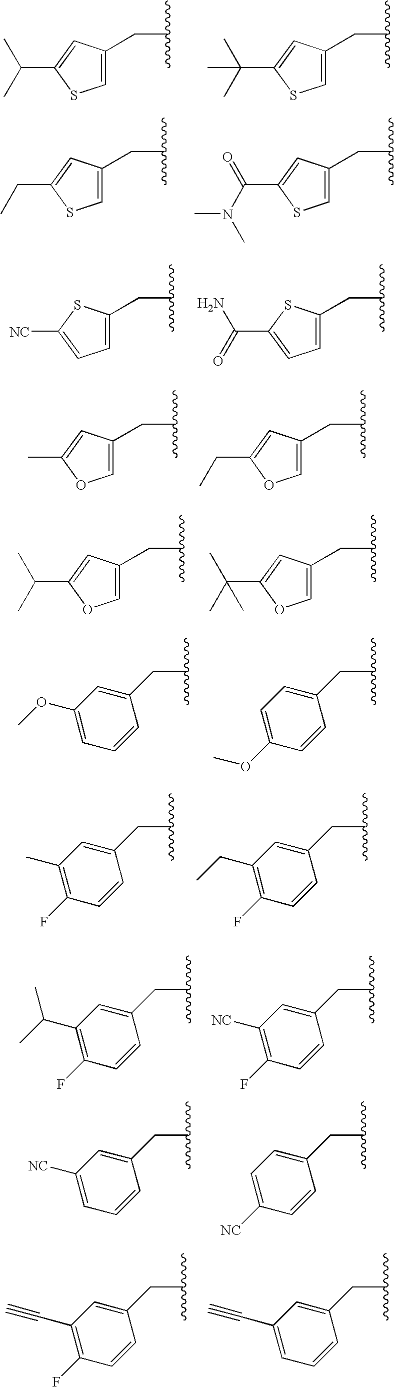

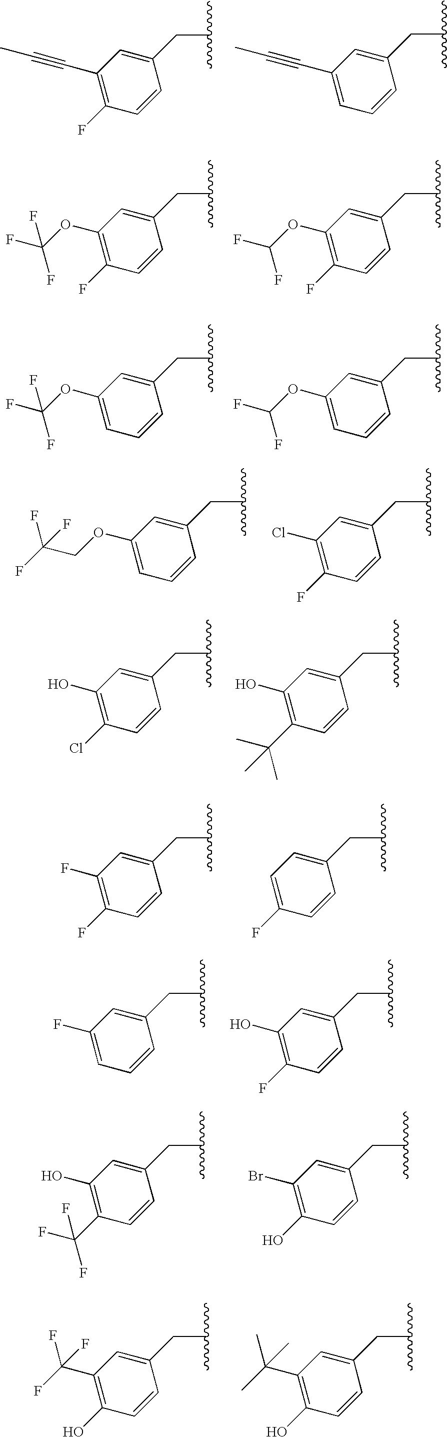

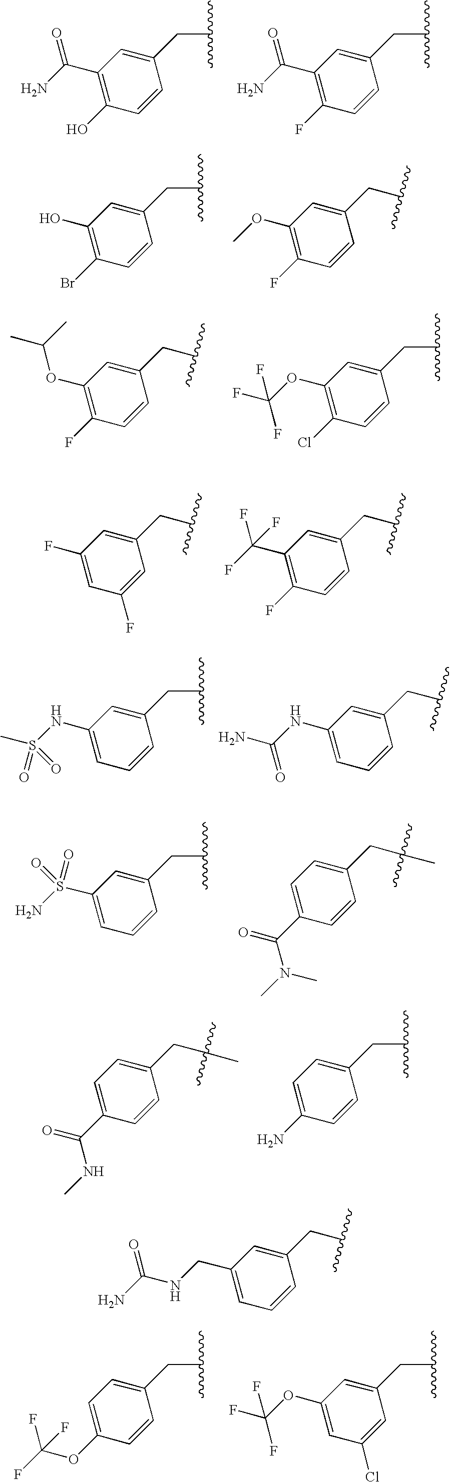

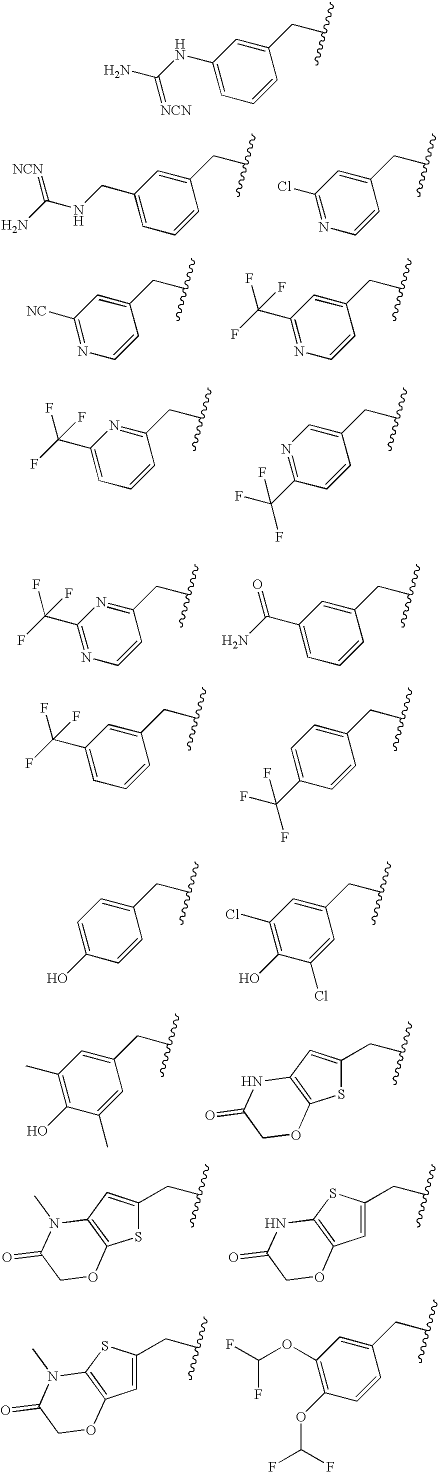

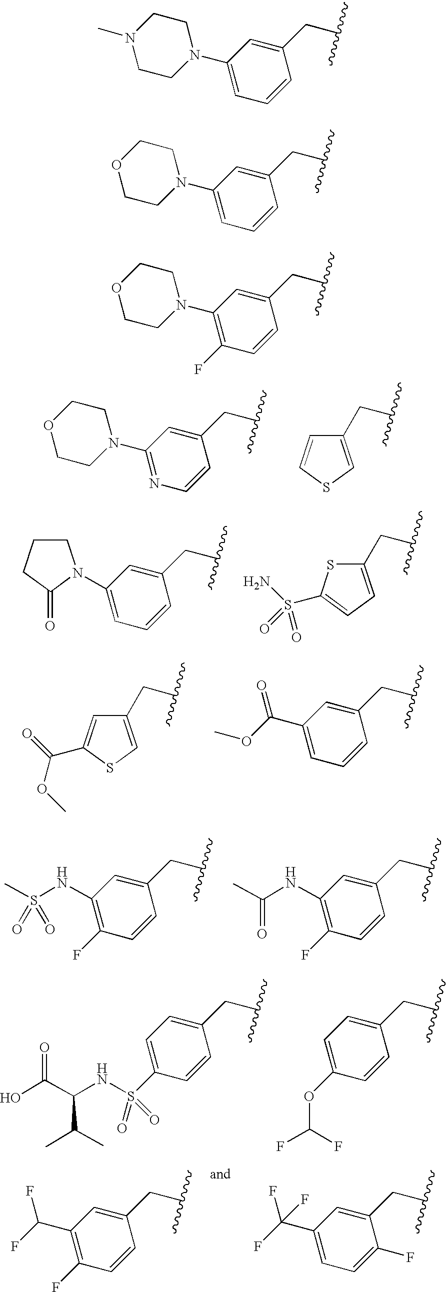

In another embodiment, in conjunction with any above or below embodiments, R¹ is selected from:

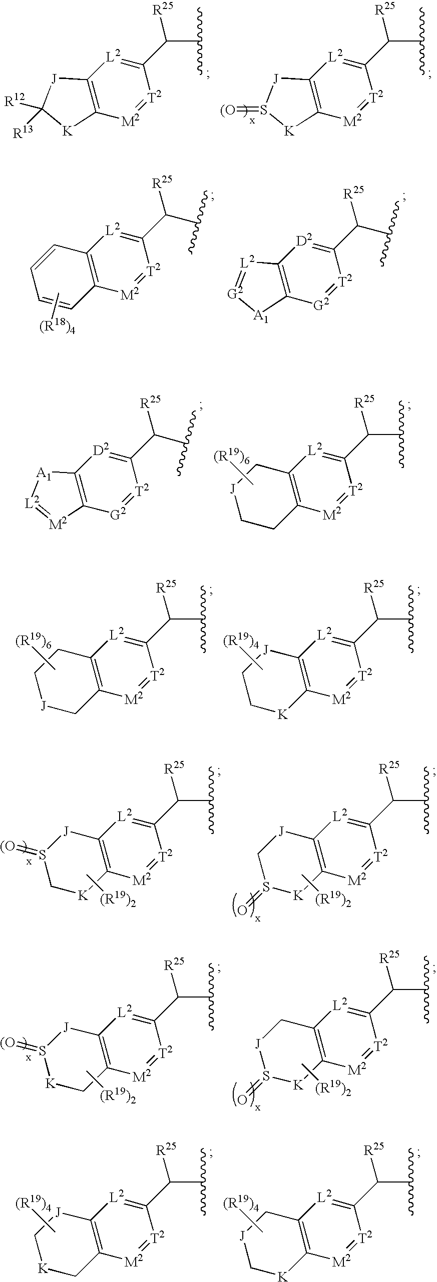

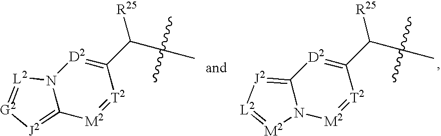

wherein:

R¹² and R¹³ are independently selected from hydrogen, alkyl and halo, wherein alkyl is optionally substituted one or more times, or optionally R¹² and R¹³ together form ═O, ═S or ═NR¹⁰;

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹¹COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

J and K are independently selected from CR¹⁰R¹⁸, NR¹⁰, O and S(O)ₓ;

A₁ is selected from NR¹¹, O and S(O)ₓ; and

D², G², J², L², M² and T² are independently selected from CR¹⁸ and N.

In another embodiment, in conjunction with any above or below embodiments, R¹ is selected from:

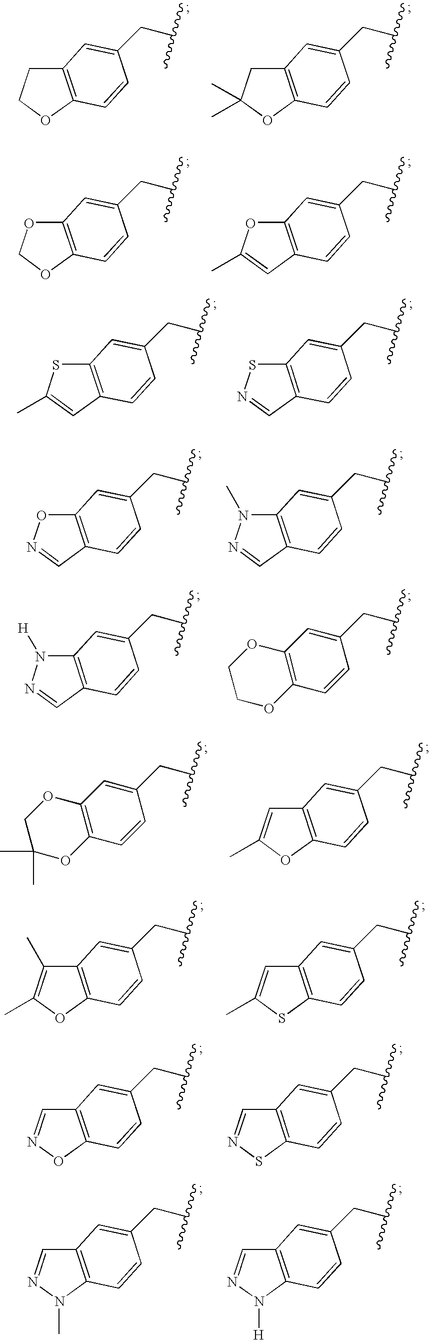

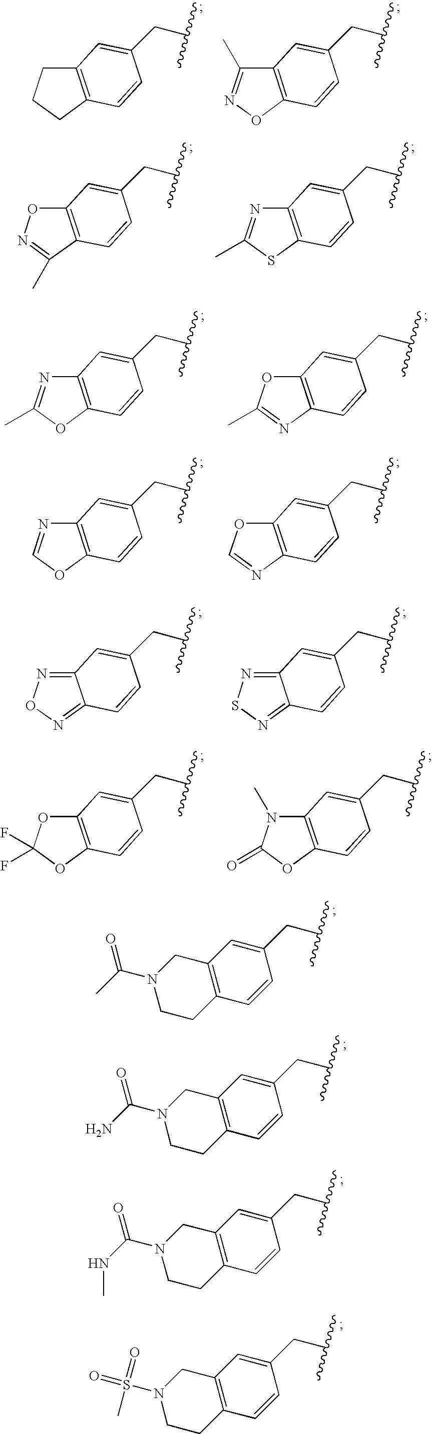

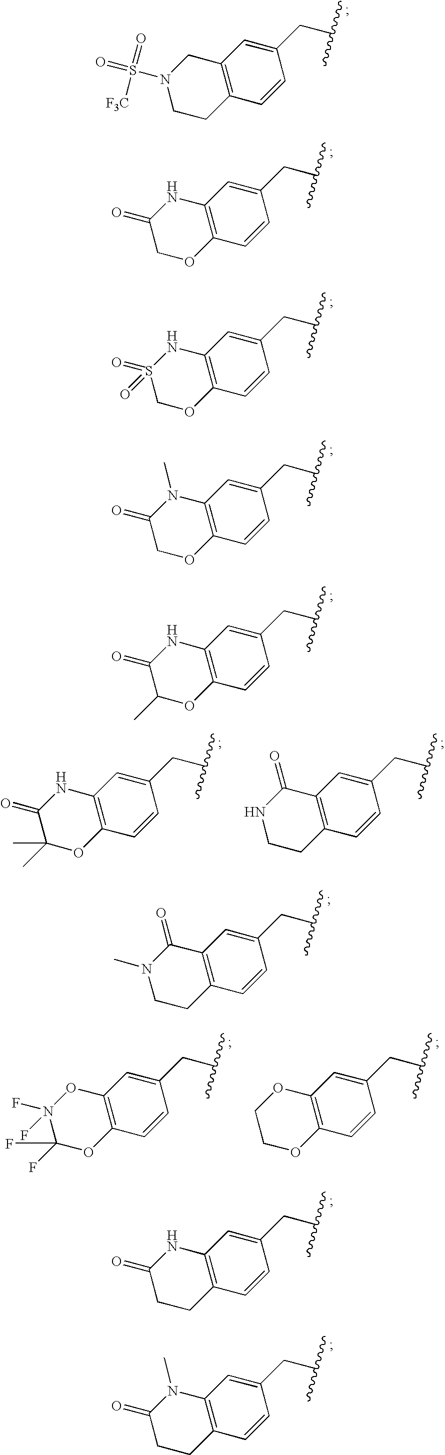

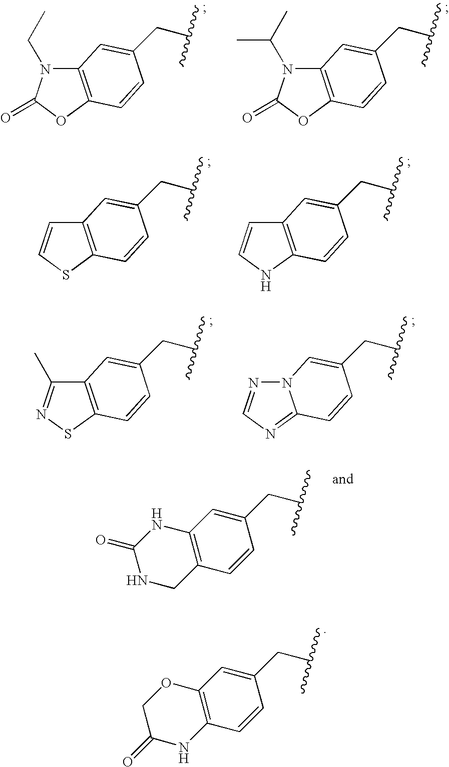

In another embodiment, in conjunction with any above or below embodiments, R¹ is selected from:

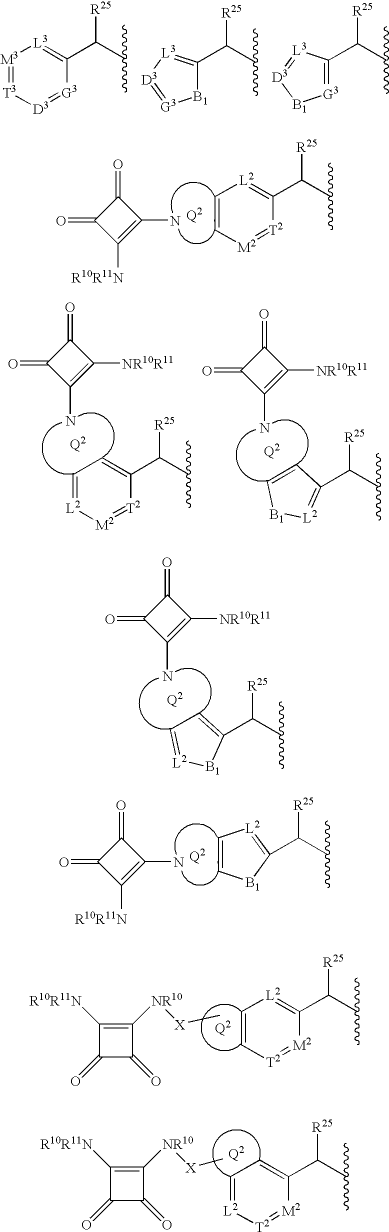

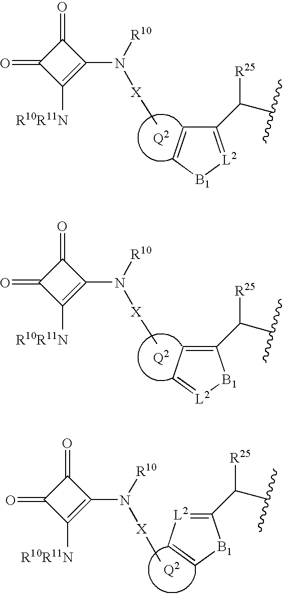

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, CONR¹⁰R¹¹ and haloalkyl, wherein alkyl, cycloalkyl and haloalkyl are optionally substituted one or more times;

L², M², and T² are independently selected from CR¹⁸ and N;

D³, G³, L³, M³, and T³ are independently selected from N, CR¹⁸, (i), and (ii),

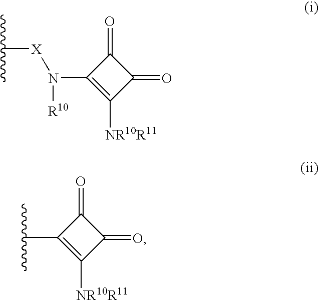

with the proviso that one of L³, M³, T³, D³, and G³ is (i) or (ii)

B₁ is selected from the group consisting of NR¹⁰, O and S(O)ₓ; and

Q² is a 5- to 8-membered ring selected from cycloalkyl, heterocycloalkyl, aryl, and heteroaryl, which is optionally substituted one or more times with R¹⁹.

In another embodiment, in conjunction with any above or below embodiments, R¹ is selected from:

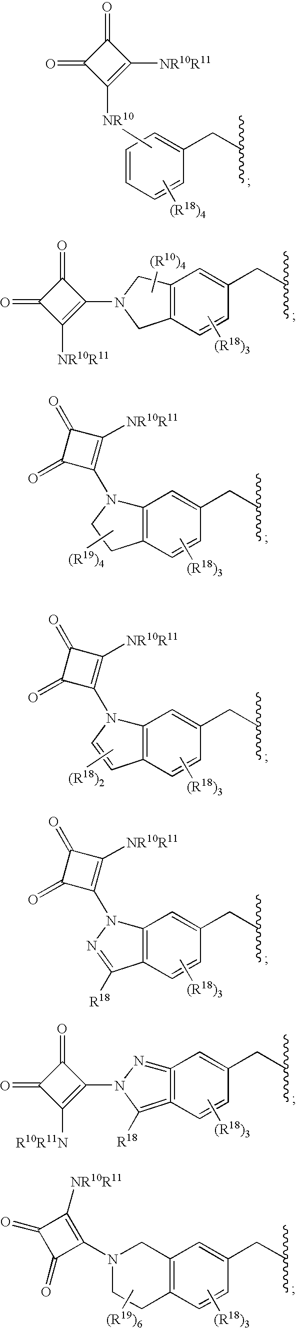

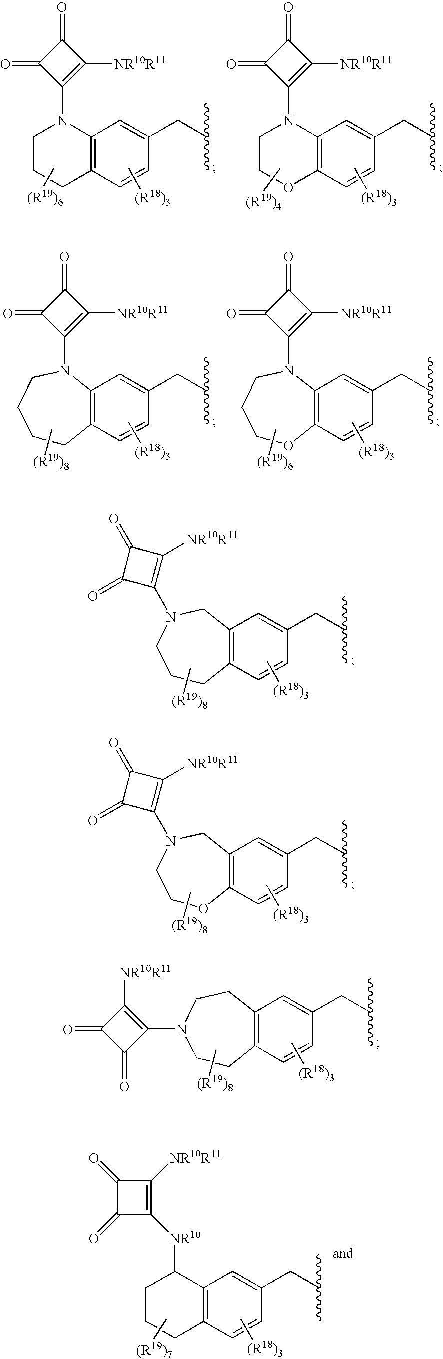

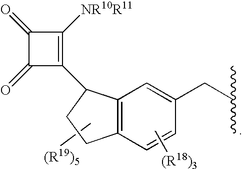

In another embodiment, in conjunction with any above or below embodiments, R¹ is selected from:

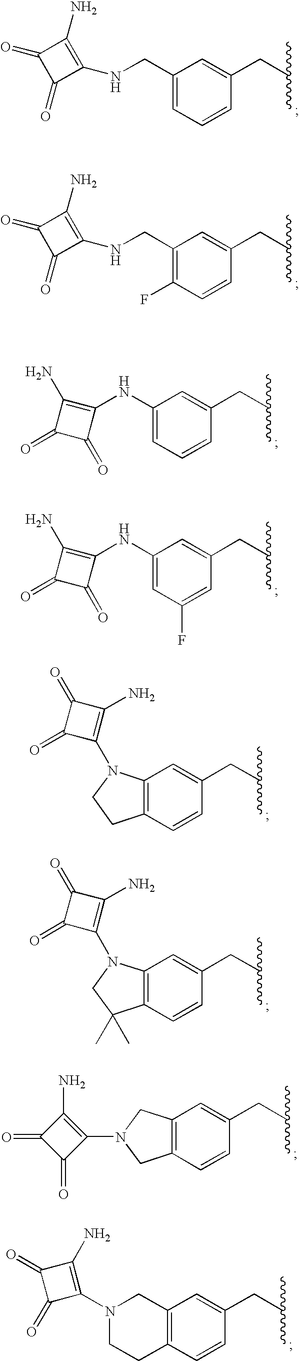

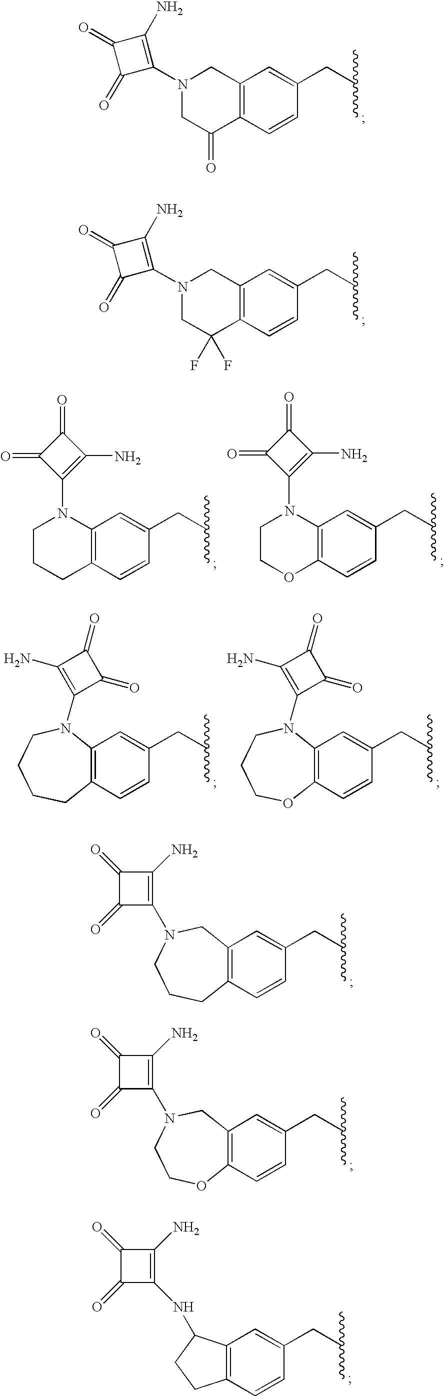

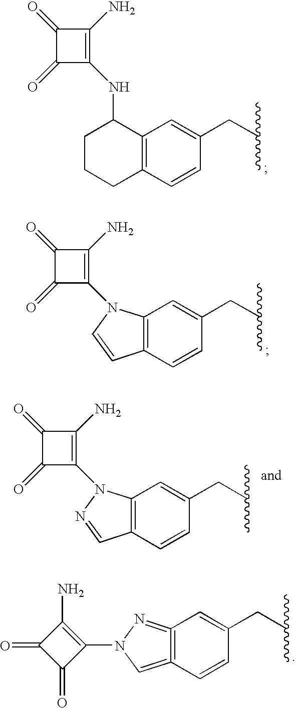

In another embodiment, in conjunction with any above or below embodiments, X¹ is selected from a bond; and R³ is selected from

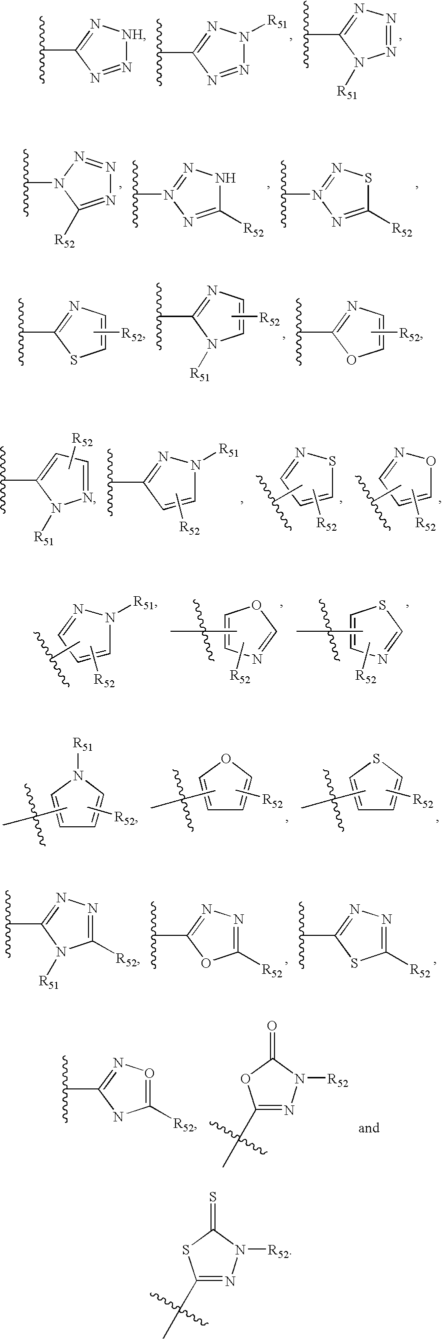

In another embodiment, in conjunction with any above or below embodiments, the compound is selected from:

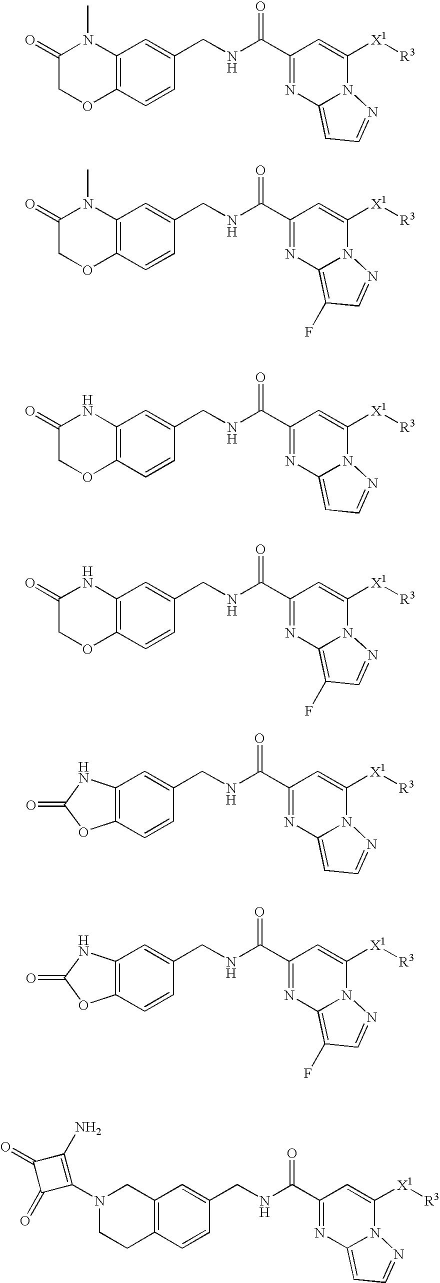

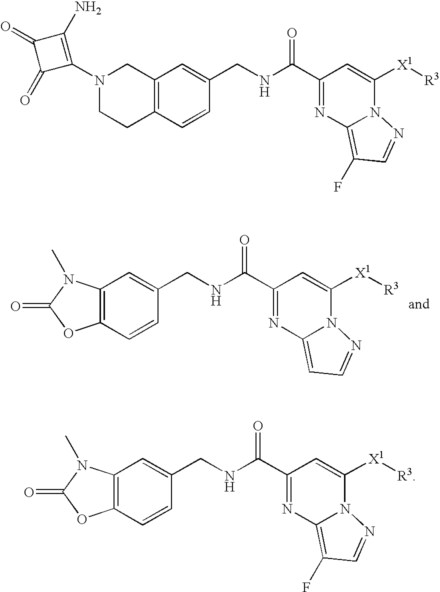

In another embodiment, in conjunction with any above or below embodiments, the compound is selected from:

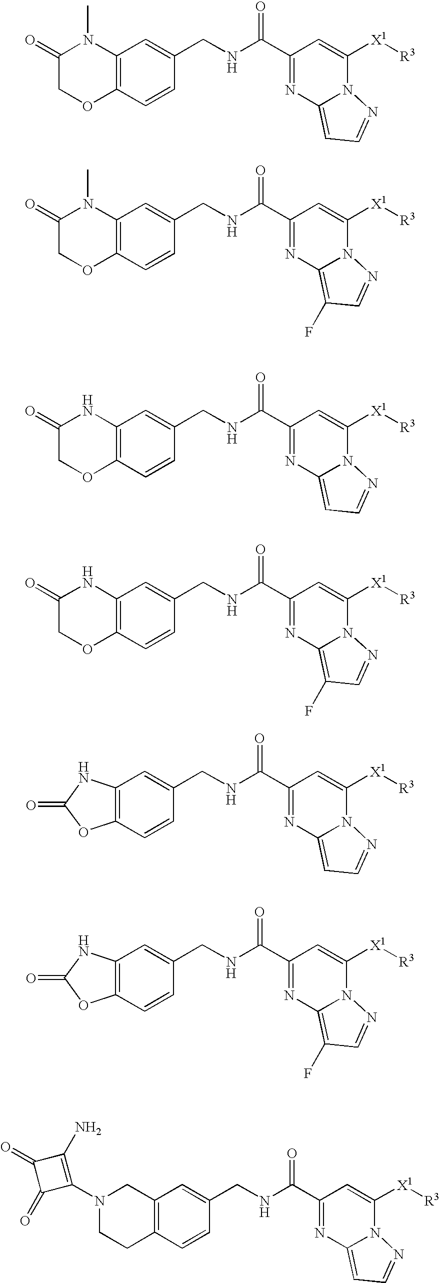

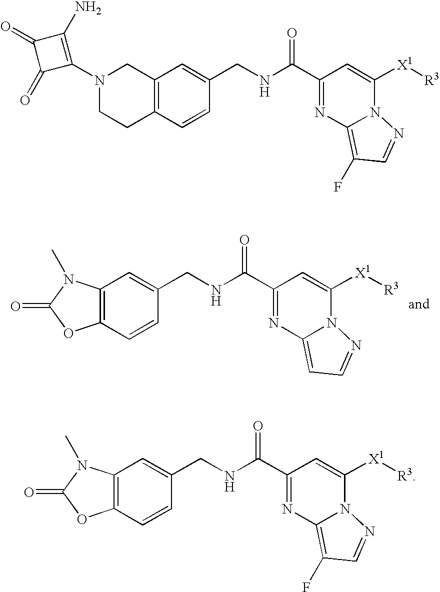

In another embodiment, in conjunction with any above or below embodiments, X¹ is selected from a bond; and R³ is selected from

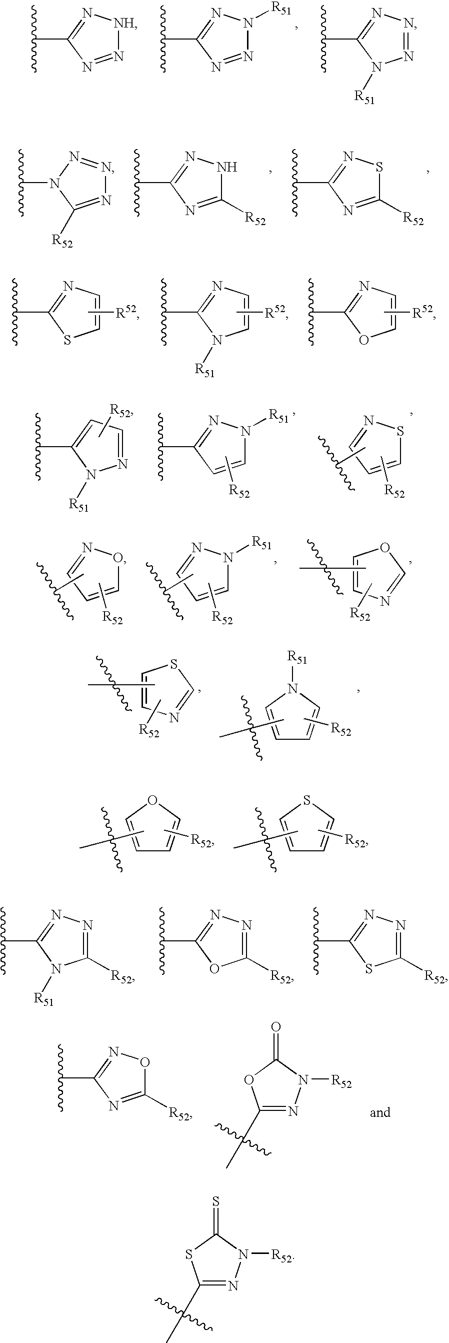

In another embodiment, the compound has the structure:

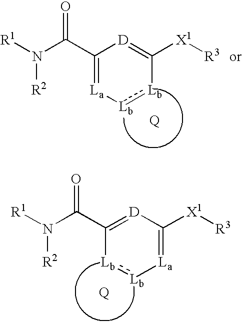

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is NR²⁰R²¹ or NR¹⁰R¹¹;

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹⁰, (C₀-C₆)-alkyl-NR¹⁰CONR¹⁰SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹⁰—CN, O—(C₀-C₆)-alkyl-C(O)NR¹⁰R¹¹, S(O)ₓ-(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

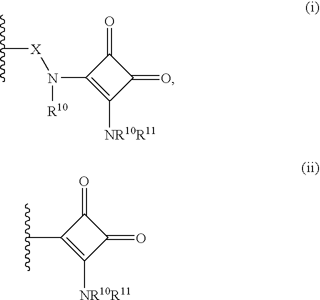

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

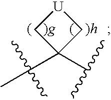

D is a member selected from CR²² and N;

Lₐ is selected from CR⁹ and N;

L_{b} is independently selected from C and N with the provisos that both L_{b} are not N, and that the bond between L_{b} and L_{b} is optionally a double bond only if both are L_{b} are carbon;

Q is a 5- or 6-membered ring selected from aryl and heteroaryl, wherein aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{W};

X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In another embodiment, in conjunction with any above or below embodiments, the compound has a structure selected from:

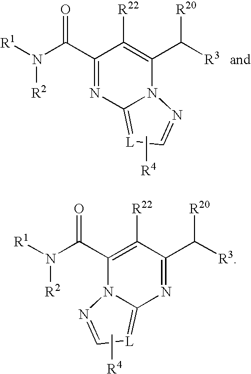

In another embodiment, in conjunction with any above or below embodiments, the compound has a structure selected from:

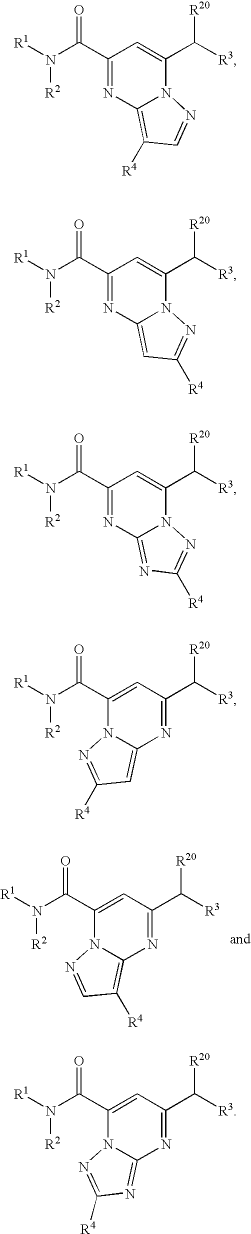

In another embodiment, in conjunction with any above or below embodiments, R³ is selected from:

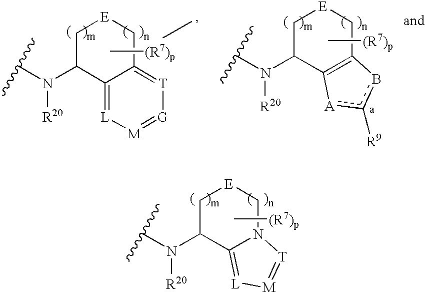

wherein

R⁷ is independently selected from hydrogen, alkyl, cycloalkyl, halo, R⁴ and NR¹⁰R¹¹, or optionally two R⁷ groups together at the same carbon atom form ═O, ═S or ═NR¹⁰;

A and B are independently selected from CR⁹, CR⁹R¹⁰, NR¹⁰, N, O and S(O)ₓ;

G, L, M and T are independently selected from CR⁹ and N;

m and n are independently selected from 0-3, provided that:
(1) when E is present, m and n are not both 3;
(2) when E is —CH₂—W¹—, m and n are not 3; and
(3) when E is a bond, m and n are not 0; and

p is selected from 0-6;

wherein the dotted line represents a double bond between one of: carbon “a” and A, or carbon “a” and B.

In another embodiment, in conjunction with any above or below embodiments, R³ is selected from:

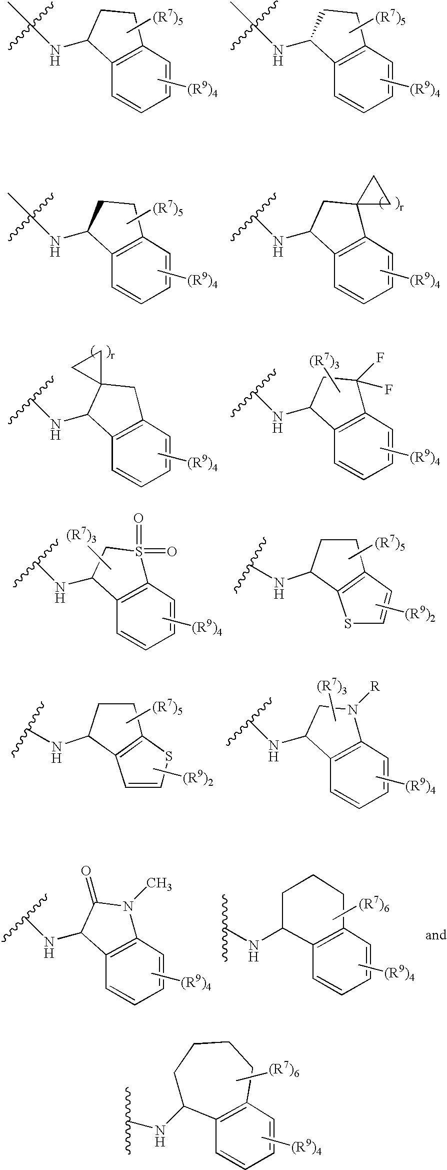

wherein:

R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹¹, CONHCH₃ and CON(CH₃)₂ are optionally substituted one or more times; and

r is selected from 1-4.

In another embodiment, in conjunction with any above or below embodiments, R³ is selected from:

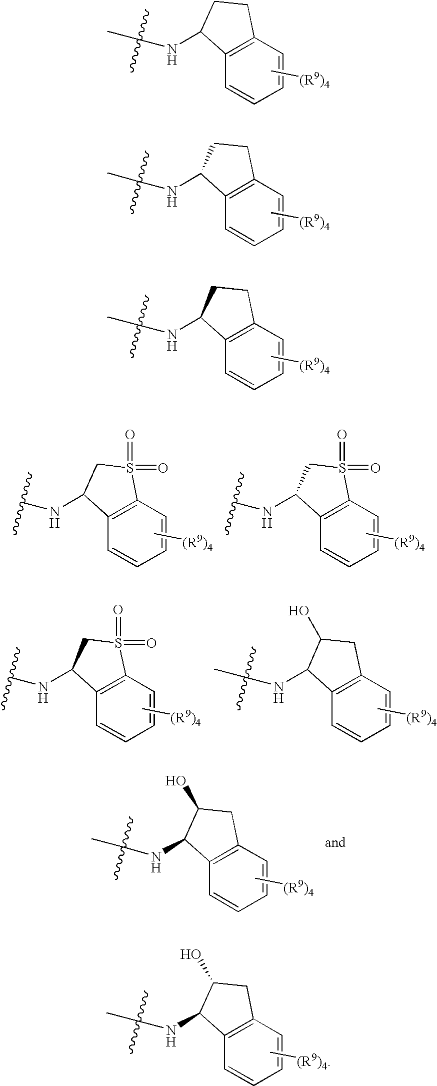

In another embodiment, in conjunction with any above or below embodiments, R⁹ is selected from:

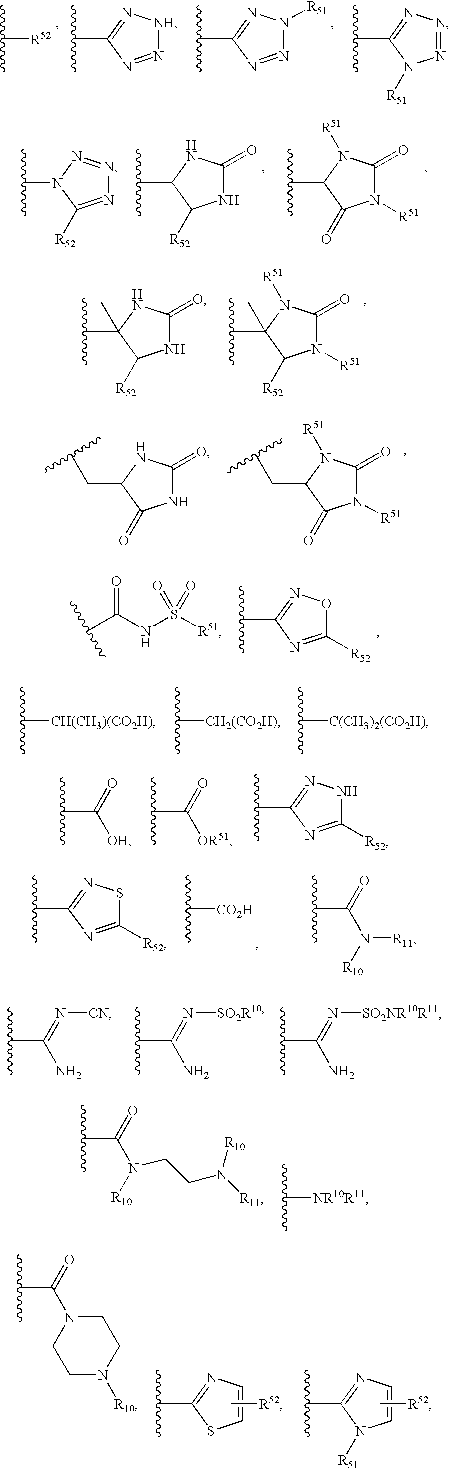

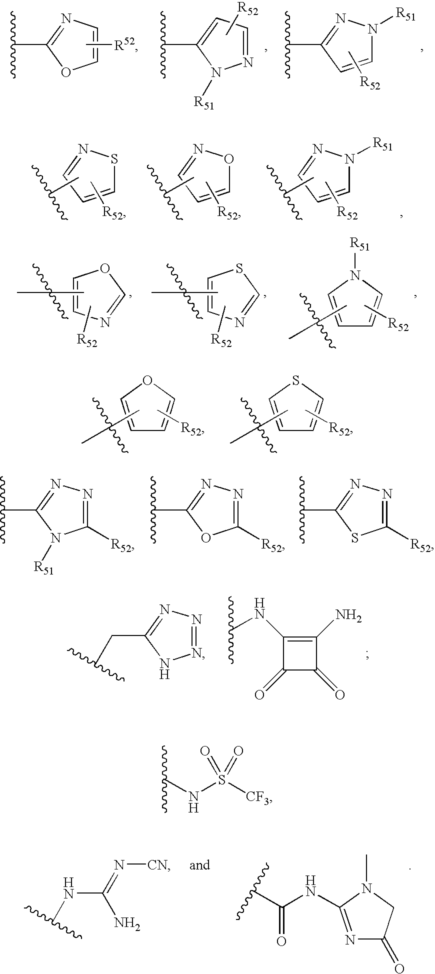

In another embodiment, in conjunction with any above or below embodiments, R³ is selected from:

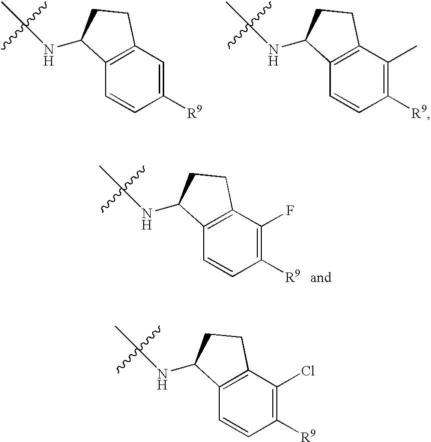

wherein:

R⁹ is selected from hydrogen, fluoro, halo, CN, alkyl, CO₂H,

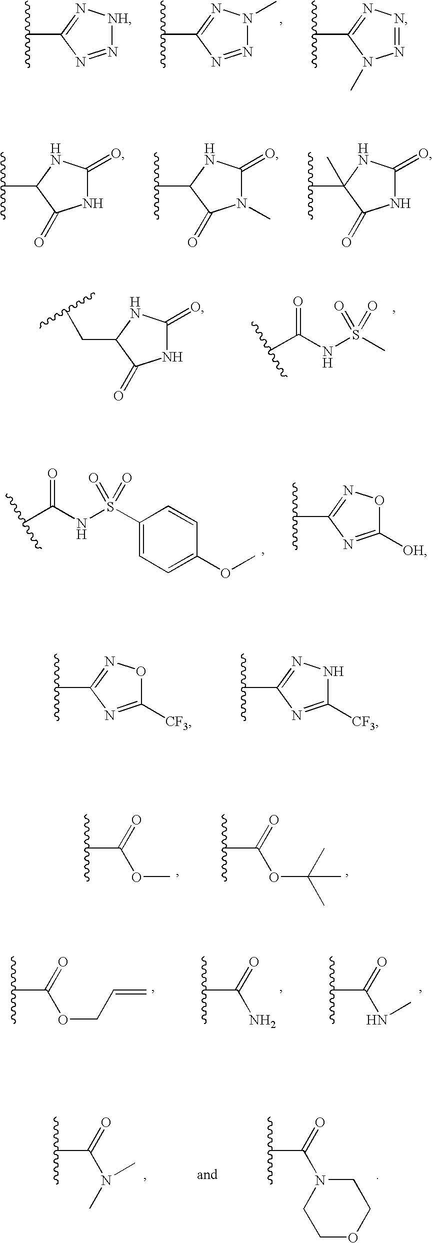

In another embodiment, in conjunction with any above or below embodiments, R¹ is selected from:

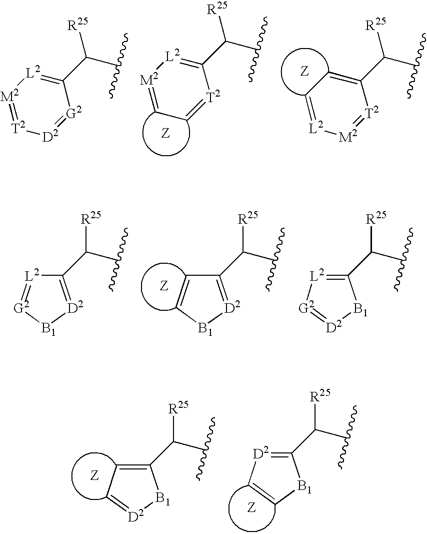

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S(O)ₓ;

D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

In another embodiment, in conjunction with any above or below embodiments, R¹ is selected from:

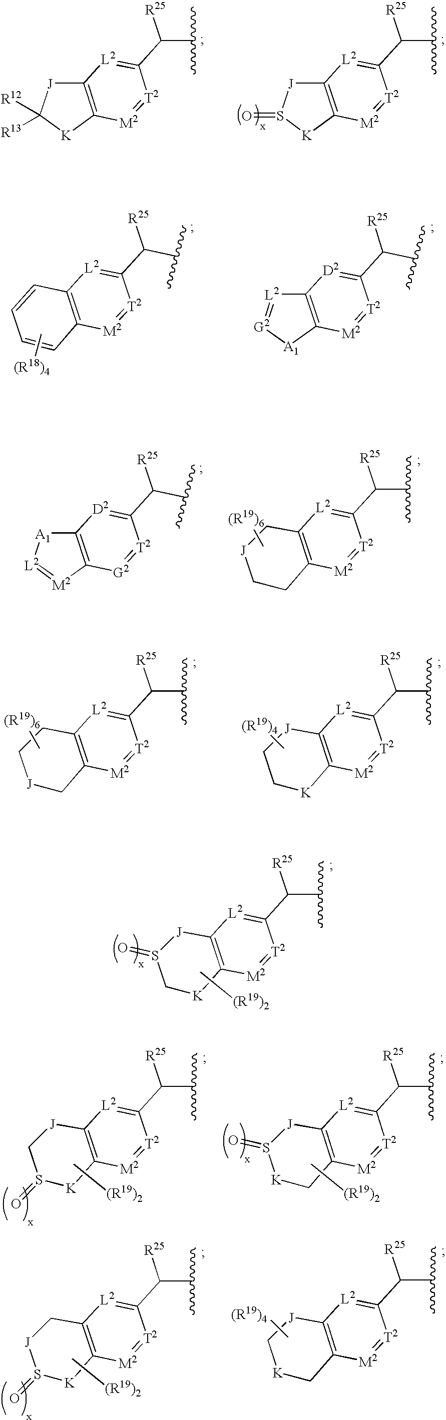

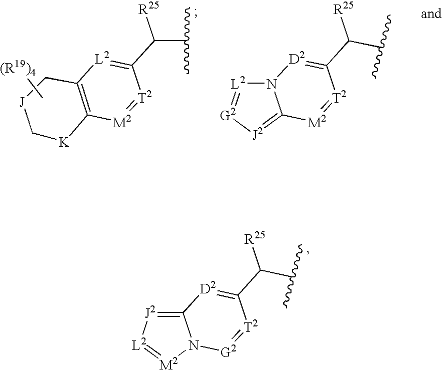

wherein:

R¹² and R¹³ are independently selected from hydrogen, alkyl and halo, wherein alkyl is optionally substituted one or more times, or optionally R¹² and

R¹³ together form ═O, ═S or ═NR¹⁰;

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹¹, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹¹, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

J and K are independently selected from CR¹⁰R¹⁸, NR¹⁰, O and S(O)ₓ;

A₁ is selected from NR¹⁰, O and S(O)ₓ; and

D², G², J², L², M² and T² are independently selected from CR¹⁸ and N.

In another embodiment, in conjunction with any above or below embodiments, R¹ is selected from:

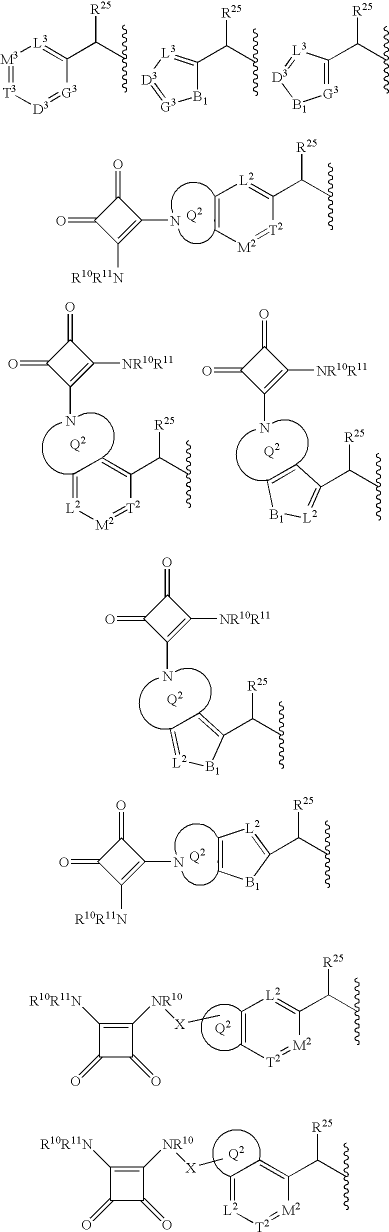

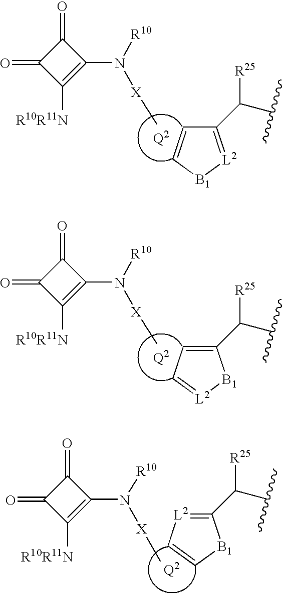

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR₁₀CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, CONR¹⁰R¹¹ and haloalkyl, wherein alkyl, cycloalkyl and haloalkyl are optionally substituted one or more times;

L², M², and T² are independently selected from CR¹⁸ and N;

D³, G³, L³, M³, and T³ are independently selected from N, CR¹⁸, (i), and (ii),

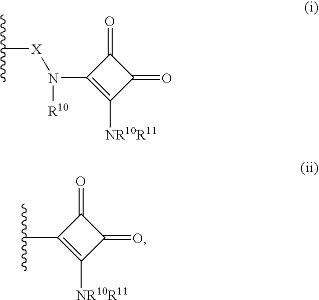

with the proviso that one of L³, M³, T³, D³, and G3 is (i) or (ii)

B₁ is selected from the group consisting of NR¹⁰, O and S(O)ₓ; and

Q² is a 5- to 8-membered ring selected from cycloalkyl, heterocycloalkyl, aryl, and heteroaryl, which is optionally substituted one or more times with R¹⁹.

In another embodiment, in conjunction with any above or below embodiments, R¹ is selected from:

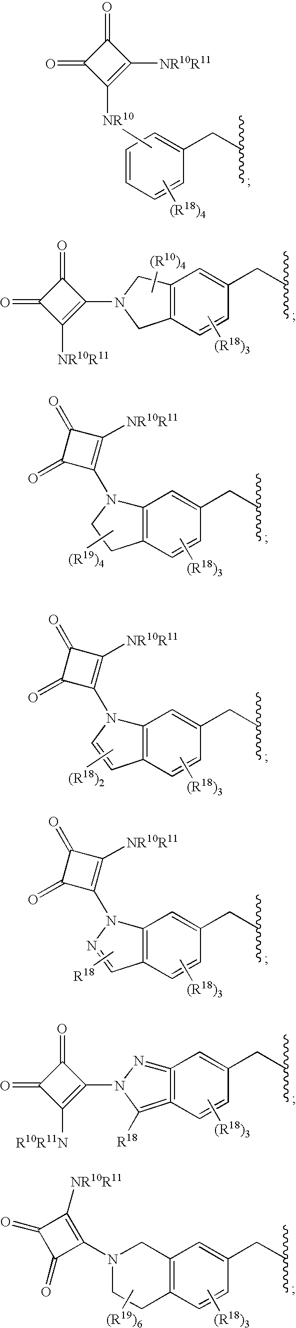

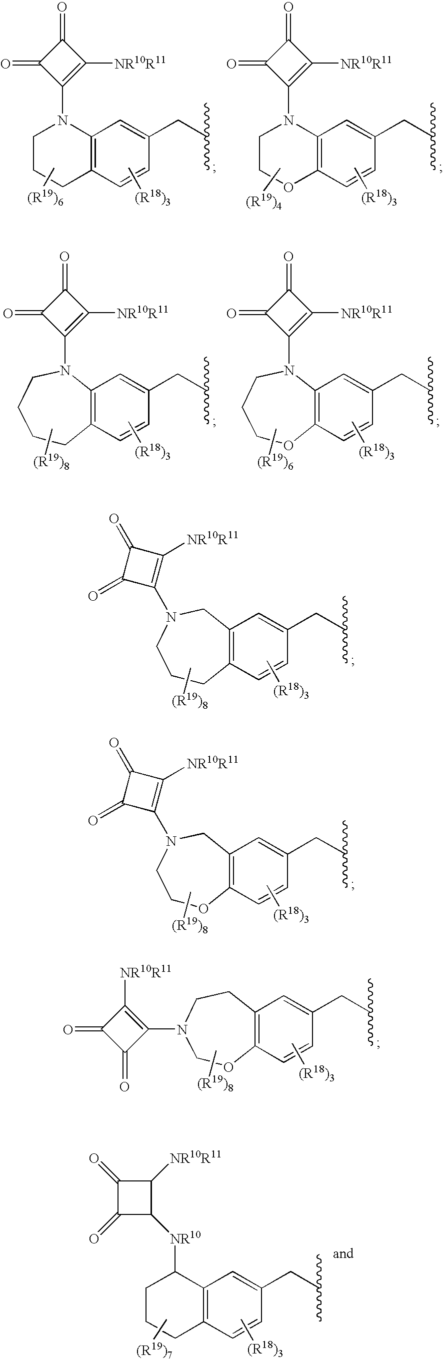

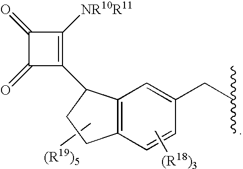

Another aspect of the invention relates to compounds having the structure:

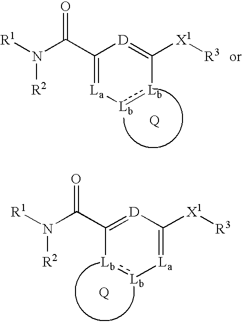

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)^{x}, or NR⁵⁰ and which is optionally substituted one or more times;

R³ is selected from

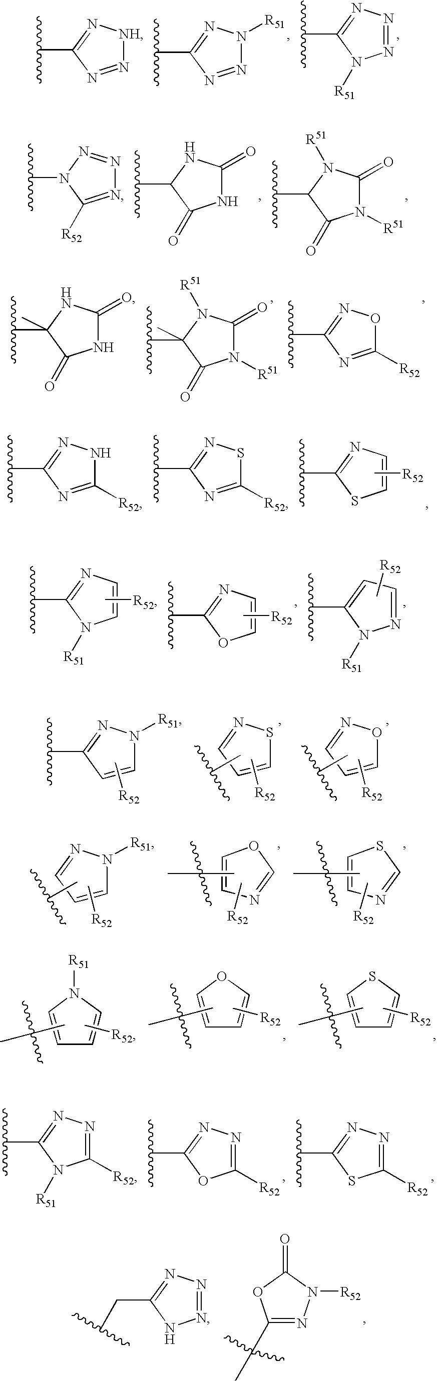

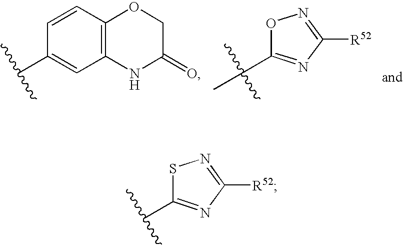

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹¹, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)_{y}R¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ-(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

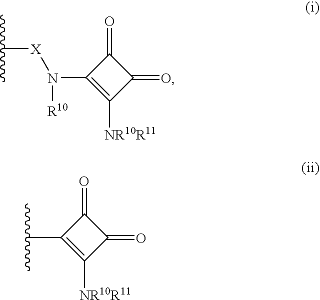

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸⁰, SO₂R⁸⁰ and SO₂NR⁸⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

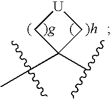

D is a member selected from CR²² and N;

Lₐ is selected from CR⁹ and N;

L_{b} is independently selected from C and N with the provisos that both L_{b} are not N, and that the bond between L_{b} and L_{b} is optionally a double bond only if both are L_{b} are carbon;

Q is a 5- or 6-membered ring selected from aryl and heteroaryl, wherein aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In another embodiment, in conjunction with any above or below embodiments, the compound has a structure selected from:

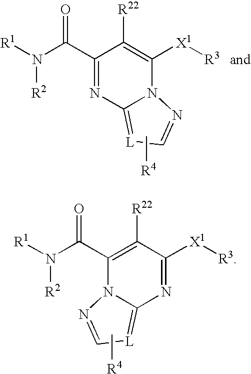

In another embodiment, in conjunction with any above or below embodiments, R¹ is selected from:

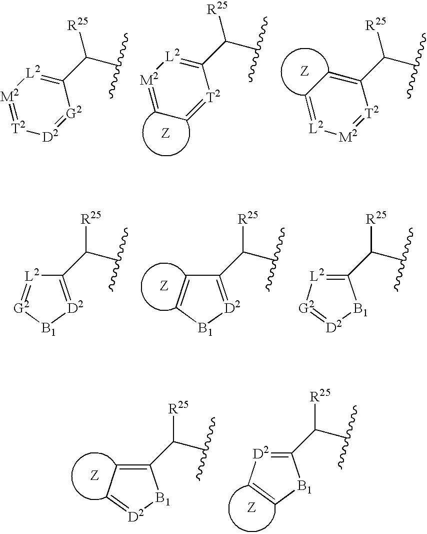

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S(O)ₓ;

D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

In another embodiment, in conjunction with any above or below embodiments, R¹ is selected from:

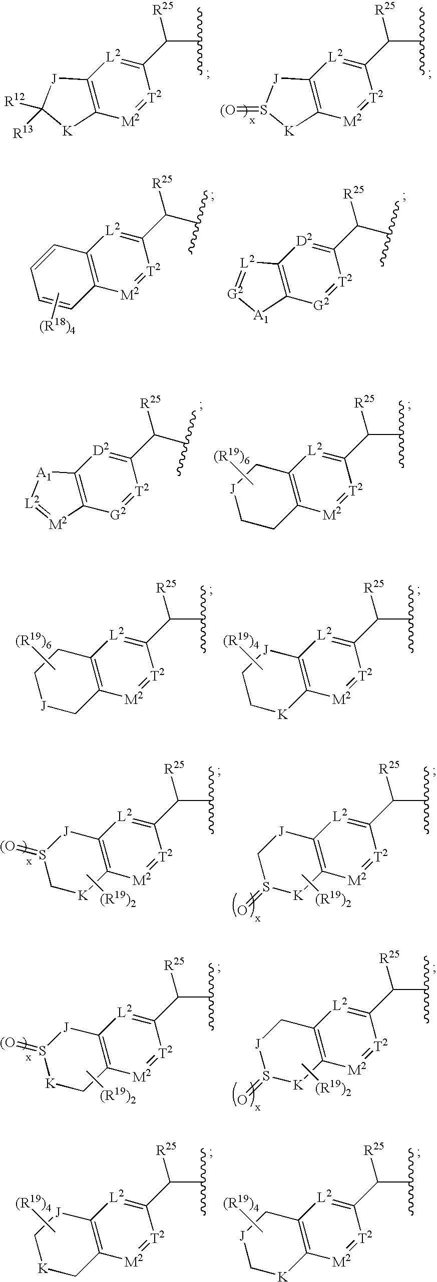

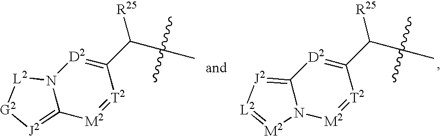

wherein:

R¹² and R¹³ are independently selected from hydrogen, alkyl and halo, wherein alkyl is optionally substituted one or more times, or optionally R¹² and R¹³ together form ═O, ═S or ═NR¹⁰;

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

J and K are independently selected from CR¹⁰R¹⁸, NR¹⁰, O and S(O)ₓ;

A¹ is selected from NR¹⁰, O and S(O)ₓ; and

D², G², J², L², M² and T² are independently selected from CR¹⁸ and N.

In another embodiment, in conjunction with any above or below embodiments, R¹ is selected from:

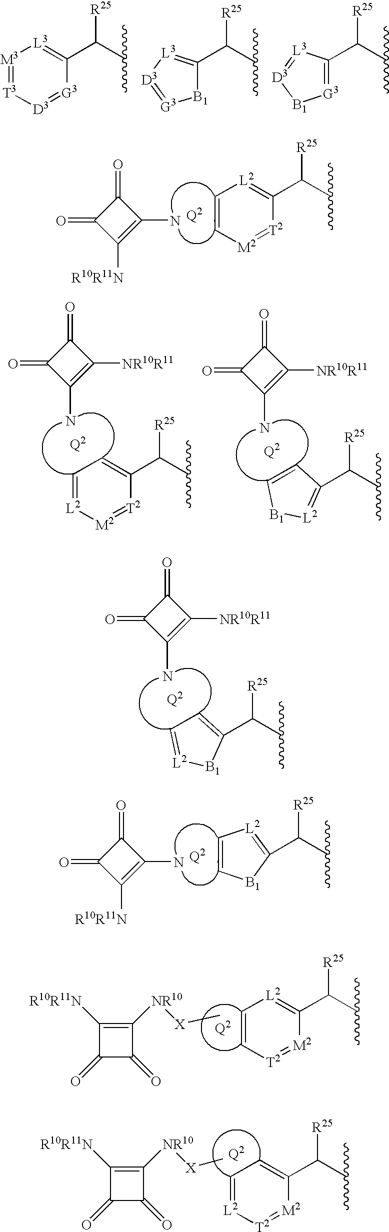

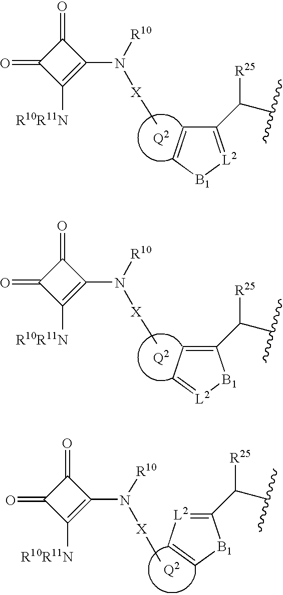

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, CONR¹⁰R¹¹ and haloalkyl, wherein alkyl, cycloalkyl and haloalkyl are optionally substituted one or more times;

L², M², and T² are independently selected from CR¹⁸ and N;

D³, G³, L³, M³, and T³ are independently selected from N, CR¹⁸, (i), and (ii),

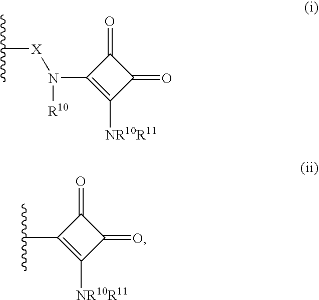

with the proviso that one of L³, M³, T³, D³, and G³ is (i) or (ii)

B₁ is selected from the group consisting of NR¹⁰, O and S(O)ₓ; and

Q² is a 5- to 8-membered ring selected from cycloalkyl, heterocycloalkyl, aryl, and heteroaryl, which is optionally substituted one or more times with R¹⁹.

In another embodiment, in conjunction with any above or below embodiments, R¹ is selected from:

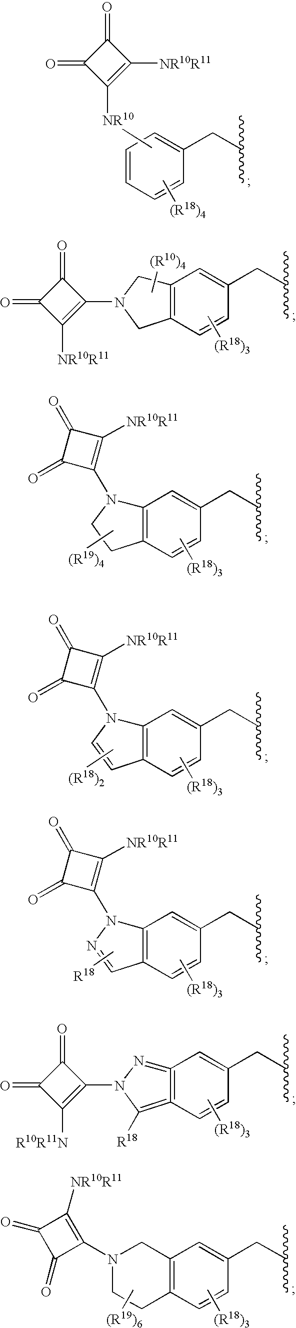

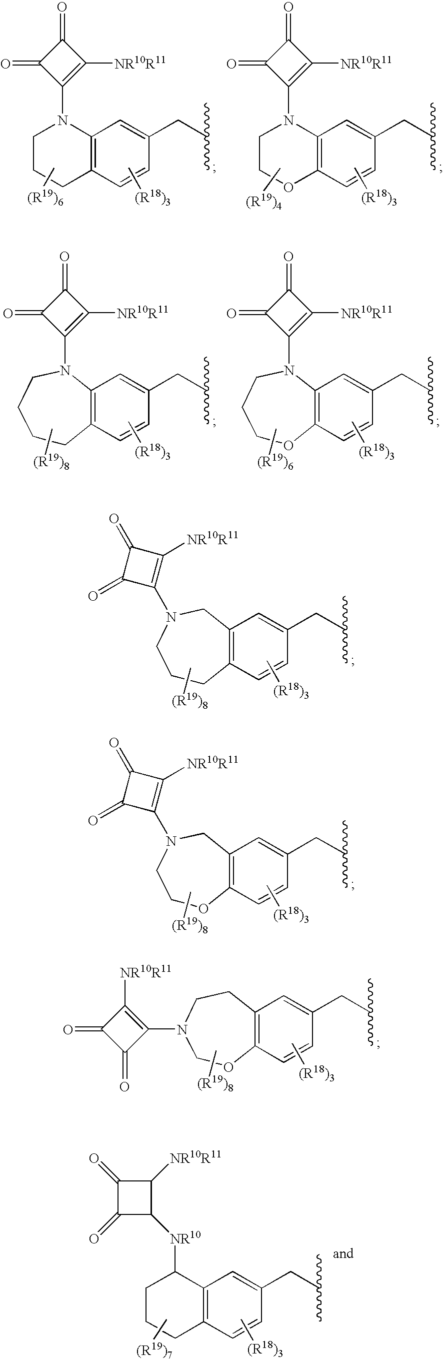

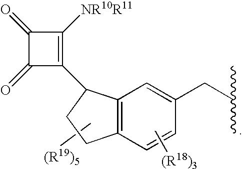

Another aspect of the invention relates to a compound having the structure:

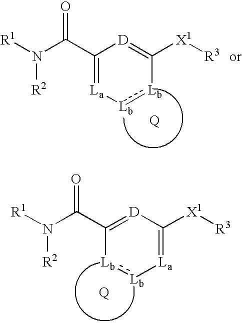

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is SO₂NR¹⁰R¹¹, SO₂NR²R²¹, PO₂R¹⁰, PO₂R²¹,

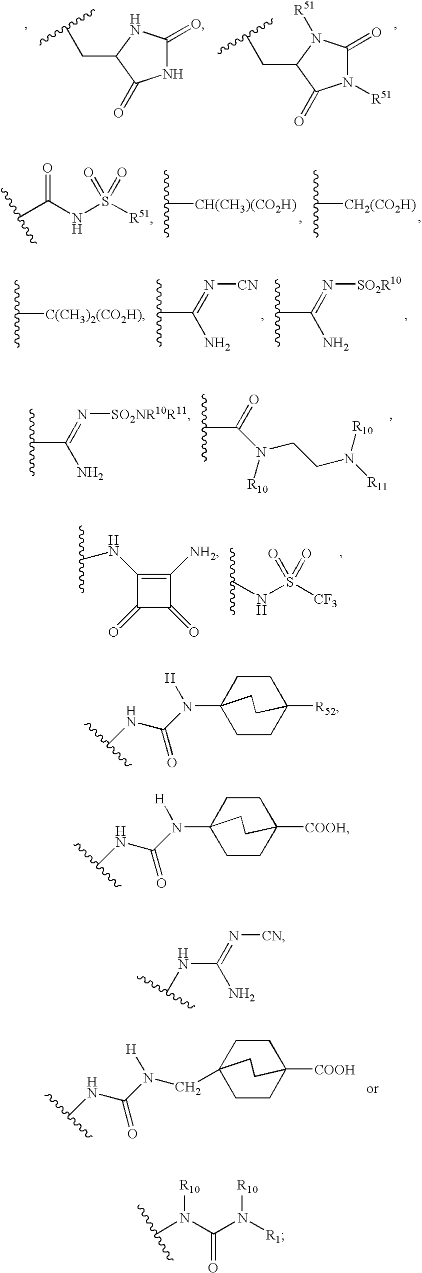

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹¹, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NRIR¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹⁰—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

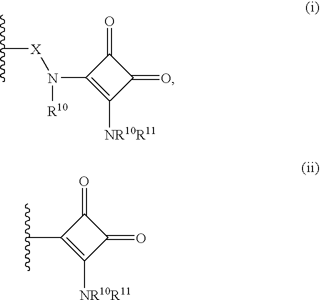

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR¹⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

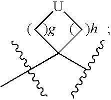

D is a member selected from CR²² and N;

Lₐ is selected from CR⁹ and N;

L_{b} is independently selected from C and N with the provisos that both L_{b} are not N, and that the bond between L_{b} and L_{b} is optionally a double bond only if both are L_{b} are carbon;

Q is a 5- or 6-membered ring selected from aryl and heteroaryl, wherein aryl and heteroaryl are optionally substituted one or more times with R⁴;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹¹);

X is selected from a bond and (CR¹⁰R¹¹)_{w}E(CR¹⁰R¹¹)_{w};

X¹ is a bond, NR¹⁰, CH₂, CH²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

In another embodiment, in conjunction with any above or below embodiments, the compound has a structure selected from:

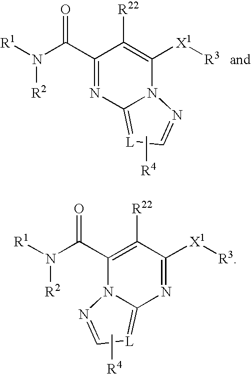

In another embodiment, in conjunction with any above or below embodiments, R¹ is selected from:

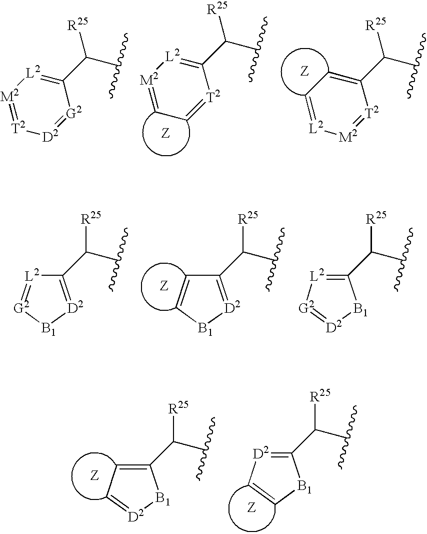

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S(O)ₓ;

D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

In another embodiment, in conjunction with any above or below embodiments, R¹ is selected from:

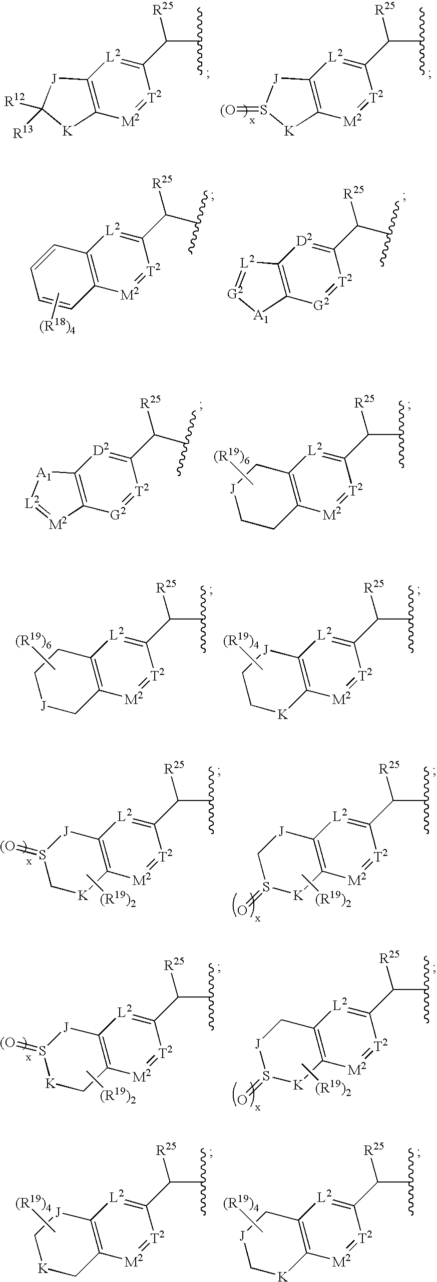

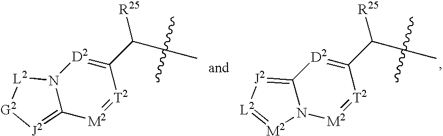

wherein:

R¹² and R¹³ are independently selected from hydrogen, alkyl and halo, wherein alkyl is optionally substituted one or more times, or optionally R¹² and R¹³ together form ═O, ═S or ═NR¹⁰;

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹,

NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹¹SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

J and K are independently selected from CR¹⁰R¹⁸, NR¹⁰, O and S(O)ₓ;

A₁ is selected from NR¹⁰, O and S(O)ₓ; and

D², G², J², L², M² and T² are independently selected from CR¹⁸ and N. Another aspect of the invention relates to a compound having the structure:

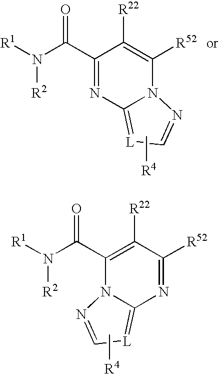

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NROR¹¹, (C₀-C₆)-alkyl-C(O)NR¹⁰SO₂R¹¹, (C₀-C₆)-alkyl-C(O)—NR¹⁰—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ-(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

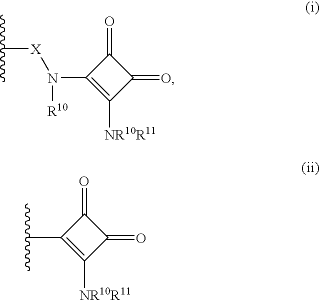

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹¹SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R¹⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, fluoroalkoxy, alkyl and haloalkyl;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹), —CH₂—W¹— and

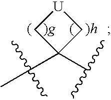

D is a member selected from CR²² and N;

L is C or N;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

X¹ is a bond, NR¹⁰, CH₂, CHR²⁰, CR²⁰R²¹, SO₂, SO, S, PO₂, O, C═S, C═NR¹, C═N—SO₂R¹¹, C═N—CN, C═N—CONR¹⁰R¹¹, C═N—COR¹¹, C═N—OR¹⁰;

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

Another aspect of the invention relates to a compound having the structure:

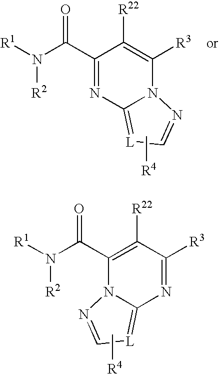

wherein:

R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is NR²⁰R²¹, NR¹⁰R¹¹, NR¹⁰SO₂R¹¹, NR¹⁰SO₂R²¹, OR¹⁰, OR²¹ or NR¹⁰NR⁹;

R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R^{3e}, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹¹, (C₀-C₆)-alkyl-NR¹⁰—C(O)OR¹⁰, (C₀-C₆)-alkyl-NR¹¹—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O —(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)_{y}R¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;

R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.

R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

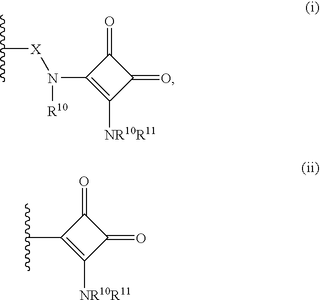

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R² are attached to a nitrogen atom they may be taken together to complete a 3- to 8-membered ring containing carbon atoms and optionally containing a heteroatom selected from O, S, or NR⁵⁰ and which is optionally substituted one or more times;

R²¹ is a monocyclic, bicyclic or tricyclic ring system wherein said bicyclic or tricyclic ring system is fused and contains at least one ring which is partially saturated and

wherein R²¹ is optionally substituted one or more times, or

wherein R²¹ is optionally substituted by one or more R⁹ groups;

R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R³⁰ is selected from alkyl and (C₀-C₆)-alkyl-aryl, wherein alkyl and aryl are optionally substituted;

R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

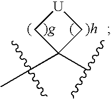

D is a member selected from CR²² and N;

L is C or N;

U is selected from C(R⁵R¹⁰), NR⁵, O, S, S═O and S(═O)₂;

W¹ is selected from O, NR⁵, S, S═O, S(═O)₂, N(R¹⁰)(C═O), N(R¹⁰)S(═O)₂ and S(═O)₂N(R¹⁰);

X is selected from a bond and (CR¹⁰R¹¹)_{w}E(CR¹⁰R¹¹)_{w};

g and h are independently selected from 0-2;

w is independently selected from 0-4;

x is selected from 0 to 2;

y is selected from 1 and 2; and

N-oxides, pharmaceutically acceptable salts, prodrugs, formulations, polymorphs, tautomers, racemic mixtures and stereoisomers thereof.

31. A compound according to claim 30, wherein R¹ is selected from:

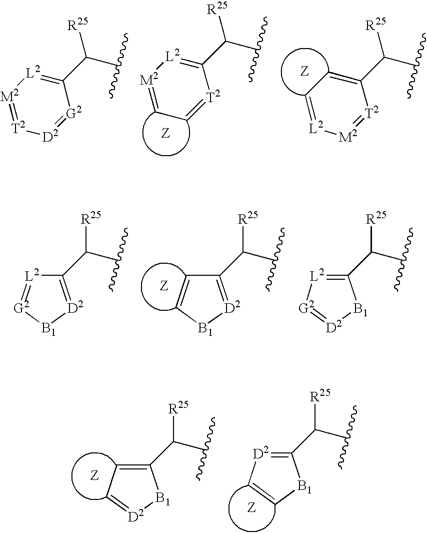

wherein:

R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S(O)ₓ;

D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

Another aspect of the invention relates to a compound selected from:

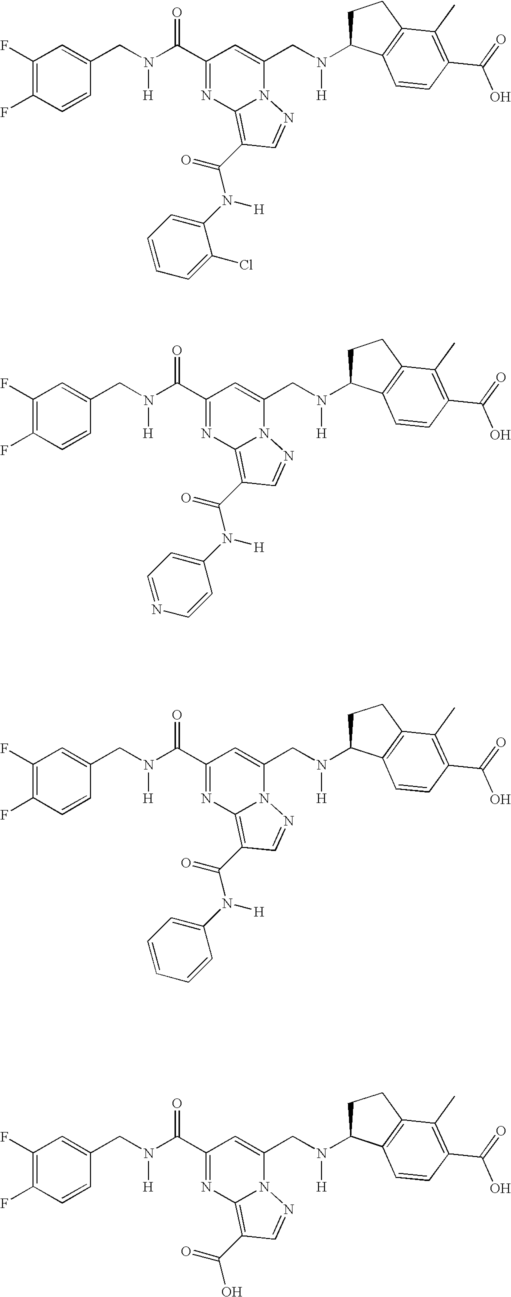

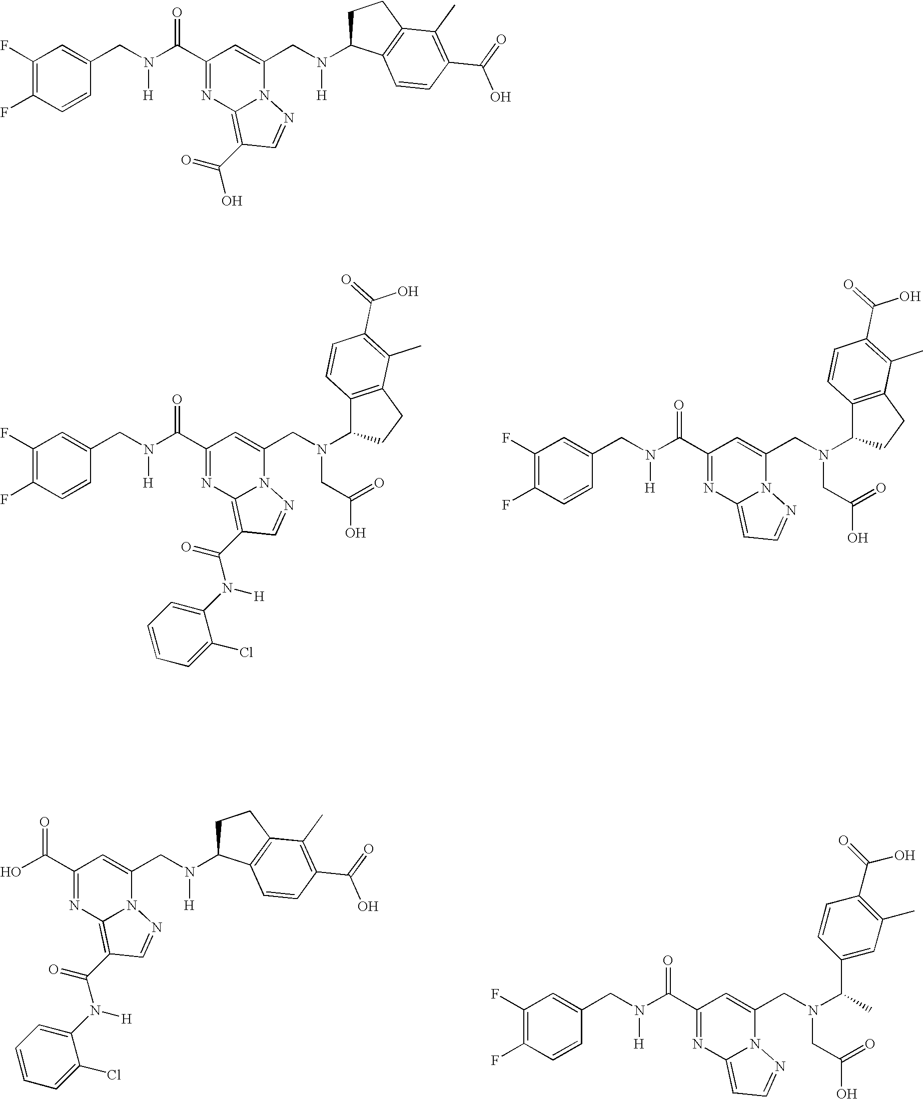

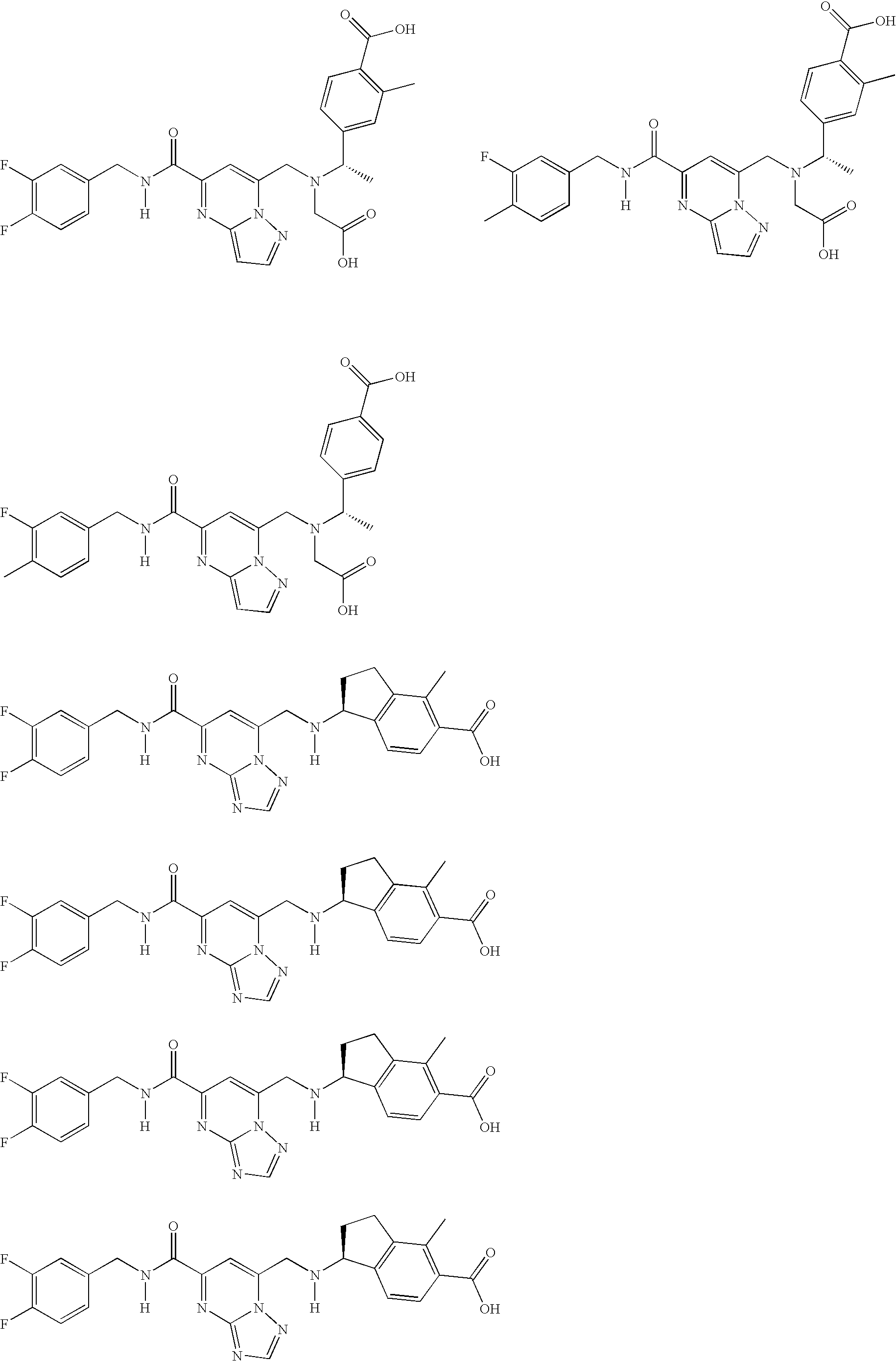

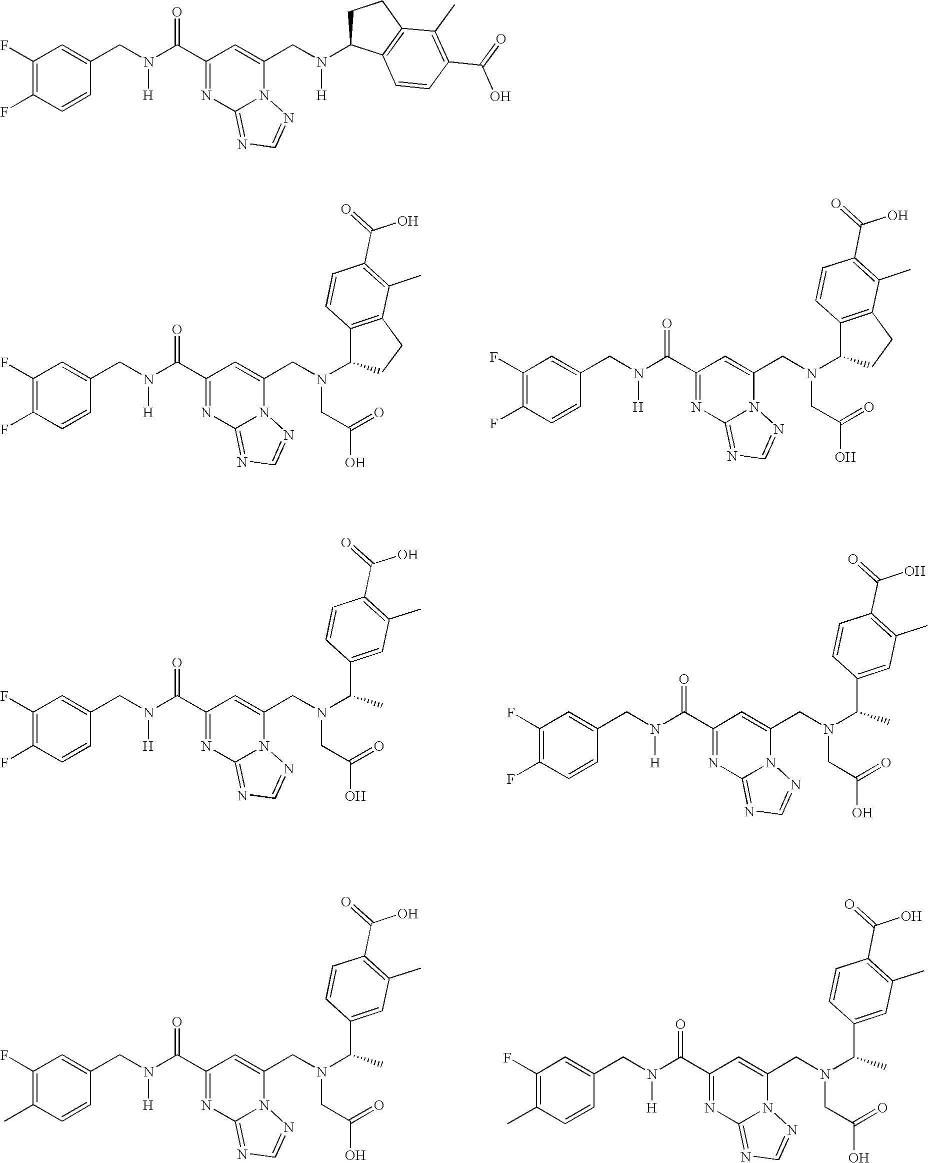

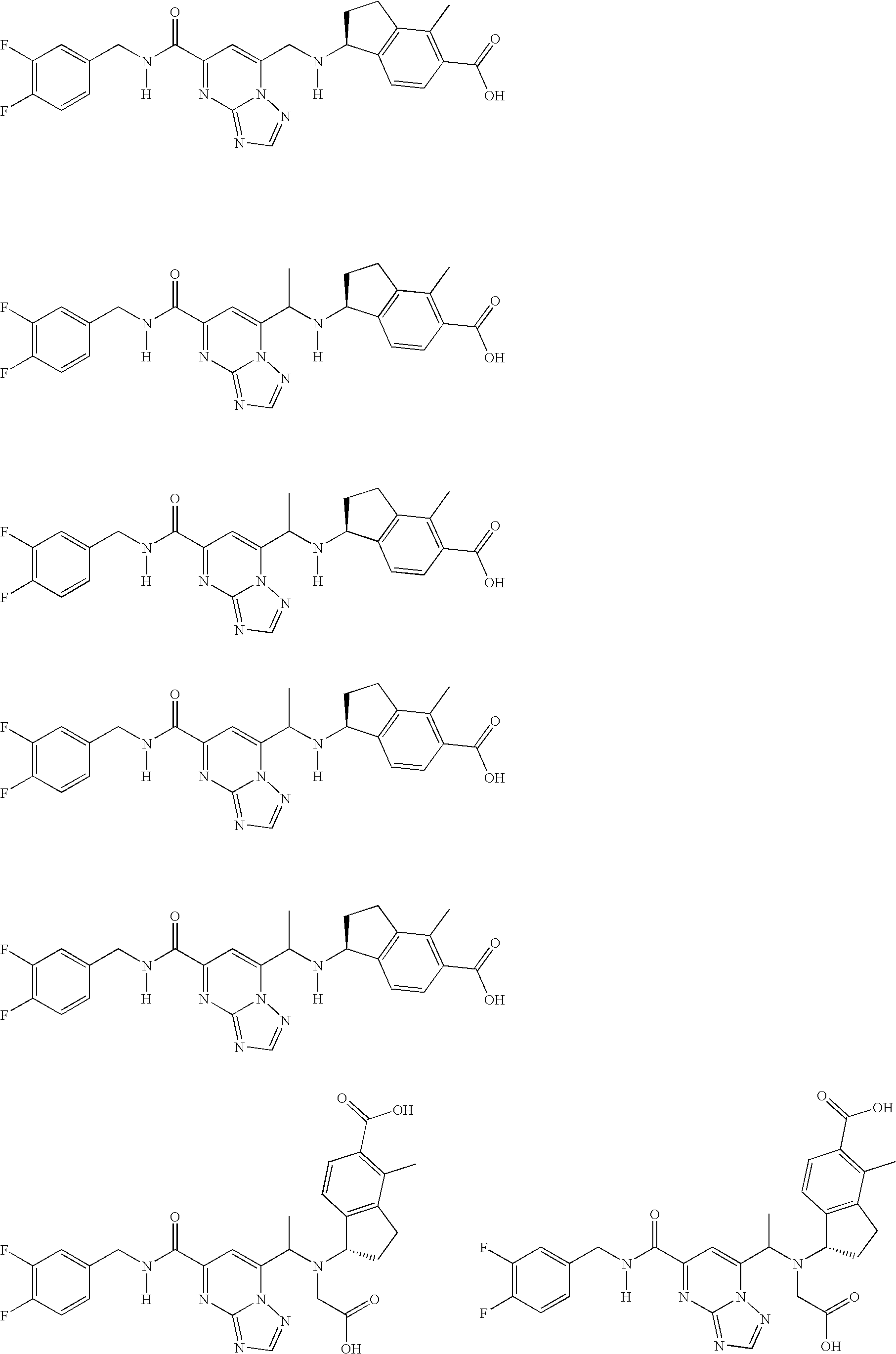

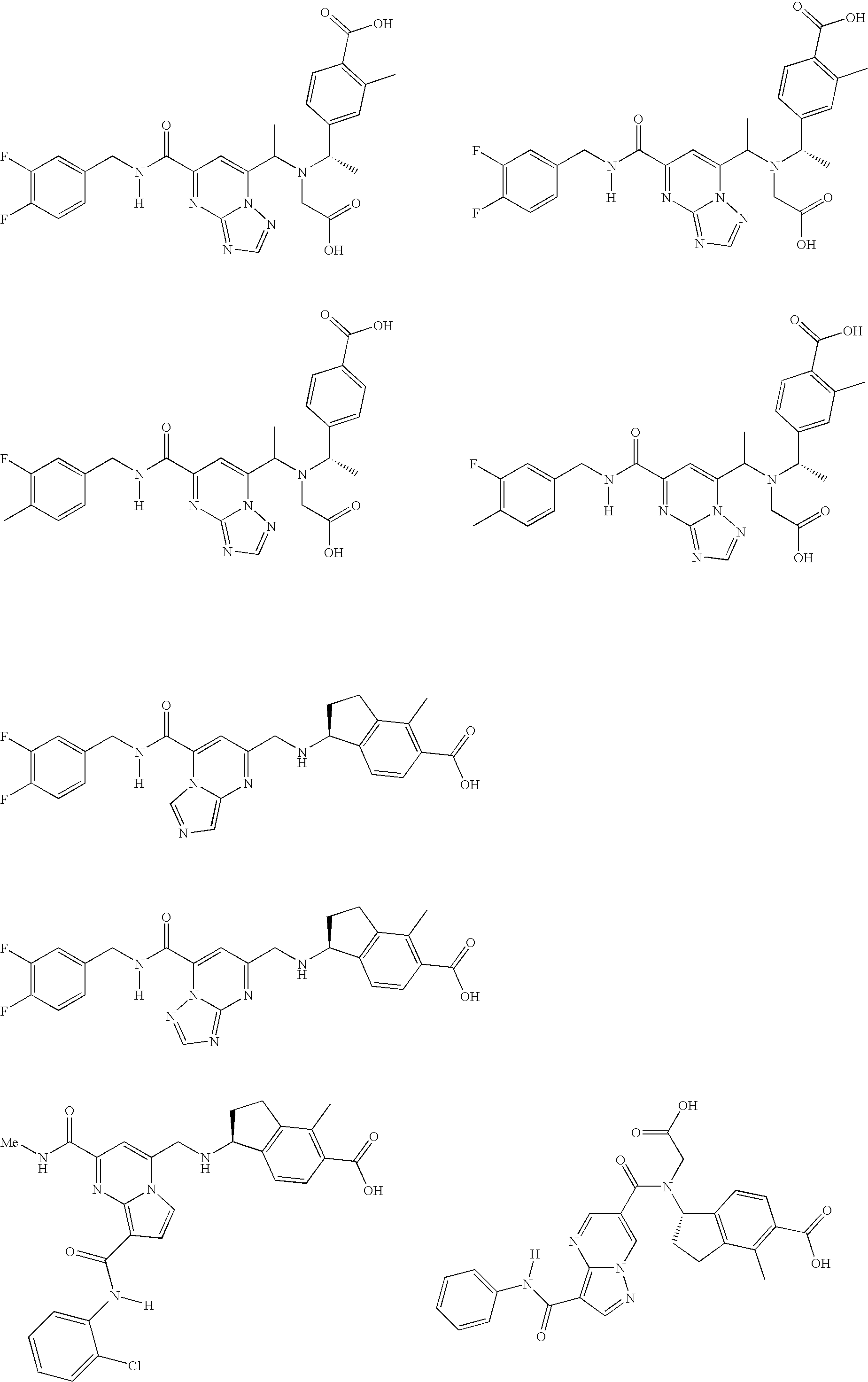

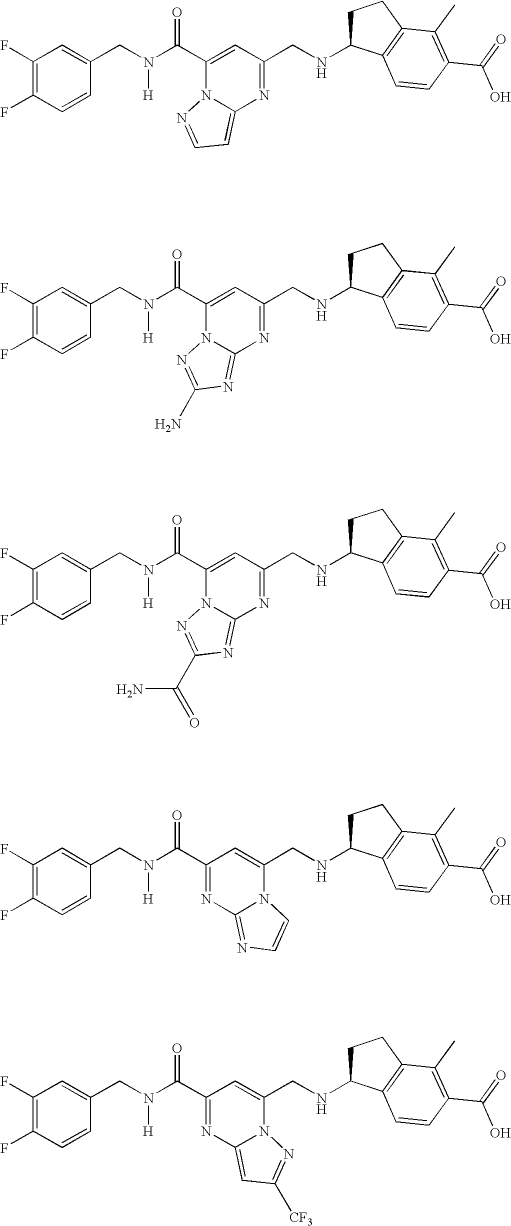

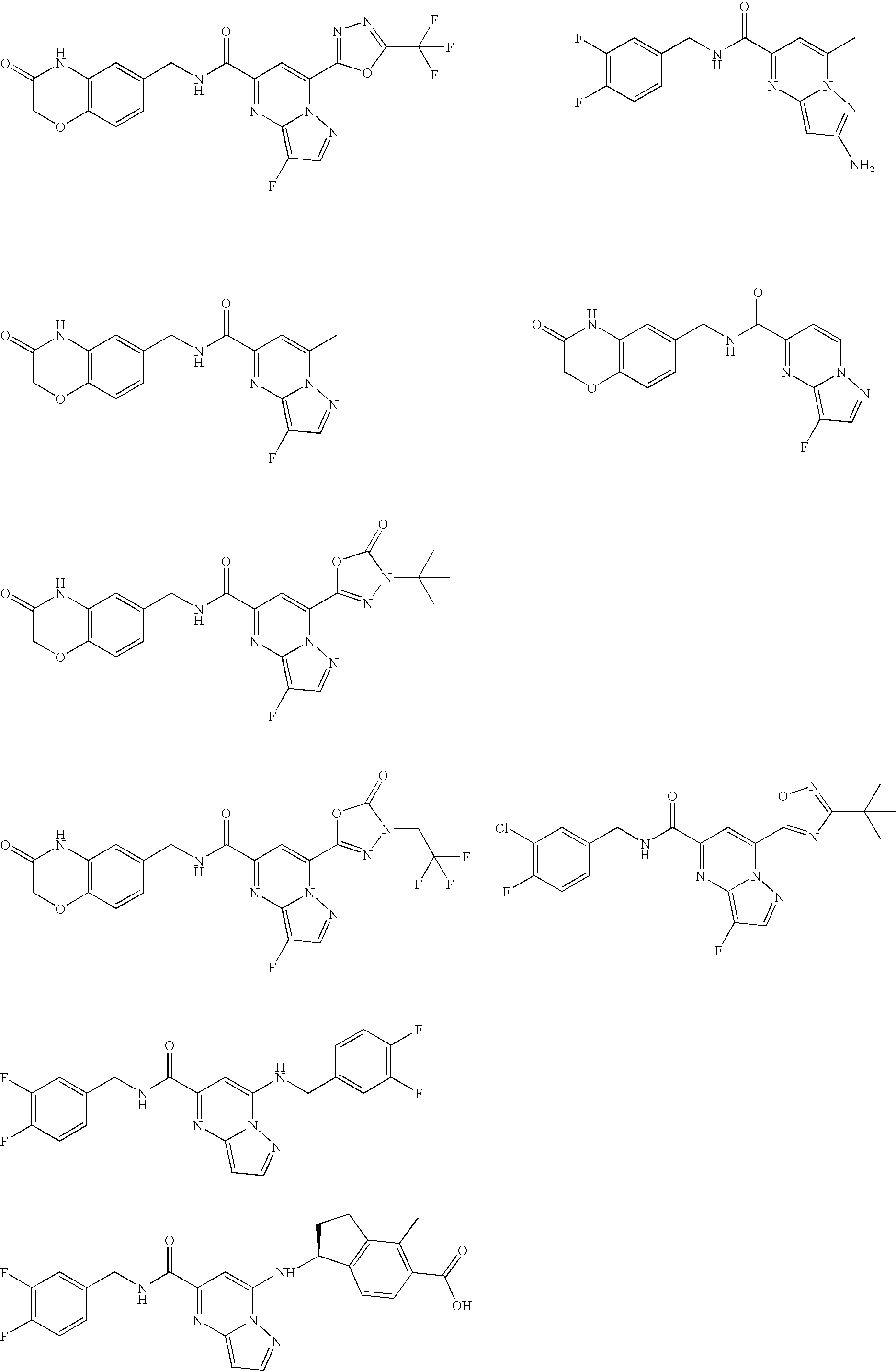

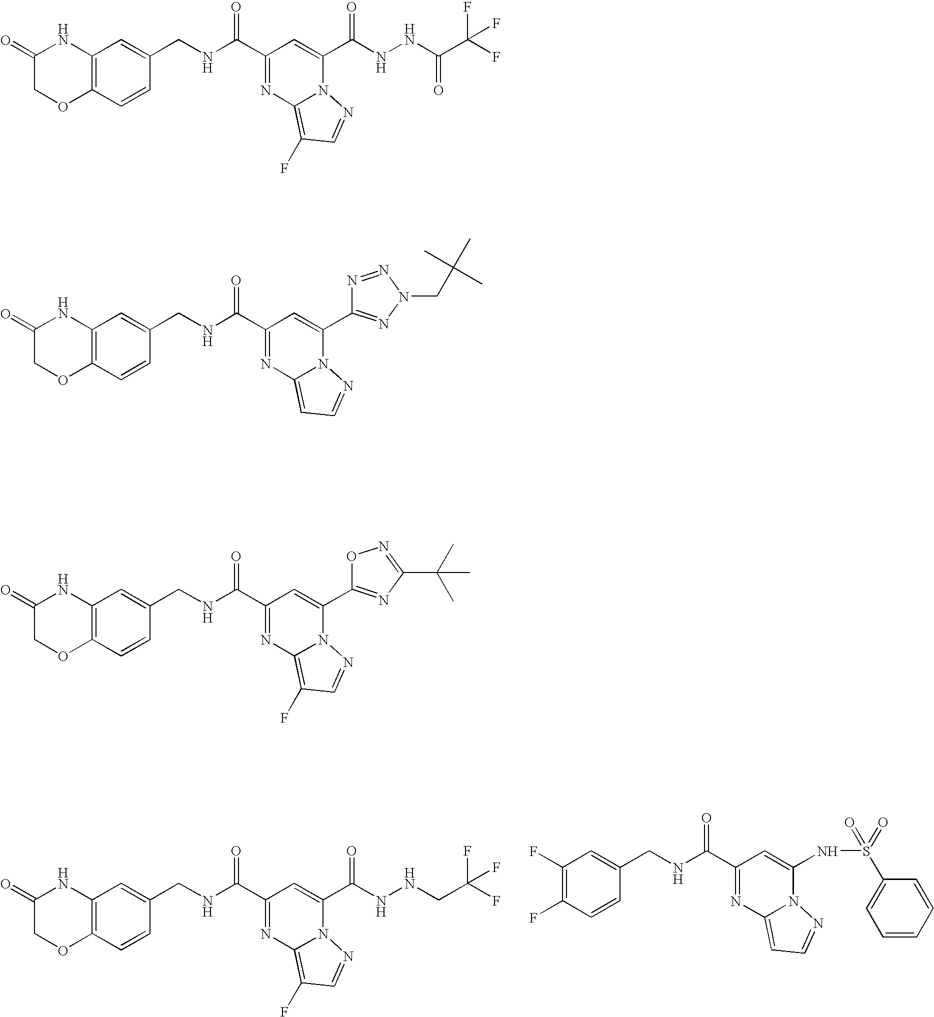

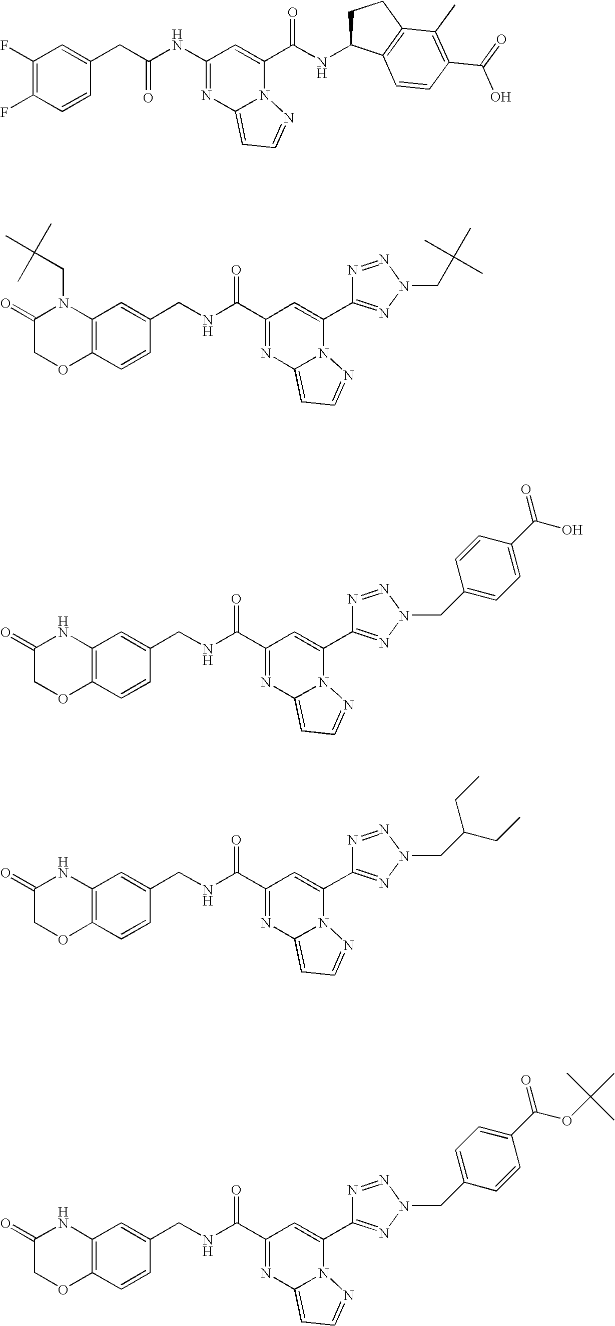

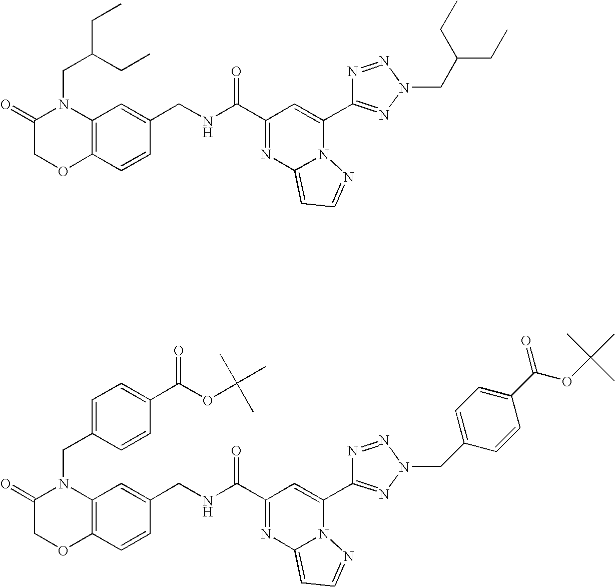

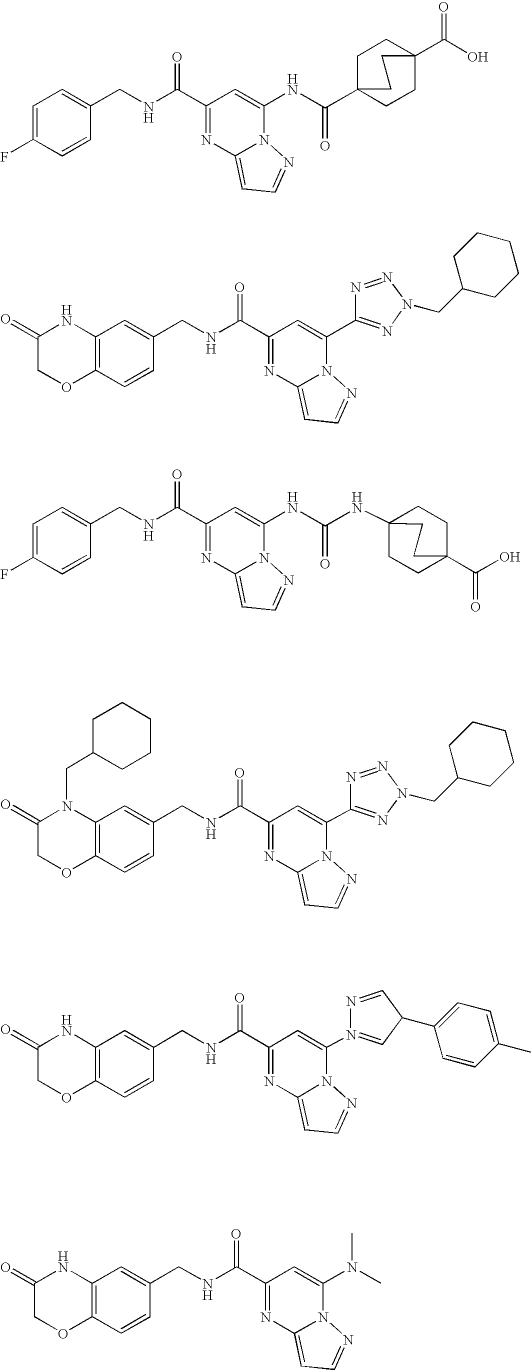

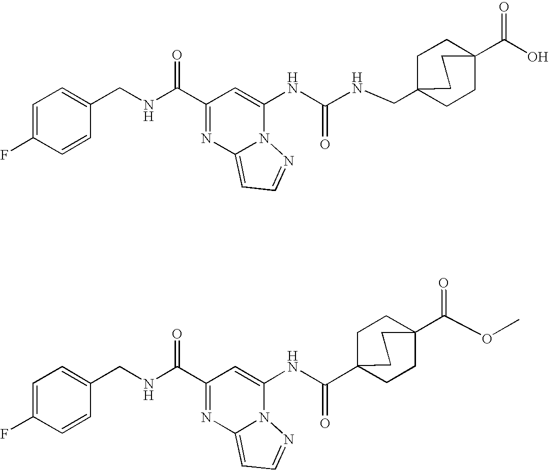

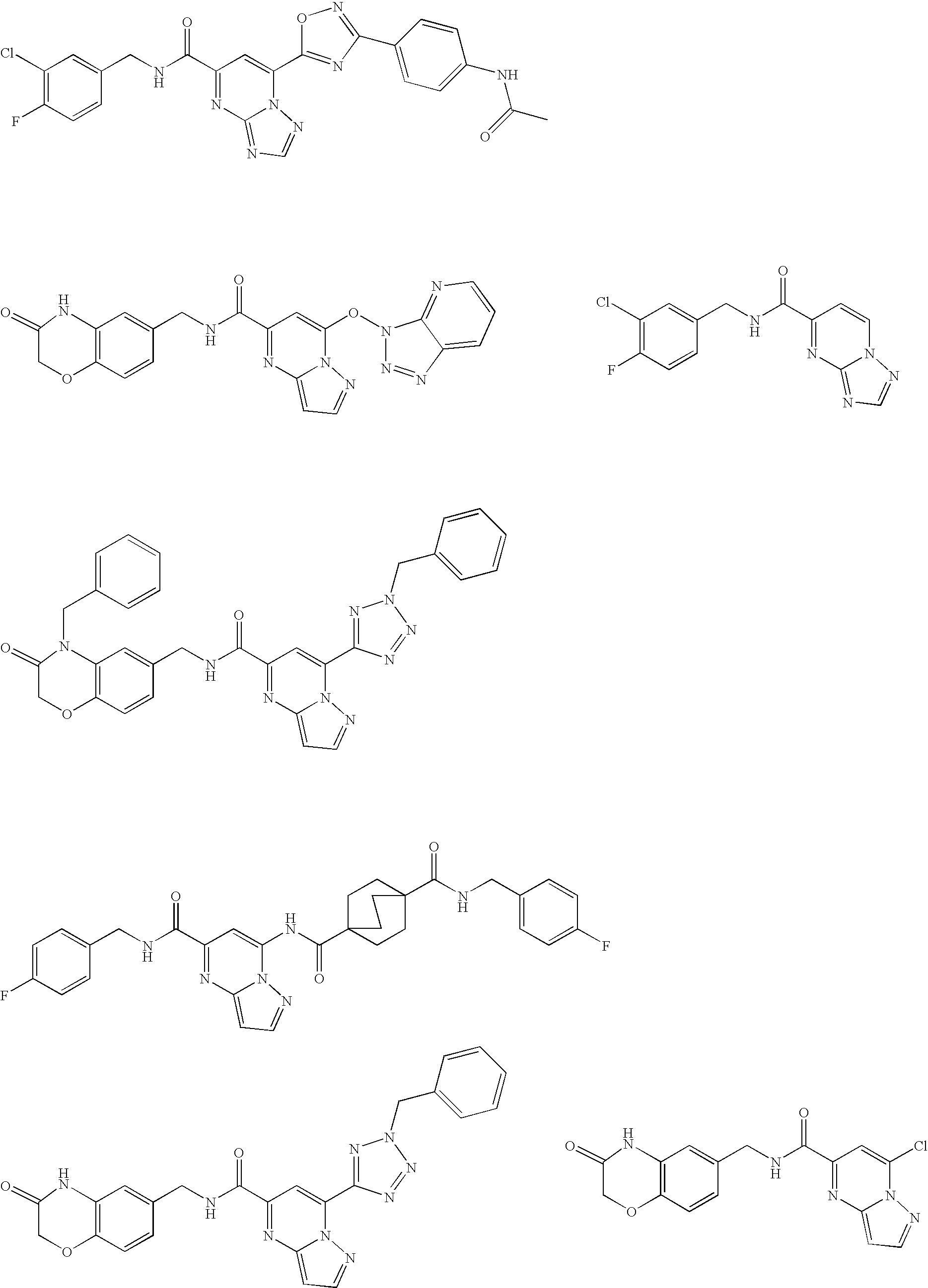

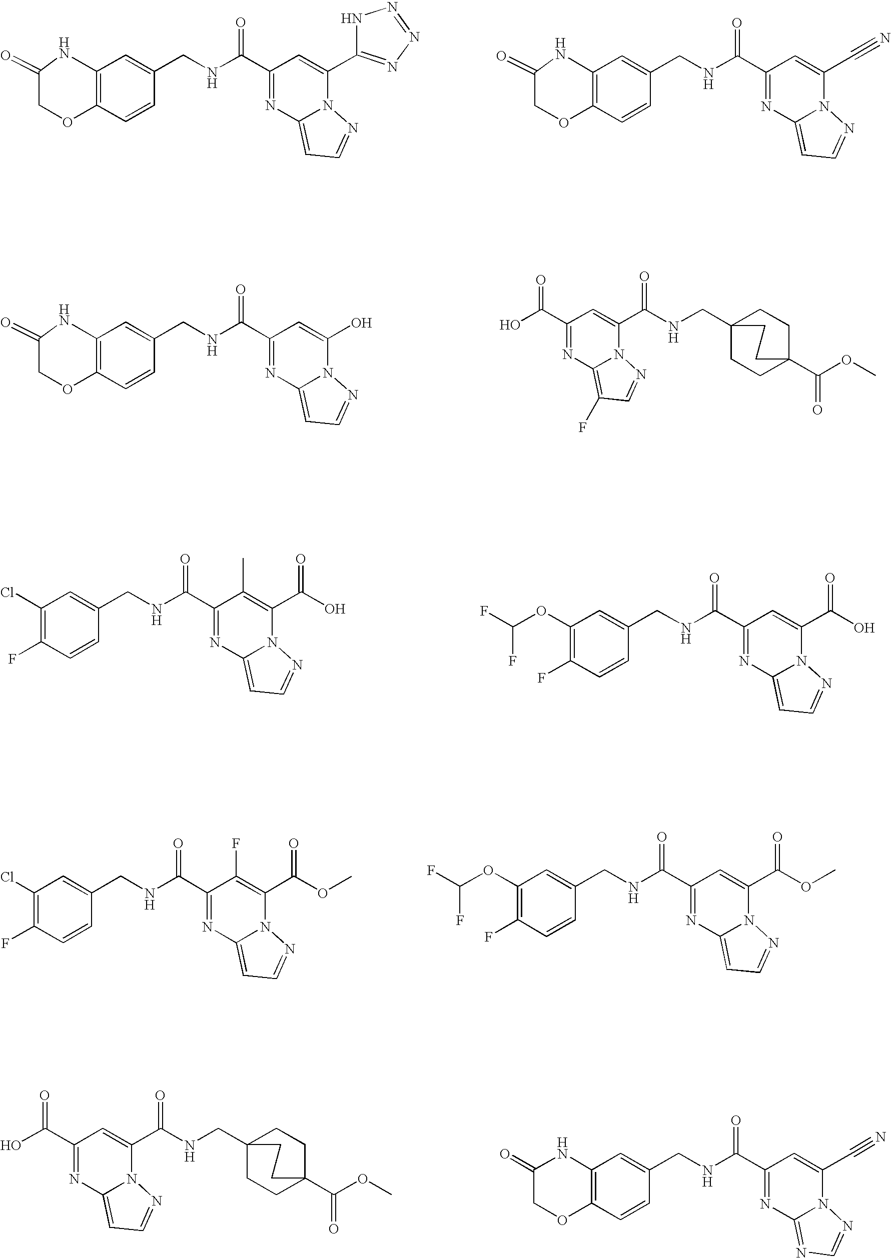

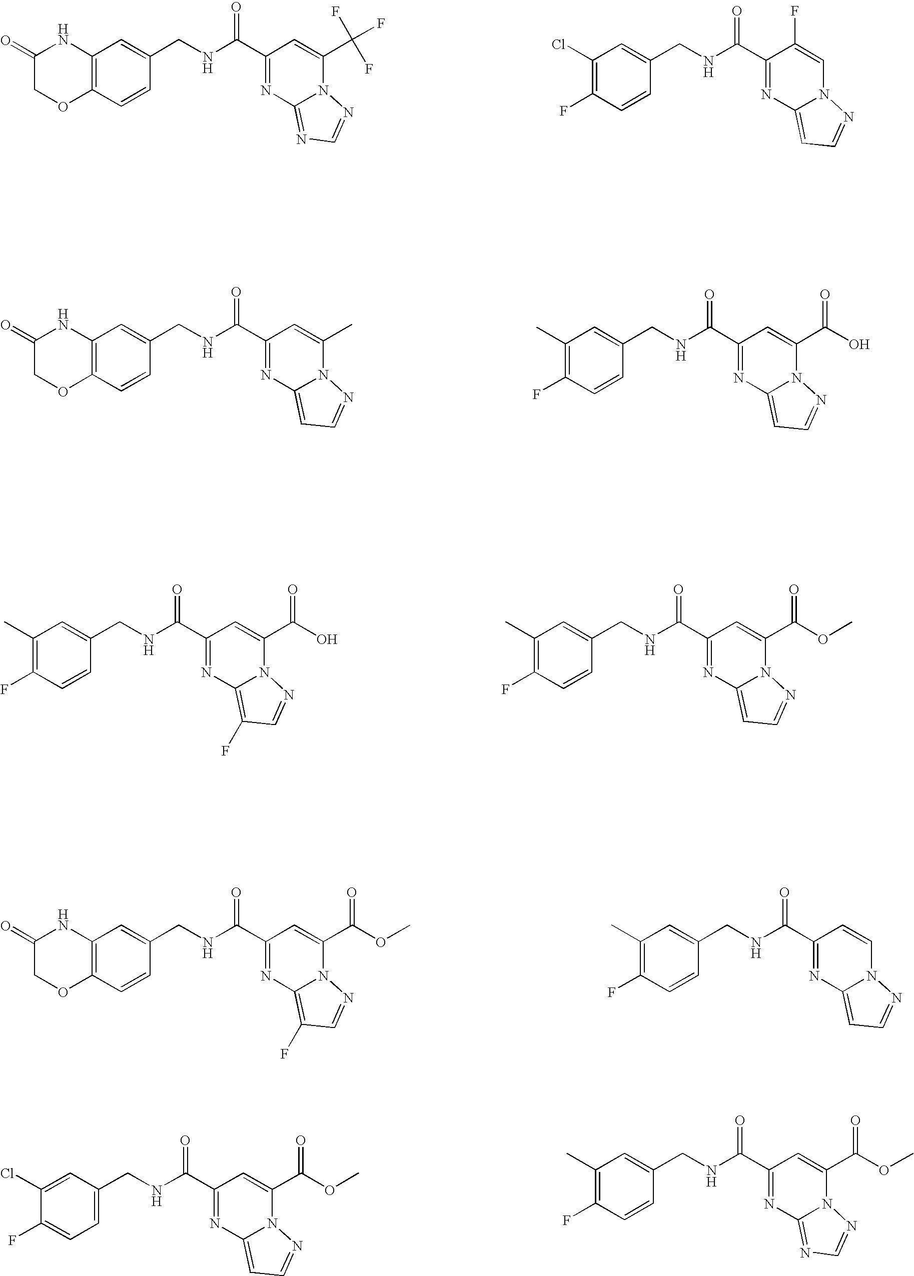

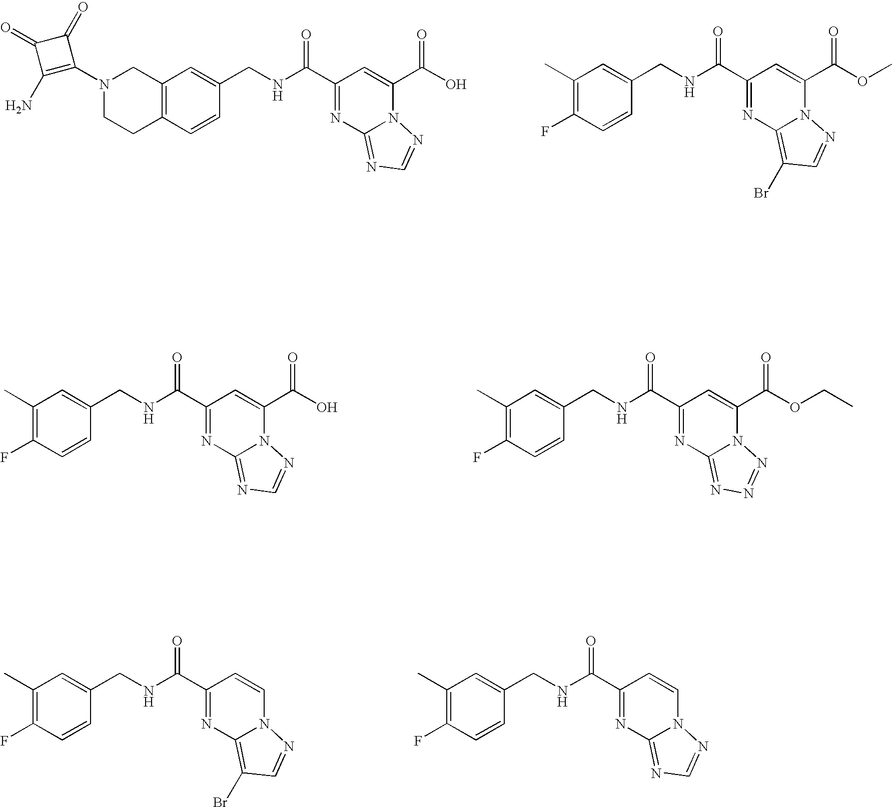

or a pharmaceutically acceptable salt thereof.

Another aspect of the invention relates to a pharmaceutical composition comprising an effective amount of the compound according to any of the above or below embodiments.

Another aspect of the invention relates to a method of treating a metalloprotease mediated disease, comprising administering to a subject in need of such treatment an effective amount of a compound according to any of the above or below embodiments.

In another embodiment, in conjunction with any above or below embodiments, the disease is selected from rheumatoid arthritis, osteoarthritis, inflammation, atherosclerosis and multiple sclerosis.

Another aspect of the invention relates to a pharmaceutical composition comprising:

A) an effective amount of a compound according to any of the above or below embodiments;
B) a pharmaceutically acceptable carrier; and
C) a drug, agent or therapeutic selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

Another aspect of the invention relates to a method of inhibiting a metalloprotease enzyme, comprising administering a compound according to any of the above or below embodiments.

In another embodiment, in conjunction with any above or below embodiments, the metalloproteinase is selected from MMP-2, MMP-3, MMP-8, and MMP-13.

In another embodiment, in conjunction with any above or below embodiments, the disease is selected from: rheumatoid arthritis, osteoarthritis, abdominal aortic aneurysm, cancer (e.g. but not limited to melanoma, gastric carcinoma or non-small cell lung carcinoma), inflammation, atherosclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, wound healing, hemorrhoid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, chronic periodontitis, periodontitis, peritonitis associated with continuous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

Another aspect of the invention relates to the use of a compound according to any of the above or below embodiments for the manufacture of a medicament for treating an metalloprotease mediated disease.

In another embodiment, in conjunction with any of the above or below embodiments, the metalloprotease mediated disease is selected from MMP-2, MMP-3, MMP-8 and MMP-13 mediated diseases.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO—wherein R¹⁰ or R¹¹ are as defined below, except that at least one of R¹⁰ or R¹¹ is not hydrogen), amino, heterocyclo, mono- or dialkylamino, or thiol (—SH).

The term “heteroalkyl” and which may be used interchangeably with the term “alkyl” denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO—wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO—wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO—wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, containing one ring with 3 to 9 carbons. Exemplary unsubstituted such groups include, but are not limited to, cyclopropyl, cyclobutyl, cyclopentyl, cyclohexyl, cycloheptyl, cyclooctyl, cyclodecyl, and cyclododecyl. Exemplary substituents include, but are not limited to, one or more alkyl groups as described above, or one or more groups described above as alkyl substituents.

The term “bicycloalkyl”, as used herein alone or as part of another group, denotes optionally substituted, saturated cyclic bridged hydrocarbon ring systems, desirably containing 2 or 3 rings and 3 to 9 carbons per ring. Exemplary unsubstituted such groups include, but are not limited to, adamantyl, bicyclo[2.2.2]octane, bicyclo[2.2.1]heptane and cubane. Exemplary substituents include, but are not limited to, one or more alkyl groups as described above, or one or more groups described above as alkyl substituents.

The term “spiroalkyl”, as used herein alone or as part of another group, denotes optionally substituted, saturated hydrocarbon ring systems, wherein two rings are bridged via one carbon atom and 3 to 9 carbons per ring. Exemplary unsubstituted such groups include, but are not limited to, spiro[3.5]nonane, spiro[4.5]decane or spiro[2.5]octane. Exemplary substituents include, but are not limited to, one or more alkyl groups as described above, or one or more groups described above as alkyl substituents.

The term “spiroheteroalkyl”, as used herein alone or as part of another group, denotes optionally substituted, saturated hydrocarbon ring systems, wherein two rings are bridged via one carbon atom and 3 to 9 carbons per ring. At least one carbon atom is replaced by a heteroatom independently selected from N, O and S. The nitrogen and sulfur heteroatoms may optionally be oxidized. Exemplary unsubstituted such groups include, but are not limited to, 1,3-diaza-spiro[4.5]decane-2,4-dione. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from N, O and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

Further examples of heterocycles include, but not are not limited to, “heterobicycloalkyl” groups such as 7-oxa-bicyclo[2.2.1]heptane, 7-aza-bicyclo[2.2.1]heptane, and 1-aza-bicyclo[2.2.2]octane.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl.

“Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more substituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The phrase “fused” means, that the group, mentioned before “fused” is connected via two adjacent atoms to the ring system mentioned after “fused” to form a bicyclic system. For example, “heterocycloalkyl fused aryl” includes, but is not limited to, 2,3-dihydro-benzo[1,4]dioxine, 4H-benzo[1,4]oxazin-3-one, 3H-Benzooxazol-2-one and 3,4-dihydro-2H-benzo[f][1,4]oxazepin-5-one.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocyclylalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The phrase “bicyclic fused ring system wherein at least one ring is partially saturated” denotes an 8- to 13-membered fused bicyclic ring group in which at least one of the rings is non-aromatic. The ring group has carbon atoms and optionally 1-4 heteroatoms independently selected from N, O and S. Illustrative examples include, but are not limited to, indanyl, tetrahydronaphthyl, tetrahydroquinolyl and benzocycloheptyl.

The phrase “tricyclic fused ring system wherein at least one ring is partially saturated” denotes a 9- to 18-membered fused tricyclic ring group in which at least one of the rings is non-aromatic. The ring group has carbon atoms and optionally 1-7 heteroatoms independently selected from N, O and S. Illustrative examples include, but are not limited to, fluorene, 10,11-dihydro-5H-dibenzo[a,d]cycloheptene and 2,2a,7,7a-tetrahydro-1H-cyclobuta[a]indene.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Examples therefore may be, but are not limited to, sodium, potassium, choline, lysine, arginine or N-methyl-glucamine salts, and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Pa., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The phrase “pharmaceutically acceptable carrier” denotes media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to poly(D,L)-lactide-co-glycolic acid copolymer (PLGA), poly(L-lactic acid) (PLA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions of the invention may also be formulated as suspensions including a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. The amount of solubility enhancer employed will depend on the amount of the compound of the present invention in the composition.

The term “formulation” denotes a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about −10-80° C., desirably about 0° C.

The term “polymorph” denotes a form of a chemical compound in a particular crystalline arrangement. Certain polymorphs may exhibit enhanced thermodynamic stability and may be more suitable than other polymorphic forms for inclusion in pharmaceutical formulations.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures.

The term “racemic mixture” denotes a mixture that is about 50% of one enantiomer and about 50% of the corresponding enantiomer relative to all chiral centers in the molecule. Thus, the invention encompasses all enantiomerically-pure, enantiomerically-enriched, and racemic mixtures of compounds of Formulas (I) through (VI).

Enantiomeric and stereoisomeric mixtures of compounds of the invention can be resolved into their component enantiomers or stereoisomers by well-known methods. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N.Y.). Enantiomers and stereoisomers can also be obtained from stereomerically- or enantiomerically-pure intermediates, reagents, and catalysts by well-known asymmetric synthetic methods.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:

C₁-C₄ alkyl;

C₂-C₄ alkenyl;

C₂-C₄ alkynyl;

CF₃;

halo;

OH;

O—(C₁-C₄ lkyl);

—OCH₂F;

OCHF₂;

OCF₃;

ONO₂;

OC(O)—(C₁-C₄ alkyl);

OC(O)—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂;

SH;

S—(C₁-C₄ alkyl);

S(O)—(C₁-C₄ alkyl);

S(O)₂—(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ alkyl);

N(CH₃)C(S)—(C₁-C₄ alkyl);

N(H)S(O)₂—(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂);

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH:

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄ alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl);

CH₂N(C₁-C₄ alkyl)₂;

aryl;

heteroaryl;

cycloalkyl; and

heterocyclyl.

In some cases, a ring substituent may be shown as being connected to the ring by a bond extending from the center of the ring. The number of such substituents present on a ring is indicated in subscript by a number. Moreover, the substituent may be present on any available ring atom, the available ring atom being any ring atom which bears a hydrogen which the ring substituent may replace. For illustrative purposes, if variable R^{X} were defined as being:

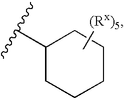

this would indicate a cyclohexyl ring bearing five R^{X} substituents. The R^{X} substituents may be bonded to any available ring atom. For example, among the configurations encompassed by this are configurations such as:

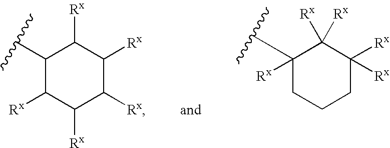

These configurations are illustrative and are not meant to limit the scope of the invention in any way.

### Biological Activity

The determination of inhibition towards different metalloproteases of the heterobicyclic metalloprotease inhibiting compounds of the present invention may be measured using any suitable assay known in the art. A standard in vitro assay for measuring the metalloprotease inhibiting activity is described in Examples 1700 to 1704. The heterobicyclic metalloprotease inhibiting compounds show activity towards MMP-3, MMP-8, MMP-12, MMP-13, ADAMTS-4 and/or ADAMTS-5.

The heterobicyclic metalloprotease inhibiting compounds of the invention have an MMP-13 inhibition activity (IC₅₀ MMP-13) ranging from below 10 nM to about 20 μM, and typically, from about 3 nM to about 2 μM. Heterobicyclic metalloprotease inhibiting compounds of the invention desirably have an MMP inhibition activity ranging from about 3 nM to about 200 nM. Table 1 lists typical examples of heterobicyclic metalloprotease inhibiting compounds of the invention that have an MMP-13 activity of lower than 200 nM (Group A) and from 201 nM to 20 μM (Group B).

TABLE 1
Summary of MMP-13 Activity for Compounds

| Group | Examples |
|---|---|
| A | 29, 32, 152, 163, 141, 160, 208 |
| B | 142, 150, 188, 190, 193, 195, 209, 211 |

The synthesis of metalloprotease inhibiting compounds of the invention and their biological activity assay are described in the following examples which are not intended to be limiting in any way.

### EXAMPLES AND METHODS

All reagents and solvents were obtained from commercial sources and used without further purification. Proton (¹H) spectra were recorded on a 250 or 400 MHz NMR spectrometer in deuterated solvents. Flash chromatography was performed using Merck silica gel, grade 60, 70-230 mesh using suitable organic solvents as indicated in specific examples. Thin layer chromatography (TLC) was carried out on silica gel plates with UV detection.

### Preparative Example 1

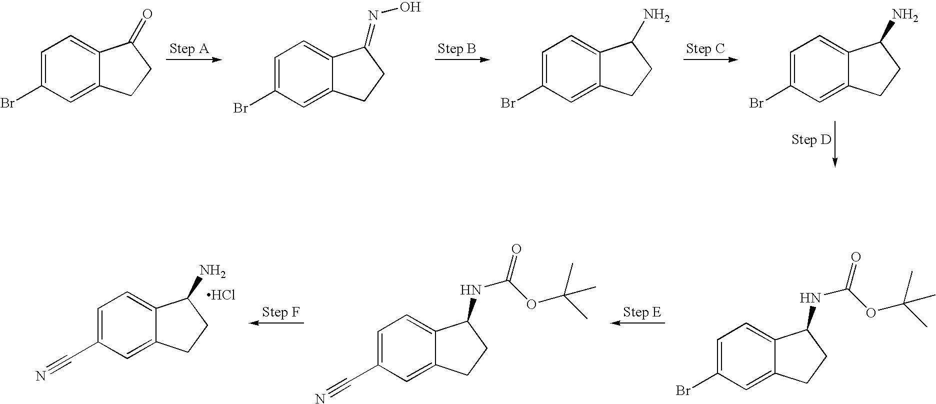

### Step A

A mixture of commercially available 5-bromo-indan-1-one (1.76 g), hydroxylamine hydrochloride (636 mg) and sodium acetate (751 mg) in methanol (40 mL) was allowed to stir for 16 h at room temperature. Water (100 mL) was added and the resulting precipitate was filtered and washed with water (3×20 mL) to afford the title compound (1.88 g; >99%) as a colourless solid. [MH]⁺=226/228.

### Step B

To a solution of the title compound from Step A above (1.88 g) in diethyl ether (20 mL) at −78° C. under an atmosphere of argon was slowly added a 1M solution of lithium aluminum hydride in diethyl ether (42.4 mL). The mixture was heated to reflux (40° C.) and allowed to stir for 5 h. The mixture was cooled to 0° C. and water (1.6 mL), 15% aqueous sodium hydroxide (1.6 mL) and water (4.8 mL) were carefully and sequentially added. The resulting mixture was filtered through Celite® and the filtrate was concentrated to give the title compound (1.65 g; 94%) as a clear oil. [MH]⁺=212/214. Celite® and the filtrate was concentrated to give the title compound (1.65 g; 94%) as a clear oil. [MH]⁺=212/214.

### Step C

To a boiling solution of the title compound from Step B above (1.13 g) in methanol (2.3 mL) was added a hot solution of commercially available N-acetyl-L-leucine (924 mg) in methanol (3 mL). The solution was allowed to cool to room temperature, which afforded a white precipitate. The solid was separated from the supernatant and washed with methanol (2 mL). The solid was recrystallized two times from methanol. To the resulting solid were added 10% aqueous sodium hydroxide (20 mL) and diethyl ether (20 mL). Once the solid was dissolved, the organic layer was separated and the aqueous layer was washed with diethyl ether. The combined organic layers were dried (MgSO₄), filtered and concentrated to give the title compound (99 mg; 18%) as a clear oil. [MH]⁺=212/214.

### Step D

To a solution of the title compound from Step C above (300 mg), di-tert-butyl dicarbonate (370 mg) and triethylamine (237 μL) in tetrahydrofuran (10 mL) was allowed to stir for 16 h at room temperature. The solution was concentrated and the remaining residue was purified by chromatography (silica, hexanes/ethyl acetate) to give the title compound (460 mg; >99%) as a clear oil. [(M-isobutene)H]⁺=256/258, [MNa]⁺=334/336.

### Step E

A mixture of the title compound from Step D above (460 mg), tetrakis triphenylphosphinepalladium (89 mg), zinc cyanide (200 mg) in N,N-dimethylformamide (5 mL) under an atmosphere of argon in a sealed vial was allowed to stir for 18 h at 1110° C. The mixture was allowed to cool to room temperature before diethyl ether (20 mL) and water (20 mL) were added. The separated aqueous layer was washed with diethyl ether (4×10 mL). The combined organic layers were washed with water (3×10 mL) and brine (10 mL), dried (MgSO₄), filtered and concentrated. The resulting residue was purified by chromatography (silica, hexanes/ethyl acetate) to afford the title compound (170 mg; 47%) as a clear oil. [MH]⁺=259, [MNa]⁺=281.

### Step F

To the title compound from Step E above (170 mg) was added a 4M solution of hydrochloric acid in dioxane (2 mL). The resulting solution was allowed to stir for 3 h at room temperature at which time a precipitate had formed. The mixture was concentrated to give 1(S)-amino-indan-5-carbonitrile hydrochloride (128 mg; >99%). [M−Cl]⁺=159.

### Preparative Example 2

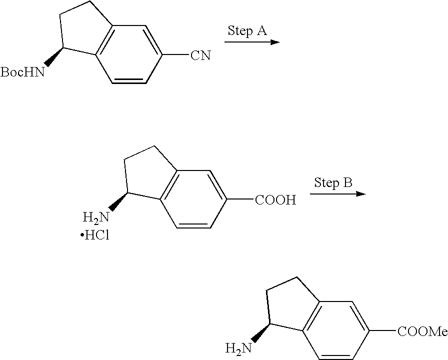

### Step A

(5-Cyano-indan-1(S)-yl)-carbamic acid tert-butyl ester (1.0 g) was suspended in 6N hydrochloric acid (50 mL) and heated to 110-112° C. for 20 h upon which the solution became homogeneous. The solvent was removed under reduce pressure to give the intermediate. [M=Cl]⁺=178.

### Step B

The intermediate from Step A above was dissolved in anhydrous MeOH (150 mL) and saturated with anhydrous hydrogen chloride gas. The reaction mixture was then heated to reflux for 20 h. After cooling to room temperature, the solvent was removed under reduced pressure to give an oil. The oil was taken up in dichloromethane and washed with saturated NaHCO₃. The organic phase was separated and dried over MgSO₄, filtered and concentrated to give 1(S)-amino-indan-5-carboxylic acid methyl ester (0.66 g, 89% over two steps) as an oil which slowly crystallized into a light brown solid.

### Preparative Example 3

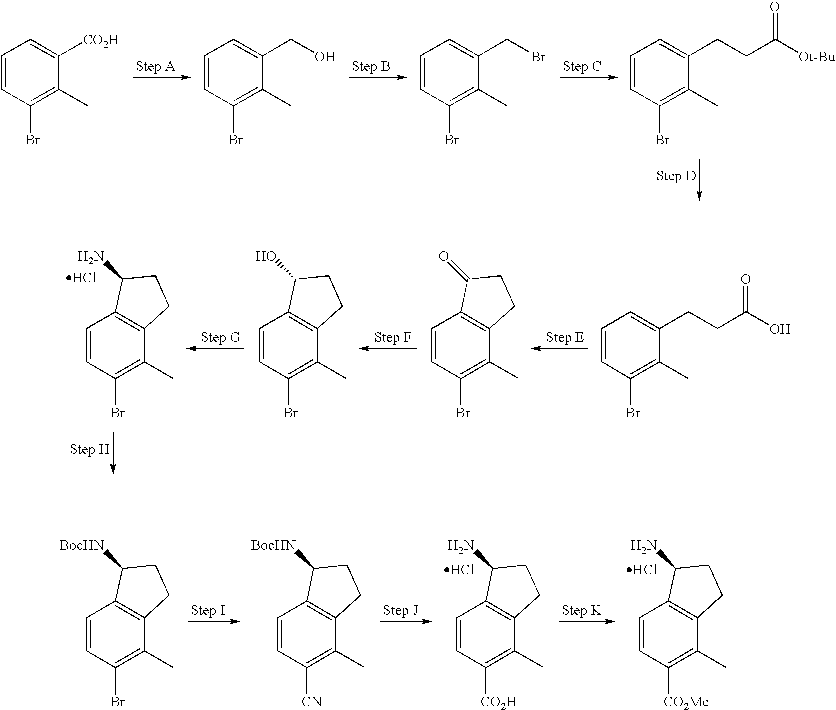

### Step A

3-Bromo-2-methyl-benzoic acid (20.0 g) was dissolved in anhydrous THF (200 mL) under nitrogen and the reaction vessel was cooled to 0° C. in an ice bath. To this cooled solution was added BH₃-THF complex (1M in THF, 140 mL) dropwise over a 3 h period. Once gas evolution had subsided, the reaction mixture was warmed to room temperature and stirred for an additional 12 h. The mixture was then poured into 1N hydrochloric acid (500 mL) cooled with ice and then extracted with Et₂O (3×150 mL). The organic extracts were combined, dried over anhydrous MgSO₄, filtered, and then concentrated to afford the intermediate (18.1 g; 97%) as a colourless solid. ¹H-NMR (CDCl₃) δ=2.40 (s, 3H), 4.70 (s, 2H), 7.10 (t, 1H), 7.30 (d, 1H), 7.50 (d, 1H).

### Step B

The intermediate from Step A above (18.1 g) was dissolved in anhydrous CH₂Cl₂ (150 mL) under nitrogen and the reaction vessel was cooled to 0° C. in an ice bath. To this cooled solution was added PBr₃ (5.52 mL) over a 10 min period. Once the addition was complete, the reaction mixture was warmed to room temperature and stirred for an additional 12 h. The mixture was cooled in an ice bath and quenched by the dropwise addition of MeOH (20 mL). The organic phase was washed with saturated NaHCO₃ (2×150 mL), dried over anhydrous MgSO₄, filtered, and then concentrated to afford the intermediate (23.8 g; 97%) as viscous oil. ¹H-NMR (CDCl₃) δ=2.50 (s, 3H), 4.50 (s, 2H), 7.00 (t, H), 7.25 (d, 1H), 7.50 (d, 1H).

### Step C

t-Butyl acetate (12.7 mL) was dissolved in anhydrous THF (200 mL) under nitrogen and the reaction vessel was cooled to −78° C. in a dry ice/acetone bath. To this cooled solution was added dropwise lithium diispropylamide (1.5M in cyclohexane, 63.0 mL) and the mixture was allowed to stir for an additional 1 h upon which a solution of intermediate from Step B above (23.8 g) was added in THF (30 mL). Once the addition was complete, the reaction mixture was gradually warmed to room temperature over a 12 h period. The mixture was concentrated and the remaining viscous oil was dissolved in Et₂O (300 mL), washed with 0.5N hydrochloric acid (2×100 mL), dried over anhydrous MgSO₄, filtered, and then concentrated to afford the intermediate (21.5 g; 80%) as a pale-yellow viscous oil. ¹H-NMR (CDCl₃) δ=1.50 (s, 9H), 2.40 (s, 3H), 2.50 (t, 2H), 3.00 (t, 2H), 7.00 (t, 1H), 7.25 (d, 1H), 7.50 (d, 1H).

### Step D

The intermediate from Step C above (21.5 g) was combined with polyphosphoric acid (250 g) and placed in a 140° C. oil bath for 10 min while mixing the thick slurry occasionally with a spatula. To this mixture was then added ice water (1 L) and the mixture was stirred for 2 h. The mixture was then filtered and the solid was washed with H₂O (2×100 mL) and dried to afford the intermediate (16.7 g; 96%). ¹H-NMR (CDCl₃) δ=2.40 (s, 3H), 2.65 (t, 2H), 3.00 (t, 2H), 7.00 (t, 1H), 7.20 (d, 1H), 7.50 (d, 1H).

### Step E

The intermediate from Step D above (11.6 g) was dissolved in anhydrous CH₂Cl₂ (100 mL) under nitrogen and the reaction vessel was cooled to 0° C. in an ice bath. To this mixture was added dropwise oxalyl chloride (12.0 mL) and the mixture was stirred for 3 h after which the mixture was concentrated under reduced pressure. The remaining dark residue was dissolved in anhydrous CH₂Cl₂ (300 mL) and to this mixture was added AlCl₃ (6.40 g). Once the addition was complete, the mixture was refluxed for 4 h upon which the mixture was poured into ice water (500 mL) and extracted with CH₂Cl₂ (2×11 mL). The combined extracts were combined, dried over anhydrous MgSO₄, filtered, and then concentrated to afford the intermediate (10.6 g; 98%) as a light brown solid. ¹H-NMR (CDCl₃) δ=2.40 (s, 9H), 2.70 (t, 2H), 3.05 (t, 2H), 7.50 (d, 1H), 7.65 (d, 1H).

### Step F

To a cooled solution of (S)-2-methyl-CBS-oxazaborolidine (1M in toluene, 8.6 mL) and boraneemethyl sulfide complex (1M in CH₂Cl₂, 43.0 mL) at −20° C. (internal temperature) in CH₂Cl₂ (200 mL) was added a solution of intermediate from Step E above (9.66 g, in 70 mL CH₂Cl₂) over a 10 h period via a syringe pump. After the addition was complete, the mixture was then quenched by the addition of MeOH (100 mL) at −20° C., warmed to room temperature and concentrated. The crude mixture was purified by flash chromatography (10% to 30% Et₂O/CH₂Cl₂ gradient) to afford the intermediate (8.7 g; 90%) as a colourless solid. ¹H-NMR (CDCl₃) δ=2.00 (m, 1H), 2.35 (s, 3H), 2.50 (m, 1H), 2.90 (m, 1H), 3.10 (m, 1H), 5.25 (m, 1H), 7.20 (d, 1H), 7.50 (d, 1H).

### Step G

To a −78° C. cooled solution of intermediate from step F above (8.7 g) in CH₂Cl₂ (200 mL) under nitrogen was added triethylamine (15.9 mL) followed by methanesulfonyl chloride (4.5 mL). This mixture was stirred for 90 min and then NH₃ (−150 mL) was condensed into the mixture using a dry ice/acetone cold finger at a rate of −3 mL/minute. After stirring at −78° C. for an additional 2 h, the mixture was gradually warmed to room temperature allowing the NH₃ to evaporate from the reaction mixture. 1N NaOH (200 mL) was added and the aqueous layer was extracted with CH₂Cl₂ (2×100 mL). The combined extracts were dried over anhydrous MgSO₄, filtered, and then concentrated to afford crude material as a light brown oil. This oil was dissolved in Et₂O (200 mL) and hydrogen chloride (4M in dioxane, 10 mL) was added and the precipitate was collected and dried to give the intermediate (9.0 g; 90%). [M−NH₃Cl]⁺=209/211.

### Step H

The intermediate from Step G above (5.2 g) was mixed in dry CH₂Cl₂ (50 mL) and cooled to 0° C. and to this cooled solution was added di-tert-butyl dicarbonate (5.0 g) followed by Et₃N (9.67 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid. ¹H-NMR (CDCl₃, free base) δ=1.80 (m, 1H), 2.30 (s, 3H), 2.60 (m, 1H), 2.80 (m, 1H), 2.90 (m, 1H), 4.30 (t, 1H), 7.00 (d, 1H), 7.40 (m, H).

### Step I

The intermediate from Step H above (7.2 g), zinc(II) cyanide (5.2 g) and Pd(PPh₃)₄ (2.6 g) were combined under nitrogen and anhydrous DMF (80 mL) was added. The yellow mixture was heated to 100° C. for 18 h and then concentrated under-reduced pressure to afford crude material which was purified by flash chromatography (20% CH₂Cl₂/EtOAc) to give the intermediate (4.5 g; 75%) as an off-white solid. ¹H-NMR (CDCl₃) δ=1.50 (s, 3H), 1.90 (m, 1H), 2.40 (s, 3H), 2.70 (m, 1H), 2.80 (m, H), 2.95 (m, 1H), 4.75 (m, 1H), 5.15 (m, 1H), 7.20 (d, 1H), 7.50 (d, 1H).

### Step J

The intermediate from Step I above (1.0 g) was suspended in 6N hydrochloric acid (20 mL) and heated to 100° C. for 12 h upon which the solution become homogeneous. The solvent was removed under reduce pressure to give the intermediate (834 mg; quantitative) as a colourless solid. [M−NH₃Cl]⁺=175.

### Step K

The intermediate from Step J above (1.0 g) was dissolved in anhydrous MeOH (20 mL) and cooled to 0° C. and anhydrous hydrogen chloride was bubbled through this solution for 2-3 min. The reaction mixture was then heated to reflux for 12 h. After cooling to room temperature, the solvent was removed under reduced pressure to give 1(S)-amino-4-methyl-indan-5-carboxylic acid methyl ester hydrochloride (880 mg; quantitative) as a colourless solid. [M−NH₃Cl]⁺=189.

### Preparative Example 4

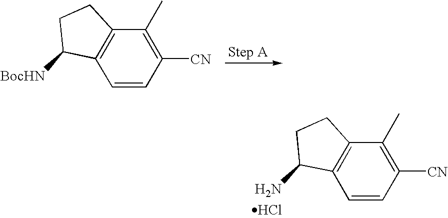

### Step A

To (5-cyano-4-methyl-indan-1(S)-yl)-carbamic acid tert-butyl ester (108 mg) was added a solution of hydrogen chloride (4M in dioxane, 2 mL) and the resulting solution was allowed to stir at 22° C. for 6 h at which time a precipitate had formed. The mixture was concentrated to give the title compound (83 mg, >99%) as a colourless powder. [M−NH₃Cl]⁺=156.

### Preparative Example 5

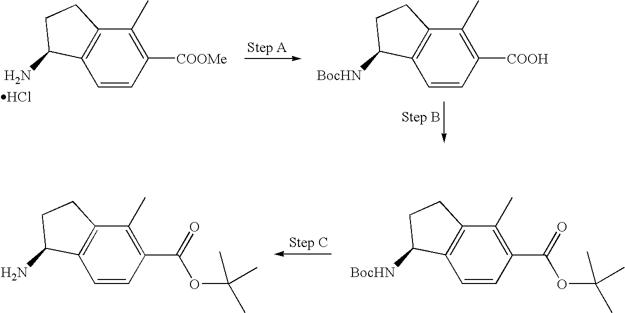

### Step A

1(S)-Amino-4-methyl-indan-5-carboxylic acid methyl ester hydrochloride (1.5 g) was mixed in dry CH₂Cl₂ (50 mL) and cooled to 0° C. and to this cooled solution was added di-tert-butyl dicarbonate (1.6 g) followed by Et₃N (1 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid which was dissolved in tetrahydrofuran (60 mL). To the mixture was added a 1M aqueous LiOH solution (60 mL) and the mixture was stirred at 50° C. for 2 h. The mixture was concentrated to dryness and redissolved in water, acidified to pH=5 with hydrochloric acid and extracted with ethyl acetate. The organic layer was dried (MgSO₄) and concentrated to afford the intermediate as colourless solid (1.87 g). [MNa]⁺=314.

### Step B

To a solution of the title compound from Step A above (1.87 g) in dry toluene (15 mL) was added Di-tert-butoxymethyl dimethylamine (6.2 mL) at 80° C. At this temperature the mixture was stirred for 3 h. After cooling to room temperature the mixture was concentrated and purified by column chromatography (silica, dichloromethane) to afford the intermediate (820 mg; 38%) as a colourless solid. [MNa]⁺=370.

### Step C

To a solution of the title compound from Step B above (820 mg) in tert-butyl acetate (40 mL) was added sulfuric acid (0.65 mL) at room temperature. The mixture was stirred for 5 h and concentrated to dryness. The residue was dissolved ethyl acetate and washed with a saturated solution of sodium hydrogen carbonate and brine. After drying (MgSO₄) 1(S)-amino-4-methyl-indan-5-carboxylic acid tert-butyl ester (640 mg; 99%) was obtained as a colourless solid. [M−NH₂]⁺=231.

### Preparative Example 6

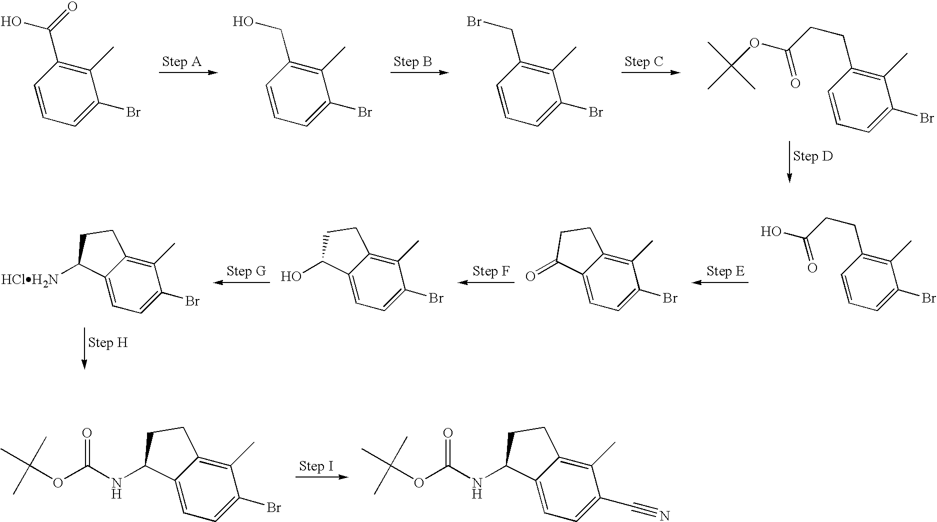

Step A

Under a nitrogen atmosphere a 1M solution of BH₃.THF complex in THF (140 mL) was added dropwise over a 3 h period to an ice cooled solution of commercially available 3-bromo-2-methyl-benzoic acid (20.0 g) in anhydrous THF (200 mL). Once gas evolution had subsided, the cooling bath was removed and mixture stirred at room temperature for 12 h. The mixture was then poured into a mixture of 1N aqueous HCl (500 mL) and ice and then extracted with Et₂O (3×150 mL). The combined organic phases were dried (MgSO₄), filtered and concentrated to afford the title compound as a colorless solid (18.1 g, 97%). ¹H-NMR (CDCl₃) δ=7.50 (d, 1H), 7.30 (d, 1H), 7.10 (t, 1H), 4.70 (s, 2H), 2.40 (s, 3H).

### Step B

Under a nitrogen atmosphere PBr₃ (5.52 mL) was added over a 10 min period to an ice cooled solution of the title compound from Step A above (18.1 g) in anhydrous CH₂Cl₂ (150 mL). The cooling bath was removed and mixture stirred at room temperature for 12 h. The mixture was cooled (0-5° C.), quenched by dropwise addition of MeOH (20 mL), washed with saturated aqueous NaHCO₃ (2×150 mL), dried (MgSO₄), filtered and concentrated to afford the title compound as a viscous oil (23.8 g, 97%). ¹H-NMR (CDCl₃) δ=7.50 (d, 1H), 7.25 (d, 1H), 7.00 (t, 1H), 4.50 (s, 2H), 2.50 (s, 3H).

### Step C

Under a nitrogen atmosphere a 1.5M solution of lithium diispropylamide in cyclohexane (63 mL) was added dropwise to a cooled (−78° C., acetone/dry ice) solution of ^{t}BuOAc in anhydrous THF (200 mL). The mixture was stirred at −78° C. for 1 h, then a solution of the title compound from Step B above (23.8 g) in THF (30 mL) was added and the mixture was stirred for 12 h while warming to room temperature. The mixture was concentrated, diluted with Et₂O (300 mL), washed with 0.5N aqueous HCl (2×100 mL), dried (MgSO₄), filtered and concentrated to afford the title compound as a pale-yellow viscous oil (21.5 g, 80%). ¹H-NMR (CDCl₃) δ=7.50 (d, 1H), 7.25 (d, 1H), 7.00 (t, 1H), 3.00 (t, 2H), 2.50 (t, 2H), 2.40 (s, 3H), 1.50 (s, 9H).

### Step D

A mixture of the title compound from Step C above (21.5 g) and polyphosphoric acid (250 g) was placed in a preheated oil bath (140° C.) for 10 min while mixing the thick slurry occasionally with a spatula. The oil bath was removed, ice and H₂O (1 L) was added and the mixture was stirred for 2 h. The precipitate was isolated by filtration, washed with H₂O (2×100 mL) and dried to afford the title compound (16.7 g, 96%). ¹H-NMR (CDCl₃) δ=7.50 (d, 1H), 7.20 (d, 1H), 7.00 (t, 1H), 3.00 (t, 2H), 2.65 (t, 2H), 2.40 (s, 3H).

### Step E

Under a nitrogen atmosphere oxalyl chloride (12.0 mL) was added dropwise to an ice cooled solution of the title compound from Step D above (11.6 g) in anhydrous CH₂Cl₂ (100 mL). The resulting mixture was stirred for 3 h and then concentrated. The remaining dark residue was dissolved in anhydrous CH₂Cl₂ (300 mL) and AlCl₃ (6.40 g) was added. The mixture was heated to reflux for 4 h, cooled and poured into ice water (500 mL). The aqueous phase was separated and extracted with CH₂Cl₂ (2×100 mL). The combined organic phases were dried (MgSO₄), filtered and concentrated to afford the title compound as a light brown solid (10.6 g, 98%). ¹H-NMR (CDCl₃) δ=7.65 (d, 1H), 7.50 (d, 1H), 3.05 (t, 2H), 2.70 (t, 2H), 2.40 (s, 3H).

### Step F

Using a syringe pump, a solution of the title compound from Step E above (9.66 g) in anhydrous CH₂Cl₂ (70 mL) was added over a 10 h period to a cooled (−20° C., internal temperature) mixture of a 1M solution of (S)-(−)-2-methyl-CBS-oxazaborolidine in toluene (8.6 mL) and a 1M solution of BH₃.Me₂S complex in CH₂Cl₂ (43.0 mL) in CH₂Cl₂ (200 mL). The mixture was then quenched at −20° C. by addition of MeOH (100 mL), warmed to room temperature, concentrated and purified by flash chromatography (silica, Et₂O/CH₂Cl₂) to afford the title compound as a colorless solid (8.7 g, 90%). ¹H-NMR (CDCl₃) δ=7.50 (d, 1H), 7.20 (d, 1H), 5.25 (m, 1H), 3.10 (m, 1H), 2.90 (m, 1H), 2.50 (m, 1H), 2.35 (s, 3H), 2.00 (m, 1H).

### Step G

Under a nitrogen atmosphere NEt₃ (15.9 mL) and methanesulfonyl chloride (4.5 mL) were added subsequently to a cooled (−78° C., acetone/dry ice) solution of the title compound from Step F above (8.7 g) in anhydrous CH₂Cl₂ (200 mL). The mixture was stirred at −78° C. for 90 min, then NH₃ (˜150 mL) was condensed into the mixture using a dry ice condenser at a rate of ˜3 mL/min and stirring at −78° C. was continued for 2 h. Then the mixture was gradually warmed to room temperature allowing the NH₃ to evaporate. 1N aqueous NaOH (200 mL) was added, the organic phase was separated and the aqueous phase was extracted with CH₂Cl₂ (2×100 mL). The combined organic phases were dried (MgSO₄), filtered and concentrated. The remaining light brown oil was dissolved in Et₂O (200 mL) and a 4M solution of HCl in 1,4-dioxane (10 mL) was added. The formed precipitate was collected and dried to give the title compound (9.0 g, 90%). [M−NH₃Cl]⁺=209/211.

### Step H

To an ice cooled solution of the title compound from Step G above (5.2 g) in anhydrous CH₂Cl₂ (50 mL) were subsequently added di-tert-butyl dicarbonate (5.0 g) and NEt₃ (9.67 mL). The resulting mixture was stirred for 3 h, concentrated, diluted with Et₂O (250 mL), washed with saturated aqueous NaHCO₃ (100 mL) and saturated aqueous NaCl (100 mL), dried (MgSO₄), filtered and concentrated to afford the title compound as a colorless solid (7.28 g, 97%). ¹H-NMR (CDCl₃, free base) δ=7.40 (m, H), 7.00 (d, 1H), 4.30 (t, 1H) 2.90 (m, 1H), 2.80 (m, 1H), 2.60 (m, 1H), 2.30 (s, 3H), 1.80 (m, 1H).

### Step I

Under a nitrogen atmosphere a mixture of the title compound from Step H above (7.2 g), Zn(CN)₂ (5.2 g) and Pd(PPh₃)₄ (2.6 g) in anhydrous DMF (80 mL) was heated to 100° C. for 18 h, concentrated and purified by flash chromatography (silica, CH₂Cl₂/EtOAc) to afford the title compound as an off-white solid (4.5 g, 75%). ¹H-NMR (CDCl₃) δ=7.50 (d, 1H), 7.20 (d, 1H), 5.15 (m, 1H), 4.75 (m, 1H), 2.95 (m, 1H), 2.80 (m, 1H), 2.70 (m, 1H), 2.40 (s, 3H), 1.90 (m, 1H), 1.50 (s, 9H).

### Preparative Example 7

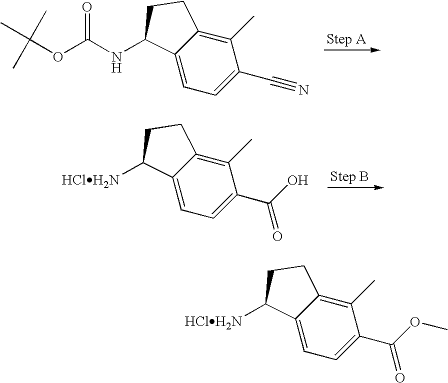

### Step A

The title compound from the Preparative Example 1, Step I (1.0 g) was suspended in 6N aqueous HCl (20 mL), heated to 100° C. for 12 h and concentrated to give the title compound as a colorless solid. (834 mg, >99%). [M−NH₃Cl]⁺=175.

### Step B

Anhydrous HCl gas was bubbled through an ice cooled solution of the title compound from Step A above (1.0 g) in anhydrous MeOH (20 mL) for 2-3 min. The cooling bath was removed, the mixture was heated to reflux for 12 h, cooled to room temperature and concentrated to give the title compound as a colorless solid (880 mg, 83%). [M−NH₃Cl]⁺=189.

### Preparative Example 8

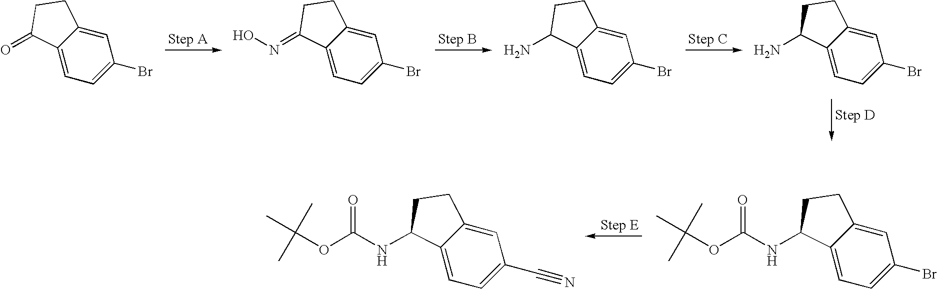

### Step A

A mixture of commercially available 5-bromo-indan-1-one (1.76 g), hydroxylamine hydrochloride (636 mg) and NaOAc (751 mg) in MeOH (40 mL) was stirred at room temperature for 16 h and then diluted with H₂O (100 mL). The formed precipitate was collected by filtration, washed with H₂O (3×20 mL) and dried to afford the title compound as a colorless solid (1.88 g, >99%). [MH]⁺=226/228.

### Step B

Under an argon atmosphere a 1M solution of LiAlH₄ in Et₂O (42.4 mL) was slowly added to a cooled (−78° C., acetone/dry ice) solution of the title compound from Step A above (1.88 g) in Et₂O (20 mL). Then the cooling bath was removed and the mixture was heated to reflux for 5 h. The mixture was cooled (0-5° C.) and H₂O (1.6 mL), 15% aqueous NaOH (1.6 mL) and H₂O (4.8 mL) were carefully and sequentially added. The resulting mixture was filtered through a plug of celites and concentrated to give the title compound as a clear oil (1.65 g, 94%). [MH]⁺=212/214.

### Step C

To a boiling solution of the title compound from Step B above (1.13 g) in MeOH (2.3 mL) was added a hot solution of commercially available N-acetyl-L-leucine (924 mg) in MeOH (3 mL). The solution was allowed to cool to room temperature, which afforded a white precipitate. The precipitate was collected by filtration, washed with MeOH (2 mL) and recrystallized from MeOH (2×). The obtained solid was dissolved in a mixture of 10% aqueous NaOH (20 mL) and Et₂O (20 mL), the organic phase was separated and the aqueous phase was extracted with Et₂O. The combined organic phases were dried (MgSO₄), filtered and concentrated to give the title compound as a clear oil (99 mg, 18%). [MH]⁺=212/214.

### Step D

To a solution of the title compound from Step C above (300 mg) in THF (10 mL) were subsequently added di-tert-butyl dicarbonate (370 mg) and NEt₃ (237 μL). The resulting mixture was stirred at room temperature for 16 h, concentrated and purified by chromatography (silica, hexanes/EtOAc) to afford the title compound as a clear oil (460 mg, >99%). [MNa]⁺=334/336.

### Step E

Under an argon atmosphere a mixture of the title compound from Step D above (460 mg), Zn(CN)₂ (200 mg) and Pd(PPh₃)₄ (89 mg) in anhydrous DMF (5 mL) was heated in a sealed vial to 110° C. for 18 h. The mixture was cooled to room temperature and diluted with Et₂O (20 mL) and H₂O (20 mL). The organic phase was separated and the aqueous phase was extracted with Et₂O (4×10 mL). The combined organic phases were washed with H₂O (3×10 mL) and saturated aqueous NaCl (10 mL), dried (MgSO₄), filtered, concentrated and purified by chromatography (silica, hexanes/EtOAc) to afford the title compound as a clear oil (170 mg, 47%). [MH]⁺=259.

### Preparative Example 9

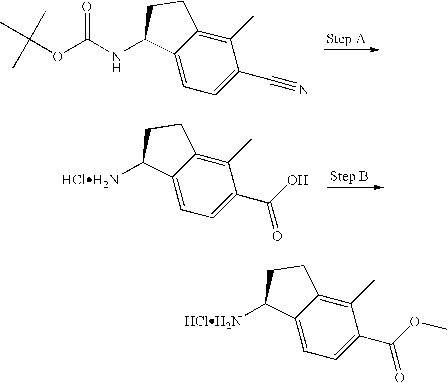

### Step A

The title compound from the Preparative Example 3, Step E (1.0 g) was suspended in 6N aqueous HCl (50 mL), heated under closed atmosphere to 110-112° C. for 20 h and concentrated to give the title compound (827 mg, >99%). [M−Cl]⁺=178.

### Step B

The title compound from Step A above (827 mg) was dissolved in anhydrous MeOH (150 mL) and saturated with anhydrous HCl gas. The resulting mixture was heated to reflux for 20 h, cooled to room temperature and concentrated. The remaining oil was taken up in CH₂Cl₂ and washed with saturated aqueous NaHCO₃, dried (MgSO₄), filtered and concentrated to give the title compound as an oil which slowly crystallized into a light brown solid (660 mg, 89%). [MH]⁺=192.

### Preparative Example 10

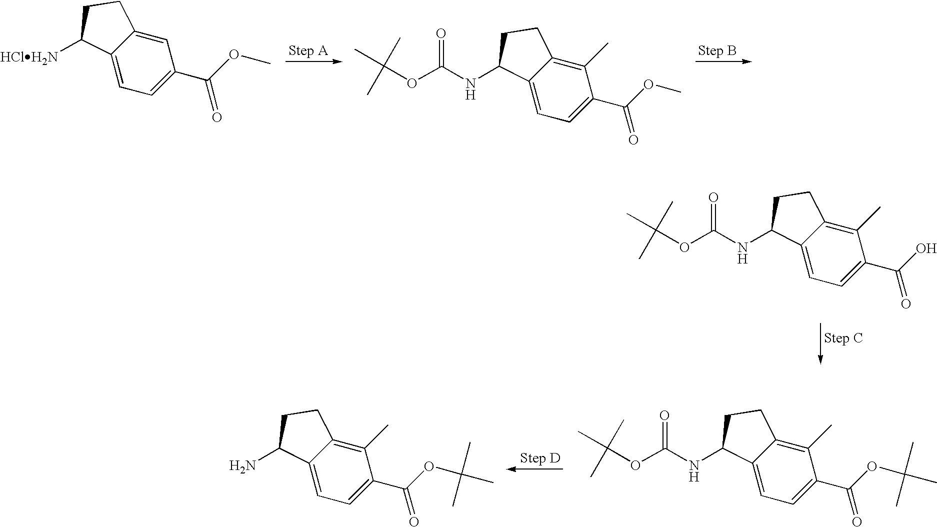

### Step A

To an ice cooled solution of the title compound from the Preparative Example 2, Step B (5.94 g) in dry CH₂Cl₂ (50 mL) were subsequently added di-tert-butyl dicarbonate (1.6 g) and NEt₃ (1 mL). The mixture was stirred for 3 h, concentrated, diluted with Et₂O (250 mL), washed with saturated aqueous NaHCO₃ (100 mL) and saturated aqueous NaCl (100 mL), dried (MgSO₄), filtered and concentrated to afford the title compound as a colorless solid (7.28 g, 97%). [MNa]⁺=328.

### Step B

To a mixture of the title compound from Step A above (7.28 g) in THF (60 mL) was added 1M aqueous LiOH (60 mL). The mixture was stirred at 50° C. for 2 h, concentrated, diluted with H₂O, adjusted to pH 5 with HCl and extracted with EtOAc. The combined organic phases were dried (MgSO₄), filtered and concentrated to afford the title compound as colorless solid (1.87 g, 27%). [MNa]⁺=314.

### Step C

At 80° C. N,N-dimethylformamide di-tert-butyl acetal (6.2 mL) was added to a solution of the title compound from Step B above (1.87 g) in dry toluene (15 mL). The mixture was stirred at 80° C. for 3 h, cooled to room temperature, concentrated and purified by chromatography (silica, CH₂Cl₂) to afford the title compound as a colorless solid (820 mg, 38%). [MNa]⁺=370.

### Step D

To a solution of the title compound from Step C above (820 mg) in ^{t}BuOAc (40 mL) was added concentrated H₂SO₄ (0.65 mL). The resulting mixture was stirred at room temperature for 5 h, concentrated, diluted with EtOAc, washed with saturated aqueous NaHCO₃ and saturated aqueous NaCl, dried (MgSO₄), filtered and concentrated to afford the title compound as a colorless solid (640 mg, 99%). [M−NH₂]⁺=231.

### Preparative Example 11

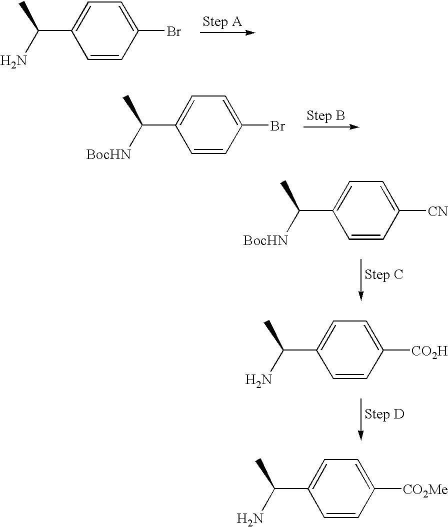

### Step A

Commercially obtained (S)-(−)-1-(4-bromophenyl)ethylamine (2.0 g, 10.1 mmol) was dissolved in 50 mL dry tetrahydrofuran (THF) and cooled to 0° C. and to this cooled solution was added di-t-butyl dicarbonate (2.0 g, 9.1 mmol) dissolved in 3.0 mL of methylene chloride (CH₂Cl₂) followed by Et₃N (2.8 mL, 20.1 mmol). The solution was allowed to warm to room temperature. After stirring for 3 hours, the mixture was concentrated and re-dissolved in 100 mL methylene chloride (CH₂Cl₂). This solution was washed with 1N HCl (2×50 mL) and saturated NaHCO₃ (1×50 mL). The CH₂Cl₂ layer was dried over anhydrous MgSO₄, filtered, and concentrated to afford 2.5 g of the Boc protected product in 92% yield as a white solid.

¹H-NMR δ (CDCl₃) 1.35 (br. s, 12H), 4.72 (br. s, 2H), 7.17 (d, 2H), 7.43 (d, 2H).

### Step B

The Boc protected product from Step A (4.0 g, 13.3 mmol), ZnCN₂ (3.0 g, 24.4 mmol), and Pd[PPh₃]₄ (1.5 g, 1.3 mmol) were combined under nitrogen and anhydrous dimethylformamide (25 mL) was added. The yellow mixture was heated to 100° C. for 18 h and then concentrated under reduced pressure to afford crude cyano compound which was purified by flash chromatography (20% hexane/CH₂Cl₂) to give 2.0 g of the desired cyano containing compound as an oil in 60% yield.

¹H-NMR δ (CDCl₃) 0.89-1.62 (br. m, 12H), 4.81 (br. s, 2H), 7.42 (d, 2H), 7.65 (d, 2H).

MH⁺=247

### Step C

The cyano compound (2.0 g, 8.1 mmol) was suspended in 6N HCl (50 mL) and heated to 100-105° C. for 20 hours upon which the solution becomes homogeneous. The solvent was removed under reduce pressure to give 1.8 g of the amino acid as the hydrochloride salt in quantitative yield as a white solid.

### Step D

The hydrochloride salt of the amino acid (1.0 g, 4.9 mmol) was dissolved in anhydrous MeOH (150 mL) saturated with anhydrous HCl gas. The reaction mixture was then heated to reflux for 20 hours. After cooling to room temperature, the solvent was removed under reduced pressure to give a solid. The solid was taken up in methylene chloride (CH₂Cl₂) and washed with saturated NaHCO₃. The organic was separated and dried over MgSO₄, filtered and concentrated to give 0.31 g of 4-(1(S)-amino-ethyl)-benzoic acid methyl ester in 35% yield as an oil which slowly crystallized into a light brown solid. MH⁺=180

### Preparative Example 12

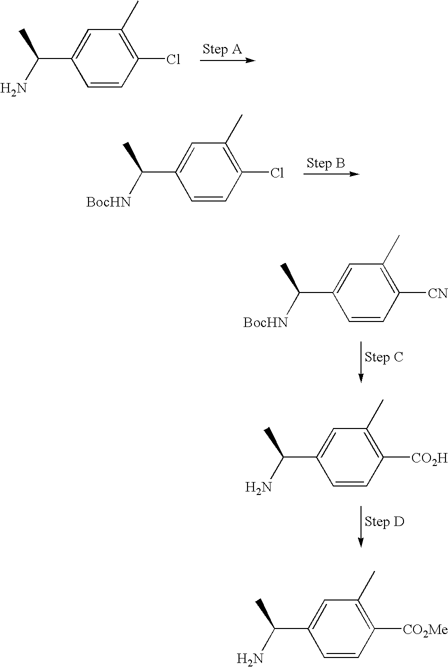

### Step A

Commercially available (S)-1-(4-chloro-3-methylophenyl)ethylamine (1.5 mmol) was dissolved in 10 mL dry Tetrahydrofuran (THF) and cooled to 0° C. and to this cooled solution was added di-t-butyl dicarbonate (1.5 mmol) dissolved in 1.0 mL of methylene chloride (CH₂Cl₂) followed by Et₃N (2.8 mL, 5 mmol). The solution was allowed to warm to room temperature. After stirring for 3 hours, the mixture was concentrated and re-dissolved in 100 mL methylene chloride (CH₂Cl₂). This solution was washed with 1N HCl (2×50 mL) and saturated NaHCO₃ (1×50 mL). The CH₂Cl₂ layer was dried over anhydrous MgSO₄, filtered, and concentrated to afford the Boc protected product.

### Step B

If to the Boc protected amine product (1 mmol) was added ZnCN₂ (2 mmol), Pd[PPh₃]₄ (0.1 mmol) and anhydrous dimethylformamide (6 mL) and the yellow mixture heated to 100° C. for 18 h and then purified by flash chromatography (20% hexane/CH₂Cl₂) one would get the desired cyano containing compound.

### Step C

If the cyano containing compound (0.5 mmol) was suspended in 6N HCl (10 mL) and heated to 100-105° C. for 20 hours until the solution becomes homogeneous and the solvent removed under reduce pressure one would get the amino acid as the hydrochloride salt.

### Step D

If the hydrochloride salt of the amino acid (0.5 mmol) was dissolved in anhydrous MeOH (50 mL) saturated with anhydrous HCl gas and then heated to reflux for 20 hours one would get the 4-(1(S)-amino-ethyl)-2-methyl-benzoic acid methyl ester.

### Preparative Example 13

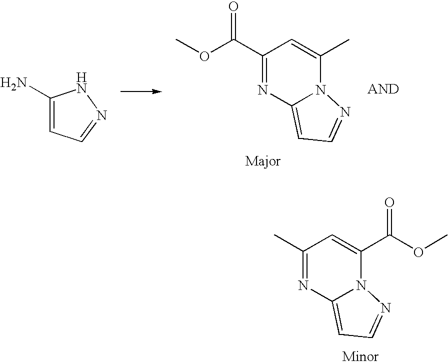

To a solution of commercially available 1H-pyrazol-5-amine (86.4 g) in MeOH (1.80 L) was added commercially available methyl acetopyruvate (50.0 g). The mixture was heated to reflux for 5 h and then cooled to room temperature overnight. The precipitated yellow needles were collected by filtration and the supernatant was concentrated at 40° C. under reduced pressure to ⅔ volume until more precipitate began to form. The mixture was cooled to room temperature and the precipitate was collected by filtration. This concentration/precipitation/filtration procedure was repeated to give 3 batches. This material was combined and recrystallized from MeOH to give the major isomer, methyl 7-methyl-pyrazolo[1,5-a]pyrimidine-5-carboxylate (81.7 g, 72%). [MH]⁺=192.

The remaining supernatants were combined, concentrated and purified by chromatography (silica, cyclohexane/EtOAc) to afford the minor isomer, methyl 5-methyl-pyrazolo[1,5-a]pyrimidine-7-carboxylate (6.8 g, 6%). [MH]⁺=192.

### Preparative Example 14

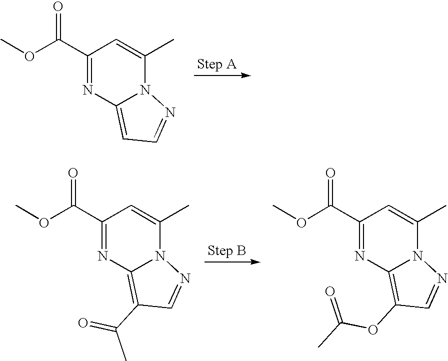

### Step A

To a solution of the major isomer of the title compound from the Preparative Example 8, Step A (2.0 g) in CH₂Cl₂ (20 mL) were added acetyl chloride (3.0 mL) and SnCl₄ (10.9 g). The resulting mixture was heated to reflux overnight, cooled and quenched with H₂O (10 mL). The aqueous phase was separated and extracted with CH₂Cl₂ (2×). The combined organic phases were concentrated and purified by chromatography (silica, cyclohexane/EtOAc) to afford the title compound (1.2 g, 49%). [MH]⁺=234.

### Step B

Trifluoroacetic anhydride (4.6 mL) was added dropwise to an ice cooled suspension of urea hydrogen peroxide (5.8 g) in CH₂Cl₂ (40 mL). The mixture was stirred for 30 min, then a solution of the title compound from Step A above (1.8 g) in CH₂Cl₂ (20 mL) was added and the mixture was stirred at room temperature overnight. NaHSO₃ (1.0 g) was added and the resulting mixture was diluted with saturated aqueous NaHCO₃ (40 mL). The aqueous phase was separated and extracted with CH₂Cl₂. The combined organic phases were concentrated and purified by chromatography (silica, cyclohexane/EtOAc) to afford 3-acetoxy-7-methyl-pyrazolo[1,5-a]pyrimidine-5-carboxylic acid methyl ester (500 mg, 26%). ¹H-NMR (CDCl₃) δ=8.40 (s, 1H), 7.47 (d, 1H), 4.03 (s, 3H), 2.84 (d, 3H), 2.42 (s, 3H).

### Preparative Example 15

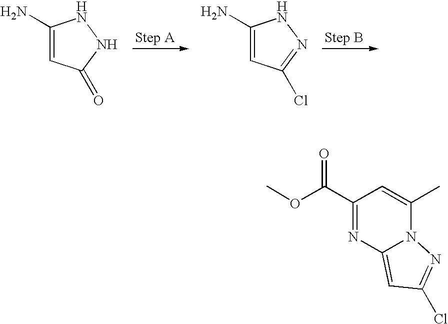

### Step A

A mixture of commercially available 5-aminopyrazolone (5 g) and POCl₃ (50 mL) was heated to 210° C. for 5 h, concentrated and quenched with MeOH (10 mL) at 0° C. Purification by chromatography (silica, hexanes/EtOAc) afforded the desired product (293 mg, 5%). [MH]⁺=118.

### Step B

A mixture of the title compound from Step A above (117 mg) and methyl acetopyruvate (144 mg) in MeOH (5 mL) was heated to reflux for 2 h and then cooled to 0° C. The formed precipitate was collected by filtration to give 2-chloro-7-methyl-pyrazolo[1,5-a]pyrimidine-5-carboxylic acid methyl ester (200 mg, 89%). [MH]⁺=226.

### Preparative Example 16

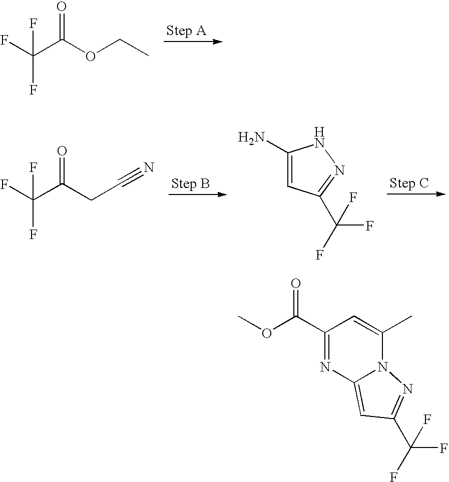

### Step A

Under a nitrogen atmosphere at 0° C. was slowly added 1,4-dioxane (350 mL) to NaH (60% in mineral oil, 9.6 g) followed by the slow addition of CH₃CN (12.6 mL). The mixture was allowed to warm to room temperature before ethyl trifluoroacetate (23.8 mL) was added. The mixture was stirred at room temperature for 30 min, heated at 100° C. for 5 h, cooled to room temperature and concentrated. The remaining solid was taken up in H₂O (400 mL), washed with Et₂O (300 mL), adjusted to pH 2 with concentrated HCl and extracted with CH₂Cl₂ (300 mL). The CH₂Cl₂ extract was dried (MgSO₄), filtered and concentrated to give a brown liquid, which was not further purified (12.5 g, 74%). [M−H]⁻=136.

### Step B

A mixture of the title compound from Step A above (12.5 g) and hydrazine monohydrate (6.0 g) in absolute EtOH (300 mL) was heated to reflux under a nitrogen atmosphere for 8 h, cooled to room temperature and concentrated. The remaining oil was taken up in CH₂Cl₂ (150 mL), washed with saturated aqueous NaCl, dried (MgSO₄), filtered, concentrated and purified by chromatography (silica, CH₂Cl₂MeOH) to give the title compound (0.25 g, 2%). [MH]⁺=152.

### Step C

Using a microwave, a mixture of the title compound from Step B above (150 mg) and commercially available methyl acetopyruvate (150 mg) in MeOH (1 mL) in a sealed vial was heated at 120° C. for 12 min, concentrated and purified by chromatography (silica, CH₂Cl₂) to give 7-methyl-2-trifluoromethyl-pyrazolo[1,5-a]pyrimidine-5-carboxylic acid methyl ester (0.15 g, 58%). [MH]⁺=260.

### Preparative Example 17

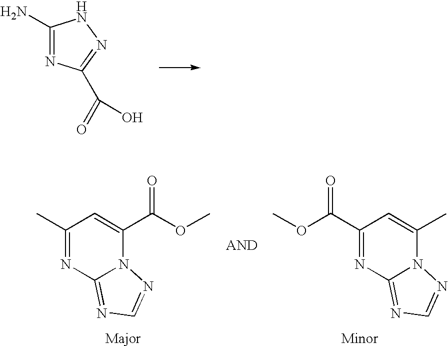

A mixture of commercially available 5-amino-1H-[1,2,4]triazole-3-carboxylic acid (20.3 g) and methyl acetopyruvate (20.0 g) in glacial AcOH (250 mL) was heated to 95° C. for 3 h. The mixture was concentrated and diluted with saturated aqueous NaHCO₃ (200 mL) and CH₂Cl₂ (500 mL). The organic phase was separated, dried (MgSO₄), filtered and concentrated to give a pale orange mixture of regioisomers (80:20, 21.3 g, 80%). Recrystallization of the crude material from hot THF (110 mL) afforded the major isomer, 5-methyl-[1,2,4]triazolo[1,5-a]pyrimidine-7-carboxylic acid methyl ester (13.0 g, 49%). [MH]⁺=193. The supernatant was concentrated and purified by chromatography (silica, hexanes/EtOAc) to afford the minor isomer, 7-methyl-[1,2,4]triazolo[1,5-a]pyrimidine-5-carboxylic acid methyl ester. [MH]⁺=193.

### Preparative Examples 18-22

Following a similar procedure as described in the Preparative Example 8, except using the amines indicated in Table I below, the following compounds were prepared.

TABLE 1

| Prep. | | Major | |
|---|---|---|---|
| Ex. # | Amine | product | Yield |
| 18 | 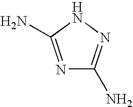 | 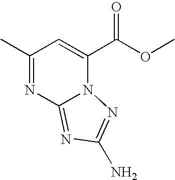 | 96%[MH]⁺ = 208 |
| 19 | 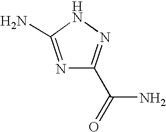 | 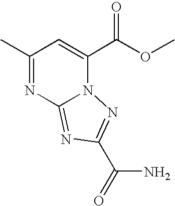 | 92%[MH]⁺ = 236 |
| 20 | 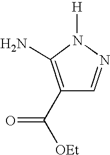 | 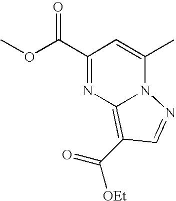 | 50%[MH]⁺ = 264 |
| 21 | 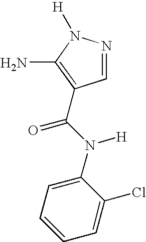 | 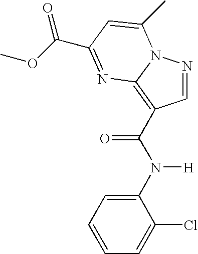 | 78%[MH]⁺ = 345 |
| 22 | 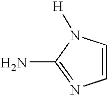 | 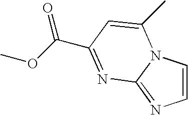 | 14%[MH]⁺ = 192 |

### Preparative Example 23

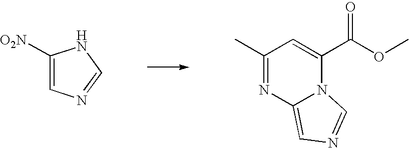

A mixture of commercially available 4-nitroimidazole (5 g) and Pd/C (10 wt %, 500 mg) in a premixed solution of acetyl chloride (4 mL) in MeOH (100 mL) was hydrogenated in a Parr shaker at 35 psi for 5 h. The mixture was filtered through celites and concentrated to give a black oil. [MH]⁺=115. This oil and methyl acetylpyruvate (6.4 g) were stirred in AcOH (70 mL) and MeOH (70 mL) at 65° C. for 18 h. The resulting mixture was absorbed on silica and purified by chromatography (silica, CH₂Cl₂/MeOH). Further purification of the resulting residue by chromatography (silica, EtOAc) afforded 2-methyl-imidazo[1,5-a]pyrimidine-4-carboxylic acid methyl ester as an orange solid (120 mg, 1.4%). [MH]⁺=192.

### Preparative Example 24

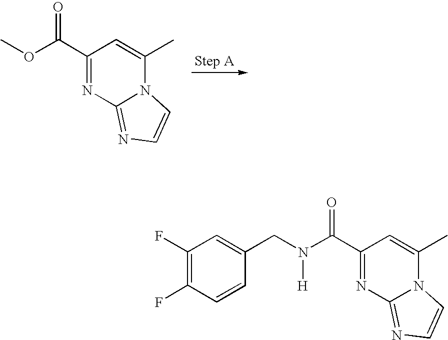

### Step A

A solution of 5-methyl-imidazo[1,2-a]pyrimidine-7-carboxylic acid methyl ester (14 mg) in THF (100 μL), MeOH (100 μL), and 1N aqueous LiOH (80 μL) was stirred at 0° C. for 2 h and then concentrated to give the free acid as a yellow residue. [MH]⁺=178. A mixture of this residue, PyBOP (42 mg), 3,4-difluorobenzylamine (11 mg), and NEt₃ (20 μL) in DMF (200 μL) and THF (400 μL) was stirred for 4 h, then absorbed on silica and purified by chromatography (silica, EtOAc/MeOH) to give 5-methyl-imidazo[1,2-a]pyrimidine-7-carboxylic acid 3,4-difluoro-benzylamide as off-white solid (12 mg, 55%). [MH]⁺=299.

### Preparative Example 25

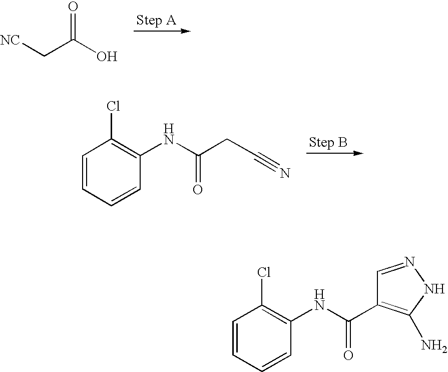

### Step A

To a 250 ml round bottom flask containing a stir bar was added 8.5 grams (0.1 mole) of alpha cyanoacetic acid and 50 ml of methylene chloride (CH₂Cl₂) and 0.2 ml of DMF and mixture chilled to −5° C. The chilled reaction mixture was added under nitrogen 10.8 ml (0.12 ml) of oxalyl chloride and mixture stirred at −5° C. and then at room temperature for 2 h. The volatile components of the reaction mixture were then removed under reduced pressure to give an oil. To the oil was then added 15 ml of THF and 15 ml of CH₂Cl₂ and mixture chilled to 0° C. and then added 12 ml (0.08 mmol) of triethylamine and 10 ml (1.11 g) of 2-chloroaniline and mixture allowed to warm to room temperature and stirring continued for 10 hours. The volatile components of the reaction mixture were removed under reduced pressure to give a solid. The solid was taken up in 400 ml of methylene chloride and organic washed twice with 200 ml of 1N aqueous HCl and then twice with saturated aqueous NaCl. The organic was separated and then dried over MgSO₄, filtered and the volatile components removed under reduced pressure to give a brown solid which was triturated with 250 ml of 50% ether-hexane and solid filtered to give 12.1 grams (62% yield) of N-(2-chloro-phenyl)-2-cyano-acetamide.

### Step B

To a round bottom flask containing a stir bar was added 11.1 g (0.057 mole) of N-(2-chloro-phenyl)-2-cyano-acetamide and 22 ml of acetic anhydride and 10.0 ml (0.06 mole) of triethylorthoformate and mixture heated and the resulting ethyl acetate distilled off at 120-130° C. After all of the ethyl acetate had distilled off, the remaining volatile components of the reaction mixture were removed under reduced pressure to give a solid. To the sold was added 100 ml of chloroform and mixture filtered through celit and the volatile components of the reaction mixture was removed under reduced pressure to give a solid. To the solid was then taken up in 10 ml of anhydrous THF and 30 ml of hydrazine monohydrate and mixture heated at 80° C. for 2 h. The volatile components of the reaction mixture were then removed under reduced pressure to give a solid. The solid was taken up in 250 ml of methylene chloride and organic washed twice with 200 ml of water, dried over MgSO₄, filtered and then the volatile components removed under reduced pressure to give a solid which was purified by column chromatography (SiO₂) to give 5.1 grams (38% yield) of the desired 5-amino-1H-pyrazole-4-carboxylic acid (2-chloro-phenyl)-amide product [MH]⁺=237.

### Example 26

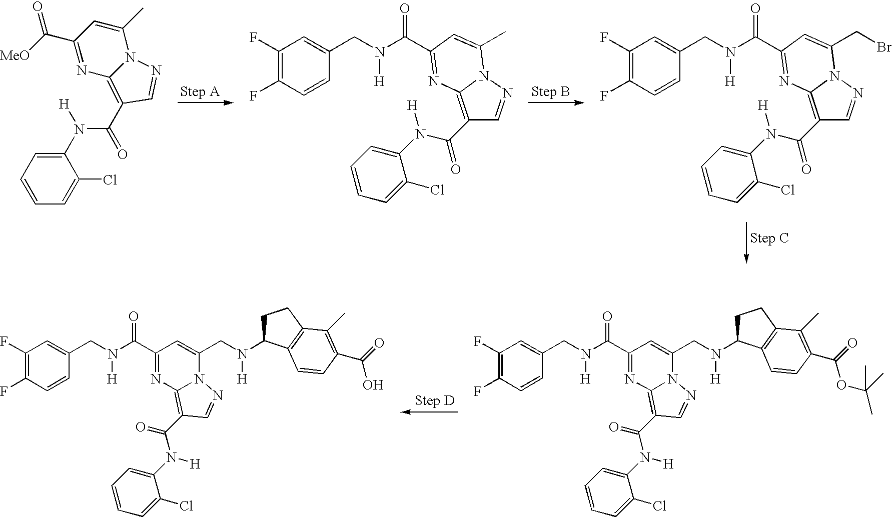

### Step A

To a round bottom flask was added 1.45 mmol (0.5 g) of 3-(2-Chloro-phenylcarbamoyl)-7-methyl-pyrazolo[1,5-a]pyrimidine-5-carboxylic acid methyl ester which was made by coupling the amino pyrazole (synthesized following standard literature procedure: Huppatz, J. L.; Aust. J. Chem., 1985, 38, 221-230) and methyl acetopyruvate as seen in Preparative Example 21 and 3.86 mmol (0.46 ml) of 3,4 difluorobenzylamine and 3 ml of dimethylformamide (DMF) and mixture heated under nitrogen at 60° C. for 10 h. The volatile components of the reaction mixture was removed under reduced pressure and the resulting residue was purified by column chromatography (SiO₂) to give 0.5 grams (75% yield) of the desired amide [MH]⁺=456.

### Step B

To a thick walled glass pressure vessel containing a stir bar was added 0.55 mmol (0.25 g) of 7-Methyl-pyrazolo[1,5-a]pyrimidine-3,5-dicarboxylic acid 3-[(2-chloro-phenyl)-amide] 5-(3,4-difluoro-benzylamide) and 3.0 ml of glacial acetic acid and 0.70 mmol of Bromine. The vessel was sealed and then heated at 150° C. for 15 minutes. The reaction mixture was cooled to room temperature and the volatile components removed under reduced pressure. The crude solid was taken up in 150 ml diethyl ether/100 ml sat. NaHCO3 and the organic separated and washed with sat. NaCl and then dried over MgSO4, filtered and the volatile components removed under reduced pressure to give a solid. The solid was purified by column chromatography (SiO₂) to give 155 mg (52% yield) of the desired mono bromide product [MH]⁺=534.

### Step C

To a round bottom flask was added 0.065 mmol (35 mg) of 7-bromomethyl-pyrazolo[1,5-a]pyrimidine-3,5-dicarboxylic acid 3-[(2-chloro-phenyl)-amide] 5-(3,4-difluoro-benzylamide), 0.22 mmol anhydrous K₂CO₃, 0.072 mmol (18 mg) of 1-amino-4-methyl-indan-5-carboxylic acid tert-butyl ester and 3 ml of anhydrous tetrahydrofuran and mixture heated at 45° C. for 10 hours under a nitrogen atmosphere. The volatile components of the reaction mixture was removed under reduced pressure and the resulting residue was purified by column chromatography (SiO₂) to give 15 mg (35% yield) of the desired secondary amine product [MH]⁺=701.

### Step D

To a 5 ml round bottom flask containing a stir bar was added 1-{[3-(2-Chloro-phenylcarbamoyl)-5-(3,4-difluoro-benzylcarbamoyl)-pyrazolo[1,5-a]pyrimidin-7-ylmethyl]-amino}-4-methyl-indan-5-carboxylic acid tert-butyl ester (0.021 mmol) and 2 ml of 50% trifluoroacetic acid in methylene chloride and solution stirred for 3 hours. The reaction mixture was concentrated under reduced pressure and the resulting oil was triturated with diethyl either to give 15 mg (80% yield) 1-{[3-(2-chloro-phenylcarbamoyl)-5-(3,4-difluoro-benzylcarbamoyl)-pyrazolo[1,5-a]pyrimidin-7-ylmethyl]-amino}-4-methyl-indan-5-carboxylic acid as the mono trifluoroacetic acid salt [MH]⁺=645.

### Preparative Example 27

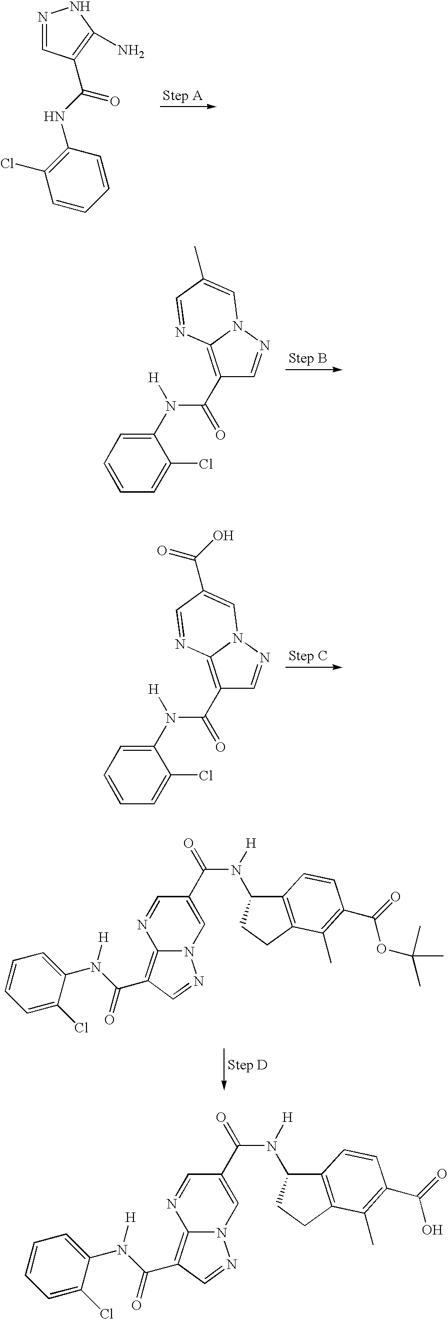

### Step A

To a solution of 0.5 grams (2.11 mmol) of the substituted amino pyrazole in MeOH (4 ml) was added 0.5 grams of commercially available 1,1,3,3-tetraethoxy-2-methyl-propane (2.11 mmol) and 0.2 ml of concentrated hydrochloric acid. The mixture was heated to reflux for 5 h and then cooled to room temperature overnight. The precipitated solid was collected by filtration to give 0.41 grams (83% yield) of the desired 6-Methyl-pyrazolo[1,5-a]pyrimidine-3-carboxylic acid (2-chloro-phenyl)-amide compound [MH]⁺=287.

### Step B

To a thick walled vessel containing a stir bar was added 0.27 g (0.94 mmol) of 6-methyl-pyrazolo[1,5-a]pyrimidine-3-carboxylic acid (2-chloro-phenyl)-amide and 1.3 grams (11.18 mmol) of selenium dioxide and 10 ml of dioxane and mixture heated via microwaves under closed atmosphere at 180° C. for 6 h. The reaction mixture was then cooled to room temperature and then 0.57 g (0.94 mmol) of the commercially available reagent oxone was added and 0.4 ml of water and mixture stirred at room temperature for 10 h. To the reaction mixture was added 100 ml of methylene chloride and mixture filtered through celite and organic washed with water, separated, dried over MgSO₄, filtered and the volatile components removed under reduced pressure to give a solid which was purified via column chromatography (SiO₂) to give the desired acid product [MH]⁺=317.

### Step C

To a round bottom flask containing 11 mg (0.034 mmol) of the acid compound was added 4 ml of methylene chloride and 0.1 ml of DMF and mixture stirred until solution was complete. To the solution was added 12 microliters (0.13 mmol) of oxalyl chloride and mixture stirred at 0° C. for 20 minutes and then for 1 h at room temperature. The volatile components of the reaction mixture were removed under reduced pressure to give a solid. To the solid was added 2 ml of tetrahydrofuran (THF) and 0.04 mmol of 1-Amino-4-methyl-indan-5-carboxylic acid tert-butyl ester and 0.08 mmol of triethylamine and mixture stirred under a nitrogen atmosphere for 10 hours. The volatile components of the reaction mixture was removed under reduced pressure to give a solid which was purified by preparative thin layer chromatography to give the desired 1-{[3-(2-Chloro-phenylcarbamoyl)-pyrazolo[1,5-a]pyrimidine-6-carbonyl]-amino}-4-methyl-indan-5-carboxylic acid tert-butyl ester in 50% yield.

### Step D

To a round bottom flask containing 20 mg (0.036 mmol) of the tert-butyl ester compound was added 2 ml of 50% trifluoroacetic acid in methylene chloride and solution stirred at room temperature for 2 hours. The volatile components of the reaction mixture were removed under reduced pressure to give a oil which was triturated with diethyl ether to give 9 mg (50% yield) of 1-{[3-(2-chloro-phenylcarbamoyl)-pyrazolo[1,5-a]pyrimidine-6-carbonyl]-amino}-4-methyl-indan-5-carboxylic acid [MH]⁺=490.

### Preparative Example 28

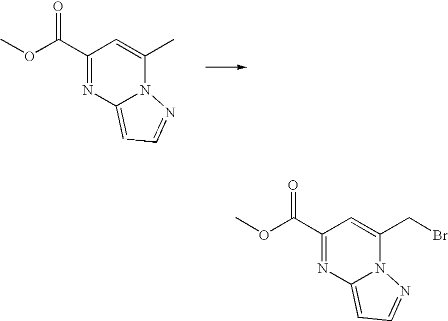

To 4-methyl-pyrrolo[1,2-a]pyrimidine-2-carboxylic acid methyl ester (6.5 mmol) in 25 ml round bottom flask containing a stir bar was added 5 ml of acetic acid and bromine (6.5 mmol) and mixture heated at 75° C. for 5-10 minutes. The volatile components of the reaction mixture were removed under reduced pressure to give an oil. The oil was taken up in 100 ml of methylene chloride and the organic washed with saturated NaHCO₃. The organic was separated, dried over MgSO₄, filtered and the volatile components removed under reduced pressure to give an oil which was purified by column chromatography (SiO₂, 10% diethyl ether-methylene chloride) to give the desired 4-bromomethyl-pyrrolo[1,2-a]pyrimidine-2-carboxylic acid methyl ester in 50% yield [MH]⁺=270.

### Example 29

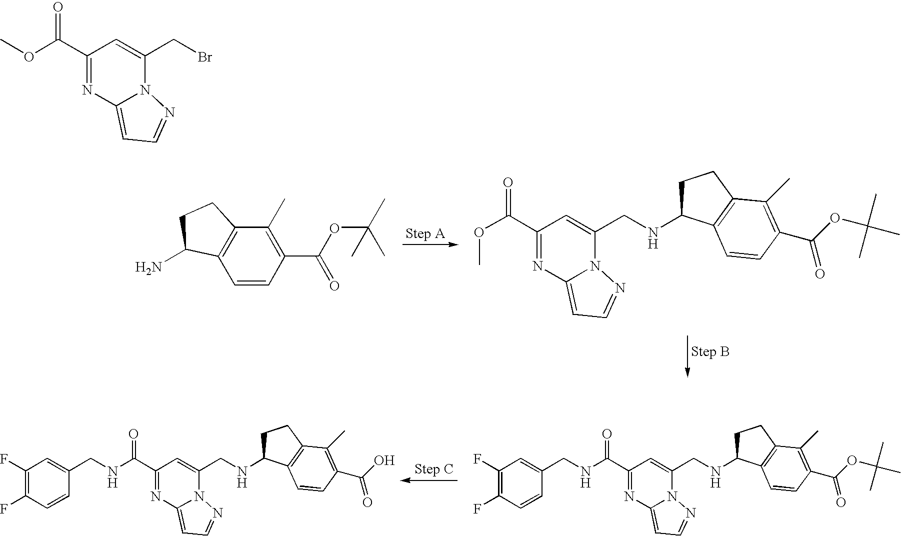

### Step A

To a 5 ml round bottom flask was added 7-bromomethyl-pyrazolo[1,5-a]pyrimidine-5-carboxylic acid methyl ester (0.2 mmol) and 1-amino-4-methyl-indan-5-carboxylic acid tert-butyl ester (0.23 mmol) and triethylamine (0.61 mmol) and 0.6 ml of dimethylformamide and mixture heated at 100° C. for 10 minutes. The reaction mixture was concentrated under reduced pressure and the resulting residue purified by column chromatography (SiO₂, 20% ether-methylene chloride) to give the desired 7-[(5-tert-butoxycarbonyl-4-methyl-indan-1-ylamino)-methyl]-pyrazolo[1,5-a]pyrimidine-5-carboxylic acid methyl ester in 67% yield (M+H=437).

### Step B

To a 5 ml thick walled vessel was added 7-[(5-tert-butoxycarbonyl-4-methyl-indan-1-ylamino)-methyl]-pyrazolo[1,5-a]pyrimidine-5-carboxylic acid methyl ester (0.09 mmol), 3,4-difluorobenzylamine (0.7 mmol) and 0.5 ml of dimethylformamide. The reaction mixture was heated via microwaves under closed atmosphere at a temperature of 120° C. for 30 minutes. The reaction mixture was concentrated under reduced pressure to give a oil residue. The residue was purified by preparative thin layer chromatography (SiO₂, 20% ether-methylene chloride) to give 1-{[5-(3,4-Difluoro-benzylcarbamoyl)-pyrazolo[1,5-a]pyrimidin-7-ylmethyl]-amino}-4-methyl-indan-5-carboxylic acid tert-butyl ester product in 55% yield (M+H=548)

### Step C

To a 5 ml round bottom flask containing a stir bar was added the tert-butyl ester (0.045 mmol) and 2 ml of 50% trifluoroacetic acid in methylene chloride and solution stirred for 3 hours. The reaction mixture was concentrated under reduced pressure and the resulting oil was triturated with diethyl either to the desired 1-{[5-(3,4-difluoro-benzylcarbamoyl)-pyrazolo[1,5-a]pyrimidin-7-ylmethyl]-amino}-4-methyl-indan-5-carboxylic acid as the mono trifluoroacetic acid salt (M+H=492)

### Preparative Example 30

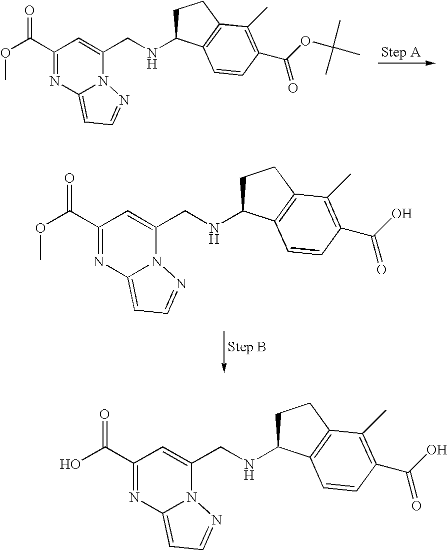

### Step A

To a 5 ml round bottom flask containing a stir bar was added the tert-butyl ester (0.045 mmol) and 2 ml of 50% trifluoroacetic acid in methylene chloride and solution stirred for 3 hours. The reaction mixture was concentrated under reduced pressure and the resulting oil was triturated with diethyl either to give the desired 7-[(5-carboxy-4-methyl-indan-1-ylamino)-methyl]-pyrazolo[1,5-a]pyrimidine-5-carboxylic acid methyl ester in 29% yield (M+H=381)

### Step B

To a 5 ml round bottom flask containing a stir bar was dissolved the methyl ester (0.013 mmol) in 50% methanol-tetrahydrofuran solution and then added an aqueous solution of lithium hydroxide (0.026 mmol) and mixture stirred at room temperature overnight. The solution was acidified with HCl and mixture centrifuged. The clear liquid was decanted and the resulting solid dried under reduced pressure to give the desired 7-[(5-carboxy-4-methyl-indan-1-ylamino)-methyl]-pyrazolo[1,5-a]pyrimidine-5-carboxylic acid (M+H=367)

### Preparative Example 31

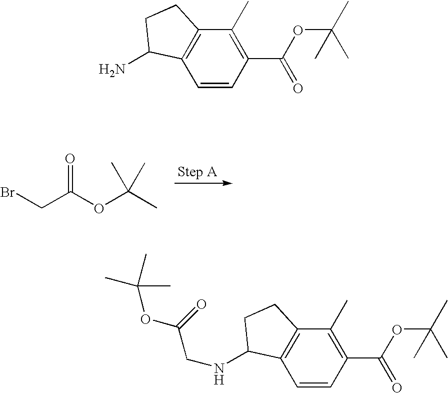

### Step A

1-Amino-4-methyl-indan-5-carboxylic acid tert-butyl ester (0.63 mmol) was added to a thick walled vessel containing a stir bar. To the vessel was then added 6 ml of tetrahydrofuran, triethylamine (1.25 mmol) and bromo-acetic acid tert-butyl ester (0.63 mmol) and mixture heated at 80° C. under closed atmosphere for 25 minutes. The volatile components were removed under reduced pressure to give a solid. The solid was purified by column chromatography (SiO₂, 20% ether-methylene chloride) to give the desired 1-(tert-butoxycarbonylmethyl-amino)-4-methyl-indan-5-carboxylic acid tert-butyl ester in 39% yield (M+H=362)

### Example 32

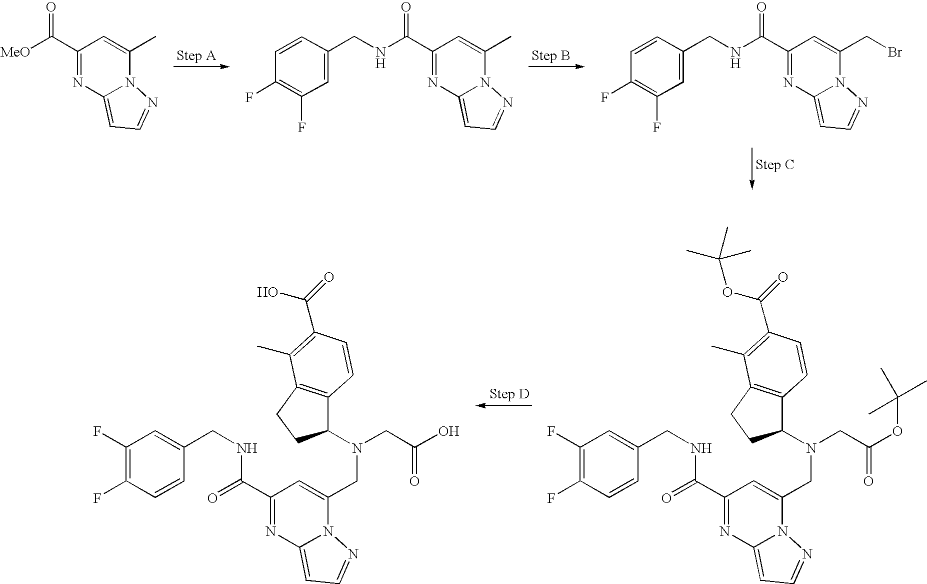

### Step A

To a 5 ml thick walled vessel was added 7-methyl-pyrazolo[1,5-a]pyrimidine-5-carboxylic acid methyl ester (0.09 mmol), 3,4-difluorobenzylamine (0.7 mmol) and 0.5 ml of dimethylformamide. The reaction mixture was heated via microwaves under closed atmosphere at a temperature of 80° C. for 30 minutes. The reaction mixture was concentrated under reduced pressure to give an oil residue. The residue was purified by preparative thin layer chromatography (SiO₂, 20% ether-methylene chloride) to give 100 mg of the resulting amide product in 42% yield [MH]⁺=303.

### Step B

To 7-methyl-pyrazolo[1,5-a]pyrimidine-5-carboxylic acid 3,4-difluorobenzylamide (1.32 mmol) in 25 ml round bottom flask containing a stir bar was added 4 ml of acetic acid and bromine (1.16 mmol) and mixture heated at 120° C. for 10 minutes. The volatile components of the reaction mixture were removed under reduced pressure to give an oil. The oil was taken up in 100 ml of methylene chloride and the organic washed with saturated NaHCO₃. The organic was separated, dried over MgSO₄, filtered and the volatile components removed under reduced pressure to give an oil which was purified by column chromatography (SiO₂, 10% diethyl ether-methylene chloride) to give the desired 7-bromomethyl-pyrazolo[1,5-a]pyrimidine-5-carboxylic acid 3,4-difluoro-benzylamide in 12% yield [MH]⁺=381.

### Step C

To a 5 ml round bottom flask was added 7-bromomethyl-pyrazolo[1,5-a]pyrimidine-5-carboxylic acid 3,4-difluoro-benzylamide (0.1 mmol) and 1-(tert-butoxycarbonylmethyl-amino)-4-methyl-indan-5-carboxylic acid tert-butyl ester (0.19 mmol) and triethylamine (0.35 mmol) and 0.5 ml of dimethylformamide and mixture heated at 80° C. for 15 minutes. The reaction mixture was concentrated under reduced pressure and the resulting residue purified by preparative thin layer chromatography (SiO₂, 20% ether-methylene chloride) to give the desired 1-{tert-butoxycarbonylmethyl-[5-(3,4-difluoro-benzylcarbamoyl)-pyrazolo[1,5-a]pyrimidin-7-ylmethyl]-amino}-4-methyl-indan-5-carboxylic acid tert-butyl ester in 19% yield [MH]⁺=662.

### Step D

To a 5 ml round bottom flask containing a stir bar was added 1-{tert-butoxycarbonylmethyl-[5-(3,4-difluoro-benzylcarbamoyl)-pyrazolo[1,5-a]pyrimidin-7-ylmethyl]-amino}-4-methyl-indan-5-carboxylic acid tert-butyl ester (0.03 mmol) and 1.5 ml of 40% trifluoroacetic acid in methylene chloride and solution stirred for 24 hours. After addition of ˜50 microliters of water the reaction mixture was concentrated under reduced pressure and the resulting oil was triturated with diethyl either to give 10 mg of the desired 1-{carboxymethyl-[5-(3,4-difluoro-benzylcarbamoyl)-pyrazolo[1,5-a]pyrimidin-7-ylmethyl]-amino}-4-methyl-indan-5-carboxylic acid product in 54% yield as the monotrifluoroacetic acid salt [MH]⁺=550.

### Preparative Examples 33-46

If one followed a similar procedure as described in Preparative Example 24 or Preparative Example 26 step A, except using the esters and amines indicated in Table II below, the following compounds could be prepared.

TABLE II

| Prep. | acid, | |
|---|---|---|
| Ex. # | amine | product |
| 33 | 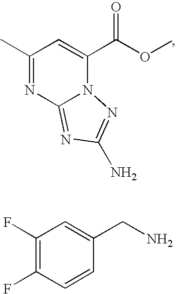 | 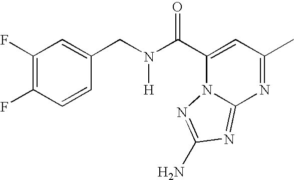 |
| 34 | 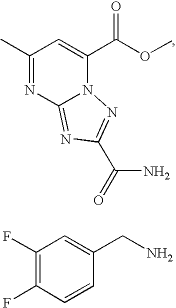 | 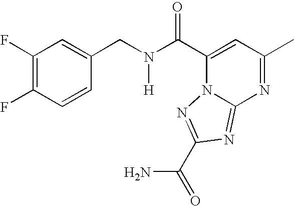 |
| 35 | 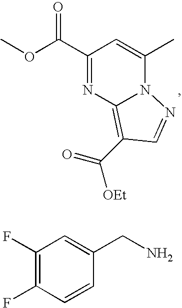 | 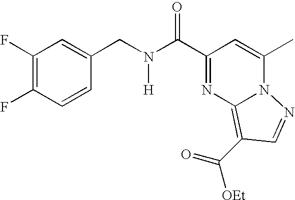 |
| 36 | 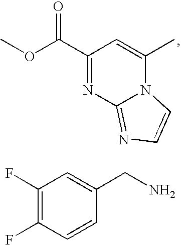 | 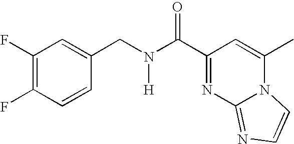 |
| 37 | 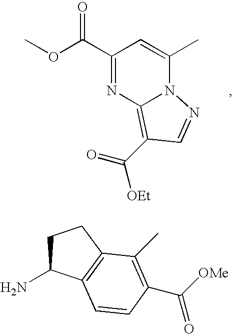 | 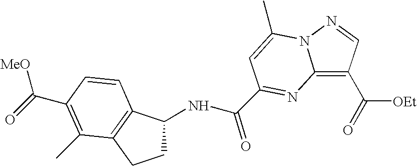 |
| 38 | 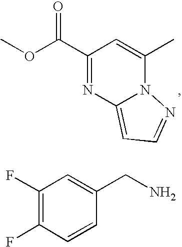 | 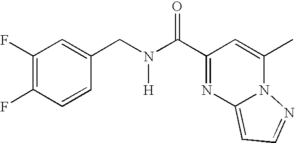 |
| 39 | 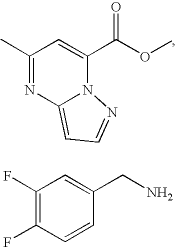 | 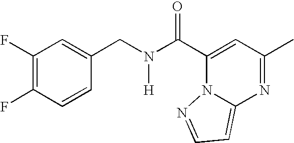 |
| 40 | 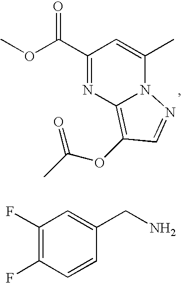 | 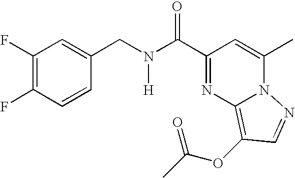 |
| 41 | 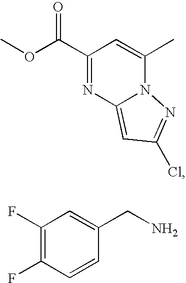 | 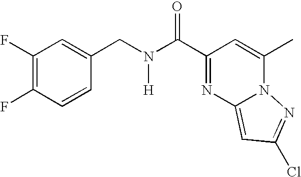 |
| 42 | 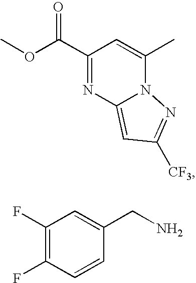 | 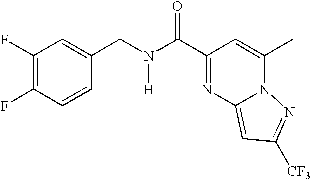 |
| 43 | 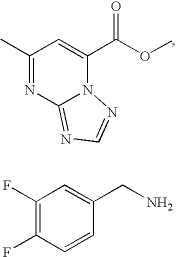 | 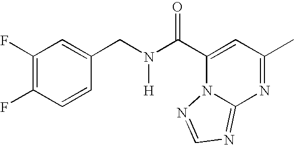 |
| 44 | 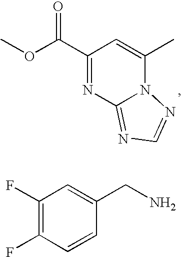 | 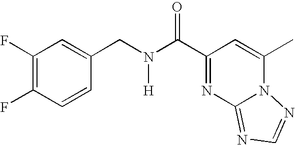 |
| 45 | 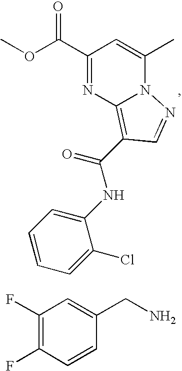 | 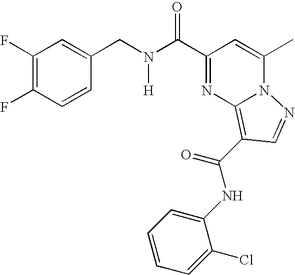 |
| 46 | 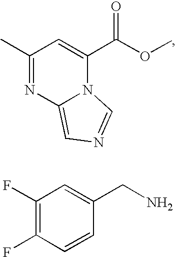 | 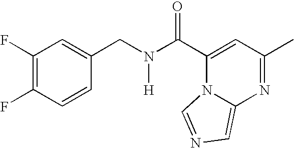 |

### Preparative Example 47

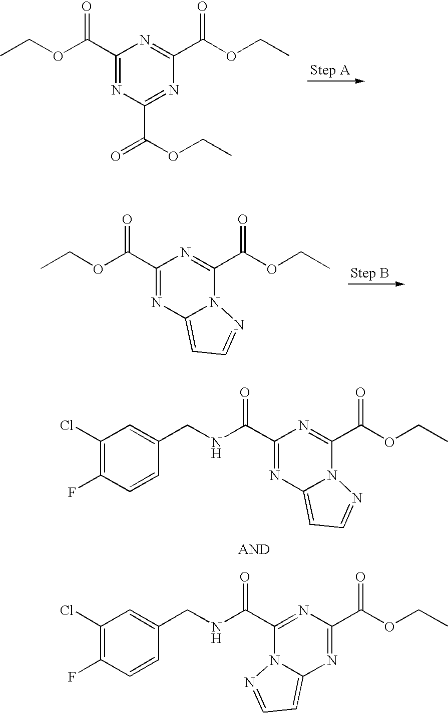

### Step A

Under an argon atmosphere a solution of commercially available [1,3,5]triazine-2,4,6-tricarboxylic acid triethyl ester (818 mg) and 3-aminopyrazole (460 mg) in dry DMF (8 mL) was heated to 100° C. overnight and then concentrated. The remaining residue was dissolved in CHCl₃, washed with 10% aqueous citric acid and saturated aqueous NaCl, dried (MgSO₄), filtered, concentrated and purified by chromatography (silica, CH₂Cl₂/MeOH) to afford the title compound as a colorless solid (409 mg, 56%). [MH]⁺=265.

### Step B

A mixture of the title compound from Step A above (203 mg) and commercially available 3-chloro-4-fluorobenzylamine (160 mg) in dry DMF (3 mL) was heated to 70° C. overnight and concentrated. The remaining residue was dissolved in CHCl₃, washed with 10% aqueous citric acid and saturated aqueous NaCl, dried (MgSO₄), filtered, concentrated and purified by preparative thin layer chromatography (silica, CH₂Cl₂/MeOH) to afford the title compound from the Example 286 and the separated regioisomers of the title compound. [MH]⁺=378.

### Preparative Examples 48-50

Following a similar procedure as described in the Preparative Example 28, except using the pyrazolopyrimidine indicated in Table III below, the following compounds were prepared.

TABLE III

| Prep. | | | |
|---|---|---|---|
| Ex. # | Pyrazolopyrimidine | Bromo Product | yield |
| 48 | 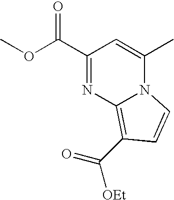 | 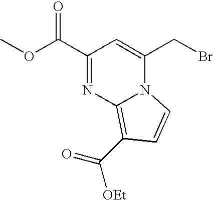 | 50%[MH]⁺ = 342 |
| 49 | 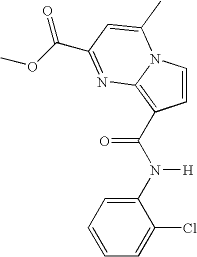 | 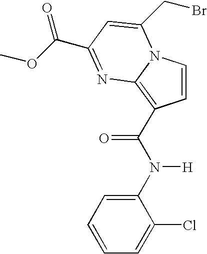 | 62%[MH]⁺ = 423 |
| 50 | 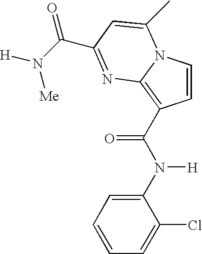 | 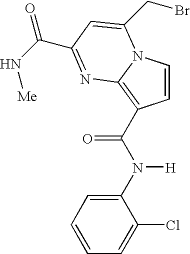 | 65%[MH]⁺ = 422 |

### Preparative Examples 51-63

If one were to follow a similar procedure as described in the Preparative Example 28, except using the pyrazolopyrimidine indicated in Table IV below, the following compounds could be prepared.

TABLE IV

| Prep. | | |
|---|---|---|
| Ex. # | Pyrazolopyrimidine | Bromo Product |
| 51 | 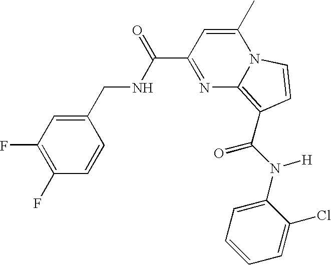 | 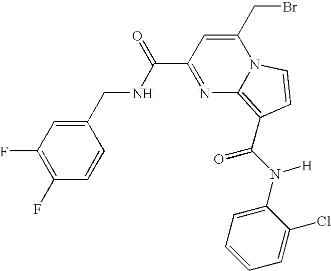 |
| 52 | 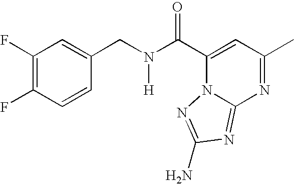 | 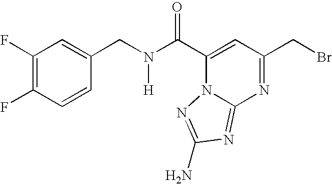 |
| 53 | 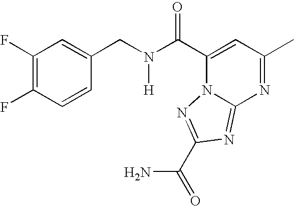 | 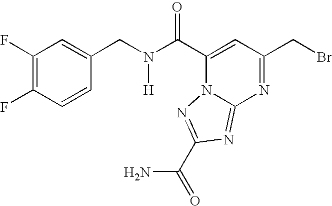 |
| 54 | 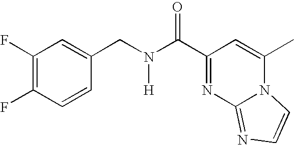 | 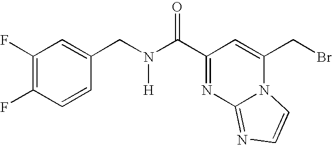 |
| 55 | 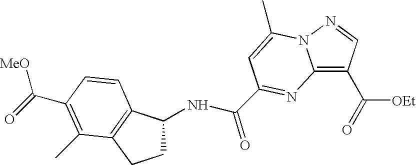 | 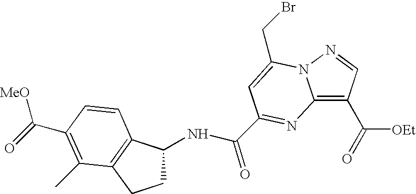 |
| 56 | 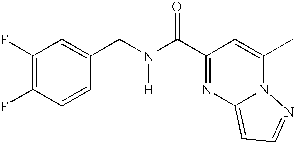 | 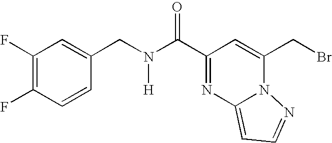 |
| 57 | 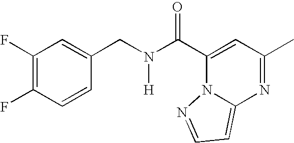 | 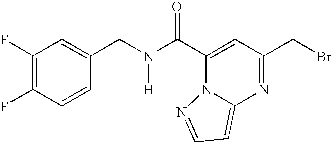 |
| 58 | 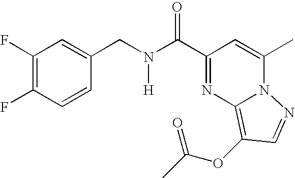 | 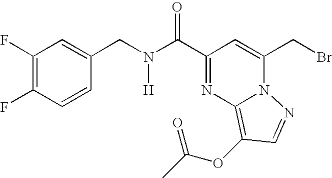 |
| 59 | 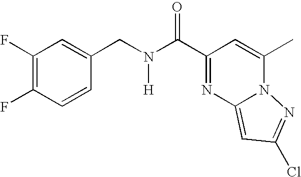 | 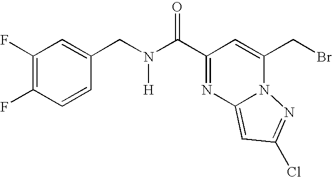 |
| 60 | 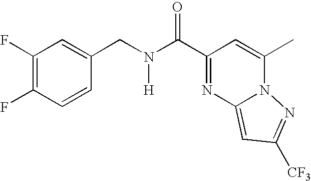 | 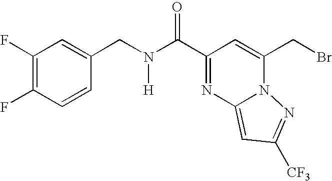 |
| 61 | 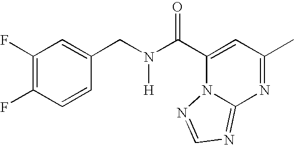 | 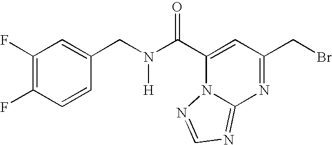 |
| 62 | 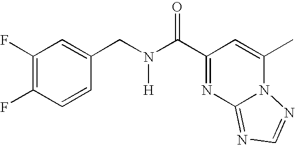 | 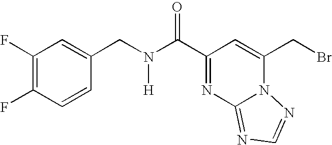 |
| 63 | 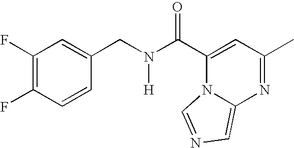 | 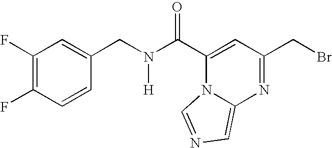 |

### Preparative Examples 64-79

If one were to follow a similar procedure as described in the Preparative Example 29, Step A, except using the bromo compounds indicated in Table V below, the following compounds could be prepared.

TABLE V

| Prep. | | |
|---|---|---|
| Ex. # | Bromide | Product |
| 64 | 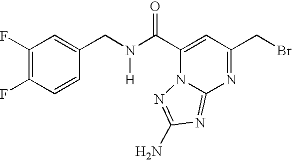 | 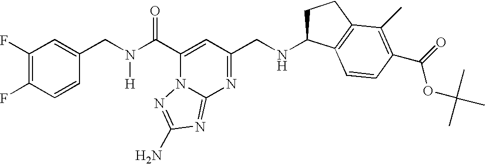 |
| 65 | 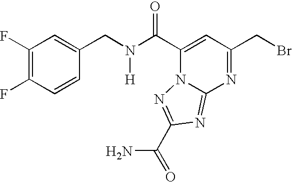 | 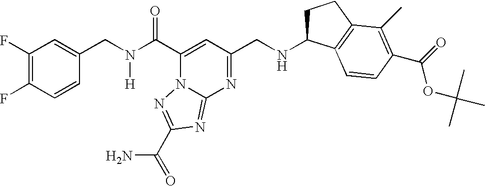 |
| 66 | 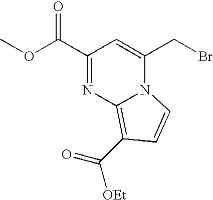 | 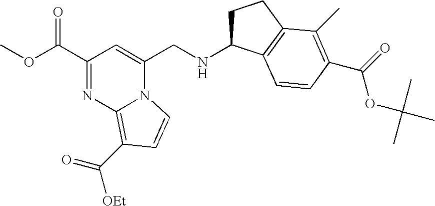 |
| 67 | 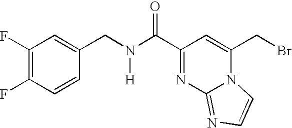 | 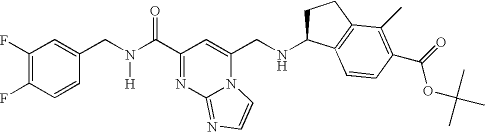 |
| 68 | 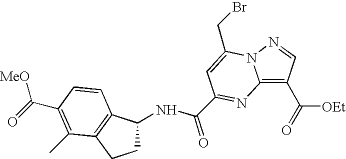 | 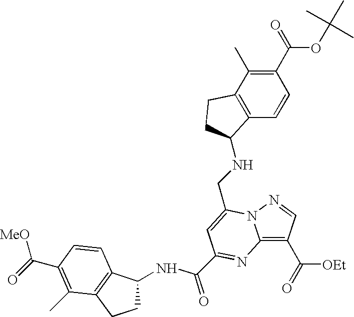 |
| 69 | 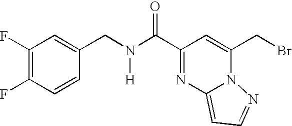 | 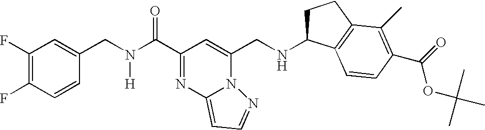 |
| 70 | 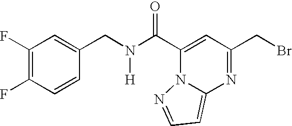 | 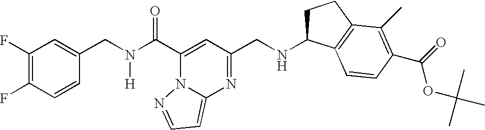 |
| 71 | 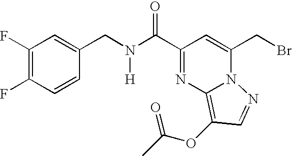 | 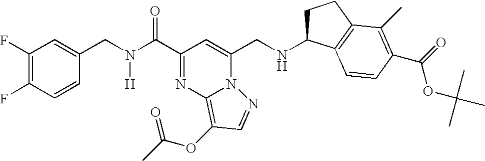 |
| 72 | 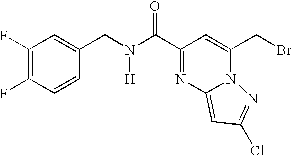 | 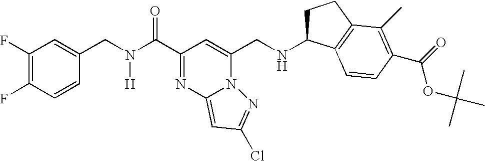 |
| 73 | 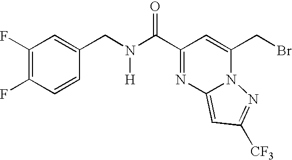 | 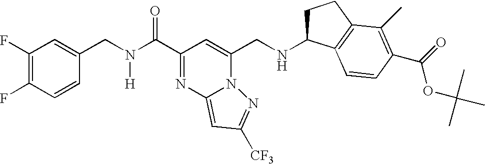 |
| 74 | 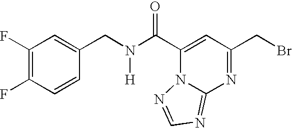 | 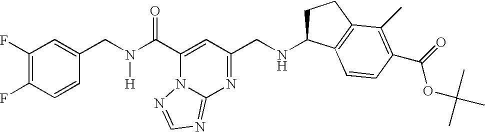 |
| 75 | 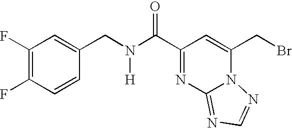 | 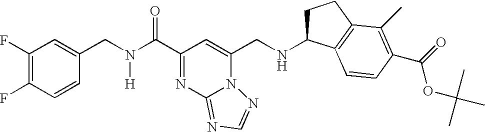 |
| 76 | 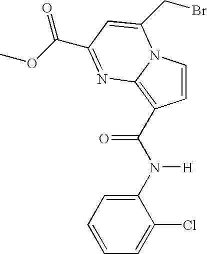 | 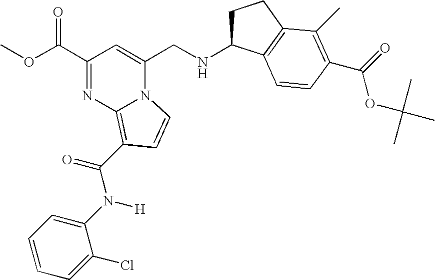 |
| 77 | 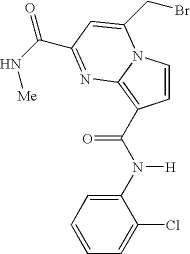 | 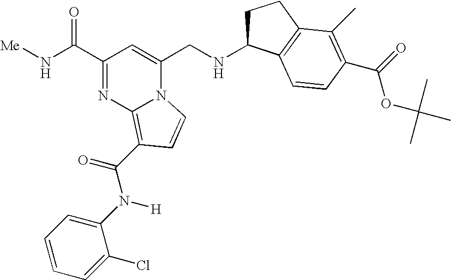 |
| 78 | 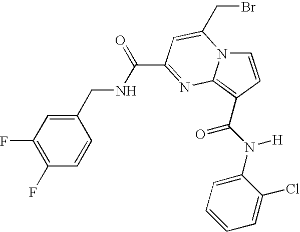 | 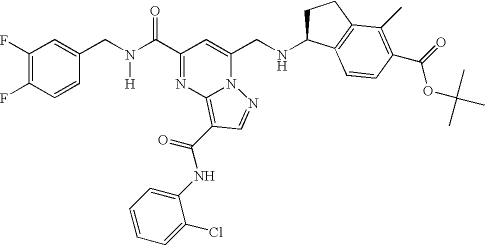 |
| 79 | 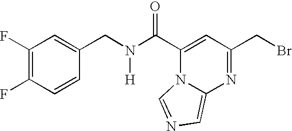 | 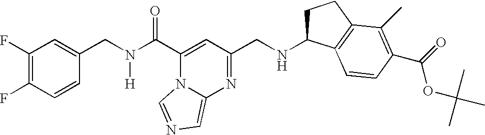 |

### Examples 80-95

If one were to follow a similar procedure as described in the Preparative Example 26, Step D, except using the tert-butyl esters indicated in Table VI below, the following compounds could be prepared.

TABLE VI

| Prep. | | |
|---|---|---|
| Ex. # | tert-Butyl ester | Product |
| 80 | 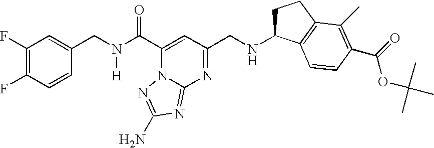 | 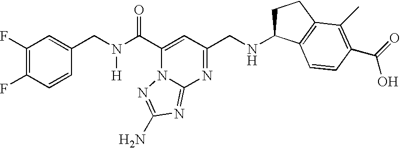 |
| 81 | 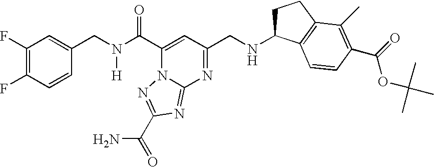 | 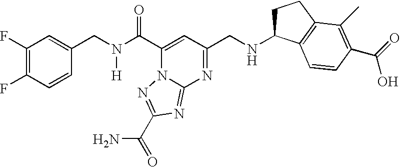 |
| 82 | 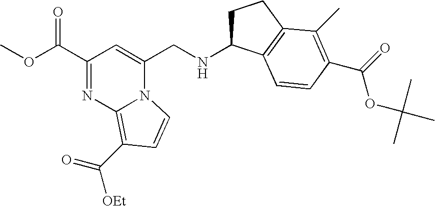 | 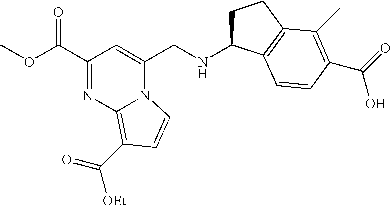 |
| 83 | 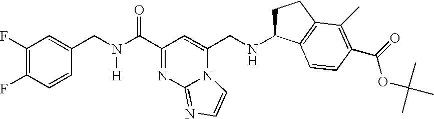 | 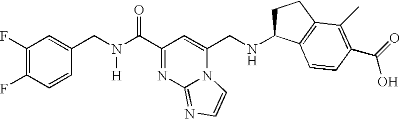 |
| 84 | 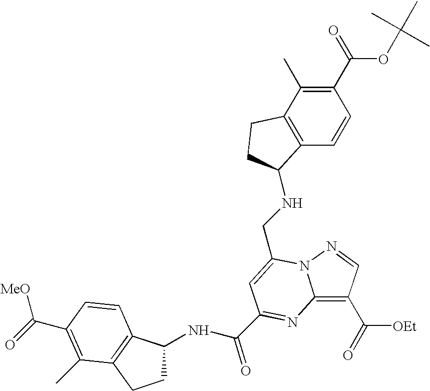 | 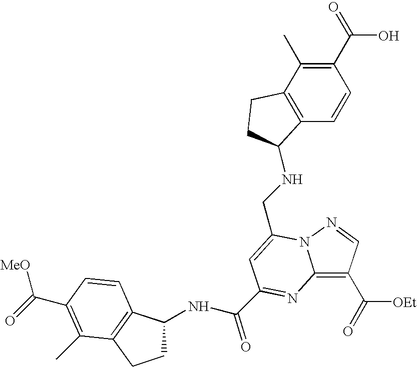 |
| 85 | 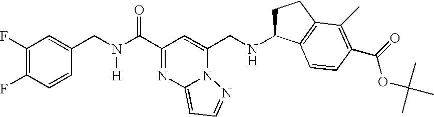 | 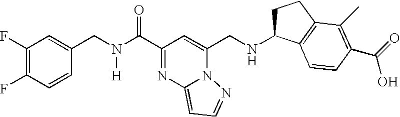 |
| 86 | 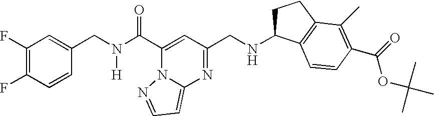 | 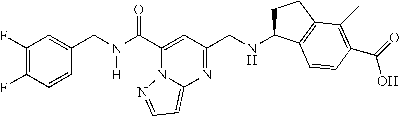 |
| 87 | 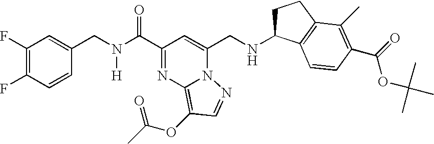 | 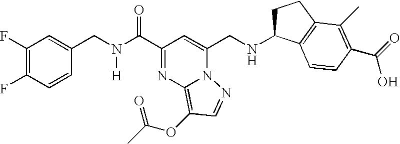 |
| 88 | 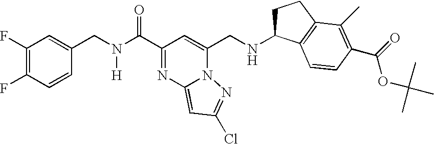 | 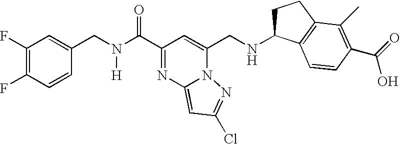 |
| 89 | 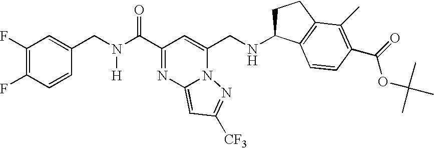 | 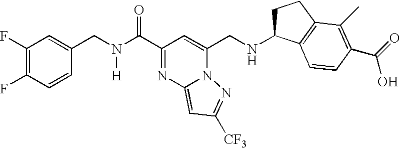 |
| 90 | 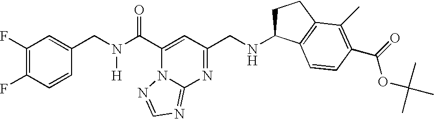 | 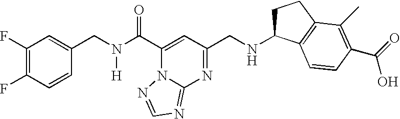 |
| 91 | 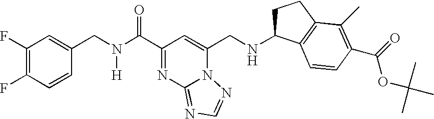 | 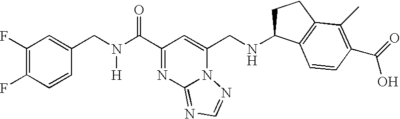 |
| 92 | 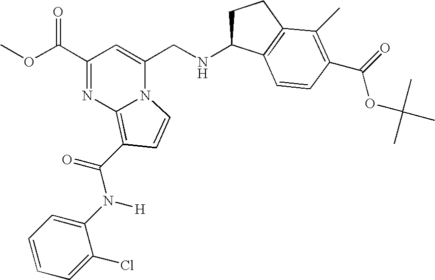 | 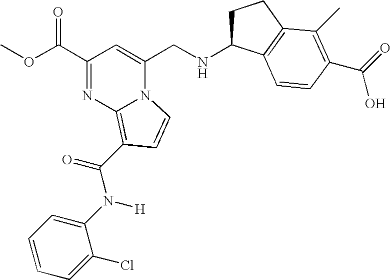 |
| 93 | 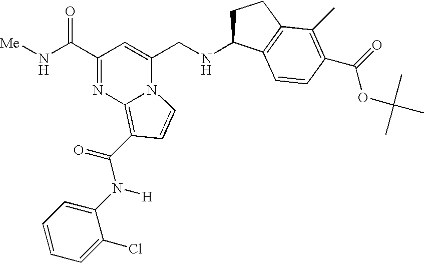 | 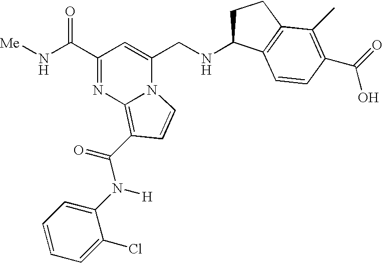 |
| 94 | 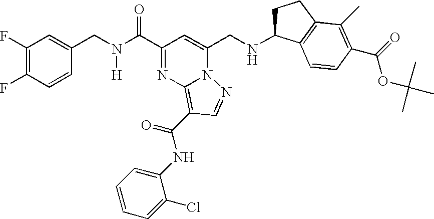 | 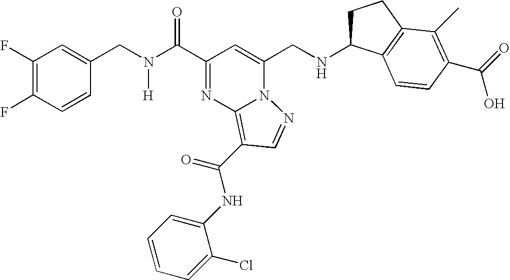 |
| 95 | 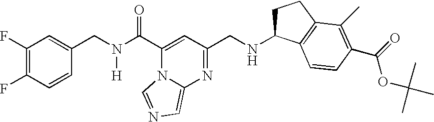 | 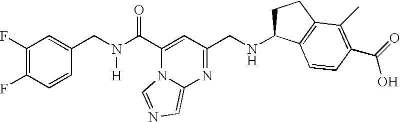 |

### Preparative Example 96

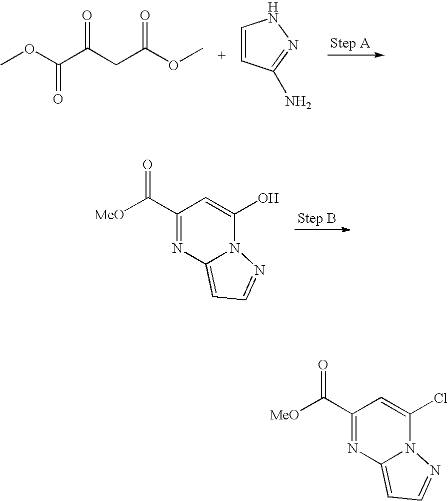

### Step A

Dimethyl 2-oxosuccinate (6.05 g, 37.8 mmol) and 1H-pyrazol-3-amine (3.14 g, 37.8 mmol) was heated to reflux in methanol (55 mL) for 16 h. After cooling down, the solid was collected by filtration and washed with methanol to afford methyl 7-hydroxypyrazolo[1,5-a]pyrimidine-5-carboxylate (2.32 g, 32%) as yellow solid. [MH]⁺=194.0

### Step B

Methyl 7-hydroxypyrazolo[1,5-a]pyrimidine-5-carboxylate (512 mg, 2.63 mmol) was added to phosphoryl trichloride (20 mL) and NAN-dimethylbenzenamine (0.126 mL). The mixture was stirred at 115° C. for 2 h, concentrated and added to ice-water and methylene chloride. The organic phase was separated and washed with NaHCO₃ twice, brine, dried over MgSO₄ and concentrated. The residue was purified by column chromatography (silica, hexane/EtOAc) to afford methyl 7-chloropyrazolo[1,5-a]pyrimidine-5-carboxylate (550 mg, 99% yield) as yellow solid. MH⁺=211.9

### Example 97

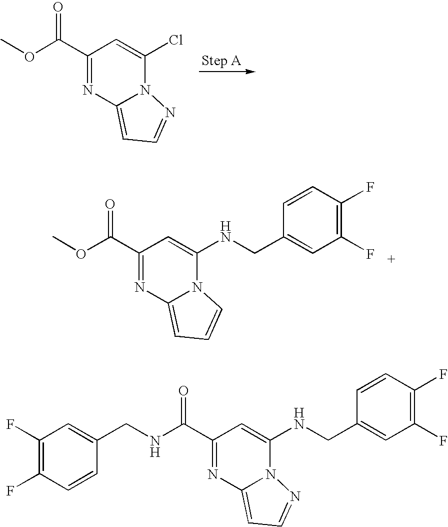

### Step A

Methyl 7-chloropyrazolo[1,5-a]pyrimidine-5-carboxylate (55 mg, 0.26 mmol) and 3,4-difluorobenzylamine (149 mg, 1.04 mmol) were dissolved in DMF (1 mL). The mixture was heated at 120° C. for 10 min in microwave and concentrated. The residue was purified by column chromatography (SiO₂, MeOH/CH₂Cl₂) to give methyl 7-(3,4-difluorobenzylamino)pyrazolo[1,5-a]pyrimidine-5-carboxylate (56 mg, 67% yield, [MH]⁺=319.1) and N-(3,4-difluorobenzyl)-7-(3,4-difluorobenzylamino)pyrazolo[1,5-a]pyrimidine-5-carboxamide (37 mg, 33% yield, [MH]⁺=430.1).

### Example 98

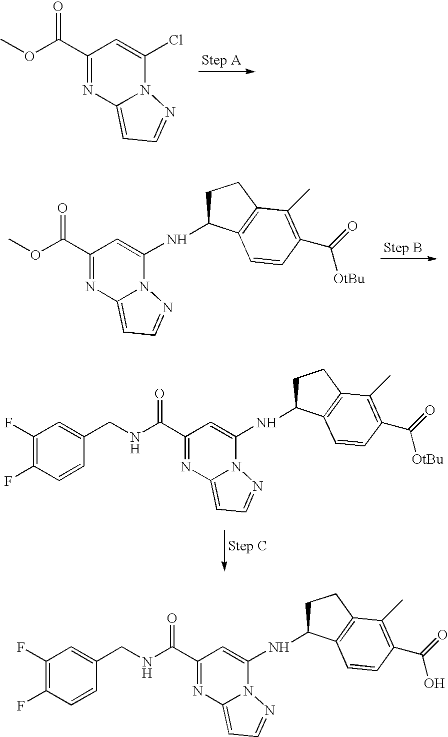

### Step A

Methyl 7-chloropyrazolo[1,5-a]pyrimidine-5-carboxylate (98 mg, 0.46 mmol) and (S)-tert-butyl 1-amino-2,3-dihydro-4-methyl-1H-indene-5-carboxylate (114 mg, 0.46 mmol) were dissolved in DMF (1.2 mL). The mixture was heated at 120° C. for 10 min in microwave and concentrated. The residue was purified by column chromatography (SiO₂, MeOH/CH₂Cl₂) to give methyl 7-((S)-5-(tert-butoxycarbonyl)-2,3-dihydro-4-methyl-1H-inden-1-ylamino)pyrazolo[1,5-a]pyrimidine-5-carboxylate (70 mg, 61% yield, [MH]⁺=423.1)

### Step B

Compound from Step A (67.5 mg, 0.16 mmol) and 3,4-difluorobenzylamine (68 mg, 0.47 mmol) were dissolved in DMF (1.3 mL). The mixture was heated at 150° C. for 2 h in microwave and concentrated. The residue was purified by column chromatography (SiO₂, MeOH/CH₂Cl₂) to give title compound (44 mg, 52% yield, [MH]⁺=534.2)

### Step C

Compound from Step B (40 mg, 0.075 mmol) was dissolved in methylene chloride (5 mL) and TFA (1 mL). The mixture was stirred for 3 h concentrated. The residue was washed by ether to give 1-[5-(3,4-difluoro-benzylcarbamoyl)-pyrazolo[1,5-a]pyrimidin-7-ylamino]-4-methyl-indan-5-carboxylic acid (35 mg, 98% yield, [MH]⁺=478.1)

### Example 99

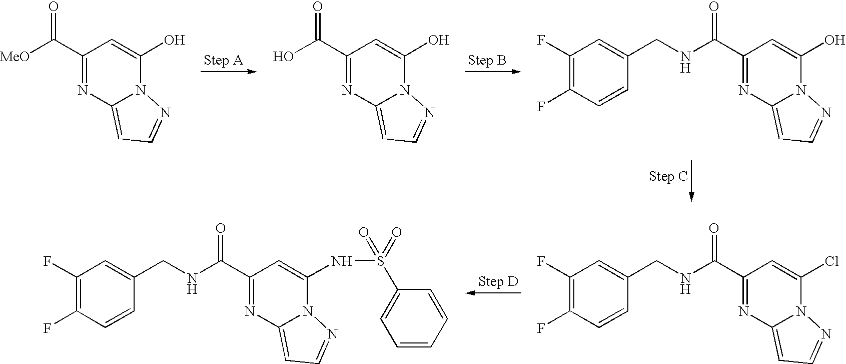

### Step A

NaOH (10 mL, 1N aq.) was added to methyl 7-hydroxypyrazolo[1,5-a]pyrimidine-5-carboxylate (663 mg, 3.28 mmol) in dioxane (30 mL). The mixture was stirred for 50 min and concentrated. The residue was purified by column chromatography (SiO₂, MeOH/CH₂Cl₂) to give 7-hydroxypyrazolo[1,5-a]pyrimidine-5-carboxylic acid (535 mg, 91% yield, [MH]⁺=180.0) as light yellow solid.

### Step B

Oxalyl chloride (0.52 mL) and DMF (2 drops) were added to the acid from Step A (364 mg, 2.03 mmol) in CH₂Cl₂ (3 mL). The reaction mixture was stirred for 30 min and concentrated under reduced pressure. The residue and 3,4-difluorobenzylamine (348 mg, 2.44 mmol) were dissolved in CH₂Cl₂ (3 mL). Triethylamine (0.85 mL) was added dropwise. The mixture was stirred for 5 h and concentrated. The residue was purified by column chromatography (silica, hexane/EtOAc) to afford title compound (307 mg, 50% yield) as light yellow solid. MH⁺=305.1

### Step C

The compound from Step B (300 mg, 1 mmol) was added to POCl₃ (4 mL) and N,N-dimethylbenzenamine (0.126 mL). The mixture was stirred at 105° C. for 6 h, concentrated and added to ice-water and methylene chloride. The organic phase was separated and washed with NaHCO₃ twice, brine, dried over MgSO₄ and concentrated. The residue was purified by column chromatography (silica, hexane/EtOAc) to afford title compound (126 mg, 39% yield) as light yellow solid. MH⁺=323.1

### Step D

The compound from step C (19.3 mg, 0.06 mmol) was mixed with benzenesulfonamide (14 mg, 0.09 mmol), palladium acetate (2.7 mg), xantphos (10.4 mg) and cesium carbonate (29.3 mg) in dioxane (2 mL). The mixture was heated to reflux for 16 h, concentrated and purified by column chromatography (silica, hexane/EtOAc) to afford 7-benzenesulfonylamino-pyrazolo[1,5-a]pyrimidine-5-carboxylic acid 3,4-difluoro-benzylamide (26 mg, 100% yield). MH⁺=444.1

### Example 100

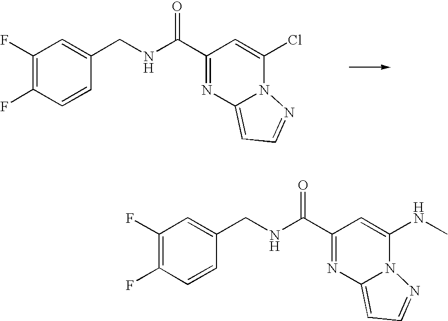

N-(3,4-difluorobenzyl)-7-chloropyrazolo[1,5-a]pyrimidine-5-carboxamide (16.4 mg) and methylamine (1 mL, 2N in MeOH) were heated at 130° C. for 5 min in microwave and concentrated. The residue was purified by column chromatography (SiO₂, MeOH/CH₂Cl₂) to give 7-methylamino-pyrazolo[1,5-a]pyrimidine-5-carboxylic acid 3,4-difluoro-benzylamide (16.1 mg, 100% yield, [MH]⁺=318.1)

### Example 101

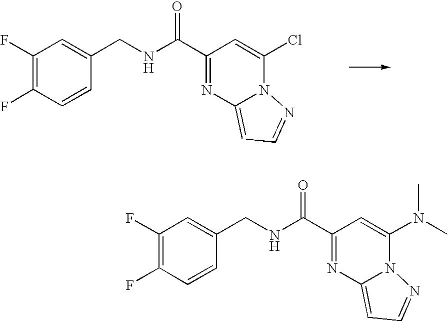

N-(3,4-difluorobenzyl)-7-chloropyrazolo[1,5-a]pyrimidine-5-carboxamide (10.5 mg) and dimethylamine (1 mL, 2N in THF) were heated at 130° C. for 10 min in microwave and concentrated. The residue was purified by column chromatography (SiO₂, MeOH/CH₂Cl₂) to give 7-dimethylamino-pyrazolo[1,5-a]pyrimidine-5-carboxylic acid 3,4-difluoro-benzylamide (9.0 mg, 83% yield, [MH]⁺=332.2)

### Example 102

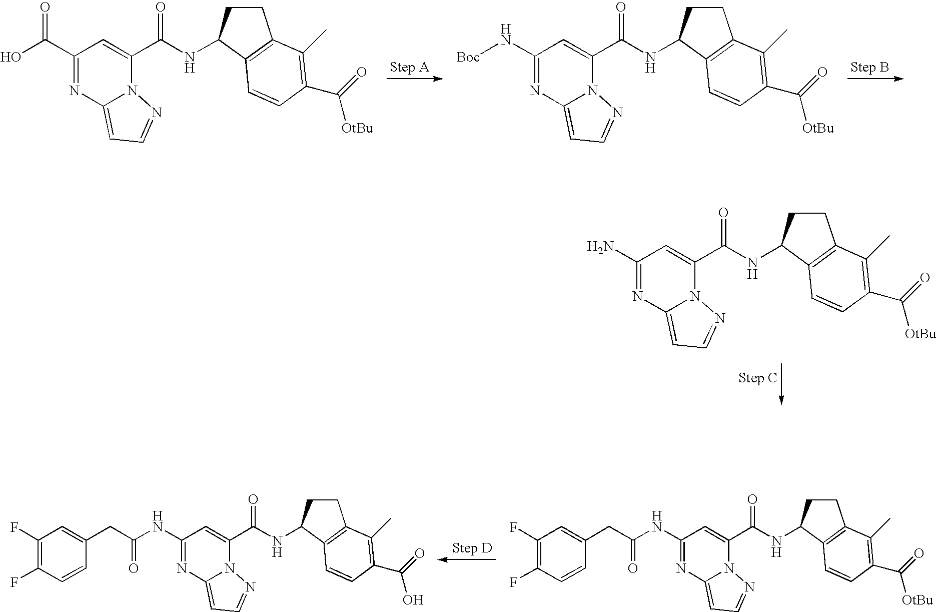

### Step A

7-((S)-5-(tert-butoxycarbonyl)-2,3-dihydro-4-methyl-1H-inden-1-ylcarbamoyl)pyrazolo[1,5-a]pyrimidine-5-carboxylic acid (418 mg, 0.96 mmol) was added to diphenylphosphoryl azide (528 mg) and triethylamine (0.294 mL) in tert-butanol (30 mL). The mixture was heated to reflux overnight, concentrated and purified by column chromatography (SiO₂, MeOH/CH₂Cl₂) to give title compound (404 mg, 83% yield, [MH]⁺=508.3)

### Step B

The compound from Step A (404 mg, 0.80 mmol) was dissolved in ethyl acetate (5 mL). HCl (2 N in Et₂O) was added slowly and reaction was monitored by TLC. When the reaction was done, the solid formed was collected by filtration to give title compound (133 mg, 41% yield, [MH]⁺=408.3)

### Step C

Oxalyl chloride (0.1 mL) and DMF (2 drops) were added to 2-(3,4-difluorophenyl)acetic acid (33 mg, 0.19 mmol) in CH₂Cl₂ (1 mL). The reaction mixture was stirred for 30 min and concentrated under reduced pressure. The residue and the compound from Step B (40 mg, 0.1 mmol) were dissolved in CH₂Cl₂ (3 mL). Triethylamine (0.57 mL) was added dropwise. The mixture was stirred for 5 h and concentrated. The residue was purified by column chromatography (silica, hexane/EtOAc) to afford title compound (37 mg, 66% yield) as light yellow solid. [M−H]-=560.5

### Step D

Compound from Step C (4.0 mg, 0.075 mmol) was dissolved in methylene chloride (1 mL) and TFA (0.5 mL). The mixture was stirred for 3 h concentrated. The residue was washed by ether to give 1-({5-[2-(3,4-difluoro-phenyl)-acetylamino]-pyrazolo[1,5-a]pyrimidine-7-carbonyl}-amino)-4-methyl-indan-5-carboxylic acid (2.6 mg, 72% yield, [M−H]-=504.3)

### Example 103

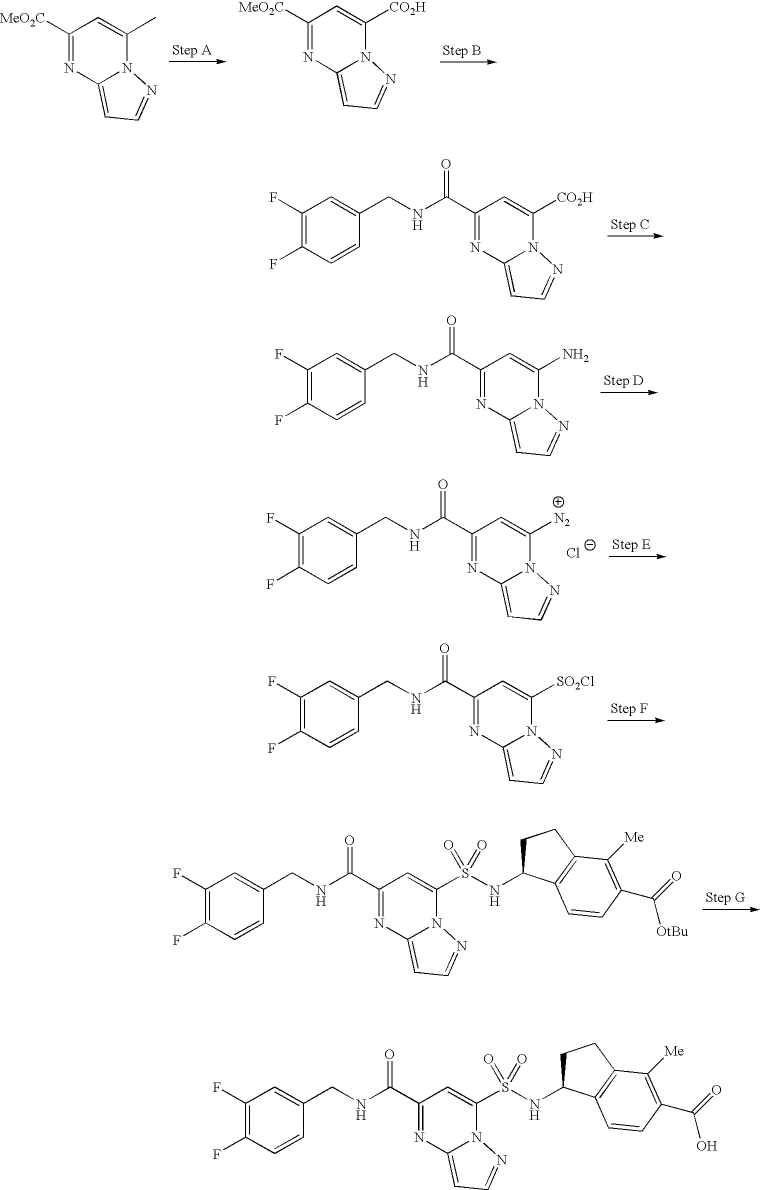

### Step A

If one were to mix 4-methyl-pyrrolo[1,2-a]pyrimidine-2-carboxylic acid methyl ester (1.38 g, 7.20 mmol) and SeO₂ (35 mmol) in dioxane (100 mL) and heat to reflux, the desired intermediate would be formed.

### Step B

If one were to mix the product of Step A (1.13 g, 5.10 mmol) and 4-fluoro-3-methylbenzylamine (0.71 g, 5.10 mmol) dissolved in DMF (20 mL) and heat to 60° C., the desired intermediate would be formed.

### Step C

If one were to mix the monoamide (0.16 g, 0.49 mmol) from above with LiOH (0.50 mL of a 1M aqueous solution) and MeOH (2 mL) and stir at room temperature until complete hydrolysis, then quench with HCl (0.50 mL of a 1M aqueous solution), and concentrate, the desired acid would be formed. If one were to mix the resulting acid, diphenylphosphoryl azide (270 mg, 1.0 mmol), and triethylamine (0.14 mL, 1.0 mmol) in 1-butanol (2 mL) and heated, then treated with HCl (4M solution in dioxane), the desired amine would be formed.

### Step D

If one were to add the amine (112 mg, 0.37 mmol) portionwise to a cooled solution of concentrated aqueous HCl (1 mL) followed by addition of a solution of sodium nitrite (27 mg, 0.39 mmol), one would obtain the desired intermediate.

### Step E

If one were to add the solution of the diazonium from Step D to a solution of copper(II) chloride (15 mg, 0.11 mmol) in glacial acetic acid (2 mL) which was saturated with sulfur dioxide, one would obtain the desired intermediate.

### Step, F

If one were to mix the sulfonyl chloride from Step E (85 mg, 0.22 mmol) in THF (1 mL) with triethylamine (92 μL, 0.66 mmol) and the amine (42 g, 0.24 mmol), one would obtain the desired intermediate.

### Step G

If one were to dissolve the product from Step F (100 mg, 0.19 mmol) in a 40% TFA/CH₂Cl₂ (1 mL) solution one would obtain 1-[5-(3,4-difluoro-benzylcarbamoyl)-pyrazolo[1,5-a]pyrimidine-7-sulfonylamino]-4-methyl-indan-5-carboxylic acid.

### Example 104

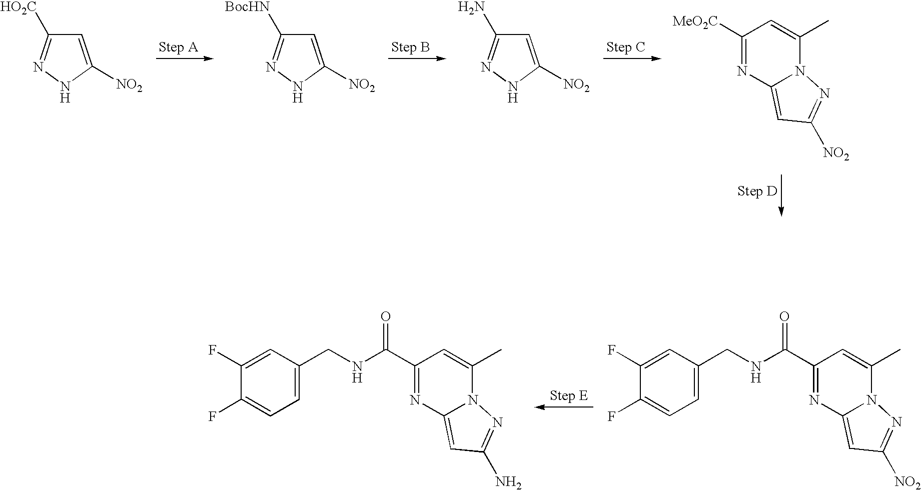

### Step A

5-Nitro-1H-pyrazole-3-carboxylic acid (1.57 g, 10 mmol), DPPA (4.3 mL, 20 mmol), triethylamine (2.8 mL, 20 mmol) and t-butanol (20 mL) was heated at 160° C. die 12 mins in microwave. The solution was concentrated to dryness after being cooled down. The crude product was purified by silica gel chromatography on Combiflash to give 3-Boc-amino-5-nitro-1H-pyrazole as white solid (1.85 g, yield 81%). MS (M+H): 158.

### Step B

To (1.45 g, 6.3 mmol) was hydrogen chloride in dioxane (4M, 15 mL). The reaction was stirred overnight, dilute with ether, and filtered to give desired product, 3-amino-5-nitro-1H-pyrazole hydrochloride salt as light brown solid (1.05 g, yield, 80%). MS (M+H): 129.

### Step C

3-Amino-5-nitro-1H-pyrazole (372 mg, 2.9 mmol) and methyl acetoacetate (419 mg, 2.9 mmol) in methanol (10 mL) were heated to reflux for 2 h and cooled down. The resulting precipitate was collected to give white solid product 7-methyl-2-nitro-pyrazolo[1,5-a]pyrimidine-5-carboxylic acid methyl ester (479 mg, yield 70%). MS (M+H): 237.

### Step D

To 7-methyl-2-nitro-pyrazolo[1,5-a]pyrimidine-5-carboxylic acid methyl ester (118 mg, 0.5 mmol) in N,N-dimethylformamide (2 mL) was added 3,4-difluorobenzyl-amine. The mixture was heated at 150° C. for 15 mins under microwave and poured in hydrochloric acid. The resulting precipitate was collected, wash with water and dried on high vacuum over potassium hydroxide to give off-white solid 7-methyl-2-nitro-pyrazolo[1,5-a]pyrimidine-5-carboxylic acid 3,4-difluoro-benzylamide (65 mg, 38% yield). MS (M+H): 348.

### Step E

To 7-methyl-2-nitro-pyrazolo[1,5-a]pyrimidine-5-carboxylic acid 3,4-difluoro-benzylamide (35 mg, 0.1 mmol) in ethanol (2 mL) was added tin (II) chloride (113 mg, 0.5 mmol) and heated to reflux. After 2 h, the reaction was cooled down and diluted with hydrochloric acid. The mixture was extracted with ethyl acetate, dried over magnesium sulfate and concentrated to give crude product, which was purified by silica gel chromatography to give desired product 2-amino-7-methyl-pyrazolo[1,5-a]pyrimidine-5-carboxylic acid 3,4-difluoro-benzylamide as white solid (17 mg, yield 53%). MS (M+H): 318.

### Example 105

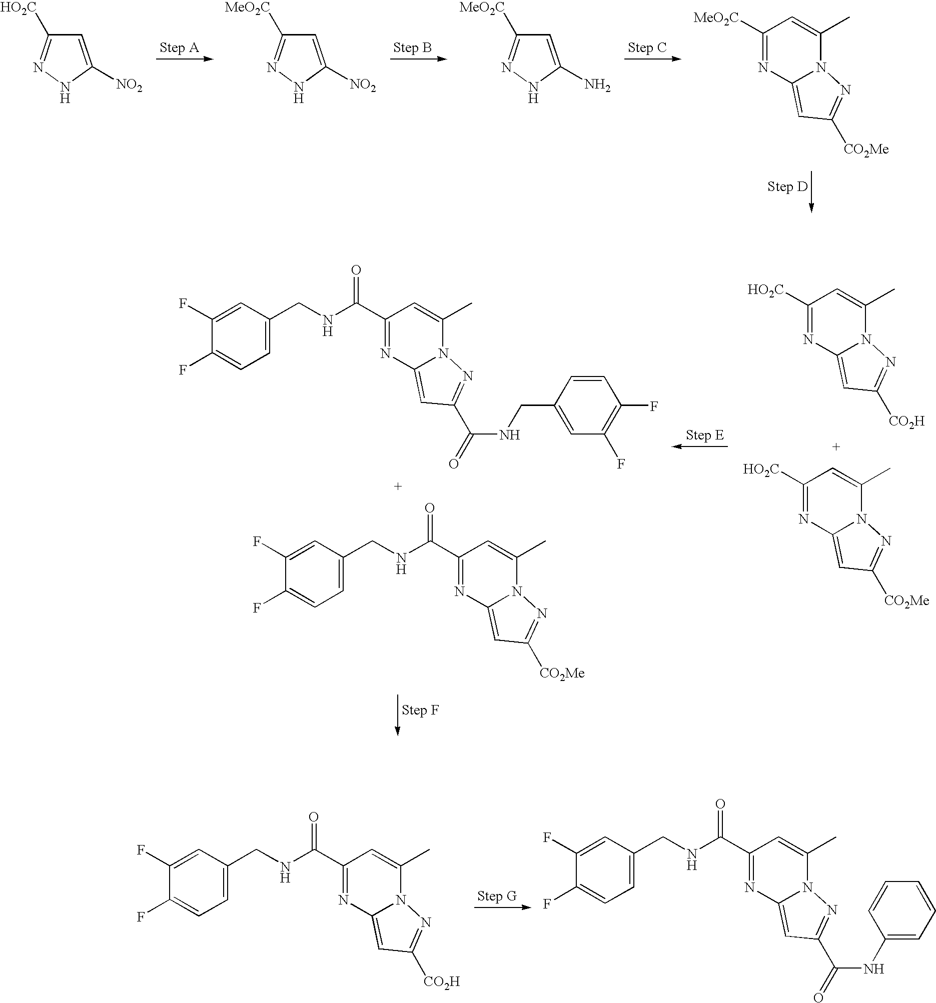

### Step A

5-Nitro-1H-pyrazole-3-carboxylic acid (1.57 g, 10 mmol) in methanol (25 mL) was added sulfuric acid (1 g, 10 mmol) and heated at 160° C. for 12 mins in microwave. The solution was concentrated to dryness after being cooled down. The crude product methyl 5-nitro-1H-pyrazole-3-carboxylate was pure enough to use without further purification. MS (M+H): 172.

### Step B

To methyl 5-nitro-1H-pyrazole-3-carboxylate (1.45 g, 6.3 mmol) in methanol (25 mL) was added palladium on carbon (106 mg, 0.1 mmol), hydrogenated for 2 h at 25 psi. The reaction mixture was filtered through a bed of celite and concentrated to give desired product, methyl 3-amino-1H-pyrazole 5-carboxylate as white solid (1.25 g, yield, 88%). MS (M+H): 142.

### Step C

Methyl 3-amino-1H-pyrazole 5-carboxylate (325 mg, 2.3 mmol) and methyl acetoacetate (330 mg, 2.3 mmol) in methanol (10 mL) were heated to reflux for 2 h and cooled down. The resulting precipitate was collected to give white solid product 7-Methyl-pyrazolo[1,5-a]pyrimidine-2,5-dicarboxylic acid dimethyl ester (356 mg, yield 62%). MS (M+H): 250.

### Step D

To a solution of methyl-pyrazolo[1,5-a]pyrimidine-2,5-dicarboxylic acid dimethyl ester (229 mg, 0.92 mmol) in dioxane (10 mL) and methanol (2 mL) was added a solution of sodium hydroxide (1N 1 mL). The solution was stirred for overnight, acidified, and the white precipitate filtered to afford the crude product as a mixture of diacid and monoacid (177 mg, 38%). MS (M+H): 236 (monoacid).

### Step E

To a mixture of the monoacid and diacid (172 mg), DMF (0.1 mL) and CH₂Cl₂ (2.5 mL) at 0° C. was added oxalyl chloride (180 μL, 2.2 mmol). The ice bath was removed and the mixture was stirred for 45 min and concentrated. The resulting residue was brought up in CH₂Cl₂ (2.5 mL) and added 3,4-difluorobenzylamine (114 mg, 0.8 mmol) and triethylamine (210 μL, 1.5 mmol) in CH₂Cl₂ (1 mL). The resulting mixture was stirred for 16 h and concentrated. The crude product was purified by silica gel chromatography to give the monoamide, 5-(3,4-difluoro-benzylcarbamoyl)-7-methyl-pyrazolo[1,5-a]pyrimidine-2-carboxylic acid methyl ester (171 mg, yield, 65%). MS (M+H): 361. The byproduct was diamide 7-Methyl-pyrazolo[1,5-a]pyrimidine-2,5-dicarboxylic acid bis-(3,4-difluoro-benzylamide) (95 mg, yield, 28%). MS (M+H): 472.

### Step F

The mixture of 5-(3,4-difluoro-benzylcarbamoyl)-7-methyl-pyrazolo[1,5-a]pyrimidine-2-carboxylic acid methyl ester (25 mg, 0.07 mmol), trimethyltin hydroxide (38.2 mg, 2.1 mmol) in 1,2-dichloroethane was heated to reflux for overnight and concentrated. The crude product was washed with hydrochloric acid and dried to give yellow solid 7-methyl-pyrazolo[1,5-a]pyrimidine-2,5-dicarboxylic acid 2-amide 5-(3,4-difluoro-benzylamide) (21 mg, yield, 86%). MS (M+H): 347.

### Step G

To a mixture of the 7-methyl-pyrazolo[1,5-a]pyrimidine-2,5-dicarboxylic acid 2-amide 5-(3,4-difluoro-benzylamide) (25 mg, 0.07 mmol), DMF (0.1 mL) and CH₂Cl₂ (2.5 mL) at 0° C. was added oxalyl chloride (18 μL, 0.22 mmol). The ice bath was removed and the mixture was stirred for 45 min and concentrated. The resulting residue was brought up in CH₂Cl₂ (2.5 mL) and added aniline (94 mg, 0.11 mmol) and triethylamine (31 μL, 0.22 mmol) in CH₂Cl₂ (1 mL). The resulting mixture was stirred for 16 h and concentrated. The crude product was purified by silica gel chromatography to give the diamide, -Methyl-pyrazolo[1,5-a]pyrimidine-2,5-dicarboxylic acid 5-(3,4-difluoro-benzylamide) 2-phenylamide (16 mg, yield, 38%). MS (M+H): 422.

### Example 106

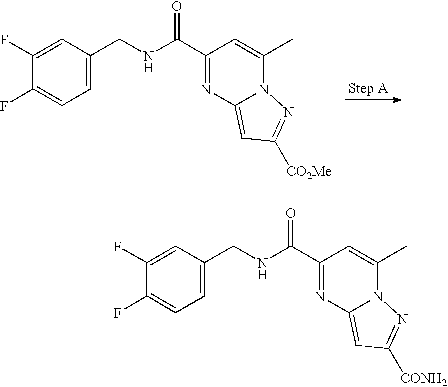

### Step A

5-(3,4-difluoro-benzylcarbamoyl)-7-methyl-pyrazolo[1,5-a]pyrimidine-2-carboxylic acid methyl ester (5 mg, 0.07 mmol) in ammonia methanol solution (7N, 2 mL) was heated to 65° C. overnight, concentrated and purified by silica gel chromatography to give 7-Methyl-pyrazolo[1,5-a]pyrimidine-2,5-dicarboxylic acid 2-amide 5-(3,4-difluoro-benzylamide) (4 mg, yield 90%). MS (M+H): 346.

### Example 107

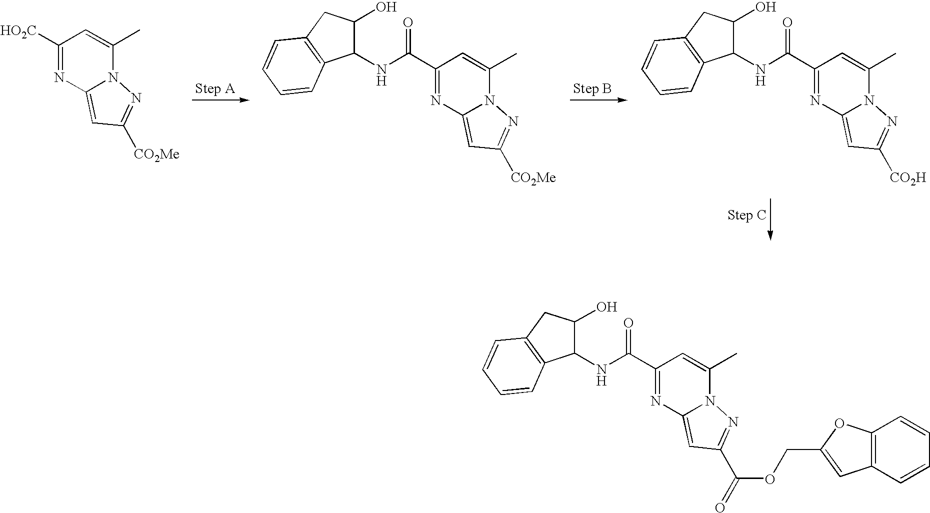

### Step A

To a solution of 7-methyl-pyrazolo[1,5-a]pyrimidine-2,5-dicarboxylic acid

2-methyl ester (47 mg, 0.2 mmol) and (1R,2S)-1-amino-2-hydroxyindane (30 mg, 0.2 mmol) in THF (3 mL) were added triethylamine (42 μL, 0.21 mmol), EDCl (40 mg, 0.21 mmol) and HOAt (15 mg, 0.21 mmol). The mixture was stirred overnight and then concentrated. The remaining residue was purified by chromatography to give (1R,2S)-5-(2-hydroxy-indan-1-ylcarbamoyl)-7-methyl-pyrazolo[1,5-a]pyrimidine-2-carboxylic acid methyl ester (60 mg, yield 82%). MS (M+H): 367.

### Step B

To a solution of the ester (60 mg) in THF (4 mL) was added a solution of LiOH (1N, 0.4 mL) in H₂O. The solution was stirred for 2 h, acidified, and filter to afford the (1R,2S)-5-(2-hydroxy-indan-1-ylcarbamoyl)-7-methyl-pyrazolo[1,5-a]pyrimidine-2-carboxylic acid as a bright yellow solid (59 mg, 99%). MS (M+H): 353.

### Step C

To a solution of 5-(2-hydroxy-indan-1-ylcarbamoyl)-7-methyl-pyrazolo[1,5-a]pyrimidine-2-carboxylic acid (29 mg, 0.08 mmol) and 2-hydroxymethylbenzofurane (13 mg, 0.09 mmol) in THF (2 mL) were added DMAP (18 mg, 0.15 mmol), EDCl (19 mg, 0.1 mmol). The mixture was stirred overnight and then concentrated. The remaining residue was purified by chromatography to give (1R,2S)-5-(2-Hydroxy-indan-1-ylcarbamoyl)-7-methyl-pyrazolo[1,5-a]pyrimidine-2-carboxylic acid benzofuran-2-ylmethyl ester (32 mg, yield 84%) as white solid. MS (M+H): 483.

### Example 108

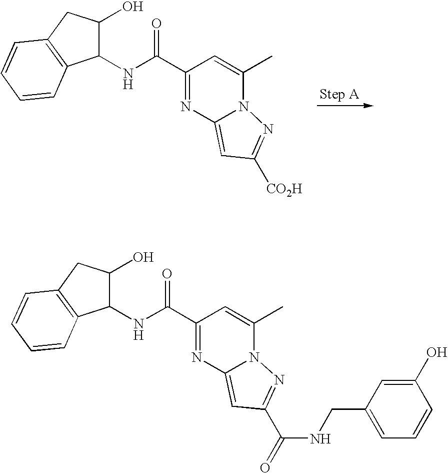

### Step A

To a solution of the 5-(2-hydroxy-indan-1-ylcarbamoyl)-7-methyl-pyrazolo[1,5-a]pyrimidine-2-carboxylic acid (29 mg, 0.08 mmol) (29 mg, 0.08 mmol) and 3-hydroxybenzylamine acetate salt (16.5 mg, 0.09 mmol) in THF (3 mL) were added triethylamine (42 μL, 021 mmol), EDCI (19 mg, 0.01 mmol) and HOAt (14 mg, 0.1 mmol). The mixture was stirred overnight and then concentrated. The remaining residue was purified by chromatography to afford 7-Methyl-pyrazolo[1,5-a]pyrimidine-2,5-dicarboxylic acid 2-(3-hydroxy-benzylamide) 5-[(2-hydroxy-indan-1-yl)-amide]as a white solid (35 mg, 90%). MS (M+H):458.

### Example 109

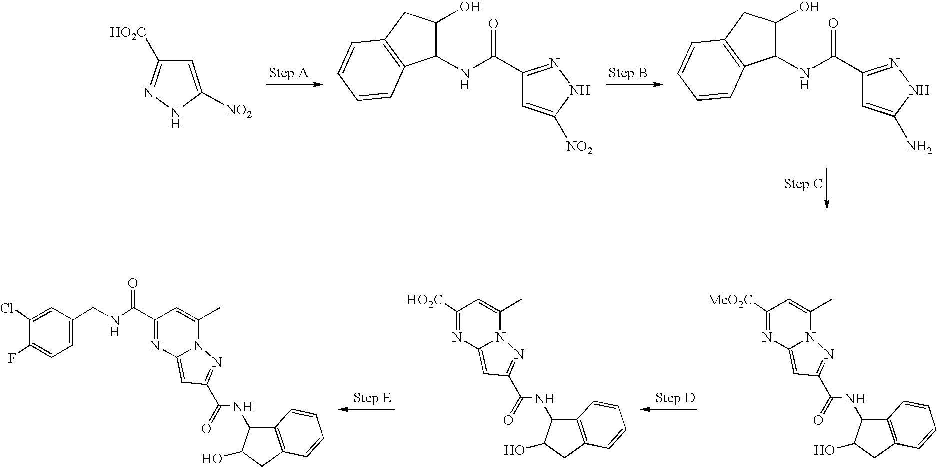

### Step A

To a solution of the 5-nitro-1H-pyrazole-3-carboxylic acid (315 mg, 2 mmol) and 1-amino-3-hydroxyindane (16.5 mg, 2 mmol) in DMF (3 mL) were added triethylamine (350 μL, 2.5 mmol), EDCI (270 mg, 2 mmol) and HOAt (40 mg, 2 mmol). The mixture was stirred overnight and then concentrated. The remaining residue was purified by chromatography to afford 5-nitro-1H-pyrazole-3-carboxylic acid (([(1R,2S)-2-hydroxy-indan-1-yl]-amide as a white solid. MS (M+H): 289.

### Step B

To 5-nitro-1H-pyrazole-3-carboxylic acid [(1R,2S)-2-hydroxy-indan-1-yl]-amide (1.45 g, 6.3 mmol) in methanol (10 mL) was added palladium on carbon (212 mg, 0.1 mmol), hydrogenated for 2 h at 25 psi. The reaction mixture was filtered through a bed of celite and concentrated to give desired product, 5-amino-1H-pyrazole-3-carboxylic acid [(1R,2S)-2-hydroxy-indan-1-yl]-amide as white solid which was used without further purification. MS (M+H): 259.

### Step C

5-Amino-1H-pyrazole-3-carboxylic acid (2-hydroxy-indan-1-yl)-amide and methyl acetoacetate (289 mg, 2 mmol) in methanol (10 mL) were heated to reflux for 2 h and cooled down. The resulting precipitate was collected to give white solid product 2-[(1R,2S)-2-hydroxy-indan-1-yl]carbamoyl)-7-methyl-pyrazolo[1,5-a]pyrimidine-5-carboxylic acid methyl ester (215 mg, yield 30% for three steps). MS (M+1): 367.

### Step D

The mixture of 2-([(1R,2S)-2-hydroxy-indan-1-yl]carbamoyl)-7-methyl-pyrazolo[1,5-a]pyrimidine-5-carboxylic acid methyl ester (30 mg, 0.08 mmol), trimethyltin hydroxide (30 mg, 0.165 mmol) in 1,2-dichloroethane was heated to reflux for overnight and concentrated. The crude product was washed with hydrochloric acid and dried to give a white solid 2-([(1R,2S)-2-hydroxy-indan-1-yl carbamoyl)-7-methyl-pyrazolo[1,5-a]pyrimidine-5-carboxylic acid (30 mg, yield, 99%). MS (M+H): 353.

### Step E

To a solution of the 2-([(1R,2S)-2-hydroxy-indan-1-yl carbamoyl)-7-methyl-pyrazolo[1,5-a]pyrimidine-5-carboxylic acid (10.6 mg, 0.03 mmol) and 3-chloro-4-fluorobenzylamine (4.8 mg, 0.03 mmol) in DMF (1 mL) were added triethylamine (6.3 μL, 0.033 mmol), EDCI (6.3 mg, 0.033 mmol) and HOAt (4.5 mg, 0.033 mmol). The mixture was stirred overnight and then concentrated. The remaining residue was purified by chromatography to afford 7-methyl-pyrazolo[1,5-a]pyrimidine-2,5-dicarboxylic acid 5-(3-chloro-4-fluoro-benzylamide) 2-{[(1R,2S)-2-hydroxy-indan-1-yl]amide} as a white solid (12.2 mg, yield, 82%). MS (M+H): 494.

### Example 110

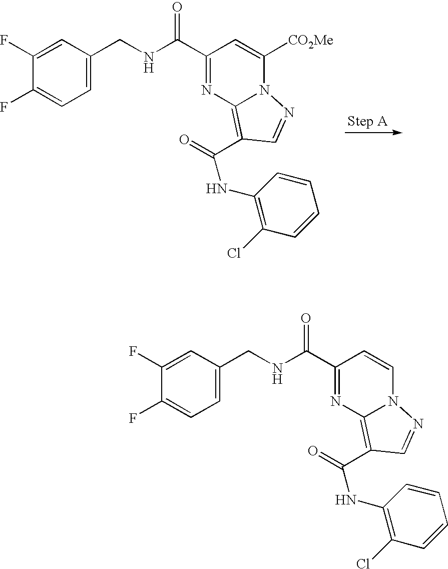

### Step A

The mixture of 3-(2-Chloro-phenylcarbamoyl)-5-(3,4-difluoro-benzylcarbamoyl)-pyrazolo[1,5-a]pyrimidine-7-carboxylic acid methyl ester (40 mg, 0.08 mmol), trimethyltin hydroxide (75 mg, 0.4 mmol) in 1,2-dichloroethane was heated to reflux for overnight and concentrated. The crude product was washed with hydrochloric acid and dried to give yellow solid pyrazolo[1,5-a]pyrimidine-3,5-dicarboxylic acid 3-[(2-chloro-phenyl)-amide] 5-(3,4-difluoro-benzylamide) (12 mg, yield, 34%). MS (M+H): 442.

### Example 111

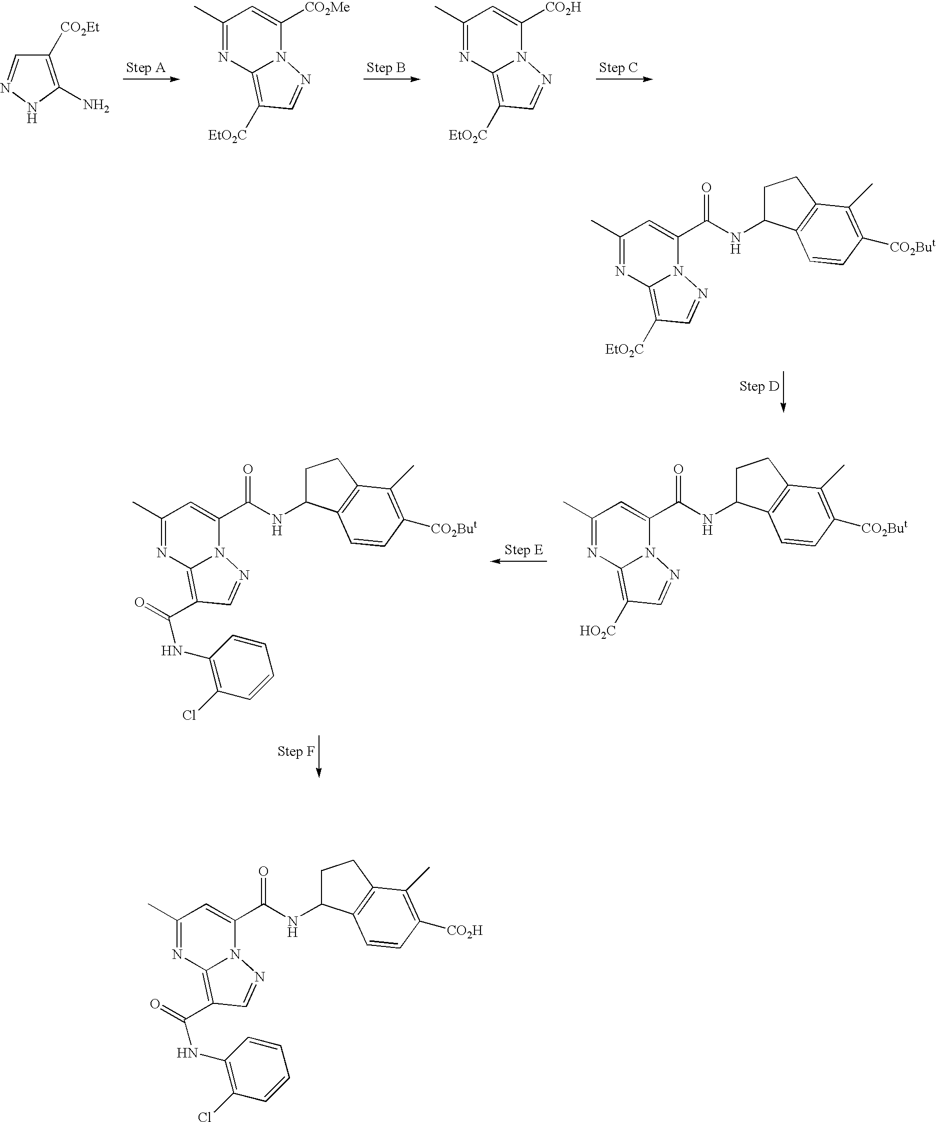

### Step A

2-Amino-1H-pyrazole-3-carboxylic acid ethyl ester (0.7 g, 5 mmol) and methyl acetoacetate (0.62 g, 5 mmol) in methanol (10 mL) were heated to reflux for 2 h and cooled down. The resulting precipitate was collected to give white solid 5-methyl-pyrazolo[1,5-a]pyrimidine-3,7-dicarboxylic acid 3-ethyl ester 7-methyl ester (0.8 g mg, yield, 60%). MS (M+H): 264.

### Step B

To a solution of 5-methyl-pyrazolo[1,5-a]pyrimidine-3,7-dicarboxylic acid 3-ethyl ester 7-methyl ester (0.53 g, 2 mmol) in dioxane (10 mL) and methanol (2 mL) was added a solution of sodium hydroxide (1N, 2.2 mL). The solution was stirred for overnight, acidified, and the orange precipitate was filtered to afford the crude product monoacid (550 mg). MS (M+H): 250.

### Step C

To a mixture of the 7-methyl-pyrazolo[1,5-a]pyrimidine-2,5-dicarboxylic acid 2-amide 5-(3,4-difluoro-benzylamide) (125 mg, 0.5 mmol), DMF (0.1 mL) and CH₂Cl₂ (5 mL) at 0° C. was added oxalyl chloride (120 μL, 1.5 mmol). The ice bath was removed and the mixture was stirred for 45 min and concentrated. The resulting residue was brought up in CH₁₂Cl₂ (5 mL) and added (1S)-1-amino-4-methyl-indan-5-carboxylic acid tert-butyl ester (170 mg, 0.5 mmol) and triethylamine (260 μL, 1.5 mmol) in CH₂Cl₂ (1 mL). The resulting mixture was stirred for 16 h and concentrated. The crude product was purified by silica gel chromatography to give, 7-((1S)-5-tert-butoxycarbonyl-4-methyl-indan-1-ylcarbamoyl)-5-methyl-pyrazolo[1,5-a]pyrimidine-3-carboxylic acid ethyl ester (216 mg, yield, 90%). MS (M+H): 479.

### Step D

The mixture of 7-((1S)-5-tert-butoxycarbonyl-4-methyl-indan-1-ylcarbamoyl)-5-methyl-pyrazolo[1,5-a]pyrimidine-3-carboxylic acid ethyl ester (40 mg, 0.08 mmol), trimethyltin hydroxide (75 mg, 0.4 mmol) in 1,2-dichloroethane (2 mL) was heated to reflux for overnight and concentrated. The crude product was washed with hydrochloric acid and dried to give a brown solid 7-((1S)-5-tert-butoxycarbonyl-4-methyl-indan-1-ylcarbamoyl)-5-methyl-pyrazolo[1,5-a]pyrimidine-3-carboxylic acid (390 mg, yield, 99%). MS (M+H): 451

### Step E

To a mixture of the 7-((1S)-5-tert-butoxycarbonyl-4-methyl-indan-1-ylcarbamoyl)-5-methyl-pyrazolo[1,5-a]pyrimidine-3-carboxylic acid (39 mg, 0.08 mmol), DMF (0.1 mL) and CH₂Cl₂ (2.5 mL) at 0° C. was added oxalyl chloride (20 μL, 0.24 mmol). The ice bath was removed and the mixture was stirred for 45 min and concentrated. The resulting residue was brought up in CH₁₂Cl₂ (2.5 mL) and added 2-chloroaniline (15 mg, 0.12 mmol) and triethylamine (17 μL, 0.12 mmol) in CH₂Cl₂ (1 mL). The resulting mixture was stirred for 16 h and concentrated. The crude product was purified by silica gel chromatography to give the (1S)-1-{[3-(2-Chloro-phenylcarbamoyl)-5-methyl-pyrazolo[1,5-a]pyrimidine-7-carbonyl]-amino}-4-methyl-indan-5-carboxylic acid tert-butyl ester (15 mg, yield, 38%). MS (M+H): 560.

### Step F

To 7-((1S)-5-tert-butoxycarbonyl-4-methyl-indan-1-ylcarbamoyl)-5-methyl-pyrazolo[1,5-a]pyrimidine-3-carboxylic acid tert-butyl ester (5 mg) was added trifluoroacetic acid and methylene chloride (1:1, 0.5 mL). The mixture was stirred for 1 h at room temperature and concentrated to dryness, The residue was washed with diethyl ether and dried to give pure product (1S)-1-{[3-(2-Chloro-phenylcarbamoyl)-5-methyl-pyrazolo[1,5-a]pyrimidine-7-carbonyl]-amino}-4-methyl-indan-5-carboxylic acid (4 mg, 90%). MS (M+Hi): 504

### Preparative Example 112

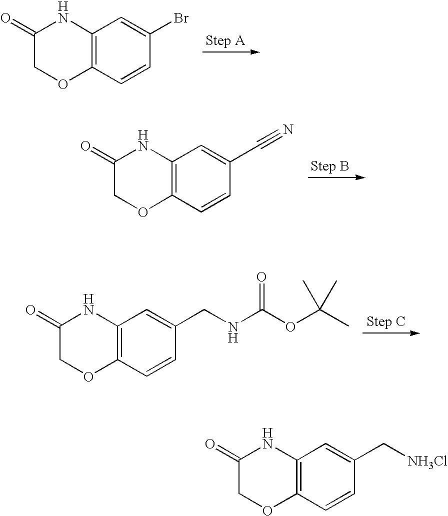

### Step A

A degassed suspension of commercially available 6-Bromo-4H-benzo[1,4]oxazin-3-one (8.39 g), Zn(CN)₂ (3.46 g) and Pd(PPh₃)₄ (2.13 g) in DMF (70 mL) was stirred in a oil bath (80° C.) overnight. The mixture was cooled to room temperature and then poured into water (500 mL). The precipitate was collected by suction, air dried, washed with pentane, dissolved in CF₁₂Cl₂/MeOH (1:1), filtered through a silica pad and concentrated to yield a the title compound (5.68 g, 89%). [MH]⁺=175.

### Step B

To an ice cooled solution of the title compound from Step A above (5.6 g), di-tert-butyl dicarbonate (14.06 g) and NiCl₂.6H₂O (1.53 g) in MeOH, NaBH₄ (8.51 g) was added in portions. The mixture was vigorously stirred for 1 h at 0° C. and 1 h at room temperature. After the addition of diethylenetriamine (3.5 mL) the mixture was concentrated, diluted with EtOAc, washed subsequently with 1N HCl, saturated aqueous NaHCO₃ and saturated aqueous NaCl, dried (MgSO₄), concentrated to afford the title compound as an off white solid (7.91 g, 88%). [M+Na]⁺=397.

### Step C

The title compound from Step B above (7.91 g) was dissolved in a 4M solution of HCl in 1,4-dioxane (120 mL), stirred for 14 h at room temperature, concentrated, suspended in Et₂O, filtered and dried to afford the title compound as an off-white solid (5.81 g, 96%). [M−NH₃Cl]⁺=162.

### Preparative Example 113

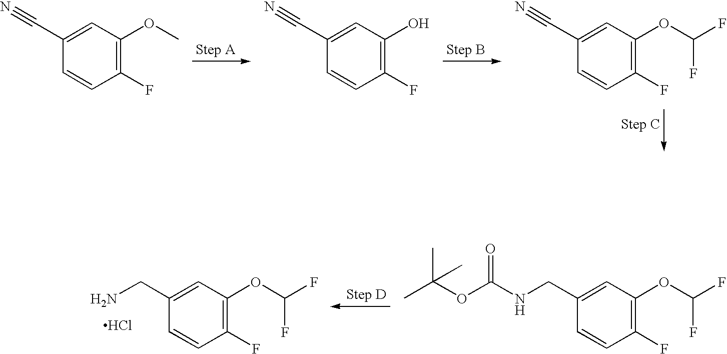

### Step A

Under an argon atmosphere a mixture of commercially available 4-fluoro-3-methoxybenzonitrile (5.0 g), AlCl₃ (8.8 g) and NaCl (1.94 g) was heated (melted) to 190° C. for 45 min, cooled, poured on ice (200 mL) and extracted with CHCl₃ (3×). The combined organic phases were washed with H₂O, dried (MgSO₄), filtered, concentrated and purified by chromatography (silica, cyclohexane/EtOAc 9:1 to 8:1) to afford the title compound as colorless needles (3.45 g, 76%). [MH]⁺=138.

### Step B

A suspension of the title compound from Step A above (8.73 g) and K₂CO₃ (8.1 g) in dry DMF (200 mL) was heated to 50° C. for 10 min and then chlorodifluoromethane (50 g) was condensed into the mixture using a dry-ice condenser and the resulting slurry was stirred oil-bath temperature of 160° C. (internal temp. much lower, but not measured) for 8 h and then at room temperature overnight without condenser. The mixture was concentrated, diluted with EtOAc, washed subsequently with 1N aqueous HCl and saturated aqueous NaCl, dried (MgSO₄), filtered and concentrated. Purification by chromatography (silica, cyclohexane/EtOAc 95:5 to 8:2) afforded the title compound as a colorless oil (9.36 g, 79%). [MH]⁺=188.

### Step C

To an ice cooled solution of the title compound from Step B above (9.3 g) in dry MeOH (250 mL) were added di-lert-butyl dicarbonate (22 g) and NiCl₂.6H₂O (700 mg), followed by the careful portionwise addition of NaBH₄ (11 g). The resulting black mixture was stirred for 20 min at 0-5° C. (ice bath), then the ice bath was removed and stirring at room temperature was continued overnight. Then diethylenetriamine was added and the mixture was concentrated to dryness. The remaining residue was suspended in EtOAc, washed subsequently with 10% aqueous citric acid, saturated aqueous NaHCO₃ and saturated aqueous NaCl, dried (MgSO₄), filtered, concentrated and purified by chromatography (silica, cyclohexane/EtOAc 9:1 to 7:3) to afford the title compound as a colorless oil (8.8 g, 99%, [MNa]⁺=314).

### Step D

To a suspension of the title compound from the Step C above (11.4 g) was added a 4M solution of HCl in 1,4-dioxane (65 mL). The reaction mixture was stirred at room temperature overnight and concentrated to afford the title compound as a colorless solid (8.8 g, 99%). [M−Cl]⁺=192.

### Preparative Example 114

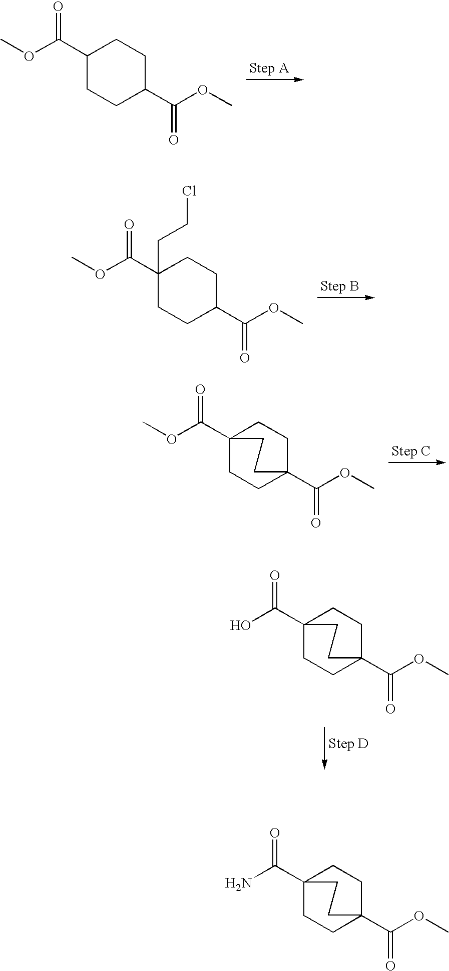

### Step A

To a cooled (−30° C.) solution of ⁱPr₂NH (16.9 mL) in THF (140 mL) was dropwise added a 2.5M solution of BuLi in hexane (43.2 mL). The mixture was stirred between −20° C. and −30° C. for 20 min and then cooled to −78° C. To this solution dry HMPA (72 mL) was added dropwise not allowing the temperature of the mixture to exceed −70° C. The resultant mixture was cooled again to −78° C. and a solution of commercially available dimethylcyclohexane-1,4-dicarboxylate (20 g) in THF (20 mL) was added dropwise over a period of 10 min. Stirring at −78° C. was continued for 40 min, then 1-bromo-2-chloroethane (10 mL) was added over a period of 5 min, the cooling bath was removed and the mixture was allowed to warm to room temperature. The mixture was then quenched with saturated aqueous NH₄Cl, the volatiles were removed by evaporation and the mixture was diluted with cyclohexane and H₂O. The aqueous phase was separated and extracted with cyclohexane (2×). The combined organic phases were washed with H₂O and saturated aqueous NaCl, dried (MgSO₄), filtered and concentrated. The remaining residue was distilled (10⁻² mbar, 100° C.) to give the title compound as a pale yellow oil (17 g, 65%). [MH]⁺=263.

### Step B

To a cooled (−30° C.) solution of ⁱPr₂NH (18.7 mL) in THF (180 mL) was dropwise added a 2.5M solution of BuLi in hexane (53.6 mL). The mixture was stirred between −20° C. and −30° C. for 20 min and then cooled to −78° C. This solution was cannulated over a period of 30 min into a cooled (−78° C.) mixture of the title compound from Step A above (32 g) and HMPA (90 mL) in THF (440 mL) not allowing the temperature of the mixture to exceed −70° C. Stirring at −78° C. was continued for 25 min and then the mixture was allowed to warm to room temperature over a period of 1½ h. The mixture was kept at room temperature for 1 h and then quenched with saturated aqueous NH₄Cl. The volatiles were removed by evaporation and the mixture was diluted with cyclohexane and H₂O. The aqueous phase was separated and extracted with cyclohexane (3×). The combined organic phases were washed with H₂O and saturated aqueous NaCl, dried (MgSO₄), filtered and concentrated. The remaining residue was recrystallized from cyclohexane to give the title compound (13.8 g, 50%). [MH]⁺=227.

### Step C

A mixture of the title compound from Step B above (20 g) and KOH (5.5 g) in MeOH/H₂O (10:1, 106 mL) was heated to reflux overnight, cooled to room temperature and concentrated. The residue was diluted with EtOAc and extracted with 1N aqueous NaOH (2×100 mL). The organic phase was dried (MgSO₄), filtered and concentrated to give the starting material as a white solid. The combined aqueous phases were adjusted with 2N aqueous HCl to pH 1-2 and extracted with EtOAc (4×250 mL). The combined turbid organic phases were filtered through a fluted filter, washed with saturated aqueous NaCl, dried (MgSO₄), filtered and concentrated to give the title compound as a colorless solid (13.1 g, 70%). [MH]⁺=213.

### Step D

To a cooled (−40° C.) solution of the title compound from Step C above (500 mg) and NEt₃ (1.23 mL) in THF (50 mL) was slowly added ethyl chloroformate (0.67 mL). The mixture was allowed to warm to −25° C. and stirred at this temperature for 1 h. A 7N solution of NH₃ in MeOH (10 mL) was added and the mixture was stirred at −20° C. for 30 min. The cooling bath was removed and the mixture was stirred at room temperature for 15 min before it was concentrated. To the remaining residue were added H₂O (10 mL) and CH₂Cl₂ (20 mL), the organic phase was separated and the aqueous phase was extracted with CH₂Cl₂ (2×10 mL). The combined organic phases were washed with 1N aqueous KOH (10 mL), dried (MgSO₄), filtered and concentrated to afford the title compound (458 mg, 92%). [MH]⁺=212.

### Preparative Example 115

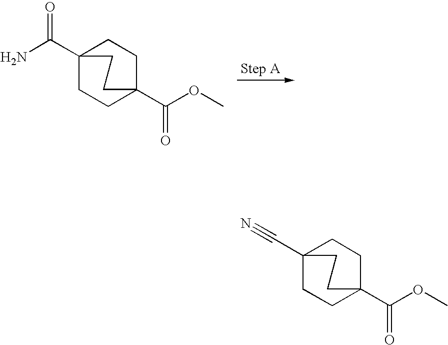

### Step A

To a cooled (0° C.) mixture of methyl 4-carbamoylbicyclo[2.2.2]octane-1-carboxylate (228 mg) and imidazole (147 mg) in pyridine (10 mL) was slowly added POCl₃ (0.40 mL). The mixture was stirred at 0° C. for 1 h and then added to a mixture of ice, NaCl and EtOAc. The organic phase was separated and washed with 1N aqueous HCl until the aqueous phase remained acidic. Drying (MgSO₄), filtration and concentration afforded the title compound (137 mg, 72%). [MH]⁺=194.

### Preparative Example 116

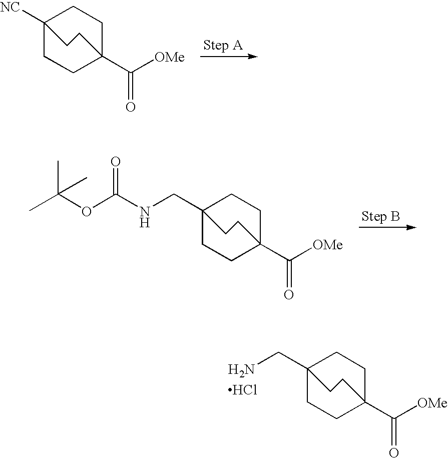

### Step A

Methyl 4-cyanobicyclo[2.2.2]octane-1-carboxylate (137 mg) was treated similarly as described in the Preparative Example 113 steps C and D to afford the title compound (163 mg, 77%). [MNa]⁺=320.

### Step B

The title compound from Step A above (882 mg) was dissolved in a 4M solution of HCl in 1,4-dioxane (15 mL), stirred for 14 h, concentrated, suspended in Et₂O, filtered and dried to afford the title compound (690 mg, >99%). [M−Cl]⁺=198.

### Preparative Example 117

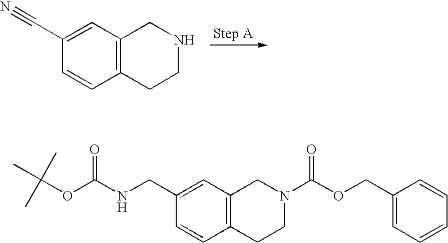

### Step A

A solution of commercially available 7-cyano-1,2,3,4-tetrahydroisoquinoline (2.75 g), K₂CO₃ (3.60 g) and benzylchloroformate (2.7 mL) in THF/H₂O was stirred overnight and then concentrated. The residue was diluted with EtOAc, washed with 10% aqueous citric acid, saturated aqueous NaHCO₃ and saturated aqueous NaCl, dried (MgSO₄) and concentrated. The residue was dissolved in MeOH (100 mL) and di-tert-butyl dicarbonate (7.60 g) and NiCl₂.6H₂O (400 mg) was added. The solution was cooled to 0° C. and NaBH₄ (2.60 g) was added in portions. The mixture was allowed to reach room temperature and then vigorously stirred overnight. After addition of diethylenetriamine (2 mL) the mixture was concentrated, diluted with EtOAc, washed subsequently with 10% aqueous citric acid, saturated aqueous NaHCO₃ and saturated aqueous NaCl, dried (MgSO₄), concentrated and purified by chromatography (silica, CH₂Cl₂/MeOH) to afford the title compound as a colorless oil (1.81 g, 26%). [MH]⁺=397.

### Preparative Example 118

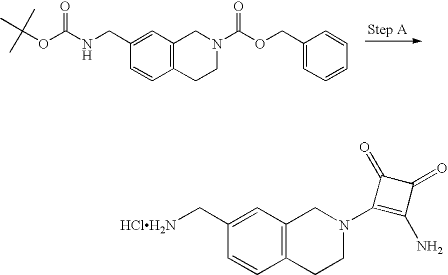

### Step A

A mixture of tert-butyl (2-((benzyloxy)carbonyl)-1,2,3,4-tetrahydroisoquinolin-7-yl)methylcarbamate (1.81 g) and Pd/C (10 wt %, 200 mg) in EtOH (50 mL) was hydrogenated at atmospheric pressure overnight, filtered and concentrated to a volume of ˜20 mL. 3,4-Diethoxy-3-cyclobutene-1,2-dione (0.68 mL) and NEt₃ (0.5 mL) were added and the mixture was heated to reflux for 4 h. Concentration and purification by chromatography (silica, cyclohexane/EtOAc) afforded a slowly crystallizing colorless oil. This oil was dissolved in EtOH (20 mL) and a 28% solution of NH₃ in H₂O (100 mL) was added. The mixture was stirred for 3 h, concentrated, slurried in H₂O, filtered and dried under reduced pressure. The remaining residue was dissolved in a 4M solution of HCl in 1,4-dioxane (20 mL), stirred for 14 h, concentrated, suspended in Et₂O, filtered and dried to afford the title compound as an off-white solid (1.08 g, 92%). [M−Cl]⁺=258.

### Preparative Example 119

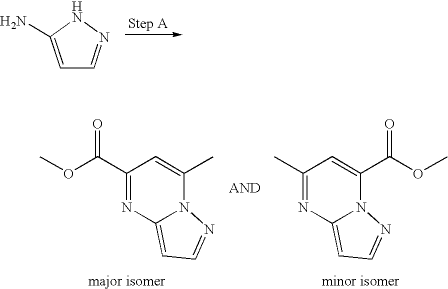

### Step A

To a solution of commercially available 1H-pyrazol-5-amine (86.4 g) in MeOH (1.80 L) was added commercially available methyl acetopyruvate (50.0 g). The mixture was heated to reflux for 5 h and then cooled to room temperature overnight. The precipitated yellow needles were collected by filtration and the supernatant was concentrated at 40° C. under reduced pressure to ⅔ volume until more precipitate began to form. The mixture was cooled to room temperature and the precipitate was collected by filtration. This concentration/precipitation/filtration procedure was repeated to give 3 batches. This material was combined and recrystallized from MeOH to give the major isomer of the title compound (81.7 g, 72%). [MH]⁺=192.

The remaining supernatants were combined, concentrated and purified by chromatography (silica, cyclohexane/EtOAc) to afford the minor isomer of title compound (6.8 g, 6%). [MH]⁺=192.

### Preparative Example 120

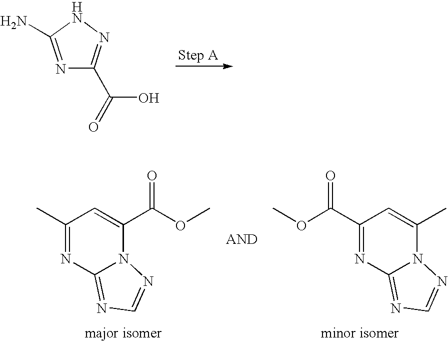

### Step A

A mixture of commercially available 5-amino-1H-[1,2,4]triazole-3-carboxylic acid (20.3 g) and methyl acetopyruvate (20.0 g) in glacial AcOH (250 mL) was heated to 95° C. for 3 h. The mixture was concentrated and diluted with saturated aqueous NaHCO₃ (200 mL) and CH₂Cl₂ (500 mL). The organic phase was separated, dried (MgSO₄), filtered and concentrated to give a pale orange mixture of regioisomers (80:20, 21.3 g, 80%). Recrystallization of the crude material from hot THF (110 mL) afforded the major isomer of the title compound (13.0 g, 49%). [MH]⁺=193. The supernatant was concentrated and purified by chromatography (silica, hexanes/EtOAc) to afford the minor isomer of title compound. [MH]⁺=193.

### Preparative Example 121

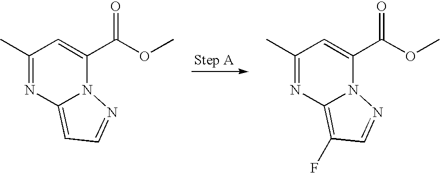

### Step A

To a solution of methyl 5-methylpyrazolo[1,5-a]pyrimidine-7-carboxylate (500 mg) in CH₃CN (10 mL) were added AcOH (2 mL) and 1-chloromethyl-4-fluoro-1,4-diazoniabicyclo[2.2.2]octane bis(tetrafluoroborate) [selectfluor®] (551 mg). The resulting mixture was stirred at 70° C. for 7 h, cooled to room temperature, concentrated and purified by chromatography (silica, cyclohexane/EtOAc) to afford the title compound (149 mg, 27%). [MH]⁺=210.

### Preparative Example 122

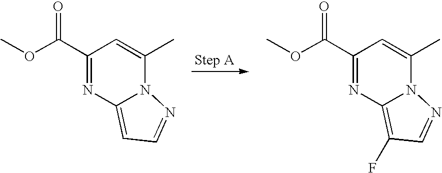

### Step A

To a suspension of methyl 7-methylpyrazolo[1,5-a]pyrimidine-5-carboxylate (10.0 g) in H₂O (1.0 L) was added 1-chloromethyl-4-fluoro-1,4-diazoniabicyclo[2.2.2]octane bis(tetrafluoroborate) [selectfluor®] (18.6 g). The resulting mixture was stirred at 50° C. for 18 h, cooled to room temperature and extracted with CH₂Cl₂ (3×350 mL). The combined organic phases were dried (MgSO₄), filtered, concentrated and purified by chromatography (silica, CH₂Cl₂/acetone) to afford the title compound (4.25 g, 39%). [MH]⁺=210.

### Preparative Example 123

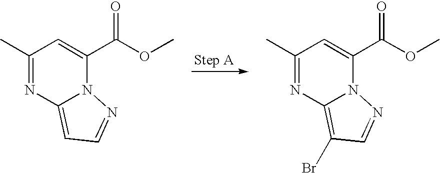

### Step A

To a suspension of methyl 5-methylpyrazolo[1,5-a]pyrimidine-7-carboxylate (500 mg) in CHCl₃ (10 mL) was added N-bromosuccinimide (465 mg). The resulting mixture was heated to reflux for 1 h, cooled to room temperature, concentrated and purified by chromatography (silica, cyclohexane/EtOAc) to afford the title compound (599 mg, 85%). [MH]⁺=270/272.

### Preparative Example 124

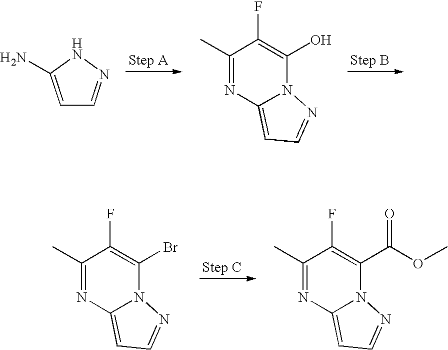

### Step A

A mixture of commercially available 2H-pyrazol-3-ylamine (2.0 g) and 2-fluoro-3-oxo-butyric acid methyl ester (4.4 g) in MeOH (15 mL) was heated at 80° C. for 16 h and then cooled to room temperature. The formed precipitate was isolated by filtration and dried to afford the title compound (4.2 g, 84%). [MH]⁺=168.

### Step B

To a mixture of the title compound from Step A above (1.67 g) in CH₃CN (150 mL) were added K₂CO₃ (4.15 g) and POBr₃ (8.58 g). The mixture was heated to reflux for 16 h, concentrated, diluted with CHCl₃, washed with saturated aqueous NaHCO₃, dried (MgSO₄), filtered, concentrated and purified by chromatography (silica, CH₂Cl₂/MeOH) to afford the title compound as a colorless solid (690 mg, 30%). [MH]⁺=230/232.

### Step C

A mixture of the title compound from step B above (600 mg), Pd(OAc)₂ (40 mg), diphenylphosphinoferrocene (200 mg) and triethylamine (851 μL) in a 1:1 mixture of DMF and MeOH (60 mL) was stirred under an atmosphere of carbon monoxide at 7 bar and 80° C. for 24 h. The mixture was concentrated and purified by chromatography (silica, Cyclohexane/EtOAc 6:4) to afford the title compound (395 mg, 70%). [MH]⁺=210.

### Preparative Example 125

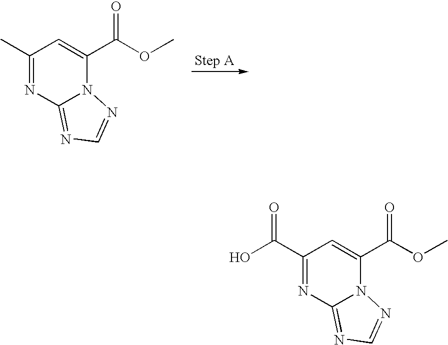

### Step A

A mixture of methyl 5-methyl-[1,2,4]triazolo[1,5-a]pyrimidine-7-carboxylate (1.34 g) and selenium dioxide (1.78 g) in 1,4-dioxane (20 mL) was heated to 120° C. under closed atmosphere for 12 h, cooled and filtered through celite®. To the filtrate were added oxone (1.70 g) and H₂O (400 μL) and the resulting suspension was stirred at room temperature overnight. Concentration and purification by chromatography (silica, CH₂Cl₂/MeOH) afforded the title compound (1 g, 64%). [MH]⁺=223.

### Preparative Examples 126-134

Following a similar procedure as described in the Preparative Example 125, except using the intermediates indicated in Table VII below, the following compounds were prepared:

TABLE VII

| Prep. | | | |
|---|---|---|---|
| Ex. # | intermediate | product | Yield |
| 126 | 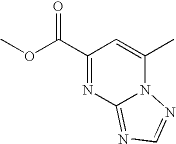 | 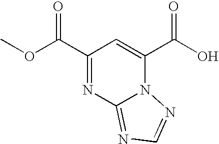 | 69%[MH]⁺ = 223 |
| 127 | 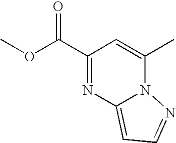 | 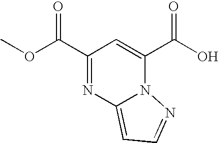 | 34%[MH]⁺ = 222 |
| 128 | 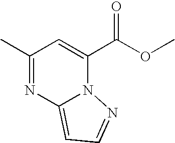 | 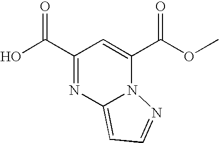 | 24%[MH]⁺ = 222 |
| 129 | 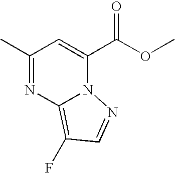 | 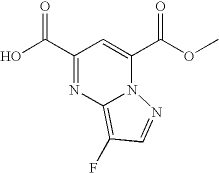 | 60%[MH]⁺ = 240 |
| 130 | 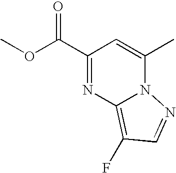 | 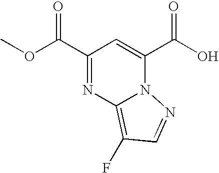 | 71%[MH]⁺ = 240 |
| 131 | 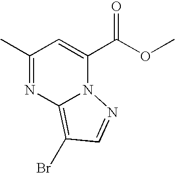 | 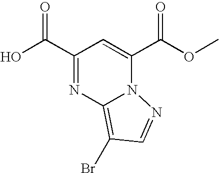 | n.d.[MH]⁺ = 300/302 |
| 132 | 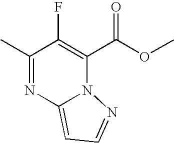 | 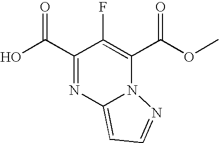 | 80%[MH]⁺ = 240 |
| 133 | 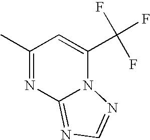 | 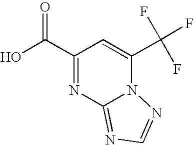 | 20%[MH]⁺ = 232 |
| 134 | 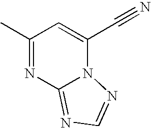 | 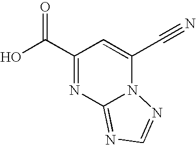 | 23%[MH]⁺ = 190 |

### Preparative Example 135

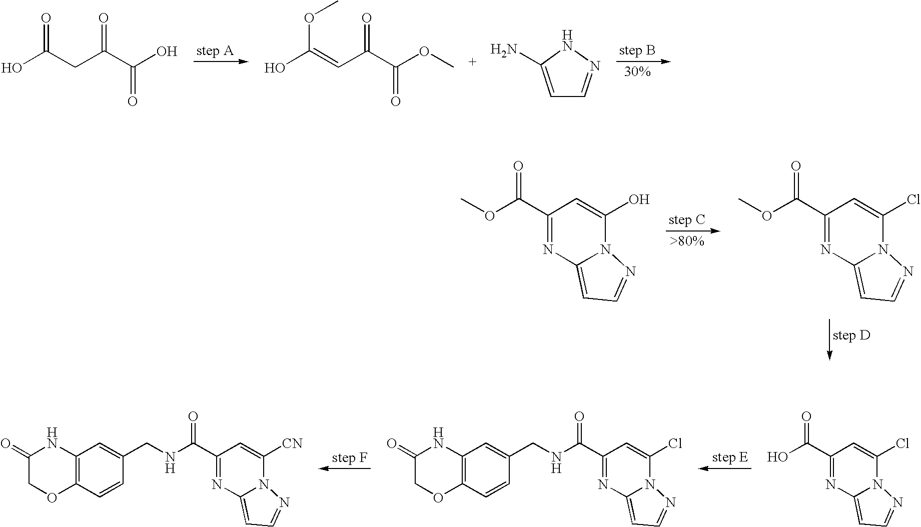

### Step A

Commercially available 2-oxo-succinic acid (15 g) was dissolved in MeOH (150 mL) and cooled to 0° C. Thionylchloride (14 mL) was carefully added and the mixture was heated to reflux for 2 h and concentrated. The residue was dried to give the title compound as a solid (16.9 g, 93%). [MH]⁺=161.

### Step B

The title compound from step A (17.5 g) above was dissolved in EtOH (100 mL) and 3-aminopyrazole (8.26 g) was added. The mixture was heated to 60° C. for 3 h and the precipitate formed was separated. The solid was dried to give the title compound (5.6 g, 26%). [MH]⁺=194.

### Step C

A mixture of the title compound from step B above (100 mg) and dimethylaniline (66 μl) in POCl₃ (4 mL) was heated to 115° C. for 3 h, cooled to room temperature and concentrated. The residue was diluted with CH₂Cl₂ and extracted with 10% aqueous citric acid water sat. NaHCO₃ and brine. The organic phase was dried (MgSO₄), filtered and concentrated to give the title compound as a solid (92 mg, 84%). [MH]⁺=212.

### Step D

A mixture of the title compound from step C above (600 mg) and trimethyltin hydroxide (1.0 g) in dichloroethane was irradiated in a microwave at 140° C. for 1 h. The mixture was filtered, concentrated and aqueous KHSO₄ was added. The precipitate formed was filtered off to afford the title compound (351 mg, 63%). [MH]⁺=198.

### Step E

A mixture of the title compound from step D above (50 mg) and DMF (5 μl) and thionylchloride (0.3 ml) was heated at 70° C. for 45 min. The mixture was concentrated to dryness and the residue dissolved in DMF (3 ml). 6-(Aminomethyl)-2H-benzo[b][1,4]oxazin-3(4H)-one hydrochloride (65 mg) and Et₃N (150 μl) were added and the resulting mixture was stirred at room temperature for 3 d. The mixture was concentrated and aqueous citric acid (10 mL) was added. The formed precipitate was separated by filtration and washed with water to afford the title compound (76 mg, 85%). [MH]⁺=358.

### Step F

The title compound from step E above (244 mg) was dissolved in DMF (5 mL) and HOAt (34 mg) and KCN (88 mg) were added. The mixture was heated to 80° C. for 3 h and concentrated. The residue was treated with 10% aqueous citric acid (10 mL) and the precipitate formed was separated by filtration. The solid was dried to give the title compound (298 mg, 99%). [MH]⁺=349.

### Preparative Example 136

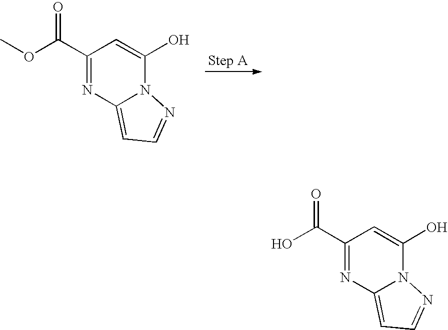

### Step A

A mixture of methyl 7-hydroxypyrazolo[1,5-a]pyrimidine-5-carboxylate (150 mg), 1M NaOH in water (770 μL) and 5 ml MeOH was stirred at room temperature. After 4 h another portion of 1M NaOH in water (1.0 mL) was added and the mixture was stirred at room temperature overnight. The mixture was acidified using 1M HCl, concentrated, suspended in water and the precipitate was separated by filtration to give the title compound (96 mg, 70%). [MH⁺]=180.

### Preparative Example 137

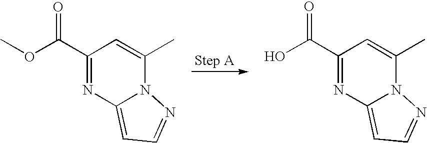

### Step A

Using a similar procedure as that described in Preparative Example 136 except using aqueous LiOH as the base, the title compound was prepared (yield 98%). [MH]⁺=178.

### Example 138

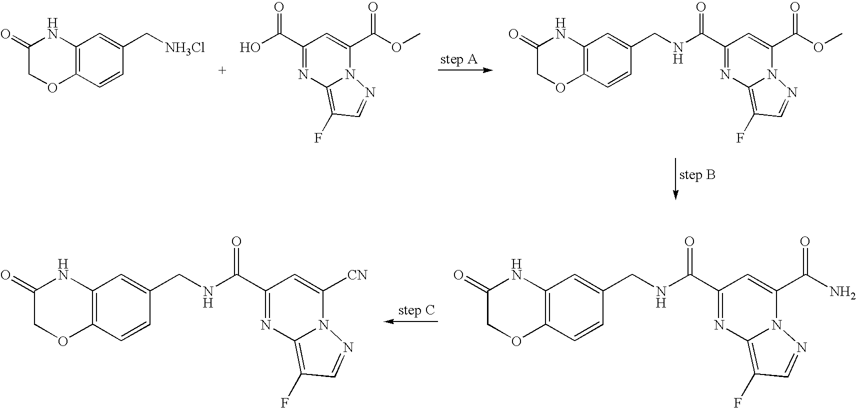

### Step A

A mixture of 6-(aminomethyl)-2H-benzo[b][1,4]oxazin-3(4H)-one hydrochloride (180 mg), 7-(methoxycarbonyl)-3-fluoropyrazolo[1,5-a]pyrimidine-5-carboxylic acid (133 mg), EDCI (330 mg), HOAt (200 mg) and NMM (400 μl) in DMF (10 ml) was stirred at room temperature overnight. The mixture was concentrated and the residue was washed with aqueous citric acid, EtOAc, saturated NaHCO₃, water and purified by column chromatography (silica) to afford the title compound (150 mg, 67%). [MH]⁺=400.

### Step B

The title compound from step A above (150 mg) was dissolved in THF (5 mL) and 7 M NH₃ in MeOH (5 mL) was added. The mixture was stirred for 15 h at room temperature and concentrated. The solid was dried to afford the title compound (155 mg, 99%). [MH]⁺=385.

### Step C

Using similar procedures as that described in Preparative Example 115 the title compound was obtained (15.2 mg, 10%). [MH]⁺=367.

### Example 139

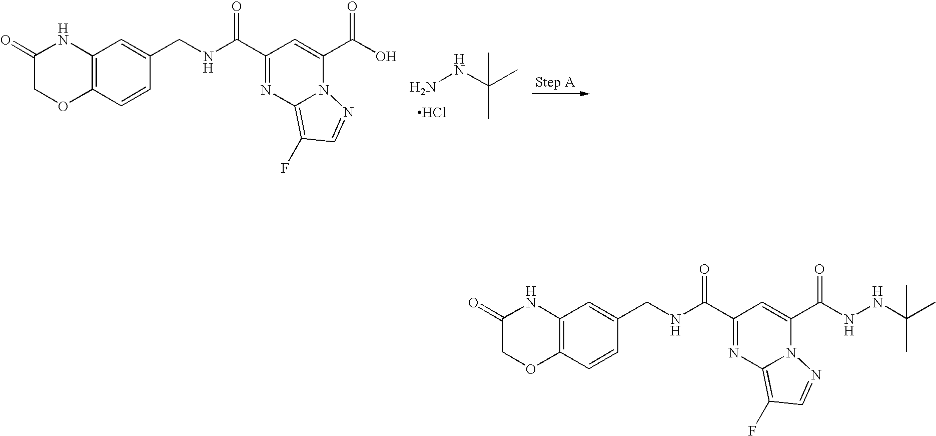

### Step A

Following similar procedures as described in the Example 138 step A except for using HATU instead of EDCI as the coupling reagent the title compound was obtained (80 mg, 97%). [MH]⁺=456.

### Example 140

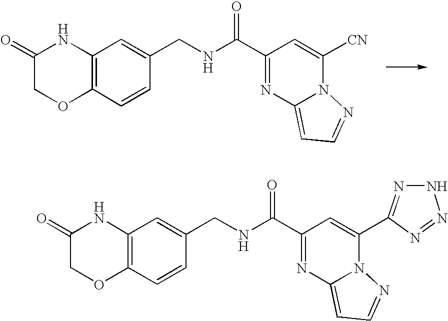

A mixture of 7-cyano-N-((3,4-dihydro-3-oxo-2H-benzo[b][1,4]oxazin-6-yl)methyl)pyrazolo[1,5-a]pyrimidine-5-carboxamide (60 mg), trimethylsilyl azide (370 μl) and dibutyltin oxide (5 mg) in toluene (10 mL) was heated at 110° C. for 24 h. The mixture was concentrated and the residue was purified by column chromatography (silica, DCM to DCM/Aceton 9:1) to give the title compound (38 mg, 57%). [MH]⁺=392.

### Example 141

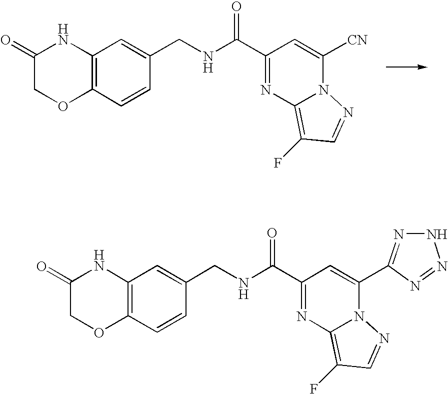

Following a similar procedure as that described in Example 140 the title compound was obtained (16 mg, 93%, [MH]⁺=410).

### Examples 142-151

A tetrazole as indicated in Table VIII below, was dissolved in DMF. K₂CO₃ and an alkyl halide were added. The mixture was stirred at room temperature up to 140° C. depending on reactivity of corresponding alkyl halide until the starting material was consumed. The mixture was cooled to room temperature, formic acid was added and concentrated. The residue was concentrated and the mixture of mono- and bis-alkylated products was separated by preparative thin layer chromatography (CH₂Cl₂/MeOH) to afford the title compounds. According to this procedure the following compounds were prepared.

TABLE VIII

| Ex. # | tetrazole, alkyl halide | products | yield |
|---|---|---|---|
| 142,143 | 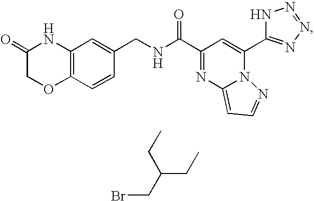 | 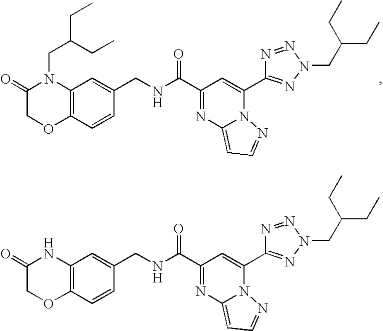 | 2%[MH]⁺ =560,35%[MH]⁺ =476 |
| 144,145 | 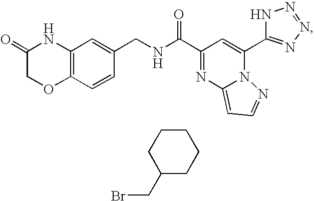 | 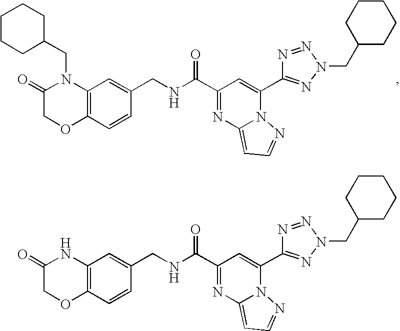 | 28%[MH]⁺ =584,33%[MH]⁺ =488 |
| 146,147 | 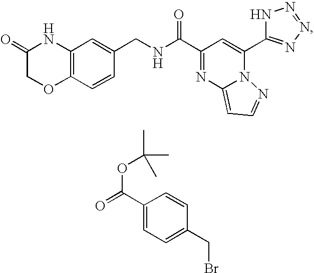 | 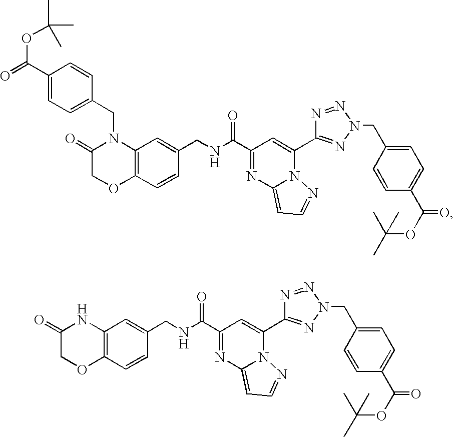 | 6%[MH]⁺ =772,34%[MH]⁺ =582 |
| 148,149 | 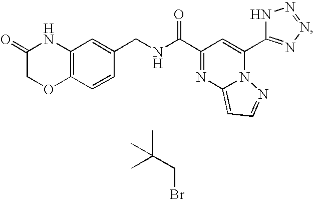 | 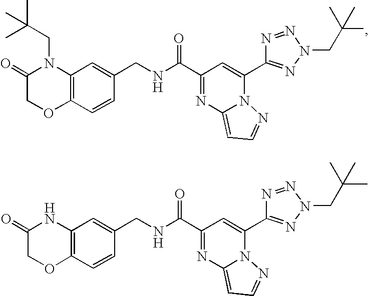 | 19%[MH]⁺ =532,17%[MH]⁺ =462 |
| 150,151 | 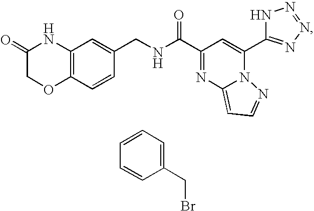 | 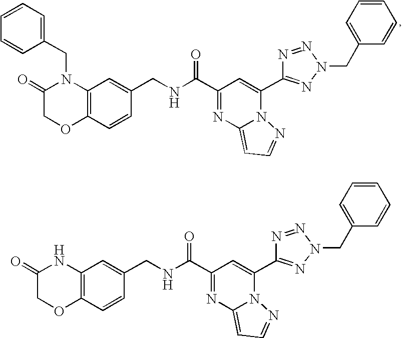 | 21%[MH]⁺ =572,20%[MH]⁺ =482 |

### Example 152

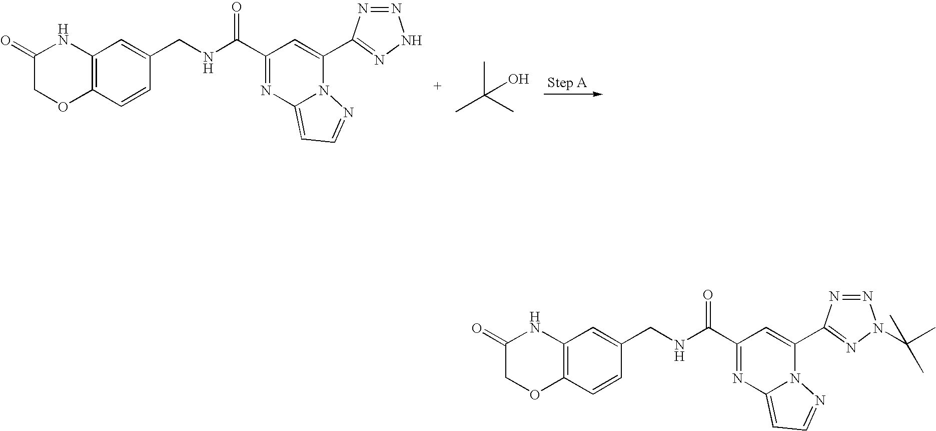

### Step A

A mixture of N-((3,4-dihydro-3-oxo-2H-benzo[b][1,4]oxazin-6-yl)methyl)-7-(2H-tetrazol-5-yl)pyrazolo[1,5-a]pyrimidine-5-carboxamide (20 mg), 2-methylpropan-2-ol (10 mg) and conc. H₂SO₄ (1 μl) in TFA (1 mL) was stirred at room temperature for 2 h. Sat. NaHCO₃ (5 mL) was added and the mixture was concentrated. The residue was purified by preparative thin layer chromatography (CH₂Cl₂/MeOH 9:1) to afford the title compound (13.4 mg, 59%). [MH]⁺=448.

### Example 153-157

Following similar procedures as described in Example 139 using EDCI or HATU as the coupling reagent, as indicated in Table IX below, following compounds were prepared:

TABLE IX

| | | | method, |
|---|---|---|---|
| Ex. # | acid, amine | product | yield |
| 153 | 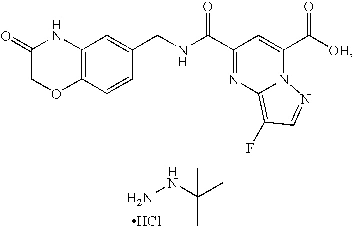 | 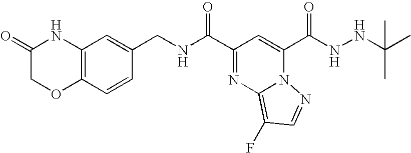 | EDCI, 97%[MH]⁺ = 456 |
| 154 | 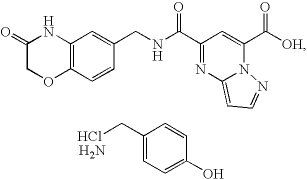 | 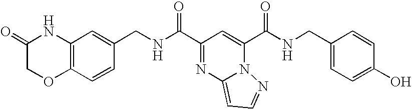 | EDCI, 31%[MH]⁺ = 473 |
| 155 | 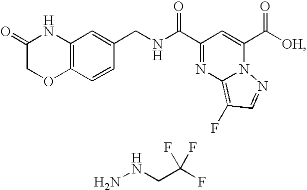 | 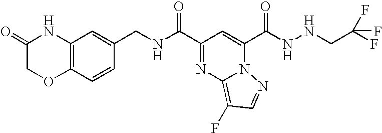 | HATU, 53%[MH]⁺ = 482 |
| 156 | 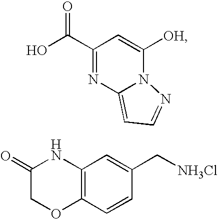 | 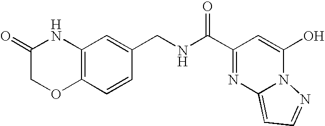 | EDCI, 43%[MH]⁺ = 340 |
| 157 | 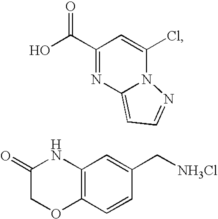 | 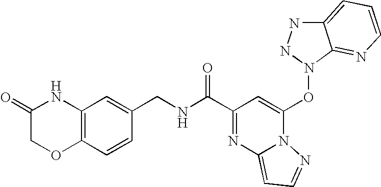 | EDCI, 31%[MH]⁺ = 358 |

### Example 158

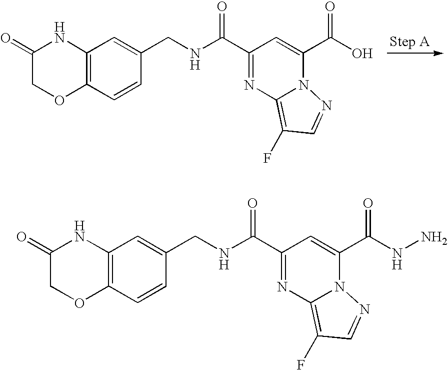

### Step A

A mixture of 5-((3,4-dihydro-3-oxo-2H-benzo[b][1,4]oxazin-6-yl)methylcarbamoyl)-3-fluoropyrazolo[1,5-a]pyrimidine-7-carboxylic acid (50 mg) and thionyl chloride (150 μl) in MeOH (5 mL) was heated at reflux for 2 h. The mixture was concentrated, dissovled in EtOH (10 mL) and hydrazine hydrate (100 μL) was added. The mixture was heated at reflux for 2 h, cooled to room temperature and the precipitate formed was separated by filtration to afford the title compound (yield n.d.). [MH]+=400.

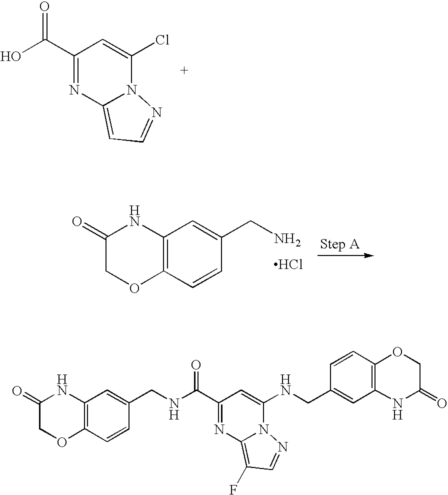

### Step A

Using a similar procedure as that described in Example 158 the title compound was obtained (146 mg, 14%). [MH]⁺=500.

### Example 160

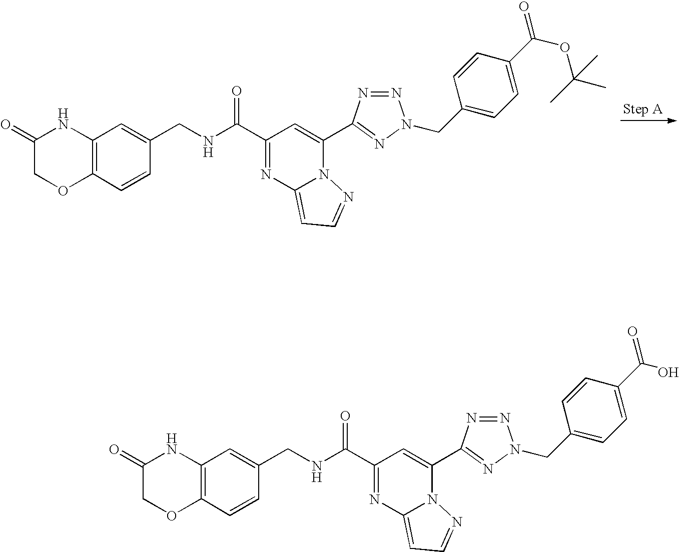

### Step A

A mixture of tert-butyl 4-((5-(5-((3,4-dihydro-3-oxo-2H-benzo[b][1,4]oxazin-6-yl)methyl-carbamoyl)-pyrazolo[1,5-a]pyrimidin-7-yl)-2H-tetrazol-2-yl)methyl)benzoate (14.5 mg) in formic acid (20 mL) was stirred for 2 h at room temperature. The mixture was concentrated to afford the title compound (12.9 mg 94%). [MH]⁺=526.

### Example 161

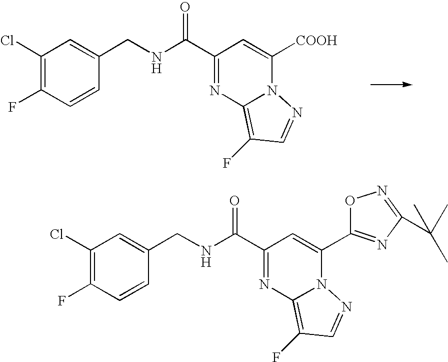

A mixture of 5-(3-chloro-4-fluorobenzylcarbamoyl)-3-fluoropyrazolo[1,5-a]pyrimidine-7-carboxylic acid (30 mg), N′-hydroxypivalamidine (14 mg), HATU (60 mg) and DIPEA (50 μL) in DMF was stirred at room temperature overnight. The mixture was irradiated in a microwave at 130° C. for 30 min, concentrated and the residue was dissolved in EtOAc. The organic layer was washed with citric acid and Brine, dried (MgSO₄), concentrated and purified by preparative TLC to afford the title compound (15.3 mg, 43%). [MH]⁺=447.

### Examples 162-164

Following similar procedures as described in the Example 161 above except using the acids and amidoximes indicated in Table X below, the following compounds were prepared:

TABLE X

| Ex. # | acid, amidoxime | product | Yield |
|---|---|---|---|
| 162 | 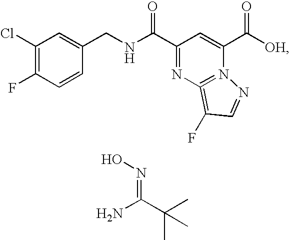 | 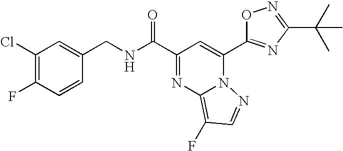 | 43%[MH]⁺ = 447 |
| 163 | 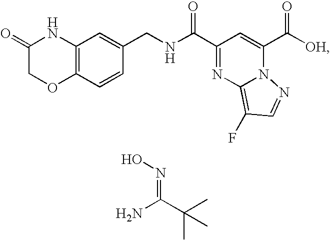 | 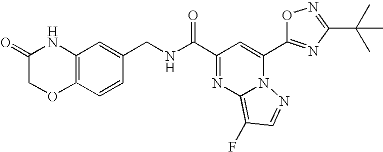 | 37%[MH]⁺ = 466 |
| 164 | 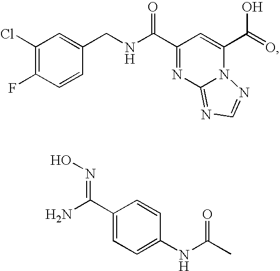 | 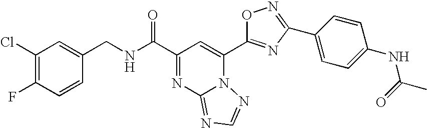 | 3%[MH]⁺ = 507 |

### Examples 165-166

In some of the Examples described in Table X, decarboxylation products were formed which were separated by preparative TLC as indicated in Table XI below.

TABLE XI

| | | | |
|---|---|---|---|
| 165 | 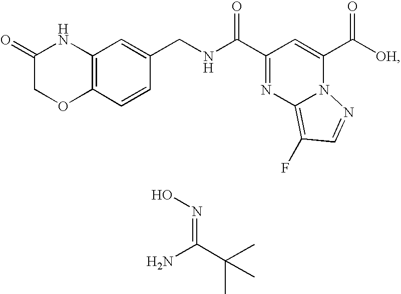 | 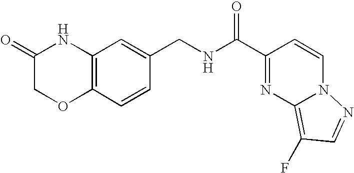 | 55%[MH]⁺ = 342 |
| 166 | 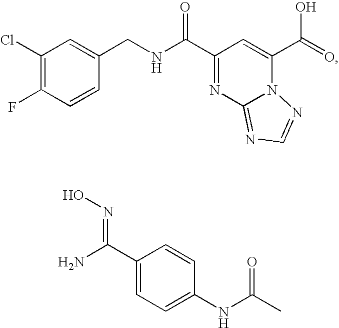 | 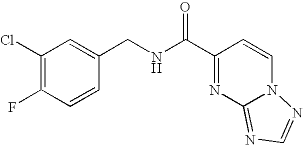 | 48%[MH]⁺ = 306 |

### Example 167

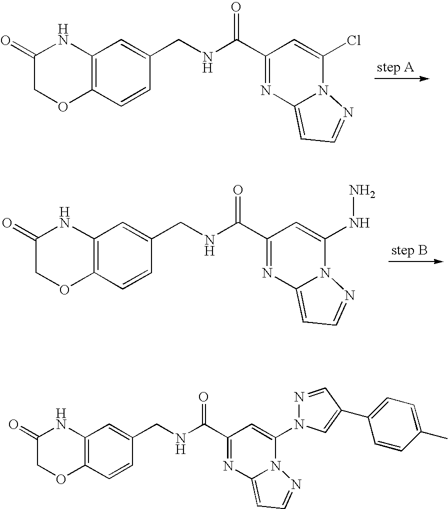

### Step A

7-Chloro-N-((3,4-dihydro-3-oxo-2H-benzo[b][1,4]oxazin-6-yl)methyl)pyrazole [1,5-a]pyridine-5-carboxamide (20 mg) was dissolved in EtOH and 35% hydrazine solution in water (10 μL) was added at 0° C. The mixture was stirred for 2 h and the precipitate formed was separated by filtration. The solid was dried to give the title compound (16 mg, 81%), [MH]⁺=354.

### Step B

The title compound from step A above (16 mg) was dissolved in EtOH and commercially available 3-Hydroxy-2-p-tolyl-propenal (8 mg) was added. The mixture was stirred for 2 h at reflux and the precipitate formed was separated. The solid was dried to give the title compound (9 mg, 42%). [MH]⁺=480.

### Example 168

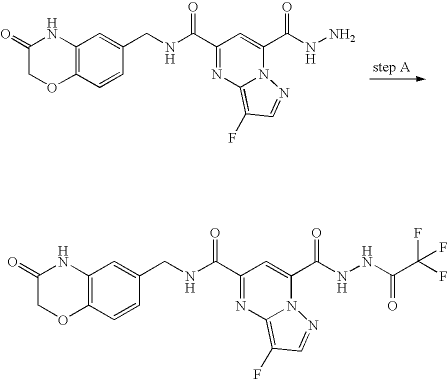

### Step A

3-Fluoro-7-hydrazinyl-N-((3,4-dihydro-3-oxo-2H-benzo[b][1,4]oxazin-6-yl)methyl)pyrazolo[1,5-a]pyrimidine-5-carboxamide (35 mg) was dissolved in CHCl₃ (2 mL) and TFAA (1 mL) was added. The mixture was stirred for 2 h at 50° C. and concentrated. The residue was dissolved in CHCl₃ and evaporated. This procedure was repeated twice. The solid was dried to give the title compound (47 mg, 99%). [MH]⁺=496.

A hydrazide as indicated in Table XII below, was dissolved in pyridine and an excess of triphosgene in CHCl₃ was added. The mixture was heated at 80° C. for 24 h and concentrated. The residue was treated with 10% citric acid and filtrated. The precipitate was purified by preparative thin layer chromatography (CH₂Cl₂/MeOH) to give the title compound. According to this procedure the following compounds were prepared.

TABLE XII

| Ex. # | Hydrazide | product | yield |
|---|---|---|---|
| 169 | 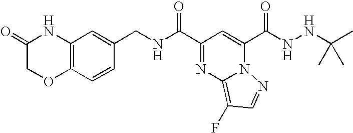 | 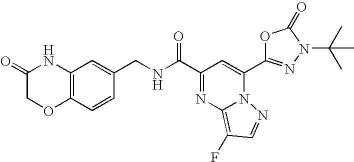 | 10%[MH]⁺ = 482 |
| 170 | 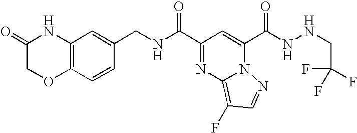 | 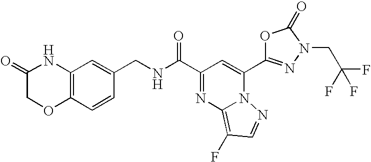 | 44%[MH]⁺ = 508 |

### Example 171

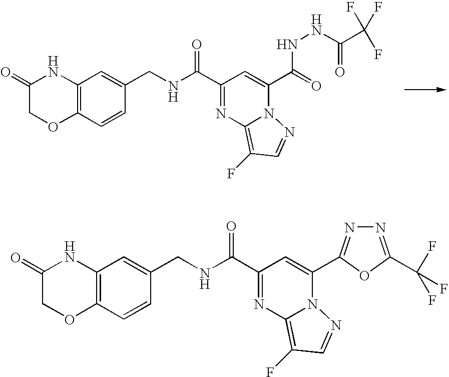

A mixture of 5-((3,4-dihydro-3-oxo-2H-benzo[b][1,4]oxazin-6-yl)methylcarbamoyl)-3-fluoro-N′-(2,2,2-trifluoroacetyl)pyrazolo[1,5-a]pyrimidine-7-carbohydrazide (45 mg), Burgess Reagent (44 mg) in THF was irradiated in a microwave for 30 min at 150° C. The mixture was concentrated and the residue purified by preparative TLC to afford the title compound (7.4 mg, 17%). [MH]⁺=478.

### Preparative Example 172

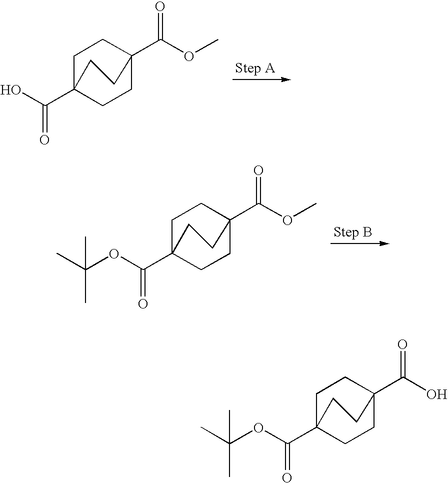

### Step A

To a refluxing mixture of 4-(methoxycarbonyl)bicyclo[2.2.2]octane-1-carboxylic acid (311 mg) in toluene di-tert-butoxy-N,N-dimethylmethanamine (1.19 g) was added over a period of 2 h. The mixture was concentrated and diluted with EtOAc. The organic layer was washed with 1M NaOH, water and Brine, dried (MgSO₄) and concentrated to afford the title compound (220 mg, 76%). [MNa]⁺=291.

### Step B

To a mixture of the title compound from step A above (50 mg) in dioxane (5 mL) a solution of NaOH (15 mg) in water (2.5 mL) was added. The mixture was concentrated to afford the title compound which was used without further purification. (Yield n.d.). [MNa]⁺=277.

### Preparative Example 173

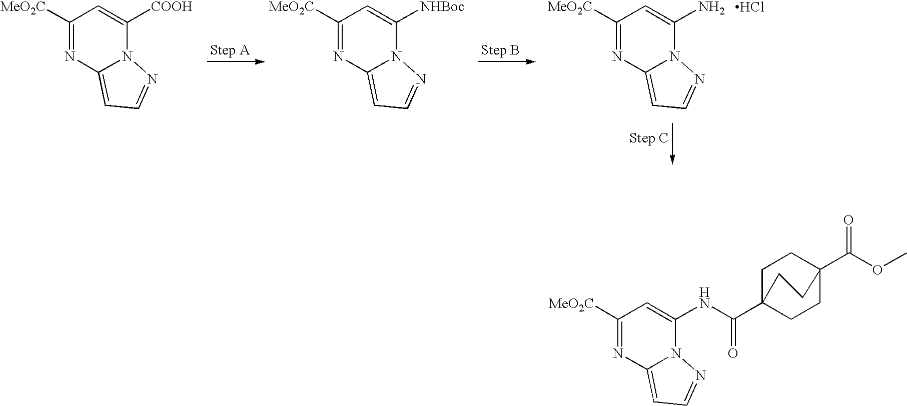

### Step A

A mixture of 5-(methoxycarbonyl)pyrazolo[1,5-a]pyrimidine-7-carboxylic acid (2.0 g) diphenylphosphoryl azide (2.36 mL), molecular sieve 4 Å (20 g) and triethylamine (1.5 mL) in t-BuOH (40 mL) was heated at reflux for 24 h. The mixture was concentrated and purified by column chromatography (silica, Cyclohexane/EtOAc 6:4) to afford the title compound (1.45 g, 55%). [MH]⁺=293.

### Step B

A mixture of 500 mg of the title compound from step A above in 4M HCl in dioxane was stirred at room temperature for 24 h. The mixture was concentrated to afford the title compound (quantitative yield). [MH]⁺=193.

### Step C

A mixture of the title compound from step B above (288 mg), 4-(methoxycarbonyl)bicyclo-[2.2.2]octane-1-carboxylic acid (212 mg), HATU (570 mg) and DIPEA (359 μL) in DMF (5 mL) was stirred at room temperature for 3 d. The mixture was concentrated and purified by column chromatography (silica, Cyclohexane/EtOAc 6:4) to afford the title compound (52 mg, 14%). [MH]⁺=387.

### Preparative Example 174

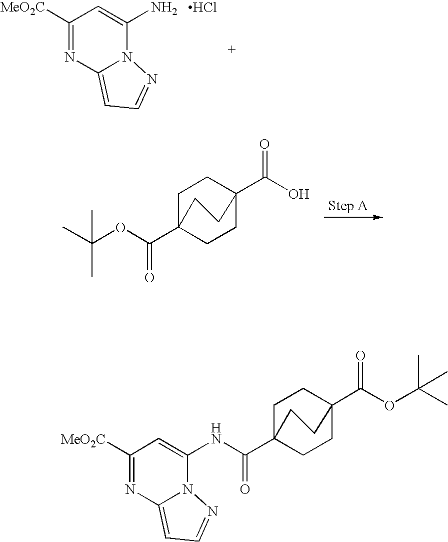

Using similar procedures as that described in Preparative Example 173 step C the title compound was obtained (yield n.d.). [MH]⁺=429.

### Preparative Example 175

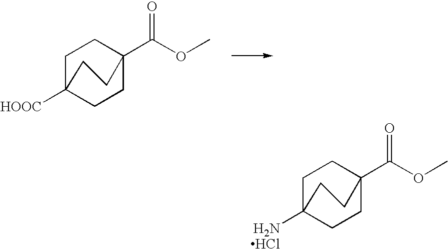

Using similar procedures as that described in Preparative Example 173 steps A and B the title compound was obtained. [MH]⁺=184.

### Preparative Example 176

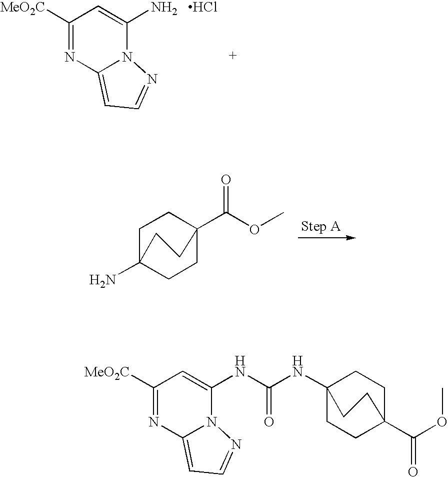

### Step A

To a solution of triphosgene (48 mg) in dichloromethane (2 mL) a mixture of methyl 7-aminopyrazolo[1,5-a]pyrimidine-5-carboxylate hydrochloride (100 mg) and DIPEA (174 μL) in dichloromethane (2 mL) was added over a period of 30 min. After 5 min at room temperature a mixture of methyl 4-aminobicyclo[2.2.2]octane-1-carboxylate hydrochloride (96 mg) and DIPEA (174 μL) in dichloromethane (2 mL) was added and the mixture was stirred at room temperature for 10 min. The mixture was concentrated, diluted with EtOAc, washed with 10% KHSO₄, 5% NaHCO₃, Brine, dried (MgSO₄) and concentrated. Preparative TLC afforded the title compound (yield n.d., [MNa]⁺=424)

### Preparative Example 177

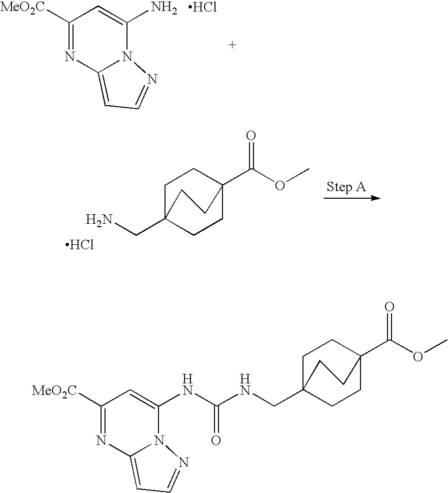

Using similar procedures as that described in Preparative Example 176 the title compound was obtained (30 mg, 56%). [MH]⁺=416.

### Example 178

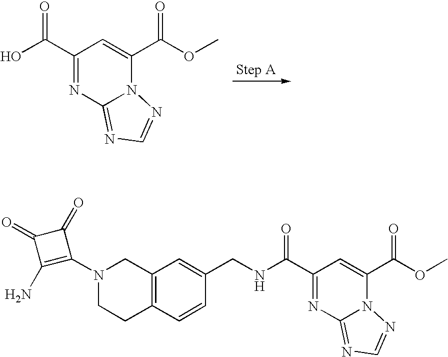

### Step A

To an ice cooled solution of 7-(methoxycarbonyl)-[1,2,4]triazolo[1,5-a]pyrimidine-5-carboxylic acid (250 mg) and 3-amino-4-(7-(aminomethyl)-3,4-dihydroisoquinolin-2(1H)-yl)cyclobut-3-ene-1,2-dione hydrochloride (329 mg) in DMF (10 mL) were added N-methylmorpholine (170 μL), HATU (570 mg) and HOAt (204 mg). The mixture was stirred overnight while warning to room temperature and then concentrated. The remaining residue was dissolved in CHCl₃, washed with saturated aqueous NaHCO₃, 1N aqueous HCl and saturated aqueous NaCl, dried (MgSO₄), filtered, absorbed on silica and purified by chromatography (silica, CH₂Cl₂/MeOH) to afford the title compound as a yellow/brown gummy solid (177 mg, 35%). [MH]⁺=462.

### Examples 179-190

Following similar procedures as described in Example 178 using either EDCI or another coupling reagent as indicated the following compounds were prepared:

TABLE XIII

| Ex. # | acid, amine | product | method, yield |
|---|---|---|---|
| 179 | 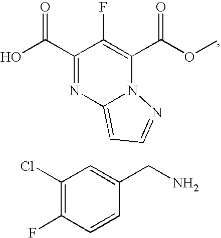 | 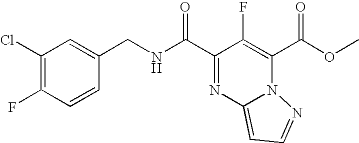 | HATU, n.d.[MH]⁺ = 381 |
| 180 | 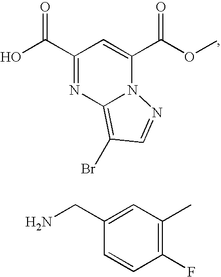 | 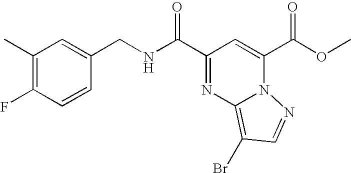 | PyBOP, n.d.[MH]⁺ = 421/423 |
| 181 | 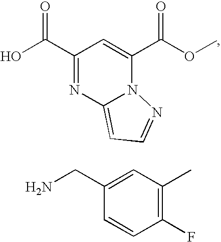 | 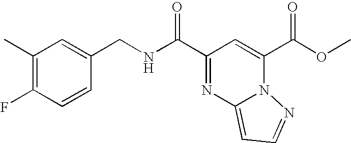 | PyBrop, 11%[MH]⁺ = 343 |
| 182 | 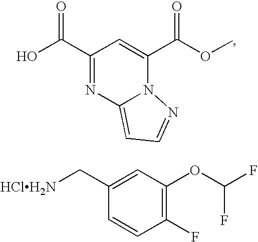 | 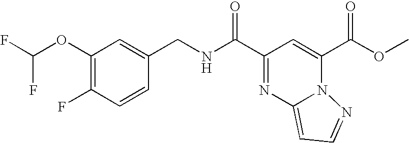 | HATU, 37%[MH]⁺ = 395 |
| 183 | 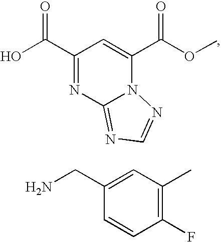 | 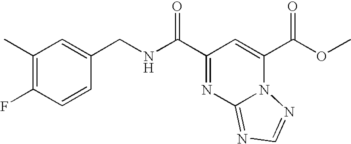 | HATU, >99%[MH]⁺ = 397 |
| 184 | 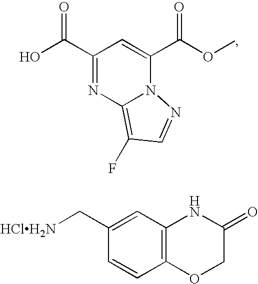 | 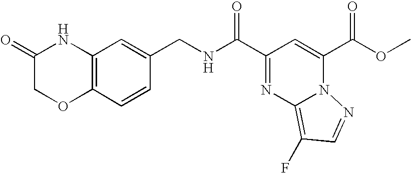 | EDCI, 82%[MH]⁺ = 400 |
| 185 | 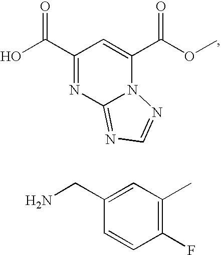 | 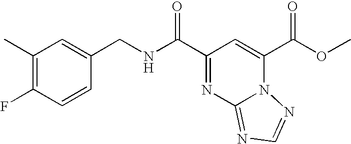 | HATU, 70%[MH]⁺ = 344 |
| 186 | 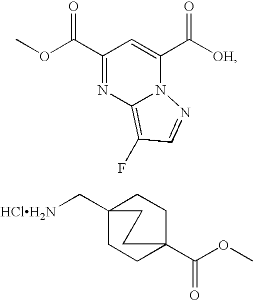 | 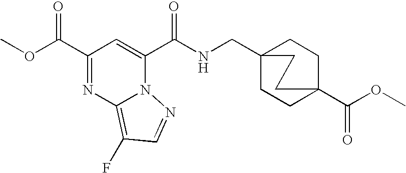 | HATU, 47%[MH]⁺ = 419 |
| 187 | 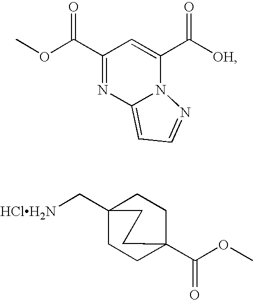 | 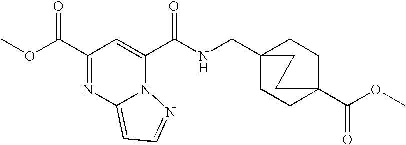 | HATU, 29%[MH]⁺ = 401 |
| 188 | 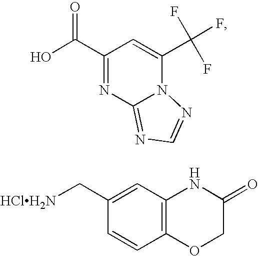 | 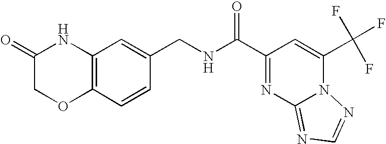 | EDCI, 81%[MH]⁺ = 393 |
| 189 | 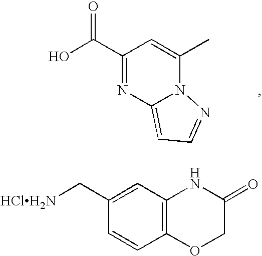 | 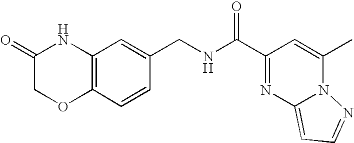 | EDCI, 85%[MH]⁺ = 352 |
| 190 | 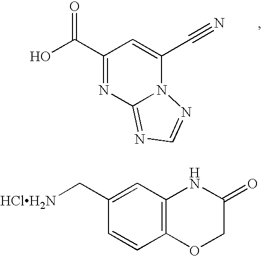 | 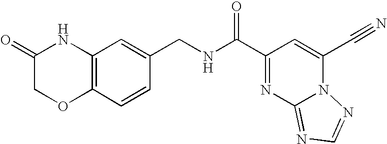 | EDCI, 18%[MH]⁺ = 350 |

### Example 191

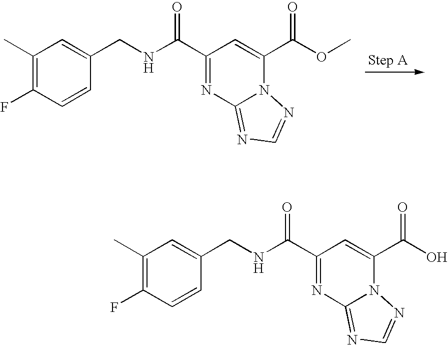

### Step A

To a solution of NaOH (24 mg) in dry MeOH (3.2 mL) was added methyl 5-(4-fluoro-3-methylbenzylcarbamoyl)-[1,2,4]triazolo[1,5-a]pyrimidine-7-carboxylate (170 mg). The resulting suspension was stirred at room temperature for 1 h, acidified with 1N aqueous HCl and concentrated. The remaining residue was dissolved in EtOAc, washed with 1N aqueous HCl, dried (MgSO₄), filtered and concentrated to afford the title compound (130 mg, 80%). [MH]⁺=330.

### Examples 192-203

Following similar procedures as described in the Example 191 using either NaOH in Methanol (method A), LiOH in aqueous dioxane (method B) or NaOH in aqueous dioxane (Method C) the following compounds were prepared:

TABLE XIV

| | | | method, |
|---|---|---|---|
| Ex. # | ester | product | yield |
| 192 | 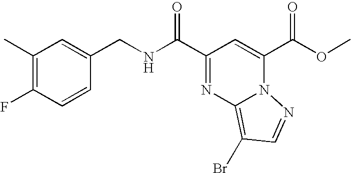 | 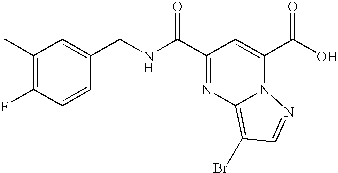 | A, n.d.[MH]⁺ =407/409 |
| 193 | 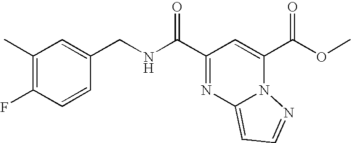 | 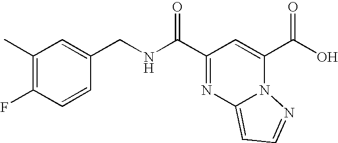 | A, 98%[MH]⁺ = 329 |
| 194 | 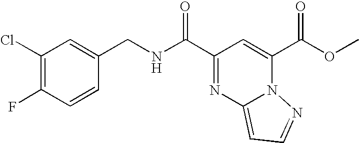 | 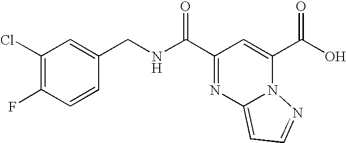 | B, 97%[MH]⁺ = 349 |
| 195 | 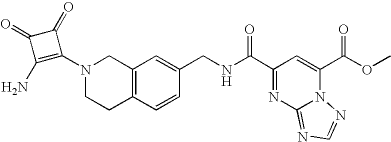 | 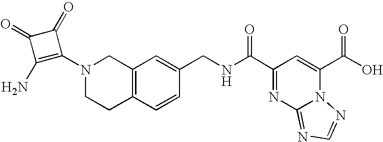 | A, 67%[MH]⁺ = 448 |
| 196 | 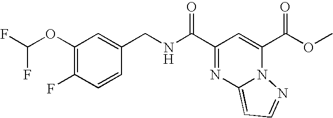 | 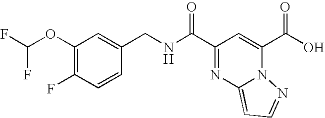 | A, 91%[MH]⁺ = 381 |
| 197 | 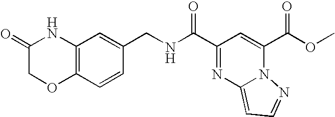 | 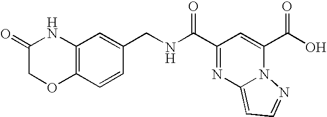 | B, 96%[MH]⁺ = 368 |
| 198 | 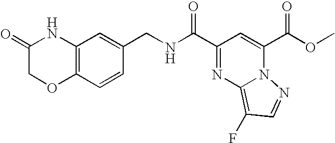 | 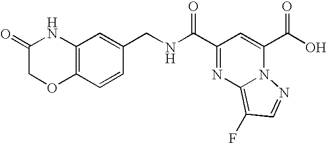 | B, 82%[MH]⁺ = 386 |
| 199 | 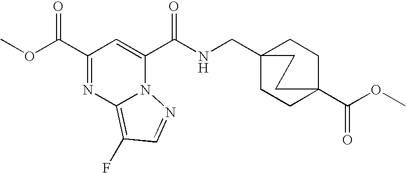 | 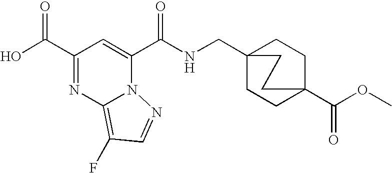 | A, 95%[MH]⁺ = 405 |
| 200 | 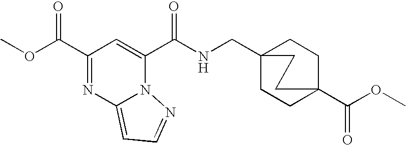 | 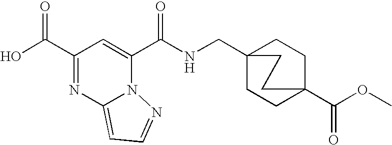 | A, 95%[MH]⁺ = 387 |
| 201 | 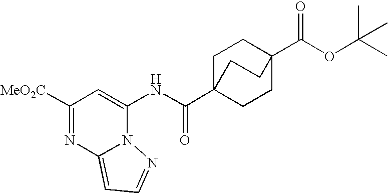 | 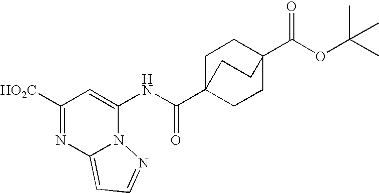 | C, n.d.[MH]⁺ = 415 |
| 202 | 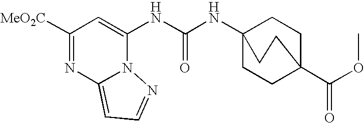 | 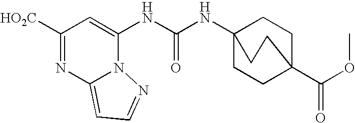 | C, n.d.[MH]⁺ = 388 |
| 203 | 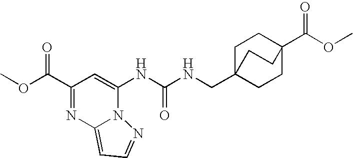 | 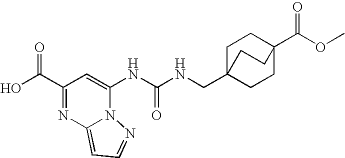 | C, n.d.[MH]⁺ = 402 |

n.d. = not determined

### Examples 204-206

Following similar procedures as described in Examples 179-190 using HATU as coupling reagent the following compounds were prepared:

TABLE XV

| | | | method, |
|---|---|---|---|
| Ex. # | acid, amine | product | yield |
| 204 | 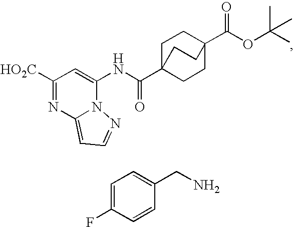 | 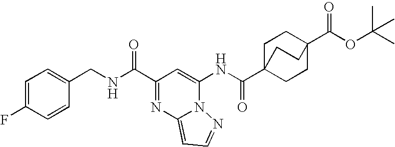 | HATU, 27%[MH]⁺ = 522 |
| 205 | 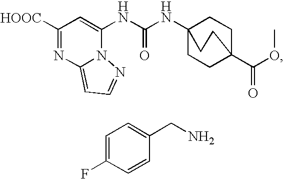 | 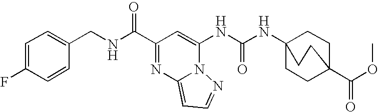 | HATU, 67%[MH]⁺ = 495 |
| 206 | 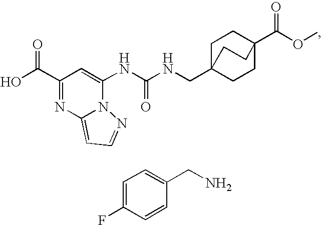 | 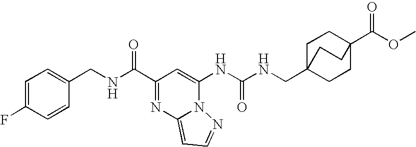 | HATU, 22%[MH]⁺ = 509 |

### Examples 207-208

Following similar procedures as described in the Examples 192-203 using LiOH in water/THF/MeOH 1:3:1 the following compounds were prepared:

TABLE XVI

| Ex. # | acid, amine | | |
|---|---|---|---|
| 207 | 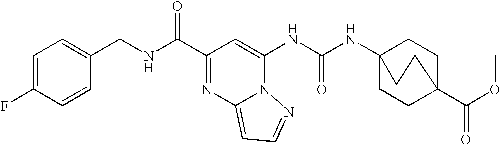 | | |
| 208 | 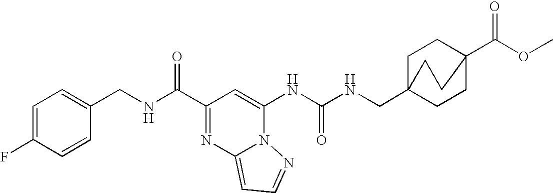 | | |
| | Ex. # | product | Yield |
| | 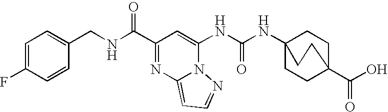 | 73%[MH]⁺ = 4815 | |
| | 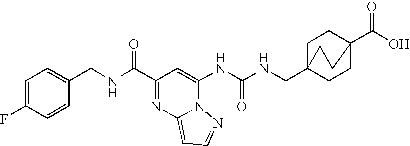 | 63%[MH]⁺ = 595 | |

### Example 209

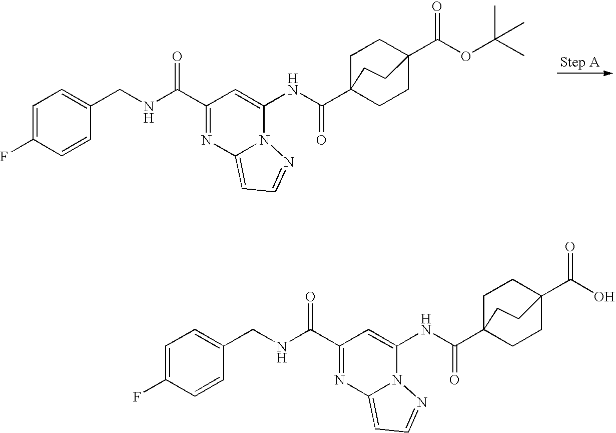

### Step A

To a solution of tert-butyl 4-(5-(4-fluorobenzylcarbamoyl)pyrazolo[1,5-a]pyrimidin-7-ylcarbamoyl)bicyclo[2.2.2]octane-1-carboxylate (10 mg) in dichloromethane (0.25 mL) TFA (0.15 mL) was added and the mixture was stirred at room temperature for 1 h. The mixture was concentrated and purified by preparative TLC (dichloromethane/MeOH 9:1) to afford the title compound (1.5 mg, 17%). [MH]⁺=466.

### Preparative Example 210

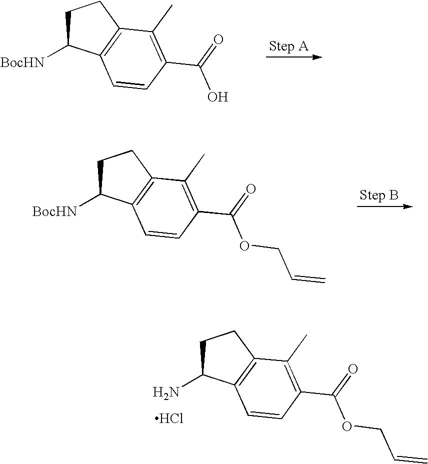

### Step A

To mixture of IS-tert-butoxycarbonylamino-4-methyl-indan-5-carboxylic acid (536 mg) and allyl bromide (1.6 mL) in CHCl₃/THF (1:1, 20 mL) were added Bu₄NHSO₄ (70 mg) and a 1M solution of LiOH in H₂O (10 mL) and the resulting biphasic mixture was stirred at 40° C. overnight. The organic phase was separated, concentrated, diluted with CHCl₃, washed with H₂O, dried (MgSO₄), filtered, concentrated and purified by chromatography (silica, cyclohexane/EtOAc) to afford the title compound (610 mg, >99%). [MNa]⁺=354.

### Step B

A mixture of the title compound from step A above in 4M HCl/dioxane was stirred at room temperature for 17 h. The mixture was concentrated to afford the title compound (202 mg, 97%) [M−NH₃Cl]⁺.

### Example 211

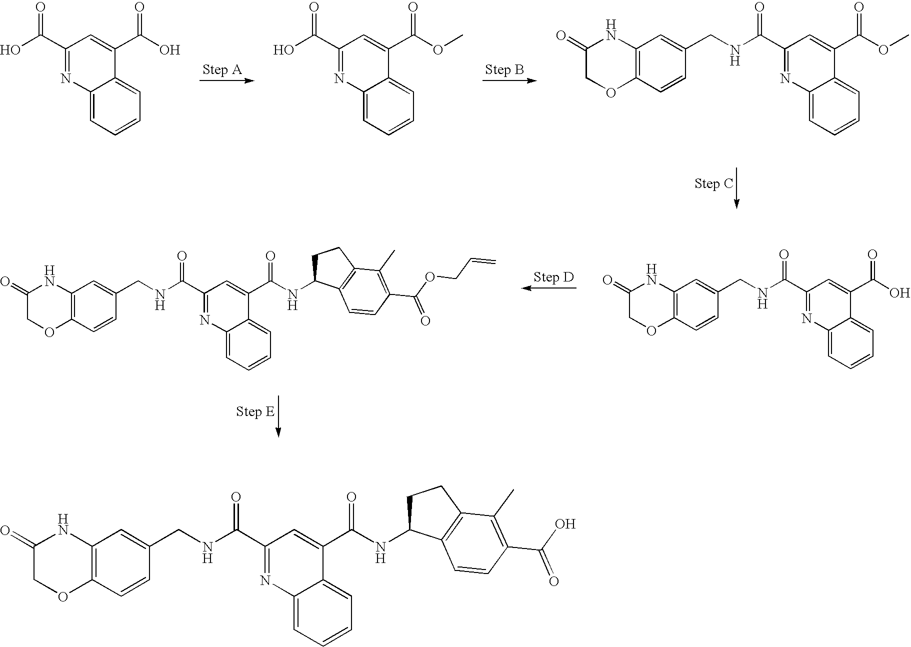

### Step A

Quinoline-2,4-dicarboxylic acid (4.4 g) was treated with dry methanol (150 mL) and conc. H₂SO₄ (10 mL) at room temperature overnight. The solution was concentrated and then ice was added and extracted with dichloromethane. The organic layer was absorbed on silica and purified by flash chromatography (hexane/ethyl acetate 1:1 to remove diester, then dichloromethane/methanol 95:5 to 85:15) to afford the desired title compound (190 mg, 4%) as colourless solid. ¹H-NMR (CDCl3): 8.76 (dd, 1H), 8.66 (s, 1H), 8.28 (dd, 1H), 7.84-7.67 (m, 2H), 4.05 (s, 3H). [MH]⁺=232.

### Step B

To a mixture of the title compound from step A above (80 mg) in DCM (10 mL) oxalyl chloride (160 μL) were added and the mixture was stirred overnight at room temperature. 6-(aminomethyl)-2H-benzo[b][1,4]oxazin-3(4H)-one hydrochloride (100 mg) and pyridine (2 mL) were added and the mixture was stirred at 60° C. overnight. The mixture was concentrated and the residue was washed with 10% citric acid, water, EtOAc and separated by filtration to afford the crude amide. This was suspended in THF and a solution of LiOH (11 mg) in water was added. The mixture was stirred overnight at room temperature, acidified, concentrated and the residue washed with water to afford the title compound (41.4 mg, 32%) [MH]⁺=378.

### Step C

A mixture of the title compound from step B above (37.9 mg), PyBrop (56 mg), NMM (30 μL) and (S)-allyl 1-amino-2,3-dihydro-4-methyl-1H-indene-5-carboxylate hydrochloride (35 mg) in DMF was stirred at overnight at room temperature. The mixture was concentrated and the residue was washed with 10% citric acid, water, EtOAc and separated by filtration to afford the title compound (43 mg, 73%). [MH]⁺=591

### Step D

To a solution of the title compound from step C above (43 mg) in THF (4 mL) were added morpholine (100 μL) and Pd(PPh₃)₄ (10 mg). The mixture was stirred for 4 h, evaporated and dissolved in chloroform, washed with 10% citric acid, dried and evaporated. The residue was triturated with methanol to afford the title compound as an off-white solid. (16 mg, 40%) [MH]⁺=551.

### Example 1700

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of catalytic domain of MMP-13 enzyme (produced by Alantos) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of MMP-13 fluorescent substrate (Calbiochem, Cat. No. 444235). The time-dependent increase in fluorescence is measured at the 320 nm excitation and 390 nm emission by automatic plate multireader. The IC₅₀ values are calculated from the initial reaction rates.

### Example 1701

### Assay for Determining MMP-3 Inhibition

The typical assay for MMP-3 activity is carried out in assay buffer comprised of 50 mM MES, pH 6.0, 10 mM CaCl₂ and 0.05% Brij-35. Different concentrations of tested compounds are prepared in assay buffer in 50 μL aliquots. 10 μL of a 100 nM stock solution of the catalytic domain of MMP-3 enzyme (Biomol, Cat. No. SE-109) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of NFF-3 fluorescent substrate (Calbiochem, Cat. No. 480-455). The time-dependent increase in fluorescence is measured at the 330 nm excitation and 390 nm emission by automatic plate multireader. The IC₅₀ values are calculated from the initial reaction rates

### Example 1702

### Assay for Determining MMP-8 Inhibition

The typical assay for MMP-8 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of activated MMP-8 enzyme (Calbiochem, Cat. No. 444229) is added to the compound solution. The mixture of enzyme and compound in assay buffer is thoroughly mixed and incubated for 10 min at room temperature. Upon the completion of incubation, the assay is started by addition of 40 μL of a 10 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### Example 1703

Assay for Determining MMP-12 Inhibition

The typical assay for MMP-12 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of the catalytic domain of MMP-12 enzyme (Biomol, Cat. No. SE-138) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### Example 1704

### Assay for Determining Aggrecanase-1 Inhibition

The typical assay for aggrecanase-1 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 75 nM stock solution of aggrecanase-1 (Invitek) is added to the compound solution. The mixture of enzyme and compound in assay buffer is thoroughly mixed. The reaction is started by addition of 40 μL of a 250 nM stock solution of aggrecan-IGD substrate (Invitek) and incubation at 37° C. for exact 15 min. The reaction is stopped by addition of EDTA and the samples are analysed by using aggrecanase ELISA (Invitek, InviLISA, Cat. No. 30510111) according to the protocol of the supplier. Shortly: 100 μL of each proteolytic reaction are incubated in a pre-coated micro plate for 90 min at room temperature. After 3 times washing, antibody-peroxidase conjugate is added for 90 min at room temperature. After 5 times washing, the plate is incubated with TMB solution for 3 min at room temperature. The peroxidase reaction is stopped with sulfurous acid and the absorbance is red at 450 nm. The IC₅₀ values are calculated from the absorbance signal corresponding to residual aggrecanase activity.

What is claimed is:

1. A compound having the structure: 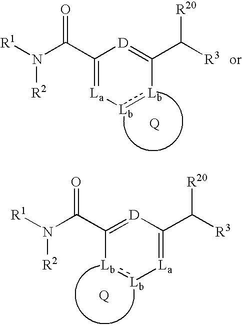
wherein:
R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is NRleR¹ or NR¹⁰R¹¹;
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ-(C₀-C₆)-alkyl-C(O)ORO, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O)ₓ—(C₀-C₆)-alkyl-C(O)NR¹⁰R1, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or,
wherein each R⁹ group is optionally substituted by one or more R¹⁴ groups;
R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 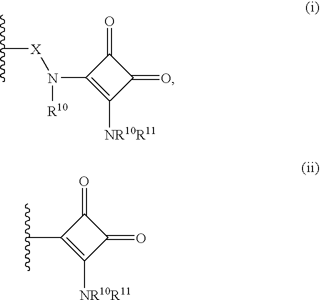
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicyclic or tricyclic ring system is fused and contains at least one ring which is partially saturated and
wherein R²¹ is optionally substituted one or more times, or
wherein R²¹ is optionally substituted by one or more R⁹ groups;
R² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹¹, and fluoroalkyl, wherein alkyl, cycloalkyl, alkoxy, alkenyl, alkynyl and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 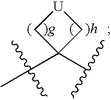
D is a member selected from CR²² and N;
Lₐ is selected from CR⁹ and N;
L_{b} is independently selected from C and N with the provisos that both L_{b} are not N, and that the bond between L_{b} and L_{b} is optionally a double bond only if both are L_{b} are carbon;
Q is a 5- or 6-membered ring selected from aryl and heteroaryl, wherein aryl and heteroaryl are optionally substituted one or more times with R⁴;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR¹⁰R¹¹)_{w};
X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;
g and h are independently selected from 0-2;
w is independently selected from 0-4;
x is selected from 0 to 2;
y is selected from 1 and 2; and
N-oxides, pharmaceutically acceptable salts, prodrugs, formulations, polymorphs, tautomers, racemic mixtures and stereoisomers thereof.

2. A compound according to claim 1, having a structure selected from:

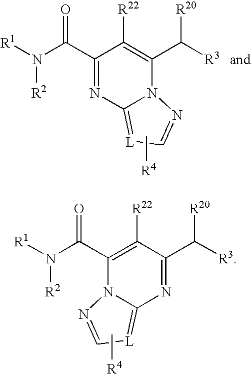

3. A compound according to claim 1, having a structure selected from:

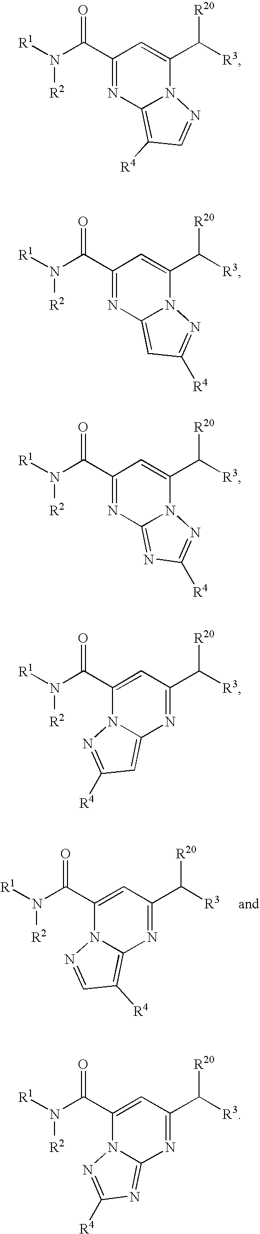

4. A compound according to claim 3, wherein R³ is selected from: 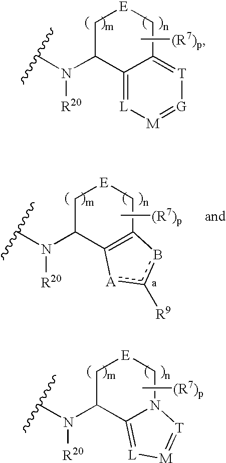
wherein:
R⁷ is independently selected from hydrogen, alkyl, cycloalkyl, halo, R⁴ and NR¹⁰R¹¹, or optionally two R⁷ groups together at the same carbon atom form ═O, ═S or ═NR¹⁰;
A and B are independently selected from CR⁹, CR⁹R¹⁰, NR¹⁰, N, O and S(O)ₓ;
G, L, M and T are independently selected from CR⁹ and N;
m and n are independently selected from 0-3, provided that:
(1) when E is present, m and n are not both 3;
(2) when E is —CH₂—W¹—, m and n are not 3; and
(3) when E is a bond, m and n are not 0; and
p is selected from 0-6;
wherein the dotted line represents a double bond between one of: carbon “a” and A, or carbon “a” and B.

5. A compound according to claim 3, wherein R³ is selected from: 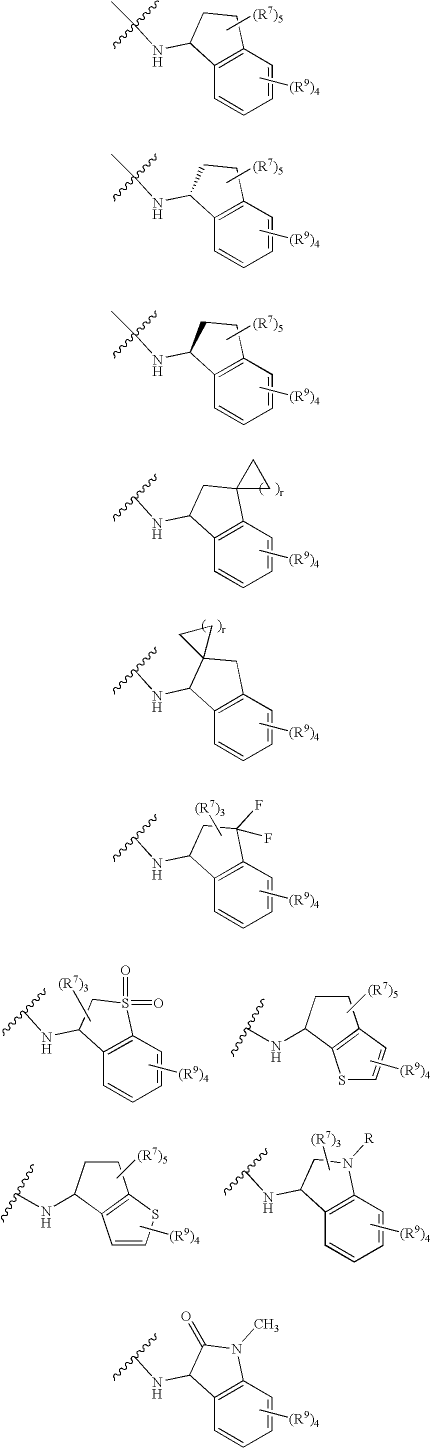 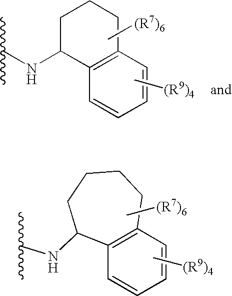
wherein:
R is selected from C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and
r is selected from 1-4.

6. A compound according to claim 3, wherein R³ selected from the group consisting of:

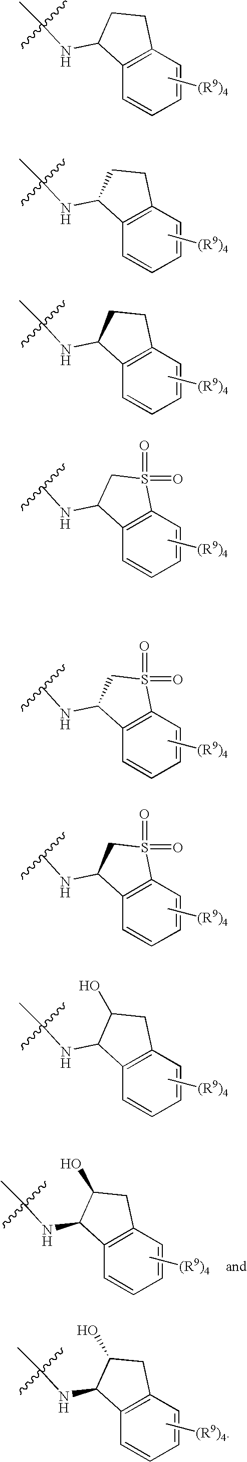

7. A compound according to claim 6, wherein R⁹ is selected from:

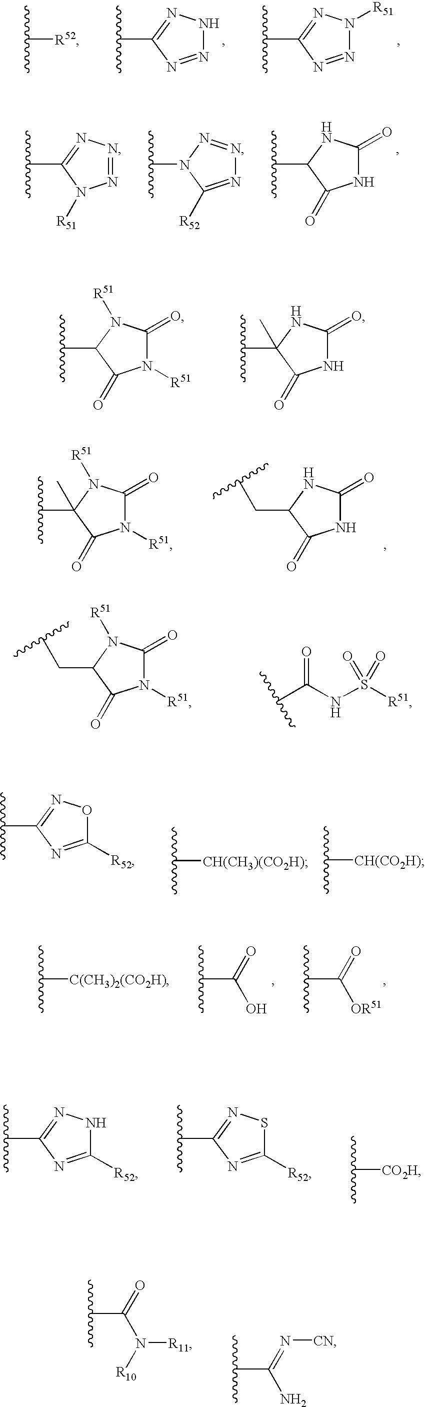

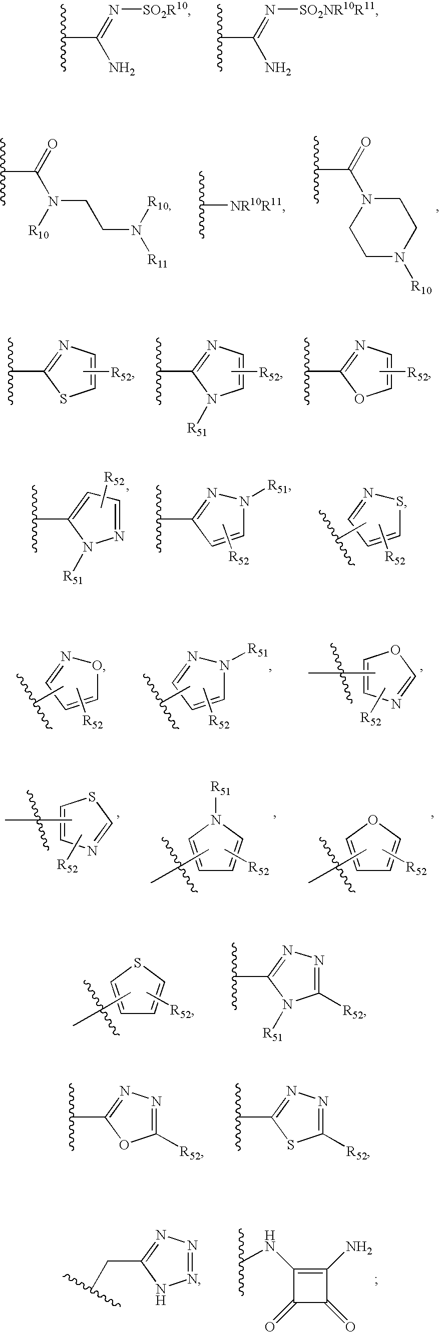

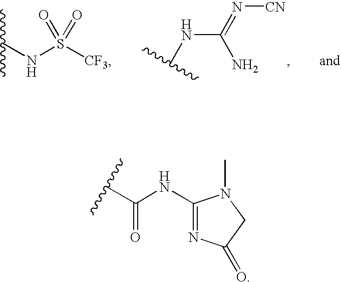

8. A compound according to claim 3, wherein R³ is selected from: 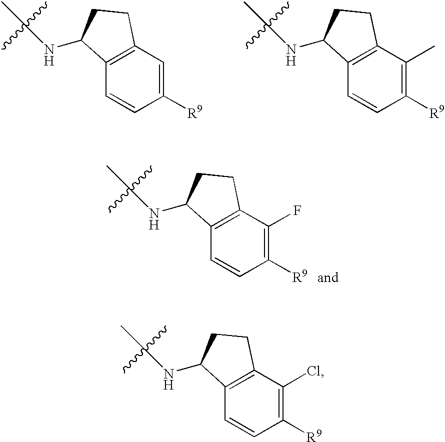
wherein:
R⁹ is selected from hydrogen, fluoro, halo, CN, alkyl, CO₂H,

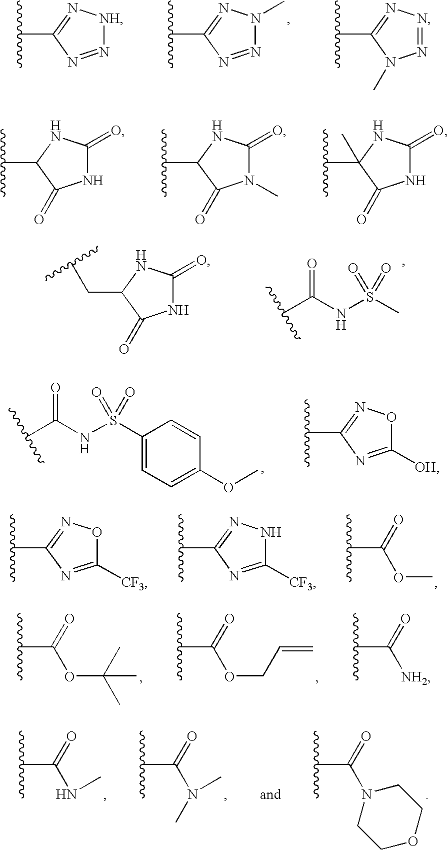

9. A compound according to claim 3, wherein R¹ is selected from: 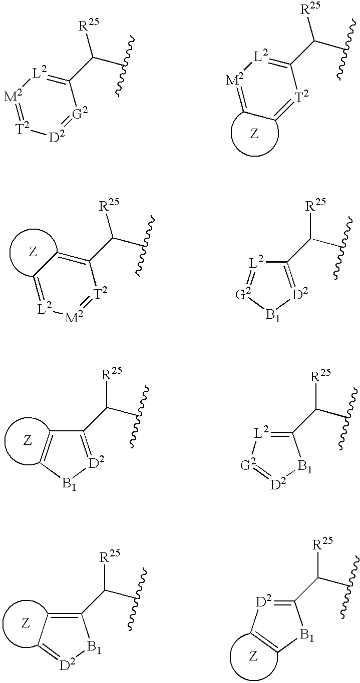
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
B₁ is selected from NR¹⁰, O and S(O)ₓ;
D², G², L², MN and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

10. A compound according to claim 3, wherein R¹ is selected from: 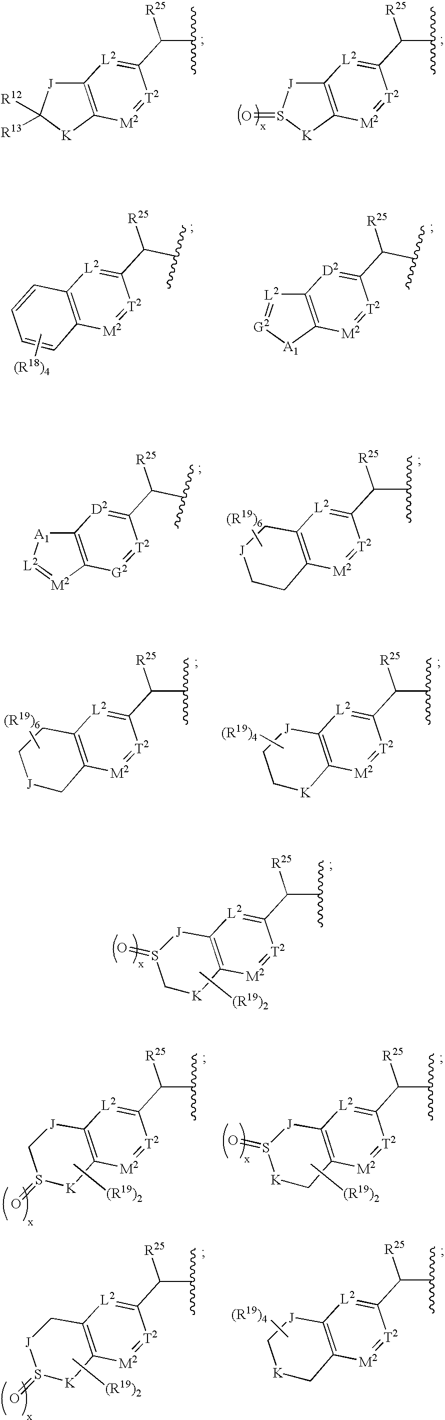 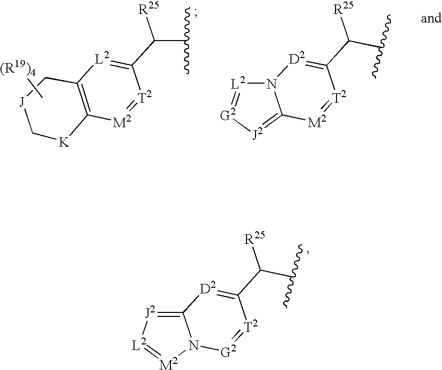
wherein:
R¹² and R¹³ are independently selected from hydrogen, alkyl and halo, wherein alkyl is optionally substituted one or more times, or optionally R¹² and R¹³ together form ═O, ═S or ═NR¹⁰;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹⁰, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹¹SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹⁰R¹⁸, NR¹⁰, O and S(O)ₓ;
A₁ is selected from NR¹⁰, O and S(O)ₓ; and
D², G2, J², L², M² and T² are independently selected from CR¹⁸ and N.

11. A compound according to claim 3, wherein R¹ is selected from: 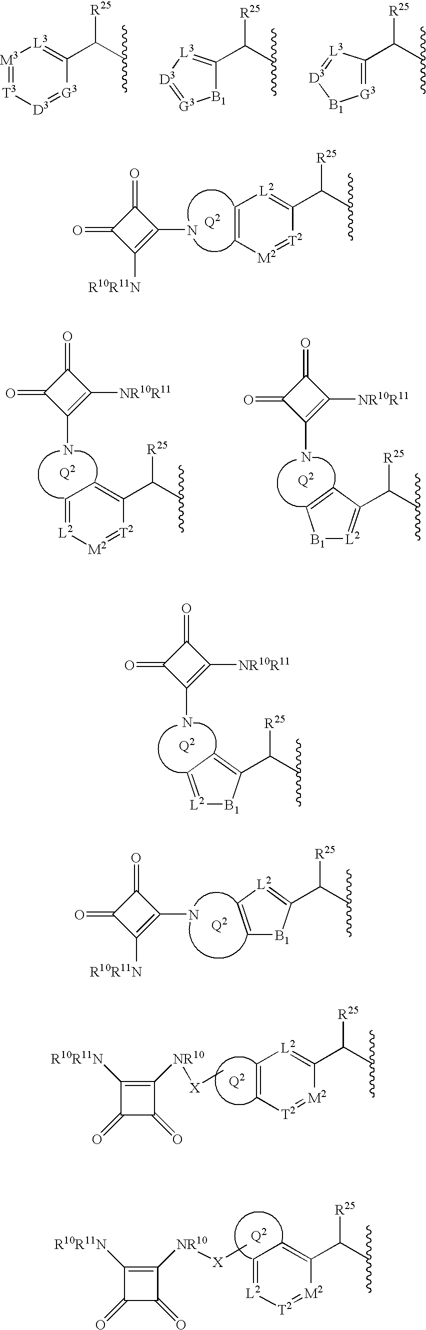 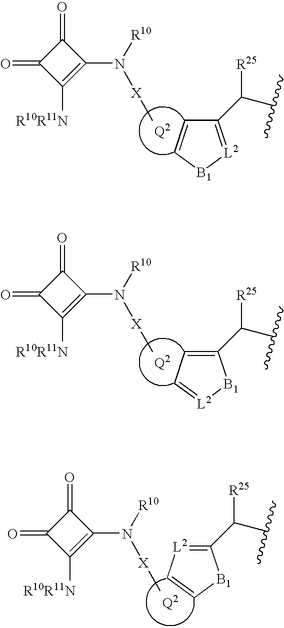
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹¹, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
D³, G³, L³, M³, and T³ are independently selected from N, CR¹⁸, (i), and (ii), 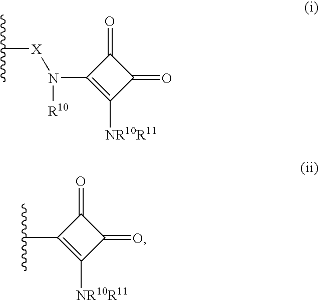
with the proviso that one of L³, M³, T³, D³, and G is (i) or (ii)
B₁ is selected from the group consisting of NR¹⁰, O and S(O)ₓ; and
Q² is a 5- to 8-membered ring selected from cycloalkyl, heterocycloalkyl, aryl, and heteroaryl, which is optionally substituted one or more times with R¹⁹.

12. A compound according to claim 3, wherein R¹ is selected from:

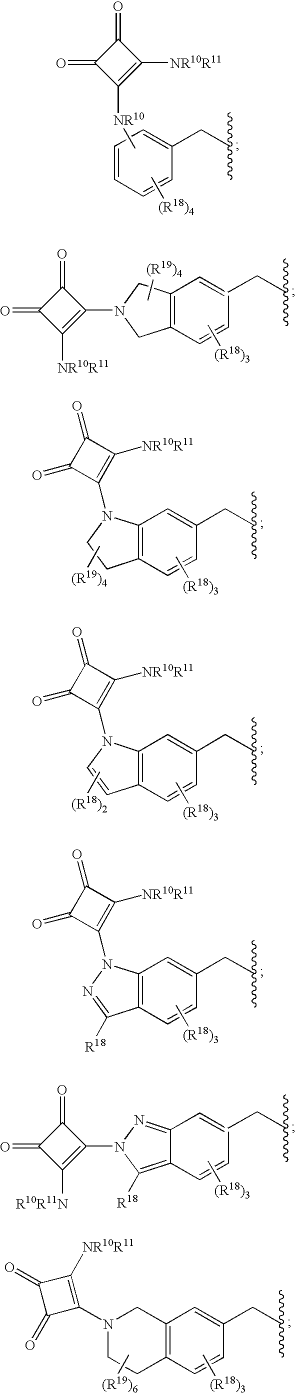

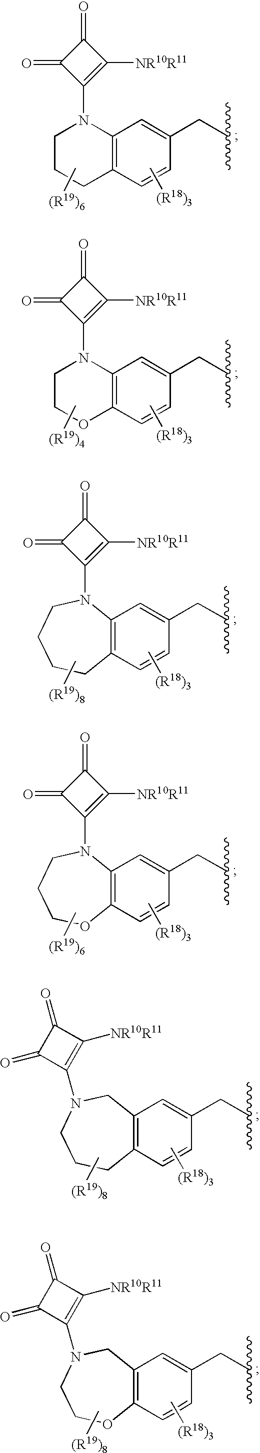

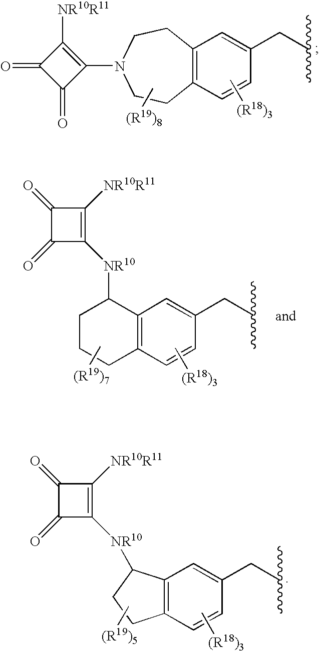

13. A compound having the structure: 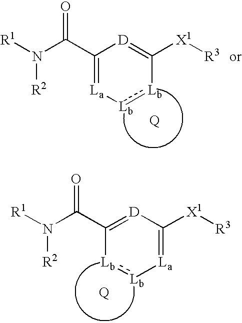
wherein:
R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is selected from 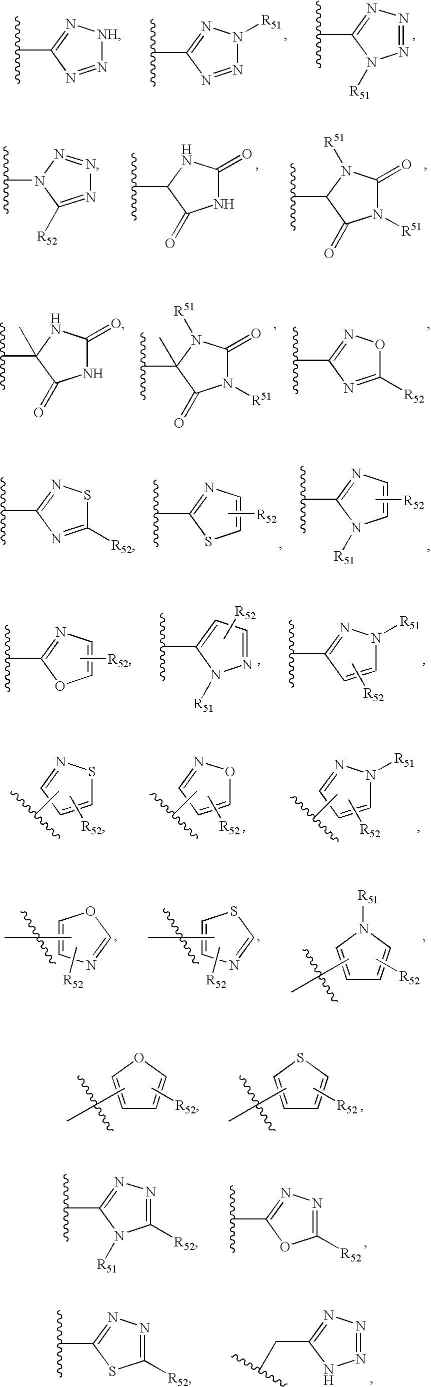 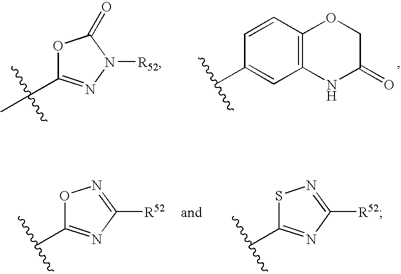
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)_{y}R¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹SO₂R^{3e}, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O), —(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 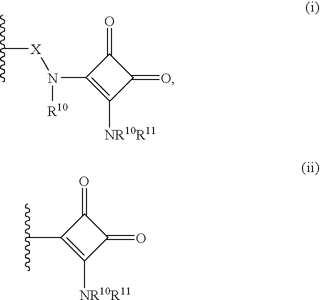
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicyclic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 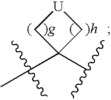
D is a member selected from CR²² and N;
Lₐ is selected from CR⁹ and N;
L_{b} is independently selected from C and N with the provisos that both L_{b} are not N, and that the bond between L_{b} and L_{b} is optionally a double bond only if both are L_{b} are carbon;
Q is a 5- or 6-membered ring selected from aryl and heteroaryl, wherein aryl and heteroaryl are optionally substituted one or more times with R⁴;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR¹⁰R¹¹)_{w};
X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;
g and h are independently selected from 0-2;
w is independently selected from 0-4;
x is selected from 0 to 2;
y is selected from 1 and 2; and
N-oxides, pharmaceutically acceptable salts, prodrugs, formulations, polymorphs, tautomers, racemic mixtures and stereoisomers thereof.

14. A compound according to claim 13, having a structure selected from:

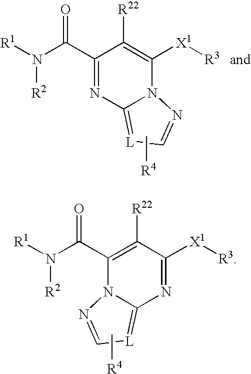

15. A compound according to claim 14, wherein R¹ is selected from: 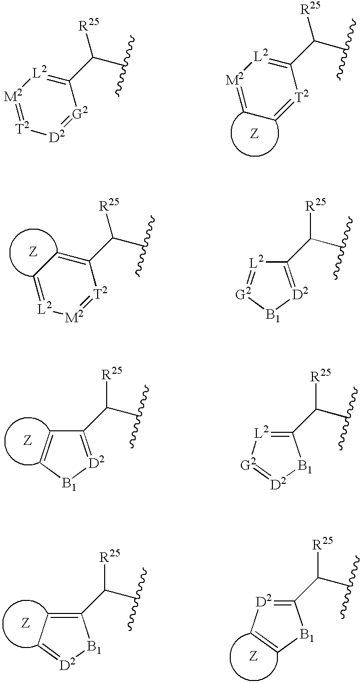
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
B₁ is selected from NR¹⁰, O and S(O)ₓ;
D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

16. A compound according to claim 14, wherein R¹ is selected from: 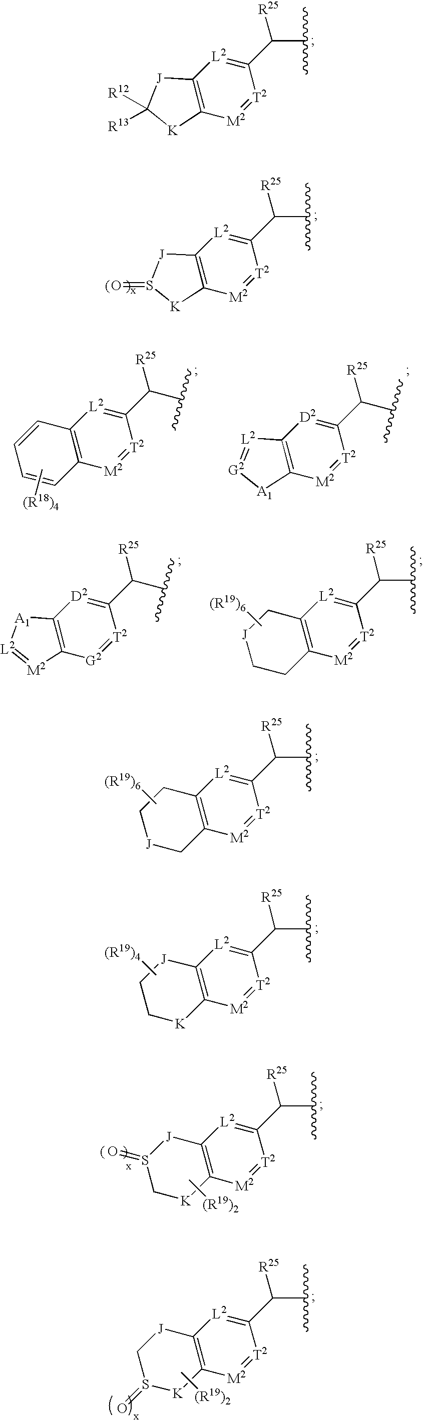 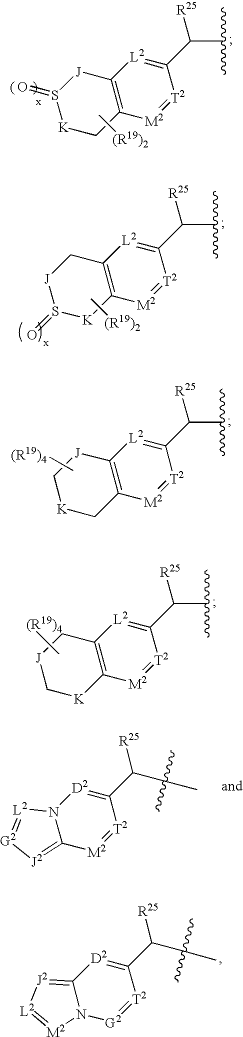
wherein:
R¹² and R¹³ are independently selected from hydrogen, alkyl and halo, wherein alkyl is optionally substituted one or more times, or optionally R¹² and R¹³ together form ═O, ═S or ═NR¹⁰;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹⁰R¹⁸, NR¹⁰, O and S(O)ₓ;
A¹ is selected from NR¹⁰, O and S(O)ₓ; and
D², G², J², L², M² and T² are independently selected from CR¹⁸ and N.

17. A compound according to claim 14, wherein R¹ is selected from: 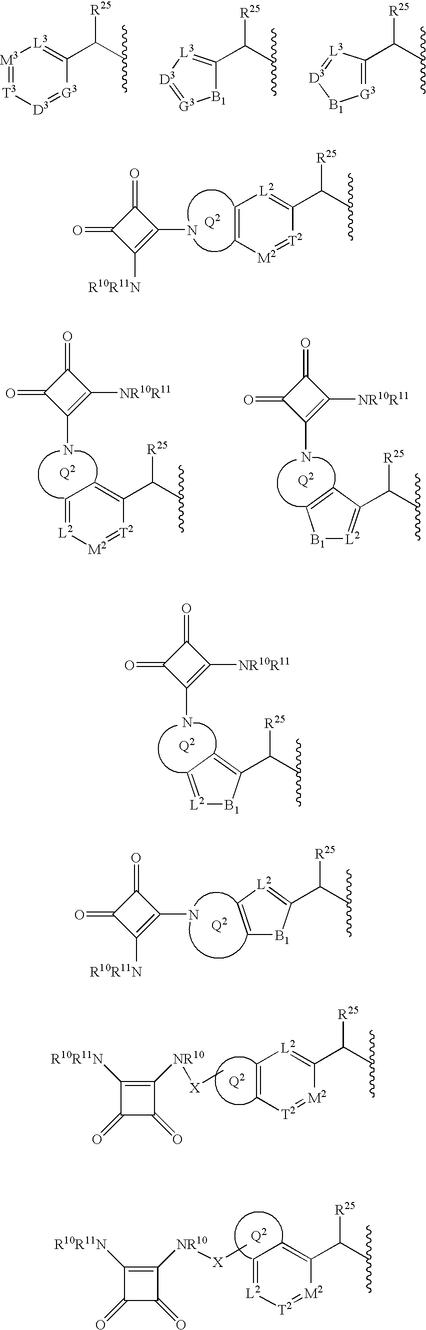 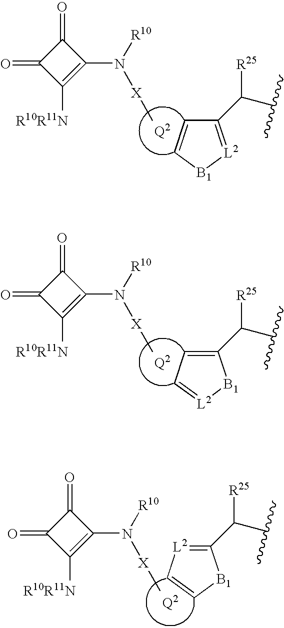
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹¹COR¹¹, NR¹⁰SO₂R¹¹, NR¹¹SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
D³, G³, L³, M³, and T³ are independently selected from N, CR¹⁸, (i), and (ii), 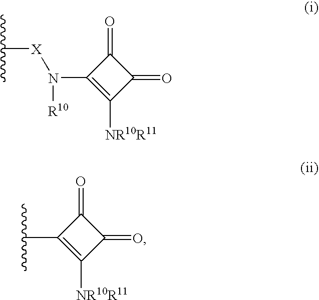
with the proviso that one of L³, M³, T³, D³, and G is (i) or (ii)
B₁ is selected from the group consisting of NR¹⁰, O and S(O)ₓ; and
Q² is a 5- to 8-membered ring selected from cycloalkyl, heterocycloalkyl, aryl, and heteroaryl, which is optionally substituted one or more times with R¹⁹.

18. A compound according to claim 14, wherein R¹ is selected from:

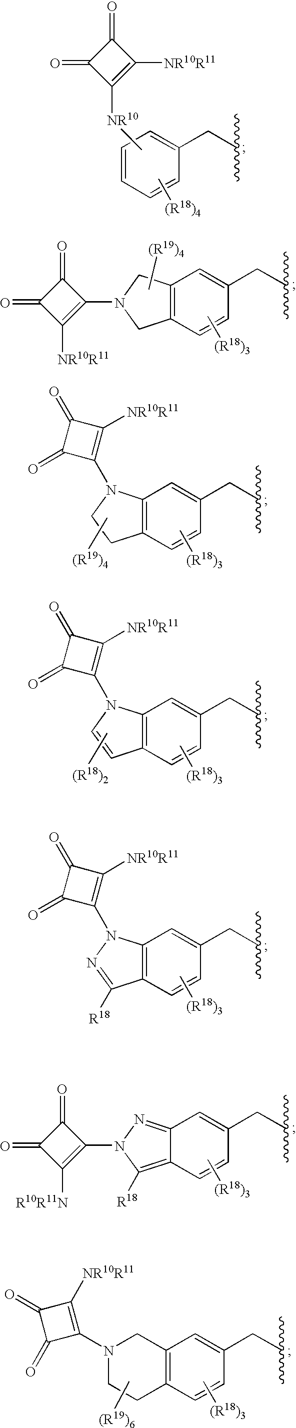

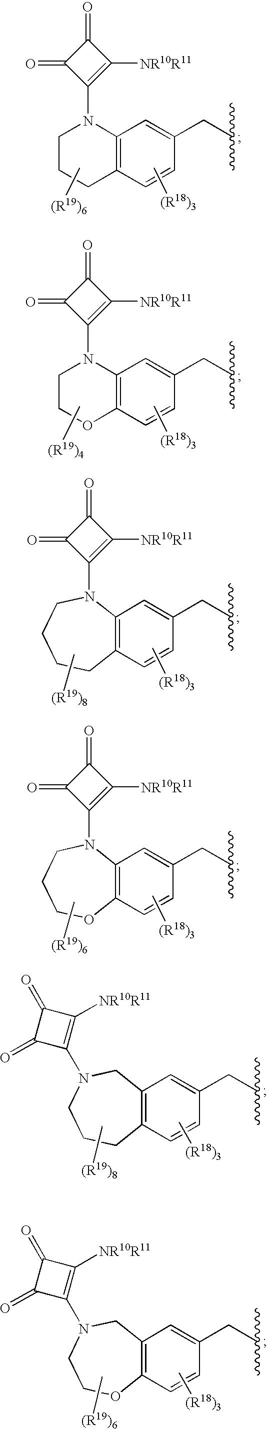

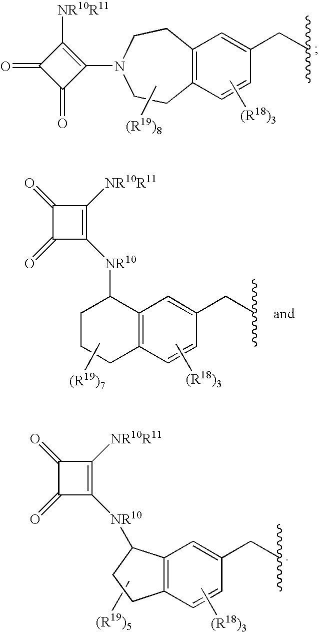

19. A compound having the structure: 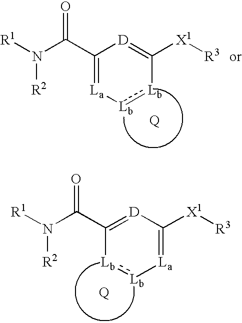
wherein:
R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiralkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is SO₂NR¹⁰R¹¹, SO₂NR²⁰R²¹, PO₂R¹⁰, PO₂R¹, 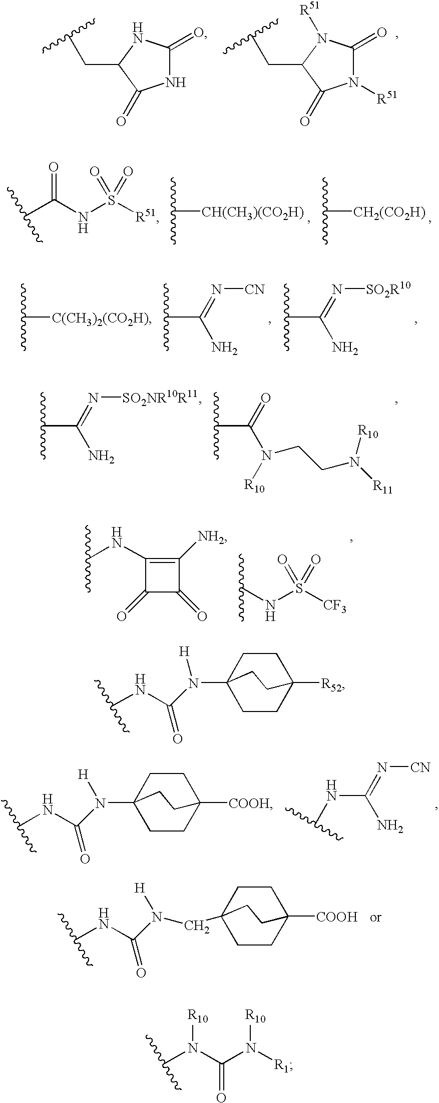
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)_{y}R¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹⁰—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 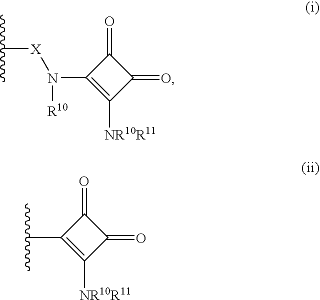
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R² are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicyclic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸¹R⁸¹, wherein alkyl, aryl, and heteroaryl are optionally substituted one or more times;
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 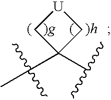
D is a member selected from CR²² and N;
Lₐ is selected from CR⁹ and N;
L_{b} is independently selected from C and N with the provisos that both L_{b} are not N, and that the bond between L_{b} and L_{b} is optionally a double bond only if both are L_{b} are carbon;
Q is a 5- or 6-membered ring selected from aryl and heteroaryl, wherein aryl and heteroaryl are optionally substituted one or more times with R⁴;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR10R¹¹)_{w};
X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;
g and h are independently selected from 0-2;
w is independently selected from 0-4;
x is selected from 0 to 2;
y is selected from 1 and 2; and
N-oxides, pharmaceutically acceptable salts, prodrugs, formulations, polymorphs, tautomers, racemic mixtures and stereoisomers thereof.

20. A compound according to claim 19, having a structure selected from:

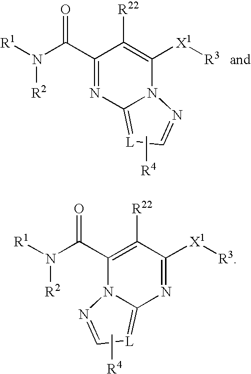

21. A compound according to claim 20, wherein R¹ is selected from: 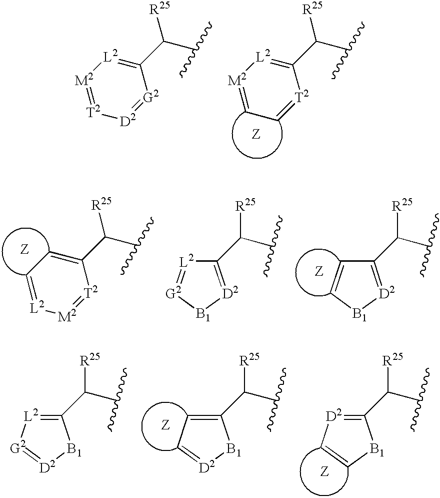
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
B₁ is selected from NR¹⁰, O and S(O)ₓ;
D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

22. A compound according to claim 20, wherein R¹ is selected from: 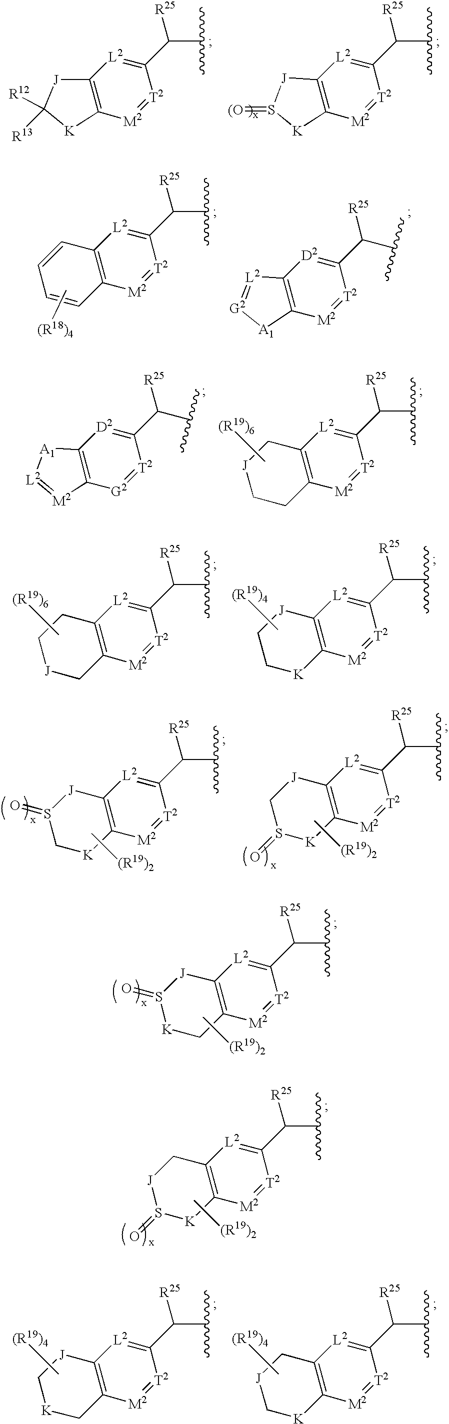 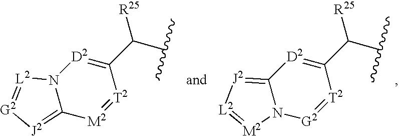
wherein:
R¹² and R¹³ are independently selected from hydrogen, alkyl and halo, wherein alkyl is optionally substituted one or more times, or optionally R¹² and R¹³ together form ═O, ═S or ═NR¹⁰;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹⁰R¹⁸, NR¹⁰, O and S(O)ₓ;
A¹ is selected from NR¹⁰, O and S(O)ₓ; and
D², G², J², L², M² and T² are independently selected from CR¹⁸ and N.

23. A compound according to claim 20, wherein R¹ is selected from: 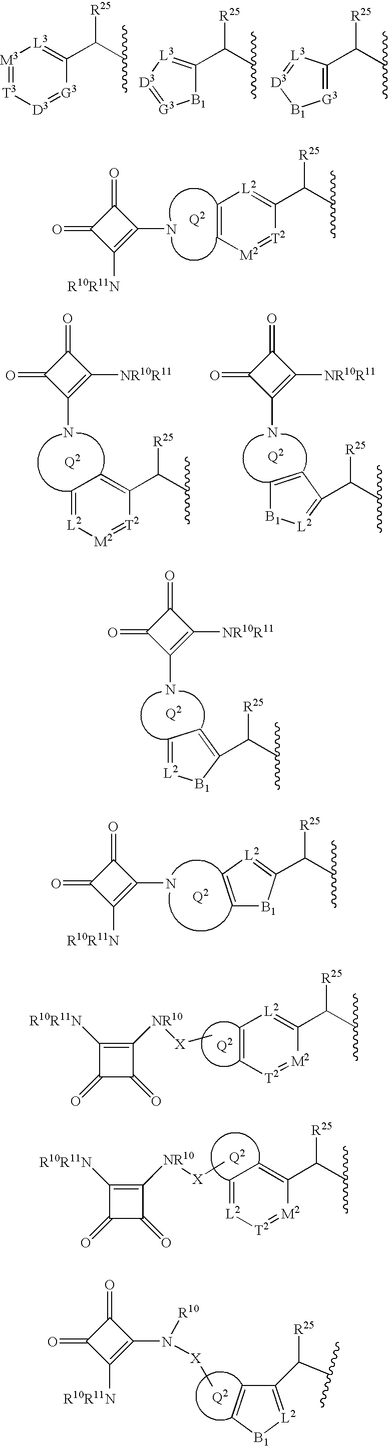 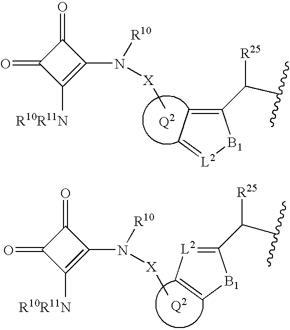
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
D³, G³, L³, M³, and T³ are independently selected from N, CR¹⁸, (i), and (ii), 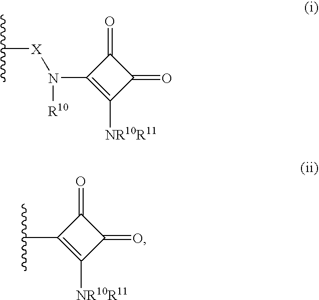
with the proviso that one of L³, M³, T³, D³, and G³ is (i) or (ii)
B₁ is selected from the group consisting of NR¹⁰, O and S(O)ₓ; and
Q² is a 5- to 8-membered ring selected from cycloalkyl, heterocycloalkyl, aryl, and heteroaryl, which is optionally substituted one or more times with R¹⁹.

24. A compound according to claim 20, wherein R¹ is selected from:

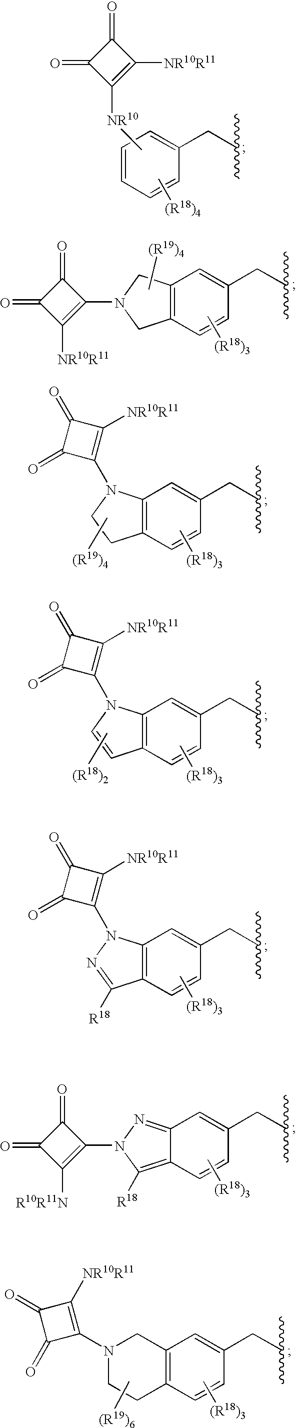

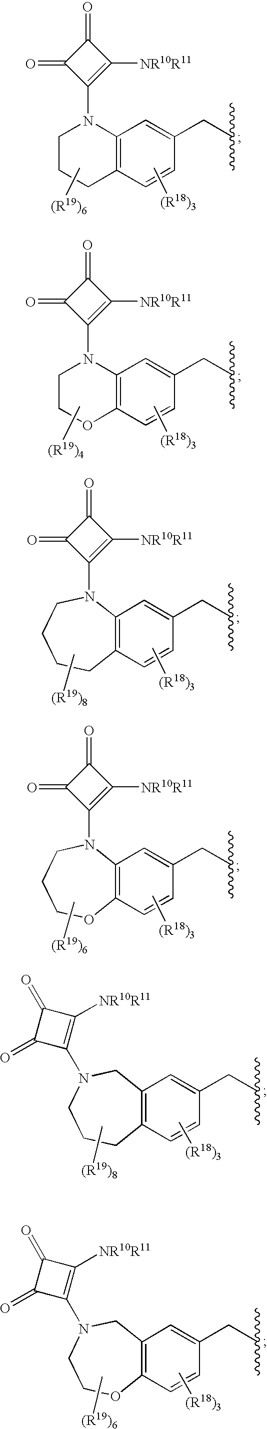

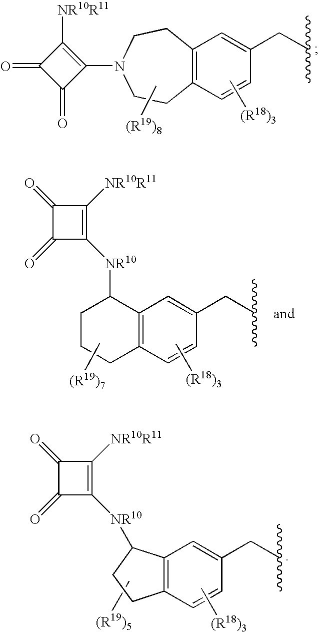

25. A compound having the structure: 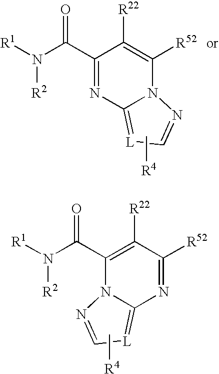
wherein:
R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)ₓNR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹⁰—CN, O—(C₀-C₆)-alkyl-C(O)NR¹⁰R¹¹, S(O)ₓ—(C₀-C₆)-alkyl-C(O)OR¹⁰, S(O), —(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 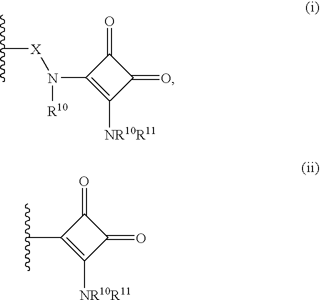
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicyclic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, fluoroalkoxy, alkyl and haloalkyl;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 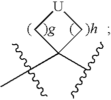
D is a member selected from CR²² and N;
L is C or N;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR¹⁰R¹¹)_{w};
X¹ is a bond, NR¹⁰, CH₂, CHR²⁰, CR²⁰R²¹, SO₂, SO, S, PO₂, O, C═S, C═NR¹, C═N—SO₂R¹⁰, C═N—CN, C═N—CONR¹⁰R¹¹, C═N—COR¹⁰, C═N—OR¹⁰;
g and h are independently selected from 0-2;
w is independently selected from 0-4;
x is selected from 0 to 2;
y is selected from 1 and 2; and
N-oxides, pharmaceutically acceptable salts, prodrugs, formulations, polymorphs, tautomers, racemic mixtures and stereoisomers thereof.

26. A compound according to claim 25, wherein R¹ is selected from: 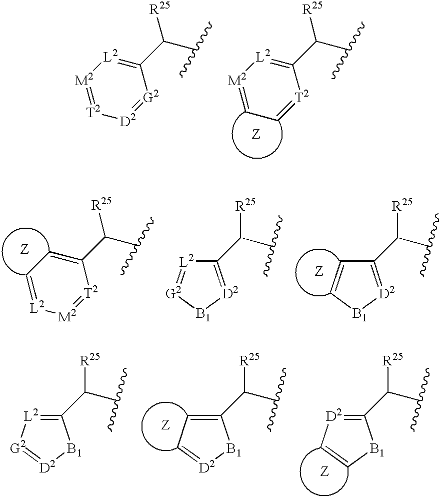
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
B₁ is selected from NR¹⁰, O and S(O)ₓ;
D², G², L², M² and T² are independently selected from CR¹⁸ and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

27. A compound according to claim 25, wherein R¹ is selected from: 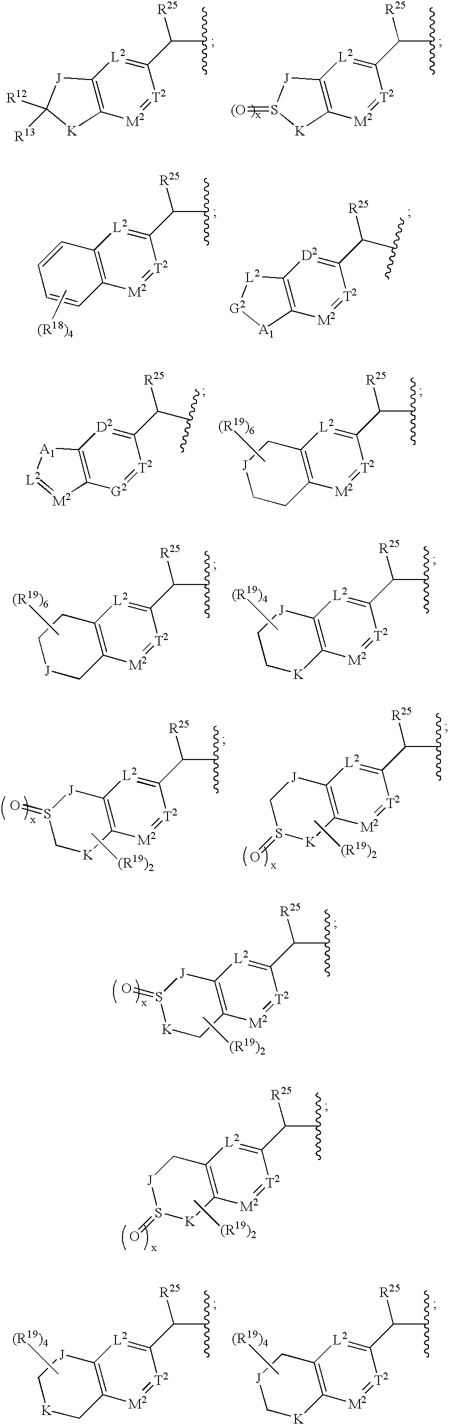 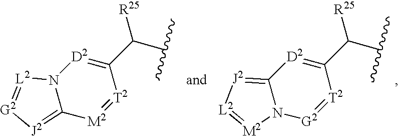
wherein:
R¹² and R¹³ are independently selected from hydrogen, alkyl and halo, wherein alkyl is optionally substituted one or more times, or optionally R¹² and
R¹³ together form ═O, ═S or ═NR¹⁰;
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹⁰R¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹¹R¹⁸, NR¹¹, O and S(O)ₓ;
A₁ is selected from NR¹⁰, O and S(O)ₓ; and
D², G², J², L², M² and T² are independently selected from CR¹⁸ and N.

28. A compound according to claim 25, wherein R¹ is selected from: 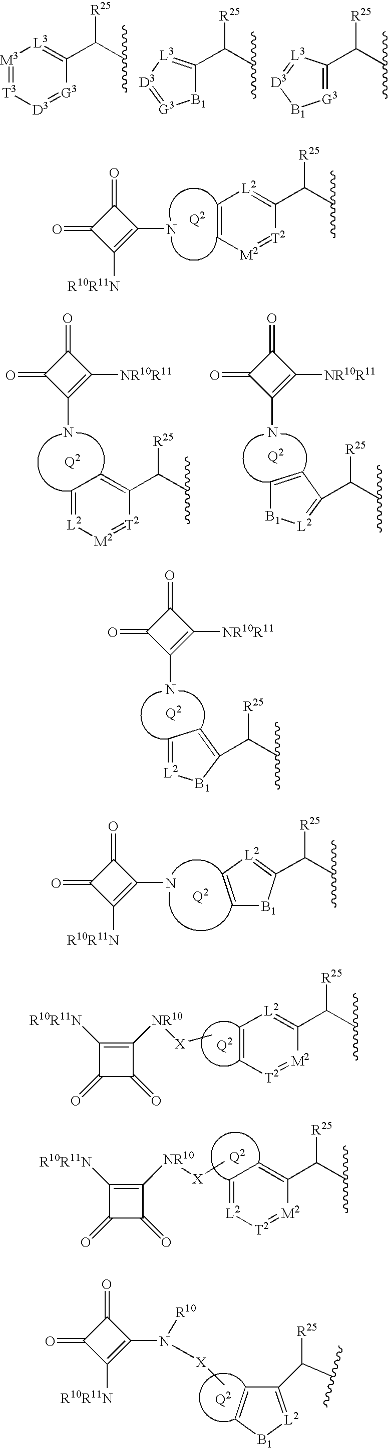 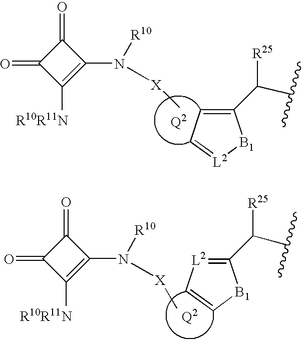
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
D³, G³, L³, M³, and T³ are independently selected from N, CR¹⁸, (i), and (ii), 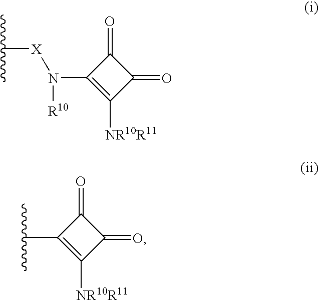
with the proviso that one of L³, M³, T³, D³, and G³ is (i) or (ii)
B₁ is selected from the group consisting of NR¹⁰, O and S(O)ₓ; and
Q² is a 5- to 8-membered ring selected from cycloalkyl, heterocycloalkyl, aryl, and heteroaryl, which is optionally substituted one or more times with R¹⁹.

29. A compound according to claim 25, wherein R¹ is selected from:

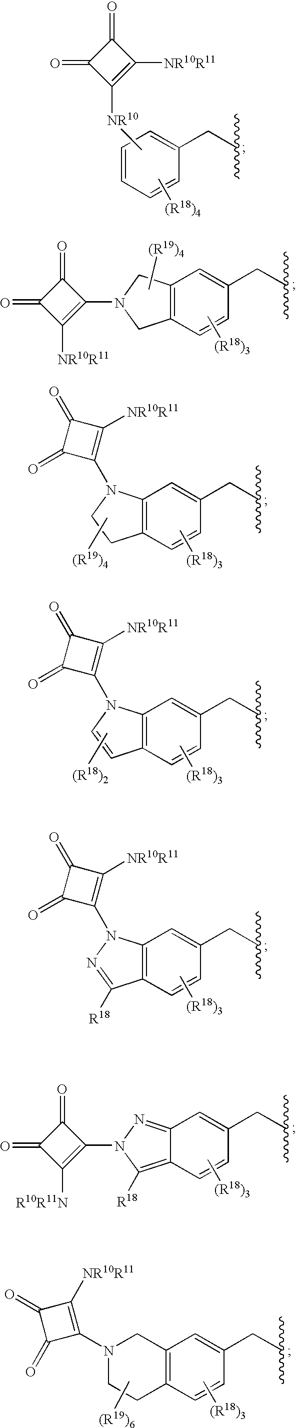

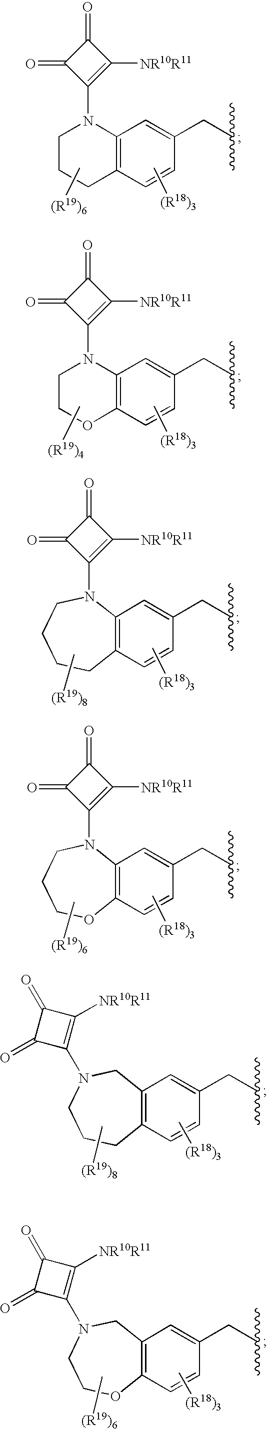

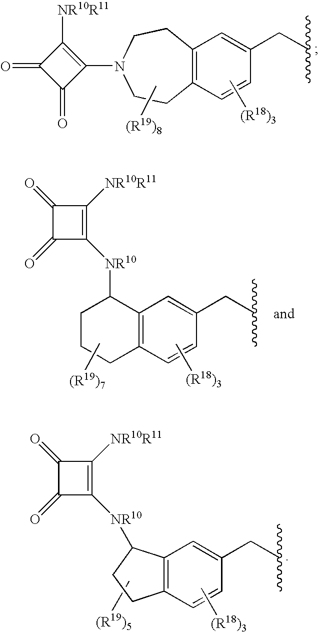

30. A compound having the structure: 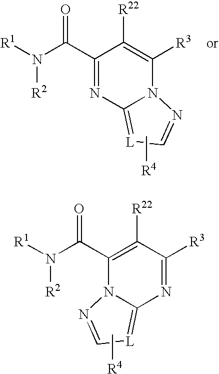
wherein:
R¹ in each occurrence is independently selected from hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² in each occurrence is independently selected from hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is NR²⁰R²¹, NR¹⁰R¹¹, NR¹⁰SO₂R¹⁰, NR¹⁰SO₂R¹¹, OR¹⁰, OR²¹ or NR¹⁰NR⁹;
R⁴ in each occurrence is independently selected from R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-allyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from hydrogen, alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein alkyl, heteroalkyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times, or when R¹⁰ and R¹¹ are attached to a nitrogen atom they may be taken together to complete a 3- to 8-membered ring containing carbon atoms and optionally containing a heteroatom selected from O, S, or NR⁵⁰ and which is optionally substituted one or more times;
R¹⁴ is independently selected from hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times.
R¹⁶ is selected from cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 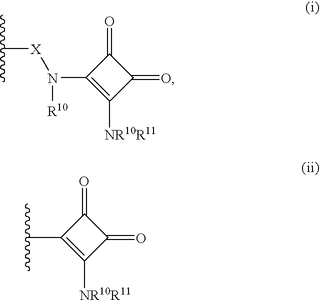
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from selected from hydrogen, alkyl, heteroalkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or when R²⁰ and R²¹ are attached to a nitrogen atom they may be taken together to complete a 3- to 8-membered ring containing carbon atoms and optionally containing a heteroatom selected from O, S, or NR⁵⁰ and which is optionally substituted one or more times;
R²¹ is a monocyclic, bicyclic or tricyclic ring system wherein said bicyclic or tricyclic ring system is fused and contains at least one ring which is partially saturated and
wherein R²¹ is optionally substituted one or more times, or
wherein R²¹ is optionally substituted by one or more R⁹ groups;
R²² is independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹¹, and fluoroalkyl, wherein alkyl, cycloalkyl, alkoxy, alkenyl, alkynyl and fluoroalkyl are optionally substituted one or more times;
R³⁰ is selected from alkyl and (C₀-C₆)-alkyl-aryl, wherein alkyl and aryl are optionally substituted;
R⁵⁰ in each occurrence is independently selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁵¹ is independently selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times;
R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, CONR¹⁰R¹¹ and haloalkyl, wherein alkyl, cycloalkyl and haloalkyl are optionally substituted one or more times;
L², M², and T² are independently selected from CR¹⁸ and N;
D³, G³, L³, M³, and T³ are independently selected from N, CR¹⁸, (i), and (ii), 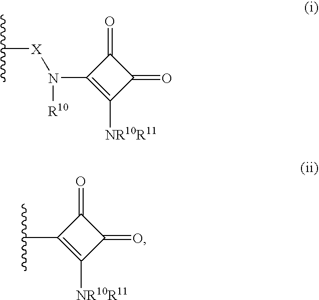
with the proviso that one of L³, M³, T³, D³, and G³ is (i) or (ii)
B₁ is selected from the group consisting of NR¹⁰, O and S(O)ₓ; and
Q² is a 5- to 8-membered ring selected from cycloalkyl, heterocycloalkyl, aryl, and heteroaryl, which is optionally substituted one or more times with R¹⁹.

34. A compound according to claim 30, wherein R¹ is selected from:
taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 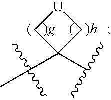
D is a member selected from CR²² and N;
L is C or N;
U is selected from C(R⁵R¹⁰), NR⁵, O, S, S═O and S(═O)₂;
W¹ is selected from O, NR⁵, S, S═O, S(═O)₂, N(R¹⁰)(C═O), N(R¹⁰)S(═O)₂ and S(═O)₂N(R¹⁰);
X is selected from a bond and (CR¹⁰R¹¹)_{w}E(CR¹⁰R¹¹)_{w};
g and h are independently selected from 0-2;
w is independently selected from 0-4;
x is selected from 0 to 2;
y is selected from 1 and 2; and
N-oxides, pharmaceutically acceptable salts, prodrugs, formulations, polymorphs, tautomers, racemic mixtures and stereoisomers thereof.

31. A compound according to claim 30, wherein R¹ is selected from: 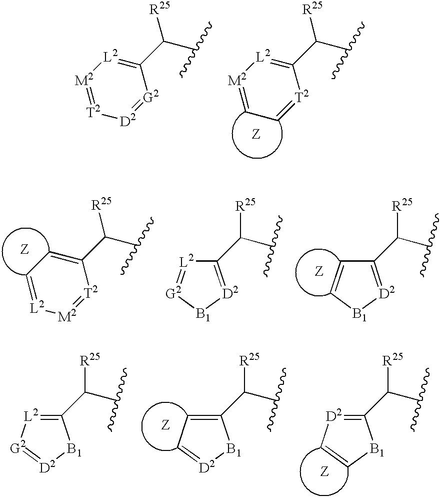
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
B₁ is selected from NR¹⁰, O and S(O)ₓ;
D², G², L², M² and T² are independently selected from CR18 and N; and
Z is a 5- to 8-membered ring selected from cycloalkyl, heterocycloalkyl, aryl and heteroaryl, wherein cycloalkyl, heterocycloalkyl, aryl and heteroaryl are optionally substituted one or more times.

32. A compound according to claim 30, wherein R¹ is selected from: 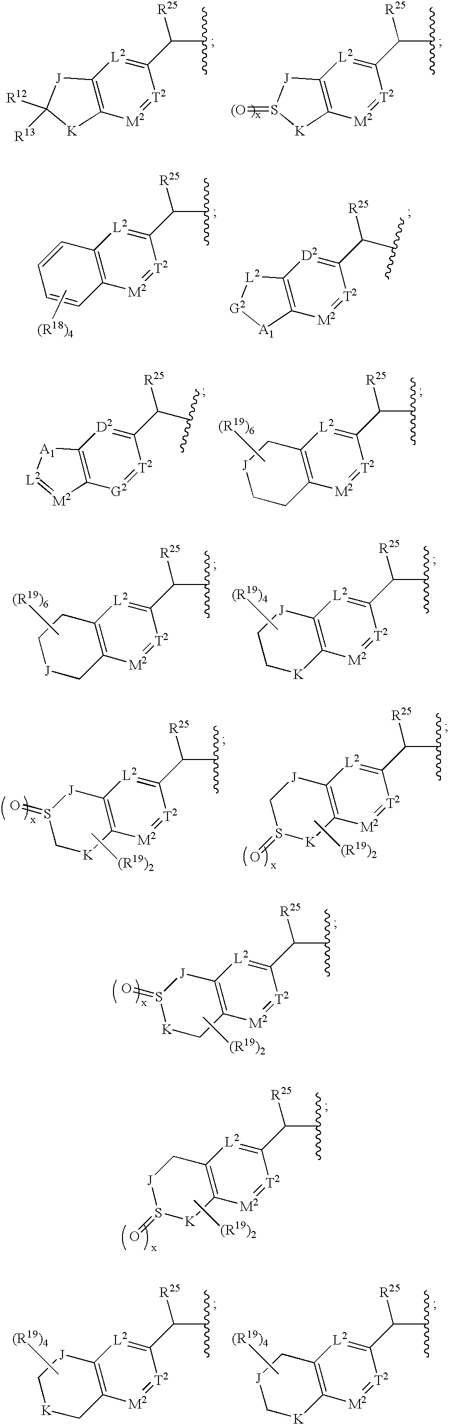 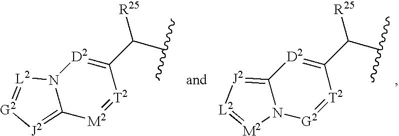
wherein:
R¹² and R¹³ are independently selected from hydrogen, alkyl and halo, wherein alkyl is optionally substituted one or more times, or optionally R¹² and
R¹³ together form ═O, ═S or ═NR¹⁰,
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from hydrogen, alkyl, cycloalkyl, C(O)NR¹⁰R¹¹ and haloalkyl, wherein alkyl, cycloalkyl, and haloalkyl are optionally substituted one or more times;
J and K are independently selected from CR¹⁰R¹⁸, NR¹⁰, O and S(O)ₓ;
A₁ is selected from NR¹⁰, O and S(O)ₓ; and
D², G², J², L², M² and T² are independently selected from CR¹⁸ and N.

33. A compound according to claim 30, wherein R¹ is selected from: 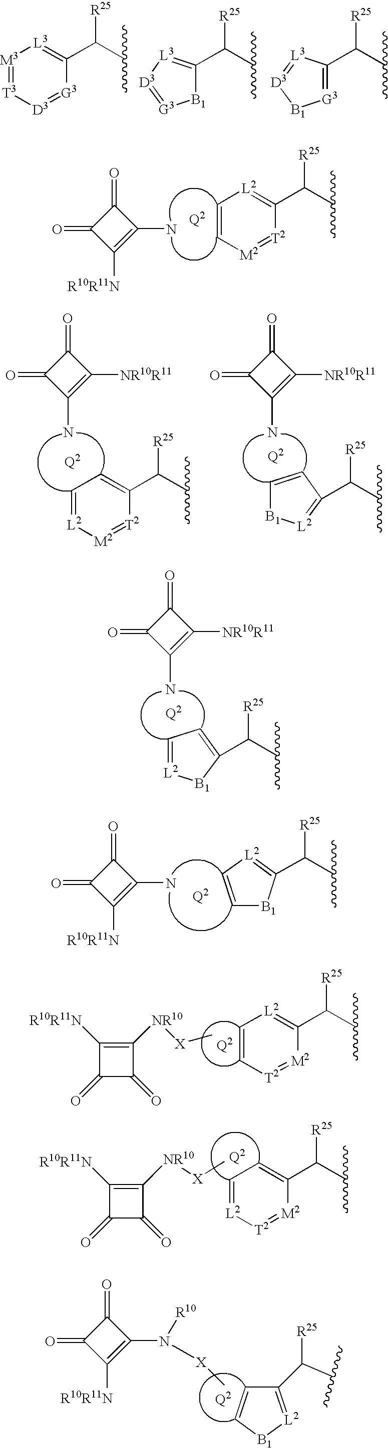 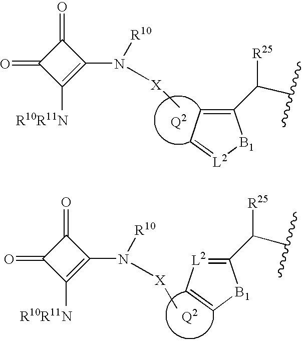
wherein:
R¹⁸ is independently selected from hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl,

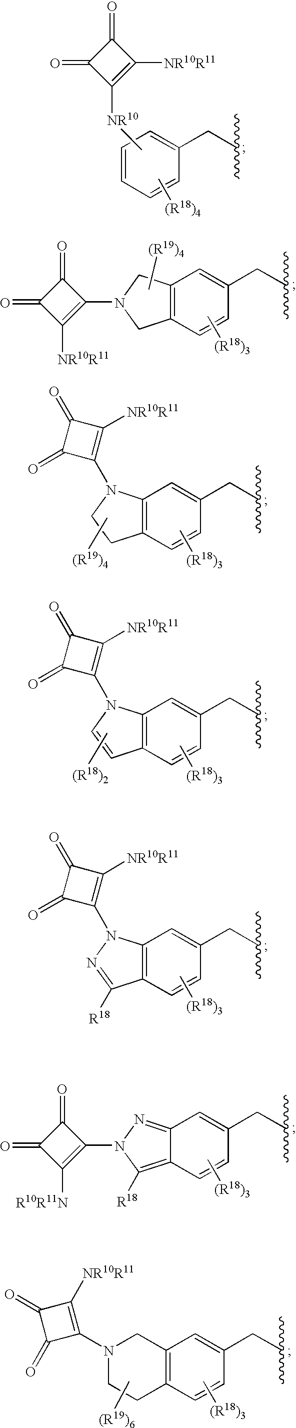

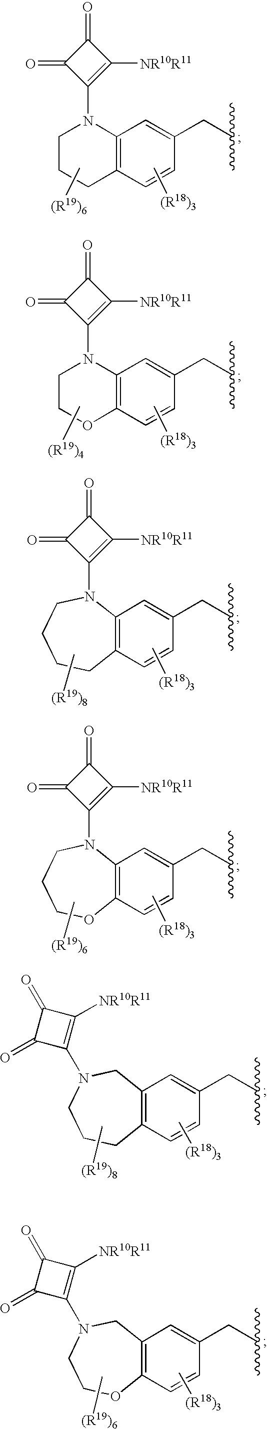

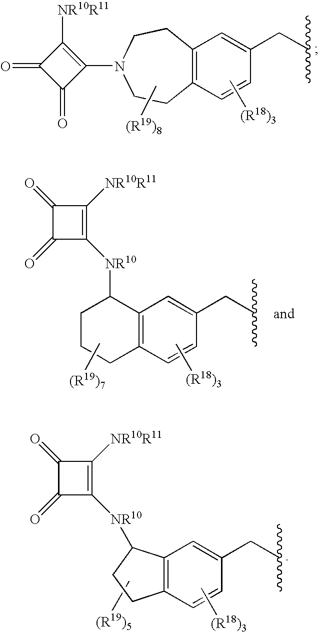

35. A compound selected from: 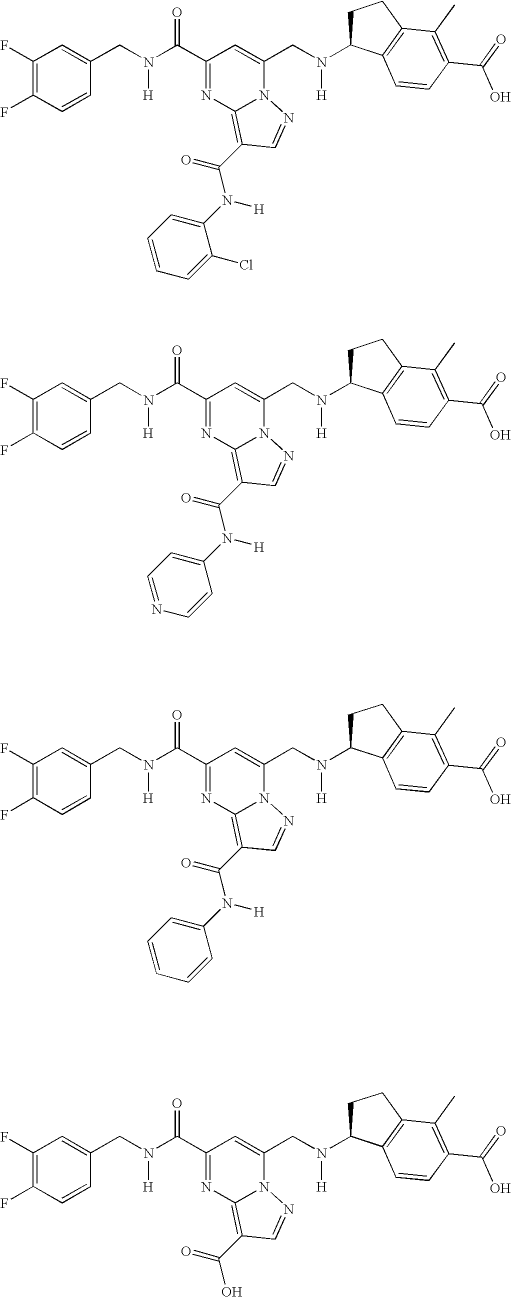 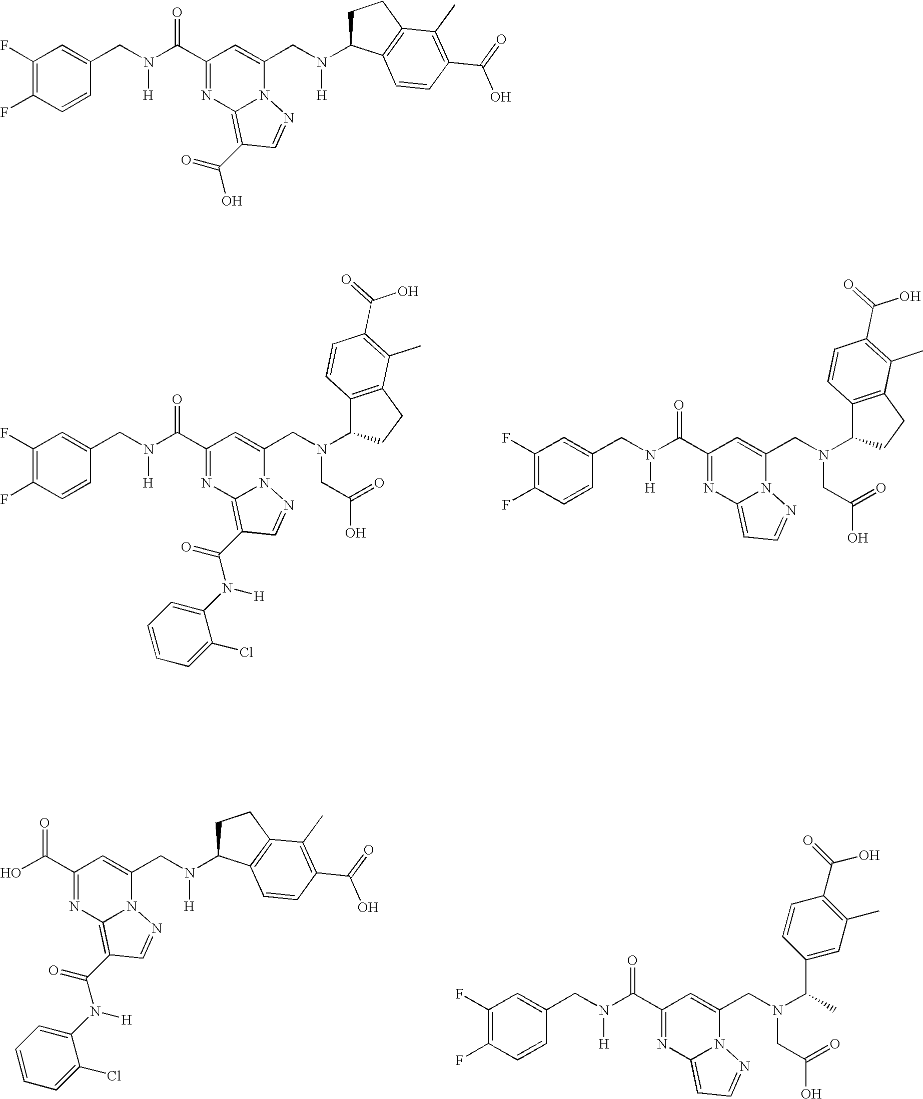 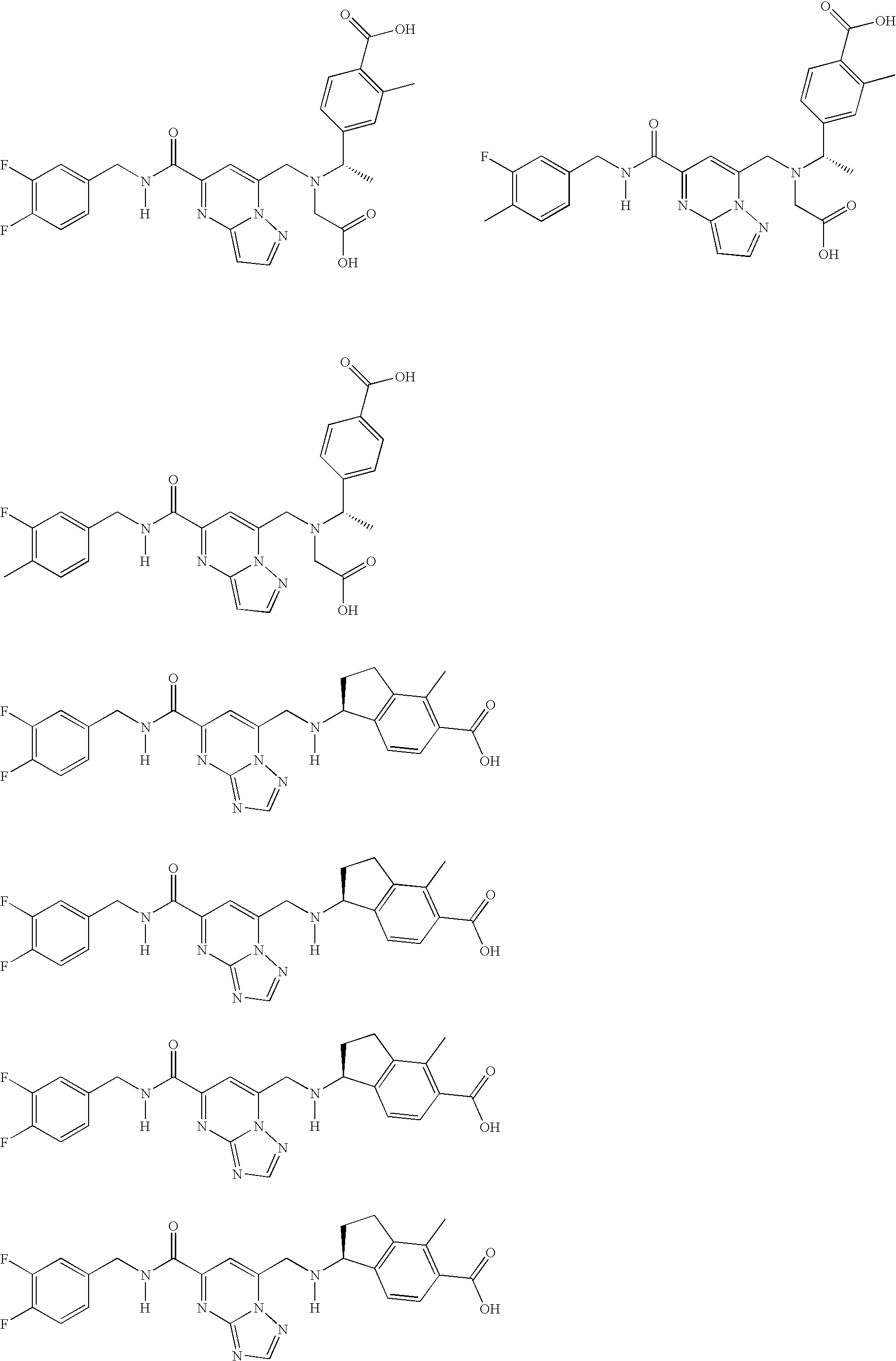 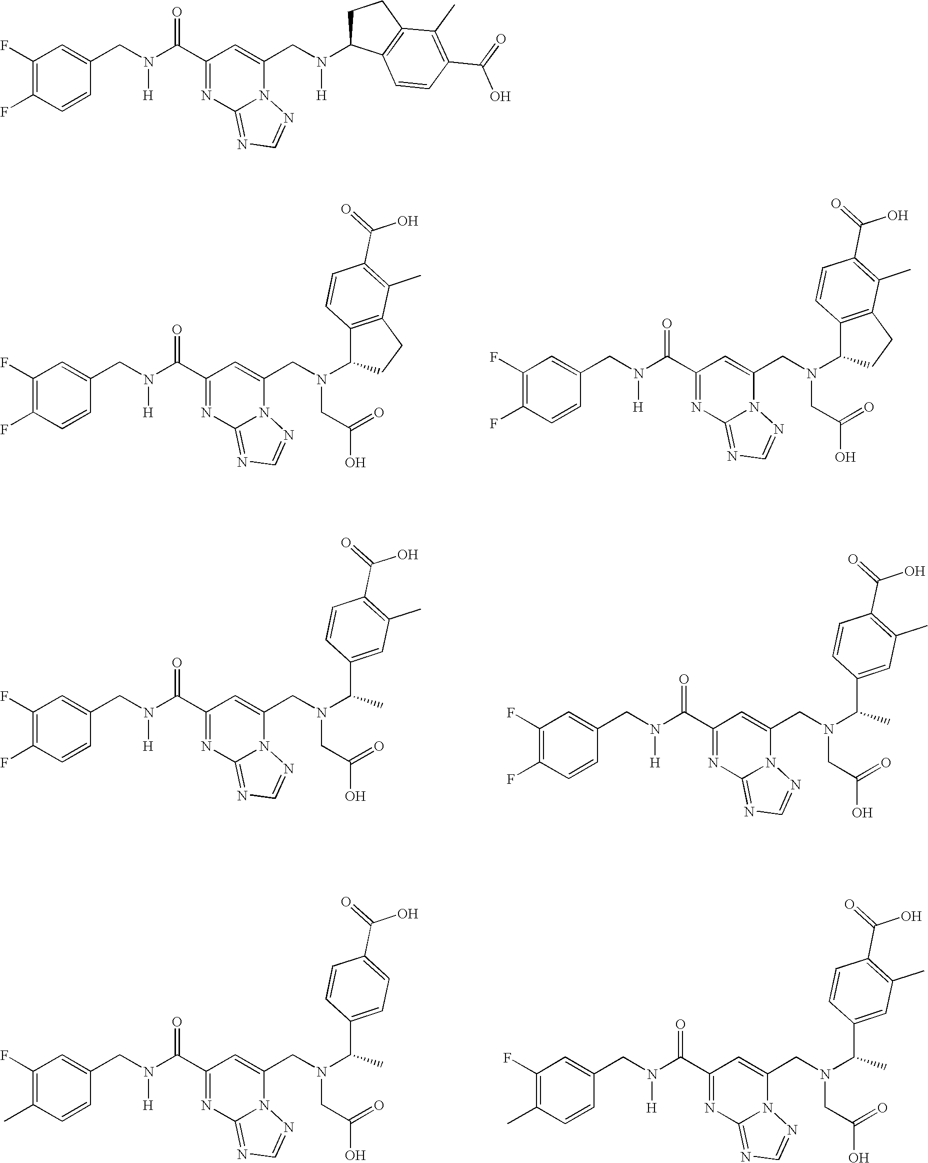 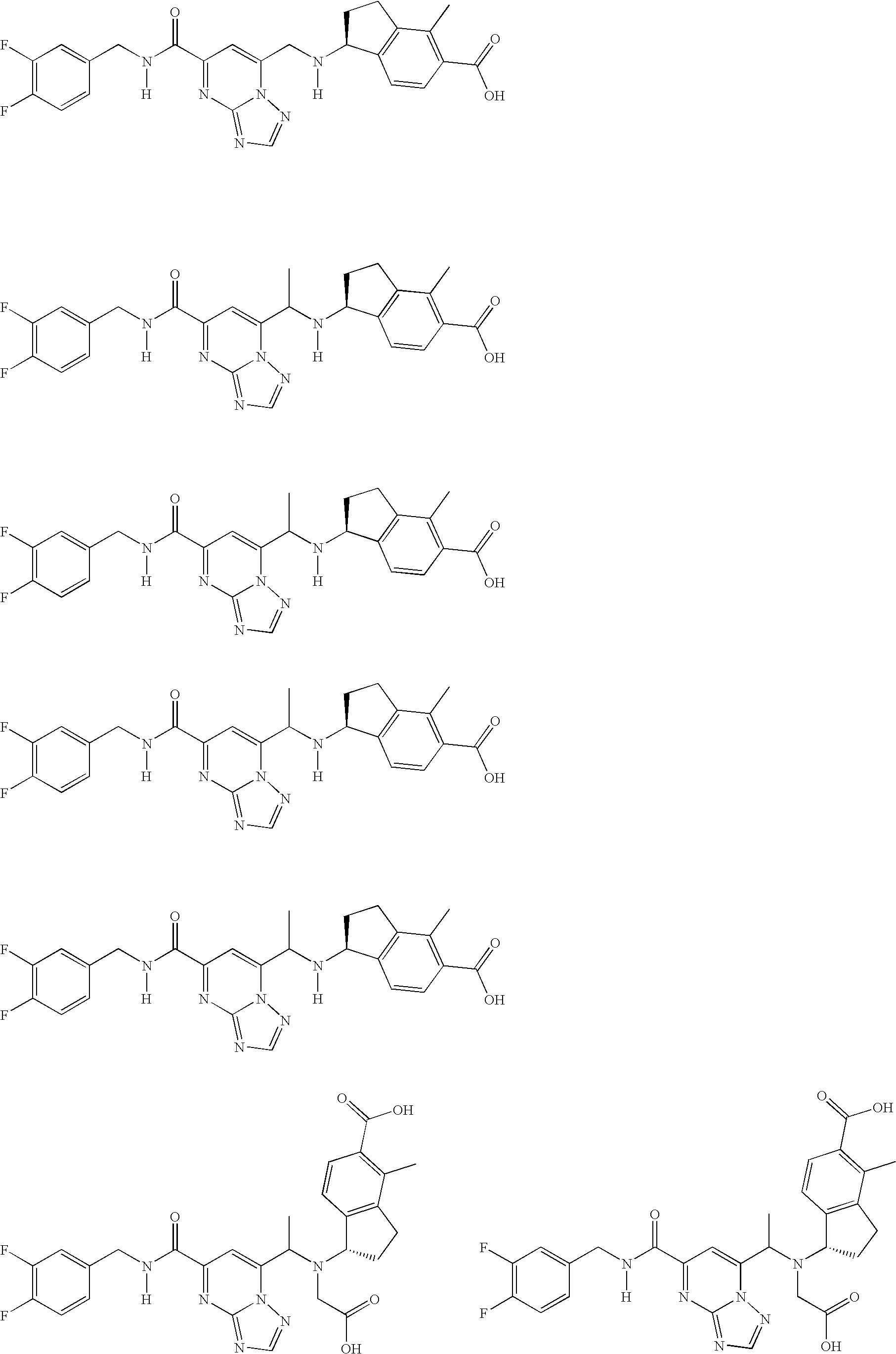 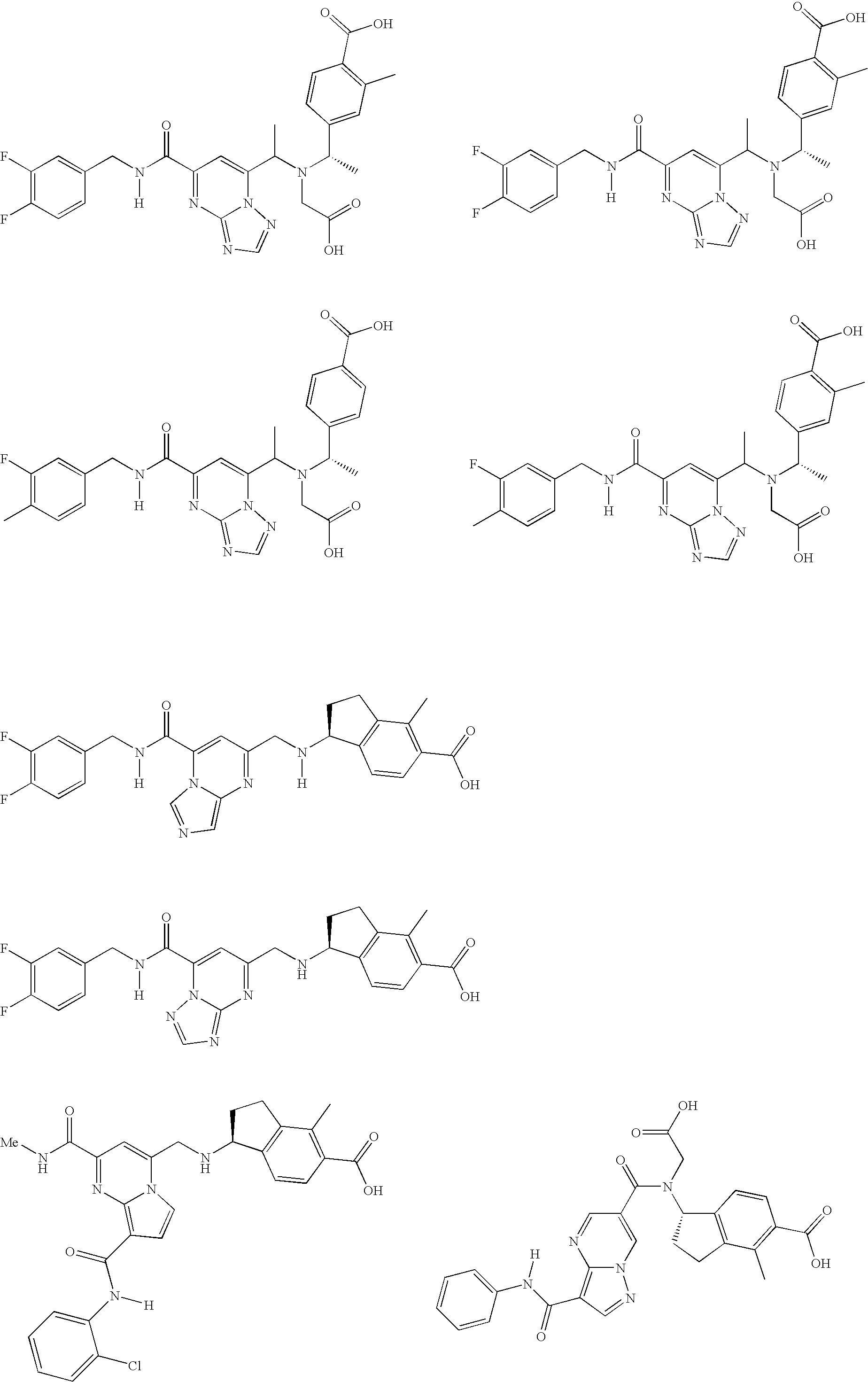 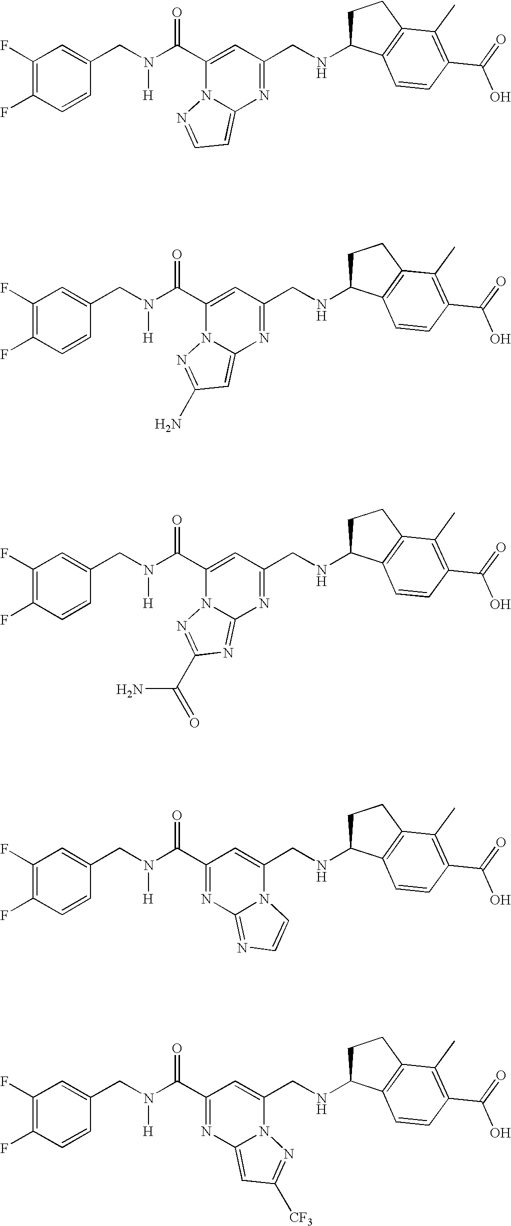 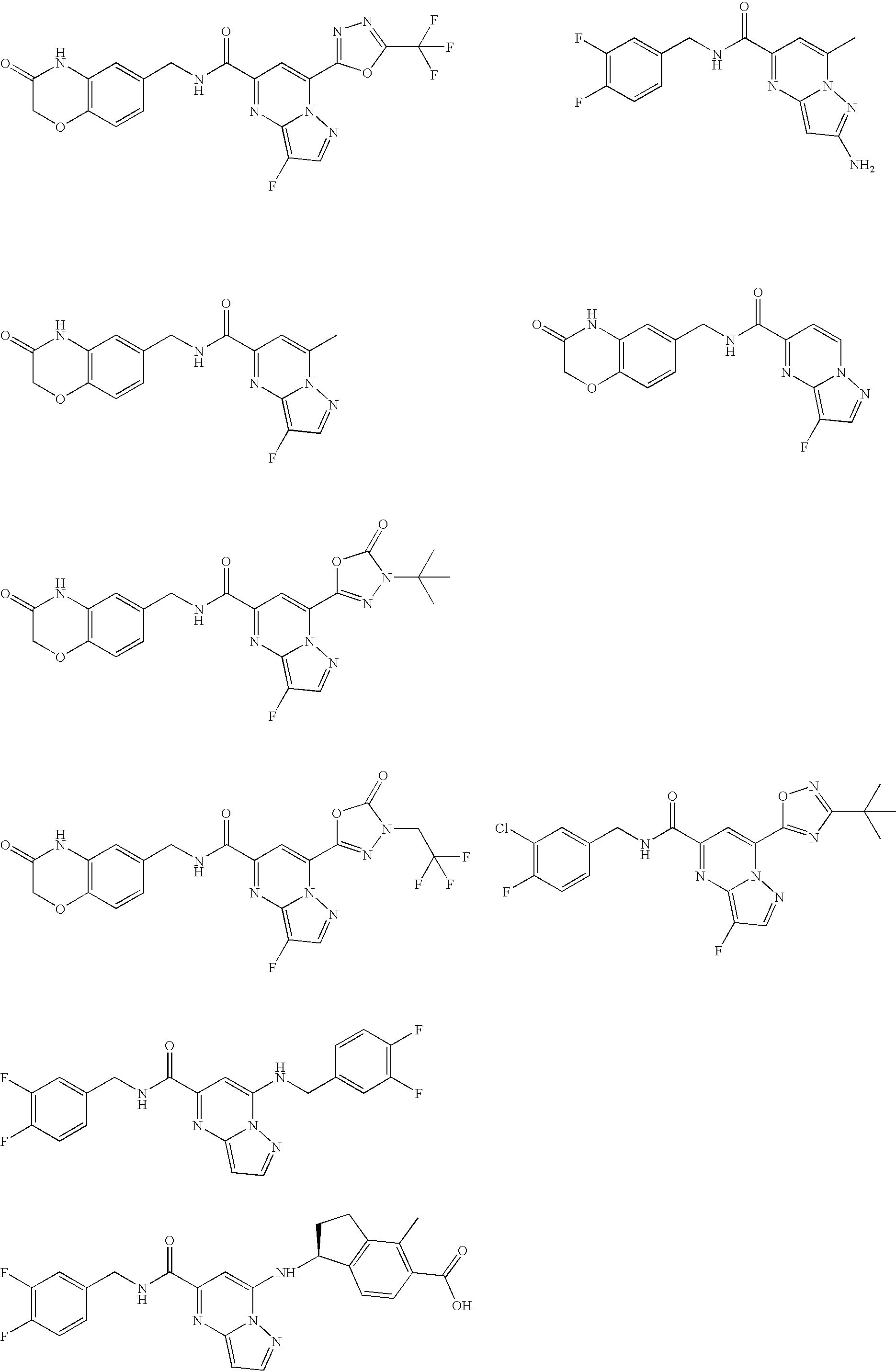 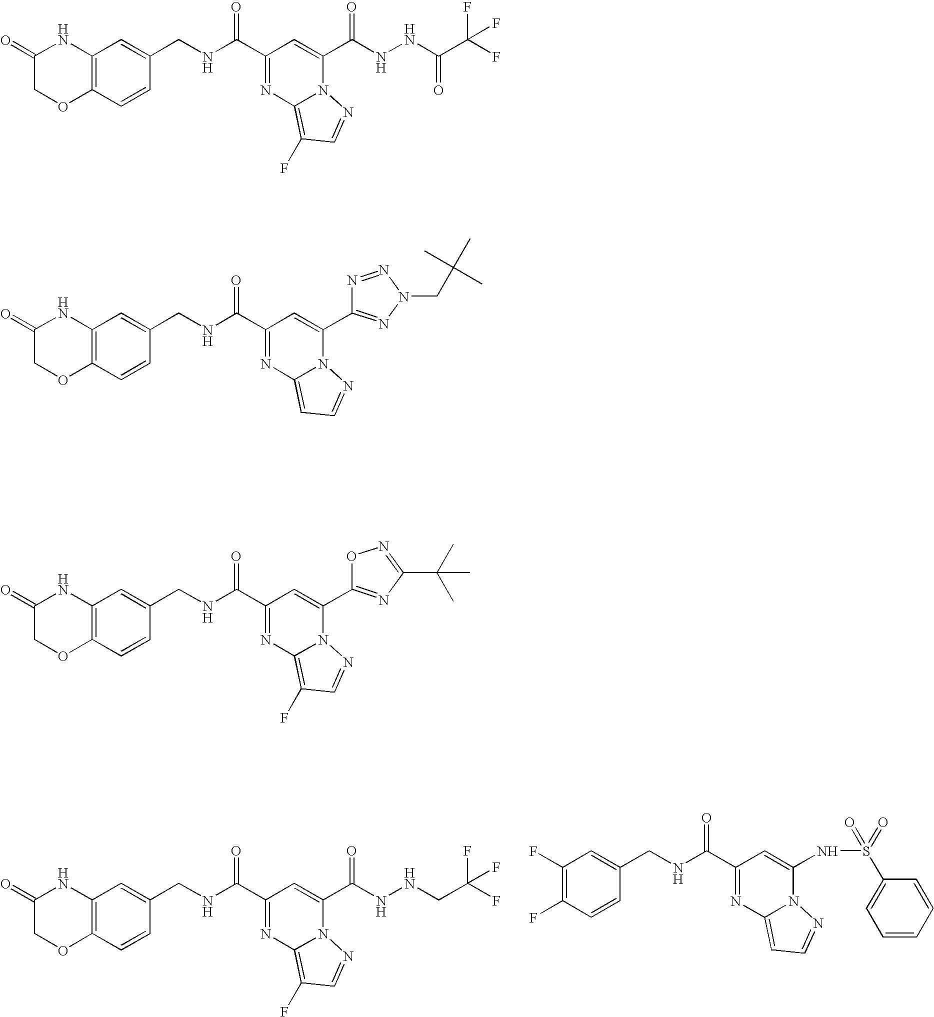 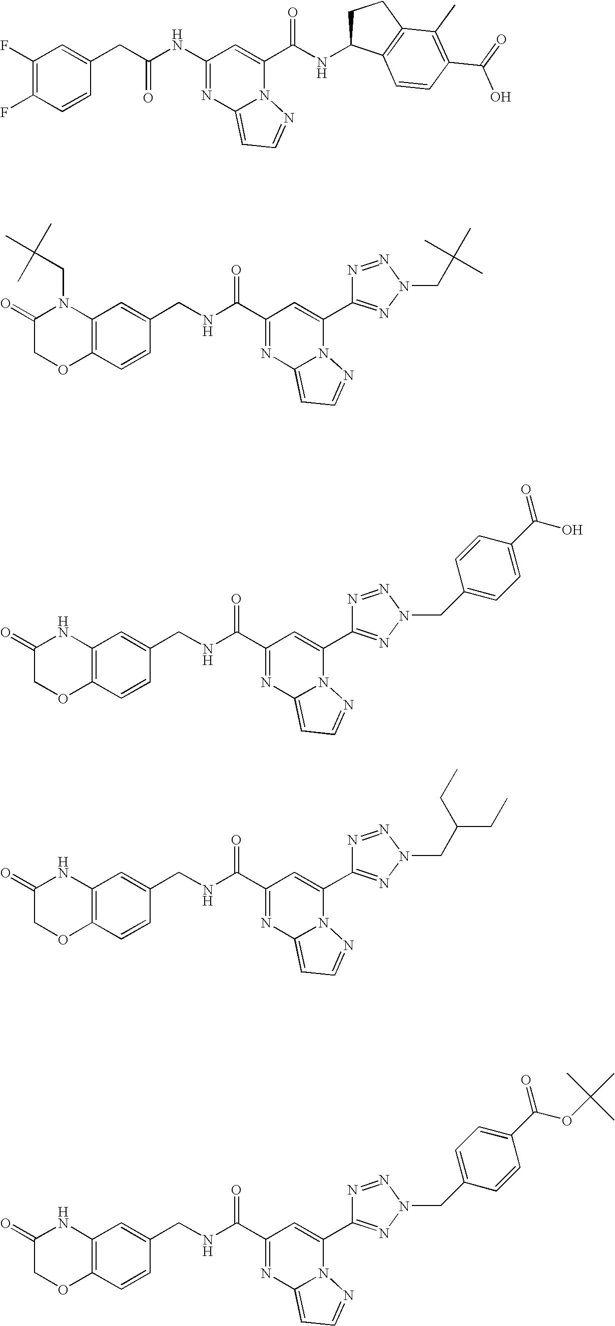 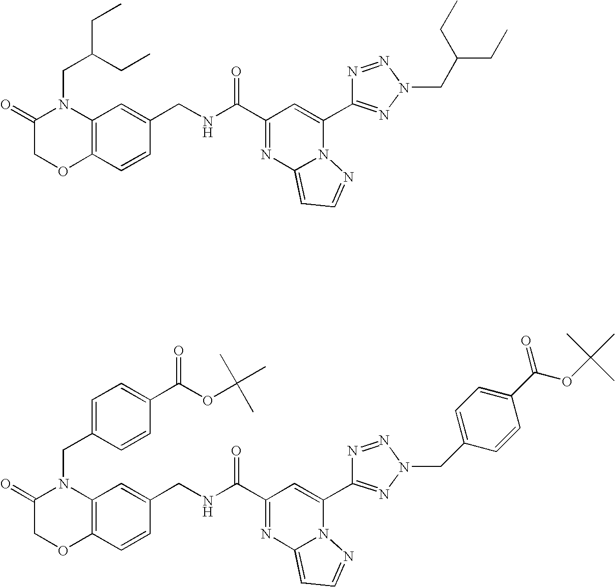 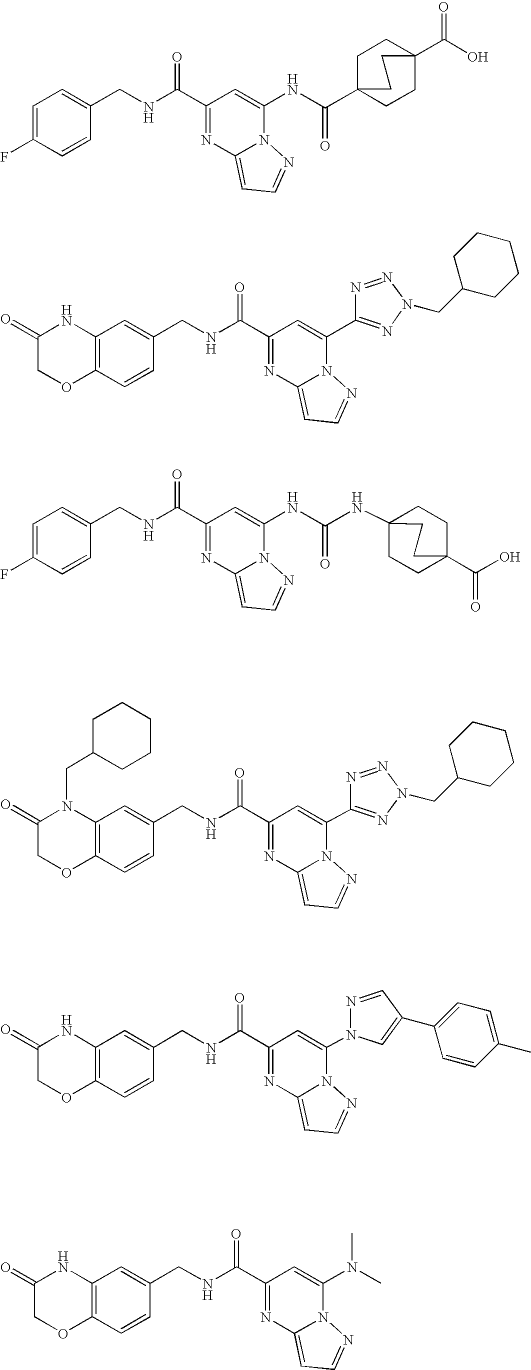 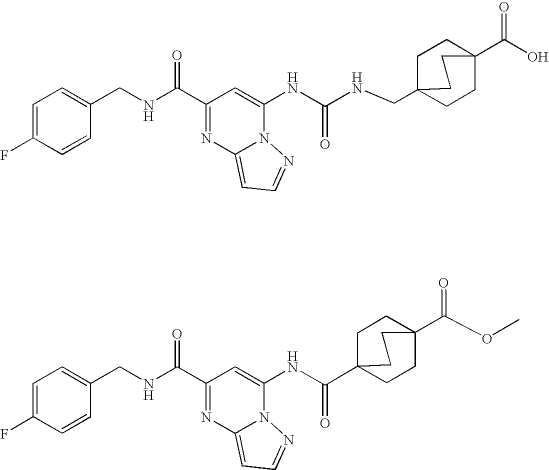 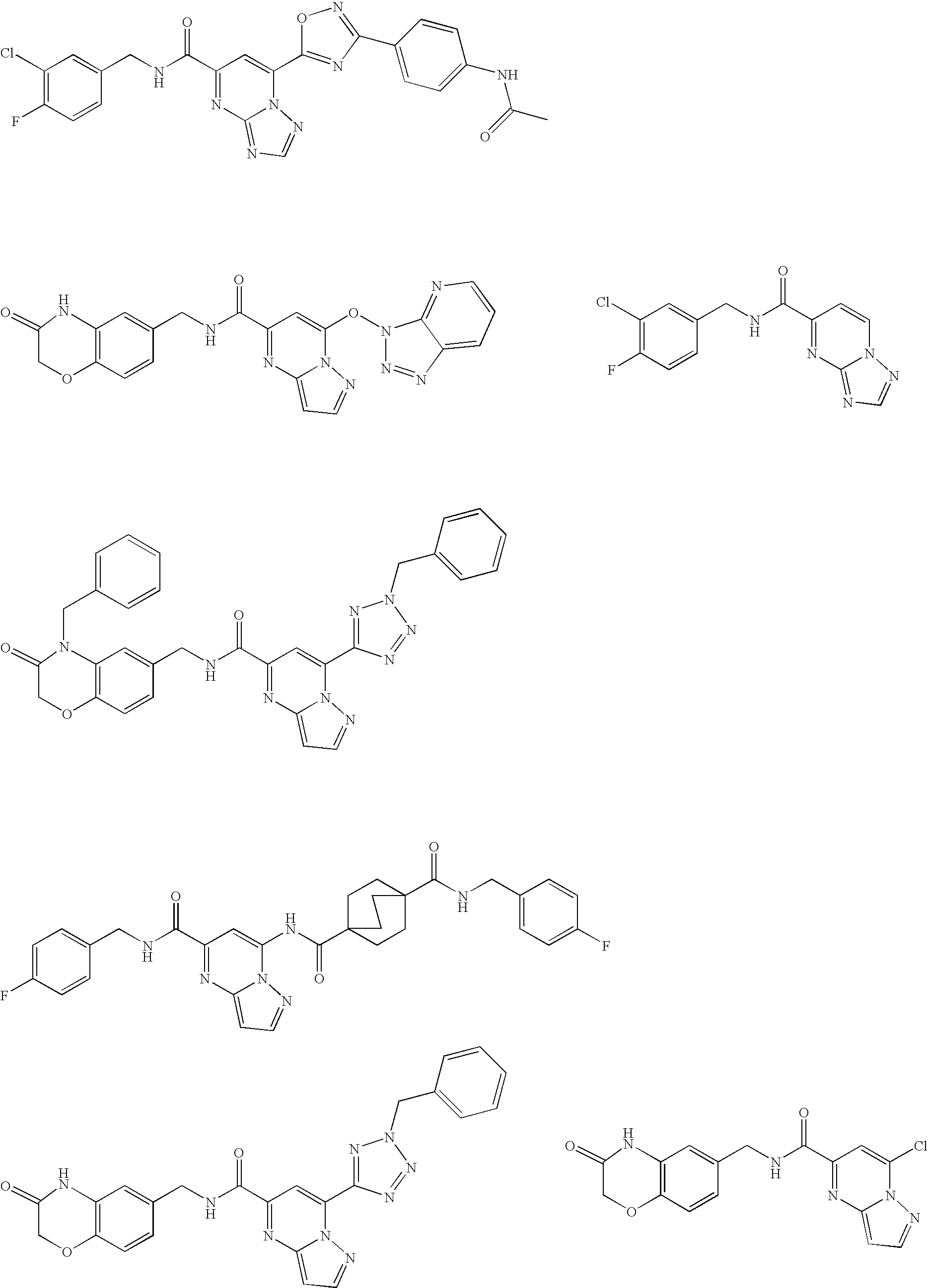 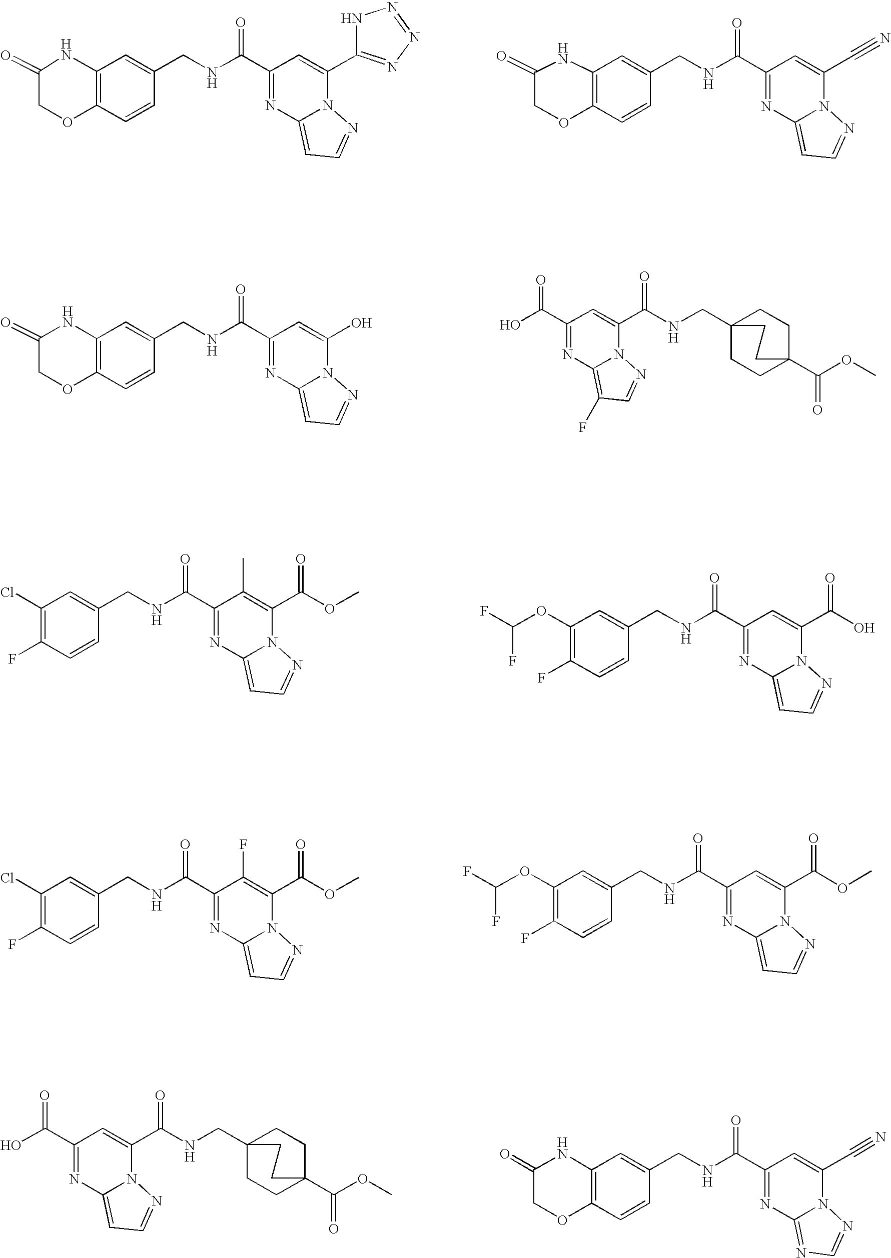 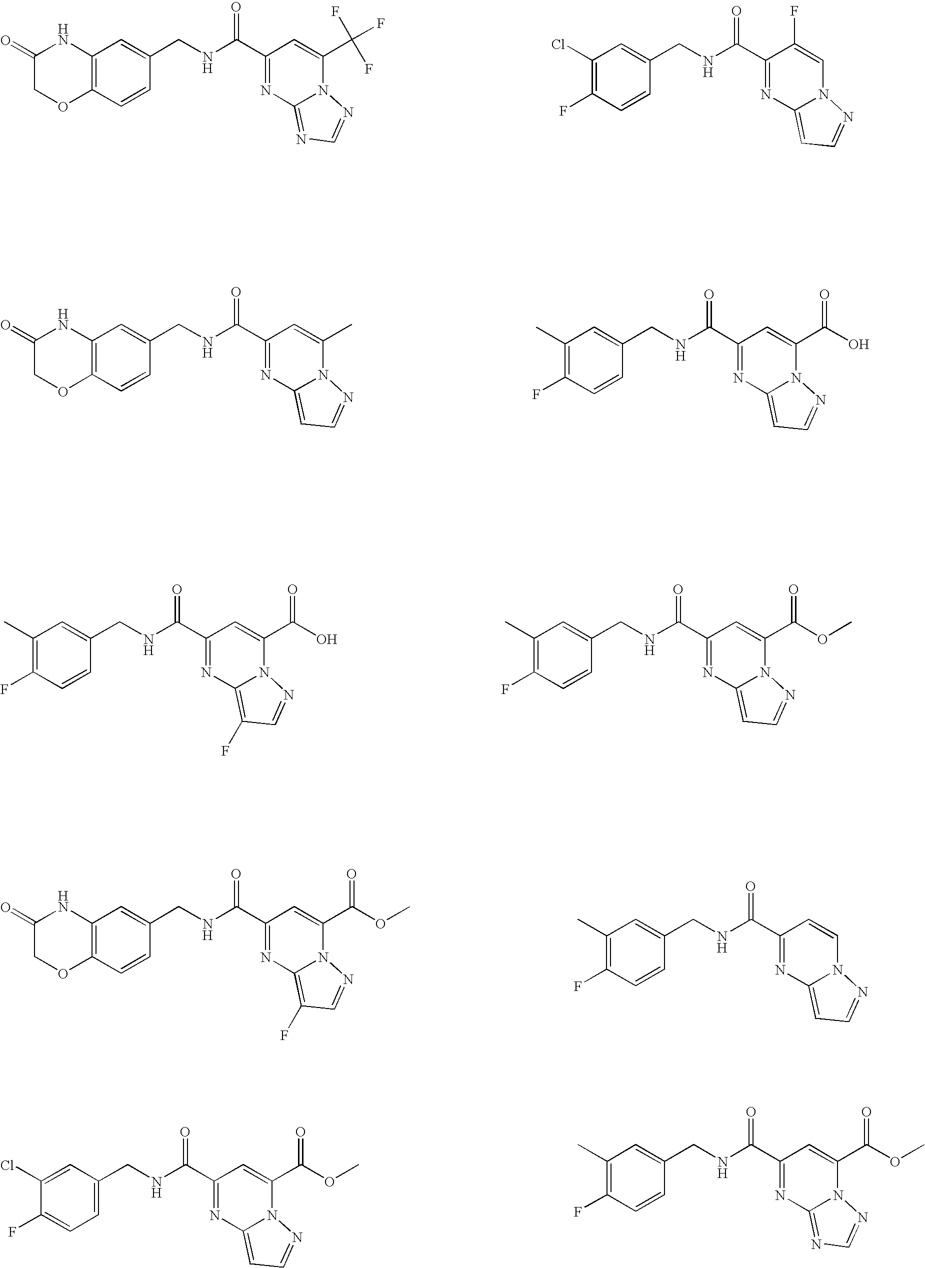 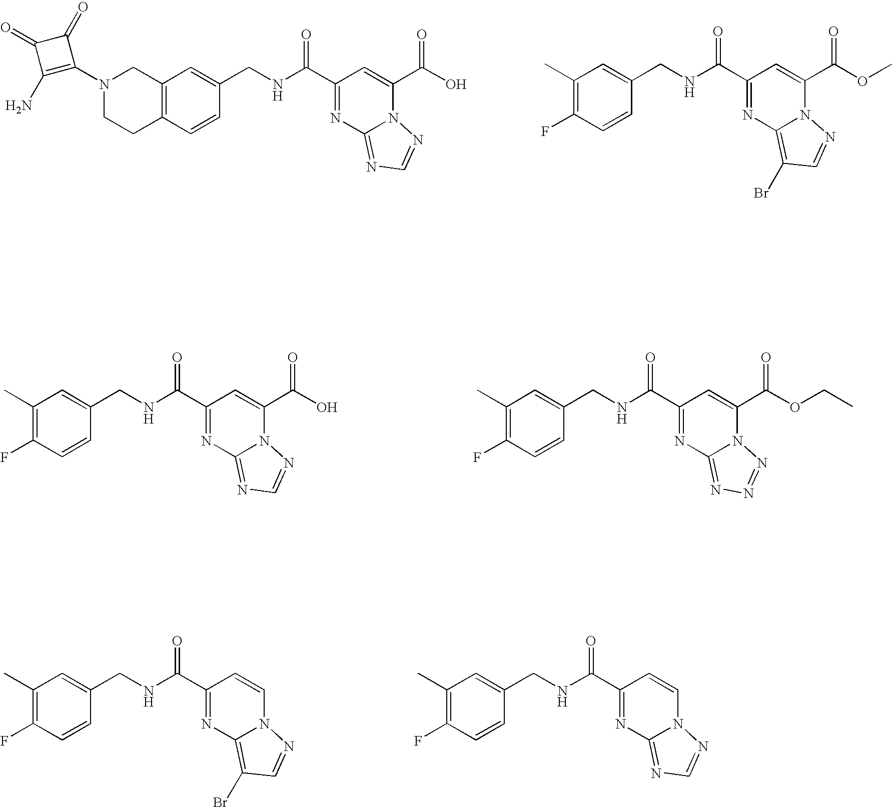
or a pharmaceutically acceptable salt thereof.

36. A pharmaceutical composition comprising an effective amount of the compound of claim 3 and a pharmaceutically acceptable carrier.

37. A method of treating a metalloprotease mediated disease, comprising administering a compound according to claim 3.

38. The method of claim 37, wherein the metalloprotease mediated disease is selected from rheumatoid arthritis, osteoarthritis, inflammation, atherosclerosis and multiple sclerosis.

39. A pharmaceutical composition comprising:
A) an effective amount of a compound according to claim 3; and
B) a pharmaceutically acceptable carrier; and
C) a member selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.
